(19) United States
(12) Patent Application Publication
(10) Pub. No.: US 2015/0368616 A1
(43) Pub. Date: Dec. 24, 2015

(54) METHODS FOR INDUCTION OF CELL FATES FROM PLURIPOTENT CELLS

(71) Applicant: THE CLEVELAND CLINIC FOUNDATION, Cleveland, OH (US)

(72) Inventors: Jan Jensen, Shaker Hts., OH (US); Mike Bukys, Lakewood, OH (US); Xiaoling Qu, Beachwood, OH (US); Solomon Afelik, Cleveland, OH (US)

(21) Appl. No.: 14/767,957

(22) PCT Filed: Feb. 14, 2014
(86) PCT No.: PCT/US14/16448

§ 371 Date: Aug. 14, 2015

Related U.S. Application Data
(60) Provisional application No. 61/764,599, filed on Feb. 14, 2013.

(51) Int. Cl. C12N 5/071; A61K 9/16; A61K 35/39

(52) U.S. Cl. CPC C12N 5/0676; C12N 5/0678; A61K 35/39; A61K 9/1652; C12N 2506/1392; C12N 2501/01; C12N 2500/38; C12N 2500/34; C12N 2501/998; C12N 2501/119; C12N 2501/11; C12N 2501/2311; C12N 2501/21

(57) ABSTRACT

A method of inducing pancreatic fates from human multipotent or pluripotent cells includes obtaining a cell population comprising human multipotent or pluripotent cells and providing the cell population with at least three of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of pancreatic precursor cells from the human multipotent or pluripotent cells.

### CONTINUING APPLICATION DATA

This application claims the benefit of U.S. Provisional Application Ser. No. 61/764,599 filed Feb. 14, 2013, which is incorporated by reference herein.

### GOVERNMENT FUNDING

This work was supported, at least in part, by grant number NIH-DK-2R01-070636 from the Department of Health and Human Services, National Institutes of Health, The United States government has certain rights in this invention.

### BACKGROUND

Islet transplantation has been shown to be an effective treatment for type I diabetes. However the availability of transplantable islets of Langerhans is the limiting factor for this treatment. Human embryonic stem (hES) cells are a promising alternative source of insulin secreting cells. ES cells have an unlimited growth potential coupled with the capacity to differentiate to all cell-types of the adult body. Combined, these qualities imply the possibility of a limitless source of insulin producing β-cells.

ES cells are derived from the inner cell mass of a blastula stage embryo. During embryogenesis this inner cell mass gives rise to the embryo proper and consequently all cells in the adult organisms are derived from it. Gastrulation is the process in which the inner cell mass travels through the primitive streak and establishes the three primary germ layers; ectoderm, endoderm and mesoderm. Distinct structures of the adult organisms are derived from each of these different germ layers, of particular interest here are the endodermally derived organs, notably the thyroid, lungs, pancreas, stomach, liver and intestines.

Human embryonic stem cells are characterized as pluripotent cells capable of generating cells representative of all three germ layers upon differentiation. Pluripotency in hES cultures are defined by the expression of a series of markers including, but not limited to SSEA3, SSEA4, Tra-1-60, Tra-1-81, Oct3/4, Sox2 and Nanog. Until recently the in vitro maintenance of the pluripotent state has been dependent on coculturing hES colonies with feeder cells usually consisting of mouse embryonic fibroblasts (MEFs).

Human embryonic stem cells have the capability of generating all cell types of the adult body, making them a highly valuable potential source of human cells for use in regenerative medicine. The key event(s) restricting their clinical application is the successful differentiation towards clinically relevant cell types and more specifically the ability to generate highly enriched cultures of specific cells or tissue types. This is especially true when the desired cell-type being produced is a pancreatic β cell. There has been considerable effort within the last several years in developing protocols designed to generate insulin producing cells from a pluripotent precursor cultures and while many of these protocols have demonstrated moderate success, none to date have manage to successfully generate large quantities of functional β cells. Currently most protocols suffer from low percentages of endocrine cells, which display coexpression of multiple hormones at there end stage.

Current efforts at the in vitro generation of insulin producing cells from a pluripotent precursor rely on the assumption that culture conditions have to mimic naturally occurring developmental stages, thus trying to direct the differentiation of hES through intermediate developmental stages representative of specific embryonic stages of development. Thus, it has become routine for investigators to describe their protocols in terms of an in vitro induced gastrulation event which generates endodermal cultures that are then regionalized towards a posterior foregut fate enabling the generation of pancreatic endoderm. Pancreatic endocrine subtypes are then derived from this in vitro derived pancreatic endoderm. Therefore, although great effort has been put forth by many groups to generate functional insulin producing cells from ES precursor cultures, the generation of sufficient qualities of β-like cells has not as of yet been realized.

### SUMMARY

Embodiments described herein relate to the induction of pancreatic fates from multipotent and pluripotent cells. The pancreatic fates can include those of the pancreatic endocrine compartment, and specifically include pancreatic insulin producing cells. In some embodiments, a method of inducing pancreatic fates from human multipotent or pluripotent cells includes obtaining a cell population comprising human multipotent or pluripotent cells. The cell population is then provided with or contacted with at least three of, at least four of, at least five of, or at least six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of pancreatic precursor cells from the human multipotent or pluripotent cells.

In some embodiments, the CXCR4 agonist includes at least one of SDF-1 or an SDF-1 peptide analogue; the EGFR agonist includes at least one of EGF, HB-EGF, TGF-α, heregulin, amphiregulin, betacellulin, epigen, epiregulin, or neuregulin 2; the FGFR agonist comprises an FGF growth factor; the Activin receptor agonist or an agent that stimulates SMAD3 comprises at least one of Activin A, TGFβ1, TGFβ2, TGFβ3, GFF8, or GDF11; the IL11R agonist or IL6R agonist comprises at least on one of IL11 or IL6; the notch agonist comprises at least one of Mfap5, Dll1, Jag1, Jag2, Dlk1, or pref1/Dlk1; the BMP inhibitor comprises at least one of Nbl1, Noggin, chordin,chordin-like proteins, follistatin, DAN, sclerostin, decorin, gremlin 1, gremlin2, ceberus, or Dand5; and/or the RXR agonist or RAR agonist comprises retinoic acid.

In other embodiments, the cell population can be provided with the at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for about 5 days to about 3 weeks, or more particularly about 7 days to about 2 weeks.

In some embodiments, the human multipotent or pluripotent cells can include human embryonic stem cells. The human embryonic stem cells can express SSEA3, SSEA4, Tra-1-60, TRa-1-80, OCT3/4, SOX2 and Nanog. The human multipotent or pluripotent cells can also include human induced pluripotent stem cells.

In other embodiments, the pancreatic precursor cells so formed can express Hnf6, Nkx6.1, and Hnf1b. The pancreatic precursor cells can further express Sox9, Pdx1, and FoxA2. In some embodiments, the pancreatic precursor cells can comprise an enriched population of Trunk progenitor cells (TrPCs).

In still other embodiments, at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor can be provided in a cell conditioned medium that is provided to the cell population. The cell conditioned medium can be prepared by exposing a cell growth medium to isolated pancreas specific mesenchymal stem cells to provide the at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor in the cell conditioned medium at a concentration effective to induce differentiation of the multipotent or pluripotent cells.

In some embodiments, the pancreas specific mesenchymal stem cells can include mouse pancreas specific mesenchymal stem cells. The mouse pancreas specific mesenchymal stem cells can be isolated and expanded from pancreas specific mouse mesenchyme of mouse embryos. The mouse pancreas specific mesenchymal stem cells can express CD90 but not PECAM and CD105. In other embodiments, the mouse pancreas specific mesenchymal stem cells do not express InhbA and can be isolated and expanded from adult mouse pancreas.

In still other embodiments, the at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor can be provided in a defined medium that is provided to the cell population. The defined medium can be prepared by adding at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor to a cell growth medium.

In some embodiments, at least one of the Activin A can be provided in the defined medium at a concentration of about 10 pg/ml to about 10 ng/ml, the Hb-EGF can be provided in the defined medium at a concentration of about 1 ng/ml to about 100 ng/ml, the CXCL12 can be provided in the defined medium at a concentration of about 10 ng/ml to about 1 μg/ml, the FGF growth factor can be provided in the defined medium at a concentration of about 5 ng/ml to about 500 ng/ml, the IL-11 can be provided in the defined medium at a concentration of about 10 ng/ml to about 1 μg/ml, the retinoic acid can be provided in the defined medium at a concentration of about 1 nM to about 10 μM, or the BMP inhibitor can be provided in the defined medium at a concentration of about 10 ng/ml to about 100 μg/ml.

Other embodiments described herein relate to a method of producing an enriched population of insulin producing cells. The method includes obtaining a cell population comprising human multipotent or pluripotent cells. The cell population is then provided with or contacted with at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of pancreatic precursor cells from the human multipotent or pluripotent cells. The pancreatic precursor cells so formed are then provided with a maturation medium that promotes differentiation of the pancreatic precursor cells to insulin producing cells.

In some embodiments, the maturation medium can include an agent that increases the generation or stabilization of Ngn3 in the pancreas precursor cells. The maturation medium can also include an agent that inhibits notching signaling of the pancreas precursor cells. The agent that promotes stabilization of Ngn3 or inhibits notch signaling can include at least one of MG132 or DAPT.

In other embodiments, the maturation medium can further include an agent that promotes the generation of intracellular cAMP, such as 8-Br-cAMP, Forskolin, an Adra2a agonist, epinephrine, adrenaline, Galanin, Galr1 activators, Glp1R agonists, Glp1, or extedin. In still other embodiments, the maturation medium can include a Ffar2 agonist, such as a propionate or butyrate. In yet other embodiments, the maturation medium can include a VDR agonist, such as Vitamin D3 or metabolites thereof. The maturation medium can also include variable concentrations or amounts of glucose.

In yet other embodiments, the pancreatic precursor cells differentiated from the human multipotent or pluripotent cells can be provided with a cell growth medium comprising a Wnt signaling pathway activation agent prior to providing the pancreatic precursor cells with a maturation medium that promotes differentiation of the pancreatic precursor cells to insulin producing cells.

Still other embodiments relate to a medium for inducing pancreatic fates from human multipotent or pluripotent cells. The medium can include at least three of, four of, five, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor at amounts effective to allow the differentiation of pancreatic precursor cells from the human multipotent or pluripotent cells. In some embodiments, the CXCR4 agonist includes at least one of SDF-1 or an SDF-1 peptide analogue; the EGFR agonist comprises at least one of EGF, HB-EGF, TGF-α, heregulin, amphiregulin, betacellulin, epigen, epiregulin, or neuregulin 2; the FGFR agonist comprises an FGF growth factor; the Activin receptor agonist or an agent that stimulates SMAD3 comprises at least one of Activin A, TGFβ1, TGFβ2, or TGFβ3; the IL11R agonist or IL6R agonist comprises at least on one of IL11 or IL6; the notch agonist comprises at least one of Mfap5, Dll1, Jag1, Jag2, Dlk1, or pref1/Dlk1; the BMP inhibitor comprises at least one of Nbl1, Noggin, chordin,chordin-like proteins, follistatin, DAN, sclerostin, decorin, gremlin 1, gremlin2, ceberus, or Dand5; and/or the RXR agonist or RAR agonist comprises retinoic acid.

In some embodiments, the human multipotent or pluripotent cells can include human embryonic stem cells. The human embryonic stem cells can express SSEA3, SSEA4, Tra-1-60, TRa-1-80, OCT3/4, SOX2 and Nanog. The human multipotent or pluripotent cells can also include human induced pluripotent stem cells.

In still other embodiments, the medium can be a cell conditioned medium and the cell conditioned medium can be prepared by exposing a cell growth medium to isolated pancreas specific mesenchymal stem cells to provide the at least three of, four of, five of, or six of Activin (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor in the cell conditioned medium at a concentration to induce differentiation of the multipotent or pluripotent cells.

In some embodiments, the pancreas specific mesenchymal stem cells can include mouse pancreas specific mesenchymal stem cells. The mouse pancreas specific mesenchymal stem cells can be isolated and expanded from pancreas specific mouse mesenchyme of mouse embryos. The mouse pancreas specific mesenchymal stem cells can express CD90 but not PECAM and CD105. In other embodiments, the mouse pancreas specific mesenchymal stem cells do not express InhbA and can be isolated and expanded from adult mouse pancreas.

In still other embodiments, the medium can be a defined medium that can be prepared by adding at least three of, four of, five of, or six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor to a cell growth medium. In some embodiments, at least one of Activin A can be provided in the defined medium at a concentration of about 10 pg/ml to about 10 ng/ml, Hb-EGF can be provided in the defined medium at a concentration of about 1 ng/ml to about 100 ng/ml, the CXCL12 can be provided in the defined medium at a concentration of about 10 ng/ml to about 1 μg/ml, FGF growth factor can be provided in the defined medium at a concentration of about 5 ng/ml to about 500 ng/ml, IL-11 can be provided in the defined medium at a concentration of about 10 ng/ml to about 1 μg/ml, retinoic acid can be provided in the defined medium at a concentration of about 1 nM to about 10 μM, or a BMP inhibitor can be provided in the defined medium at a concentration of about 10 ng/ml to about 100 μg/ml.

Still other embodiments described herein relate to a method of inducing organ specific fates from human multipotent or pluripotent cells. The method includes obtaining a cell population of human multipotent or pluripotent cells. A cell growth medium is exposed to isolated organ specific mesenchymal stem cells to prepare a cell conditioned medium. The cell population is provided with the cell conditioned medium for a time effective to allow the differentiation of organ specific precursor cells from the human multipotent or pluripotent cells.

In some embodiments, the organ specific mesenchymal stem cells can include mouse organ specific mesenchymal stem cells. The mouse organ specific mesenchymal stem cells can be isolated and expanded from organ specific mouse mesenchyme of mouse embryos. The organ pancrease specific mesenchymal stem cells can express CD90 but not PECAM and CD105. In other embodiments, the mouse organ specific mesenchymal stem cells do not express InhbA. The mouse organ specific mesenchymal stem cells can be isolated and expanded from adult mouse organ.

Still other embodiments relate to a method of inducing or enriching the formation of pancreatic insulin producing cells by administering a stabilized form of Ngn3 to pancreatic precursor cells. In some embodiments, the pancreatic precursor cells can include TrPCs. In other embodiments, the pancreatic precursor cells can be derived from, or reside in the adult pancreas. In still other embodiments, the stabilized NGN3 can be administered by delivering a vector that encodes stabilized NGN3 and expresses the stabilized NGN3 in pancreatic precursor cells upon delivery to the pancreas in-vivo. In other embodiments, the stabilized NGN3 is administered to the cells by direct protein delivery to the pancreas in-vivo. The direct protein delivery to the pancreas in-vivo can be in a manner where the protein is designed to cross the plasma membrane.

Yet other embodiments relate to a method of treating a subject that includes administering a pancreatic precursor cells produced by the method described herein to the subject. In some embodiments, the pancreatic precursor cells can be provided in an immunoprotective barrier and the immunoprotected cells can be administered to the subject. In some embodiments, the immunoprotective barrier can be a form of microencapsulation, such as a microencapsulation device.

The use of a population of TrPCs and/or pancreatic insulin producing cells as disclosed herein provides advantages over existing methods because the TrPCs and/or pancreatic insulin producing cells can be differentiated from multipotent or pluripotent stem cells, e.g., human embryonic stem cells or induced pluripotent stem cells obtained or harvested from the subject. This is highly advantageous as it provides a renewable source of a definitive population of TrPCs and/or pancreatic insulin producing cells that can be differentiated in vitro or in vivo to insulin producing cells (e.g., pancreatic β-like cells or cells with pancreatic β-cell characteristics) for transplantation into a subject.

### BRIEF DESCRIPTIONS OF THE DRAWINGS

FIG. 1(A-F) shows the derivation of pancreas-specific mesenchymal cultures. Panel A is a schematic outlining the procedure. Pancreas-specific mesenchymal cultures were established from E13.5 litters. Pooled pancreatic rudiments microdissected from E13.5 embryos were washed with cold PBS followed by a combination of mechanical and enzymatic digestion to establish a seeding culture. Cultures were established and maintained in DMEM supplemented with 10% fetal bovine serum, 1% nonessential amino acids and 1% penicillin/streptomycin. Passage of cultures was performed through 0.05% trypsin digestion followed by a 1 to 3 dilution of the cultures. Cultures were generally passaged three times within a 6 day period before being used to condition medium. The unconditioned medium composition was DMEM/F12 supplemented with 1% nonessential amino, 1% L-glutamine and 0.0007% beta mercaptoethanol. Panel B shows 5× brightfield magnification of a typical MEFPancreas culture before being used to condition medium. Panel C-E shows immunohistochemistry of several mesenchymal markers as indicated in the figure. Panel C shows Vimentin (GenScript A01193) expression, panel D shows both the Smooth Muscle Actin (Dako clone1A4) and Nestin (Abcam AB-5968) expression and panel D shows the Fibronectin (NeoMarkers RB-077-AO) and Smooth Muscle Actin expression within a typical Pan-MEF culture. All IHC shown were counterstained with Dapi using Vectashield mounting medium. Panel F shows the percentage of cells present in sample MEFPancreas cultures which express various mesenchymal markers. Percentages were calculated through counting the number of total nuclei present and comparing to the number expressing the individual mesenchymal markers shown.

FIGS. 2(A-X) shows various organ-specific mesenchymal cultures were stained for different mesenchymal markers. Organ specific mouse embryonic fibroblast cultures were established for several different endodermal organs by the method described in FIG. 1. Organ-specific mesenchymal cultures shown include: lung, stomach, pancreas, liver and intestines. Cultures of mouse embryonic fibroblasts (MEF) derived from the body trunk of E13.5 mouse embryos are also shown as an IHC controls. The organ of origin of the different MEF cultures is defined on the left hand side of the panel while the antigen being detected is shown at the top of the panel. Panels A-F show the expression of Smooth Muscle Actin (Dako clone1A4) and Nestin (Abcam AB-5968) within the various cultures. Panels G-L show the expression of Vimentin (GenScript A01193) within the various cultures. Panels M-R show the expression of Fibronectin (NeoMarkers RB-077-AO) and Smooth Muscle Actin within the various cultures. All IHC shown were counterstained with Dapi using Vectashield mounting medium. Panels S-X show bright field images for all of the various cultures described.

FIG. 3 shows RT-PCR comparative analysis between organ-specific cultures and embryonic organs. RNA was extracted from the various organ-specific MEF cultures and from the corresponding embryonic organs. Samples were subjected to 30 rounds of RT-PCR with primers designed to detect transcripts which are either expressed in the epithelial or mesenchymal components of the various organs. Genes being assayed for are indicated to the left of the figure. The tissues or cultures the RNA samples were derived from are as follows; column A is a MEFs culture as derived from the E13.5 body trunk, column B is MEFs derived from E13.5 lung, column C is derived from MEFs derived from E13.5 stomachs, column D is derived from E13.5 pancreas, column E is MEFs derived from E13.5 livers, column F is MEFs derived from E13.5 Intestines. Column G is derived from E13.5 whole lung extracts, column H is derived from whole E13.5 stomach extracts, column I is derived from whole E13.5 whole pancreas extracts, column J is derived from whole E13.5 liver extracts and column K is derived from whole E13.5 intestines extracts.

FIG. 4(A) shows Hox code expression within differing organ-specific MEF Cultures. Organ-specific MEF cultures were derived from E13.5 embryos and maintained in culture for 3 passages (6 days) before RNA was extracted from the various samples. RNA samples were subjected to Mouse-6 Illumina Chips and expression patterns for the various Hox genes were extracted. A schematic representation of the various hox genes is presented at the top of the figure followed by the expression patterns present in the various organ-specific MEF cultures. The organ of origin of the various MEF cultures is indicated at the top left of each Hox-code block and the genes being expressed throughout the various cultures are highlighted.

FIG. 4B shows expression of cell fate determinants of organ-specific mesenchymal embryonic feeders. Illumina Gene expression data are shown for particular transcription factor encoding genes, that are highly differentially expressed between organ-specific MEF cells are shown.

FIG. 4C shows expression of cell fate determinants of organ-specific mesenchymal embryonic feeders. Illumina Gene expression data are shown for particular members of the Collagen-domain containing family.

FIG. 5 shows dendrogram of organ-specific MEFs. RNA extractions derived from third passage organ-specific MEF cultures derived from embryonic lung, pancreas, liver, stomach and intestines were compared to MEFs derived from embryonic body trunks through microarray analysis and the representative dendrogram is presented above. Samples clearly segregated into two distinct branches; one composed of the samples derived from the Stomach-MEF, Pancreas-MEF and Liver-MEF cultures and a second branch composed of by the samples of the Lung-MEF, Intestine-MEF and body trunk-MEF cultures.

FIG. 6 (A-B) shows organ specific mesenchymal cultures cannot sustain pluripotency. Organ-specific mesenchymal cultures were established from E13.5 litters for several endodermal organs (lung, stomach, pancreas liver and intestines). Organ-specific mesenchyme were expanded in short-term cultures (one week or less in culture) and then either the H1 cell line was directly seeded onto irradiated organ-specific mesenchymal cultures or conditioned medium from the respective organ-specific mesenchyme cultures was supplemented onto feeder-free H1 cultures maintained on Matrigel. In both cases RNA extractions of the hES cultures were preformed a week later. Panel A shows transcript analysis of pluripotent markers from the direct seeding of H1 cell line on the respective organ-specific mesenchyme cultures. While panel B shows transcript analysis of pluripotent markers from hES feeder-free system supplemented with conditioned medium obtained from the respective organ-specific cultures. In both cases transcripts representative of pluripotency substantial decreased over the course of the week as compared to the control cultures which were either seeded directly onto MEFs derived from E13.5 body trunks or supplemented with conditioned medium from MEFs derived from E13.5 body trunks. Here and in all cases throughout this application condition medium from the various organ-specific cultures was generated by overnight incubation of DMEM/F12 supplemented 1% non essential amino acids and 0.7% L-glutamine on third passage (6 days ex vivo culture) organ-specific cultures. The conditioned medium obtained from the various samples was supplemented with 2% Knock Out Serum Replacer and filtered before use on pluripotent cultures. Experimental cultures being treated with organ-specific conditioned medium always received fresh medium daily during differentiation experiments.

FIG. 7(A-B) shows organ specific mesenchymal cultures induces forward differentiation. H1 hES colonies were seeded onto Matrigel coated plates and supplemented with conditioned medium derived from the various organ-specific MEF cultures as indicated in the figure legend in panel A. Fresh medium was supplied to the cultures daily for a week at which time RNA was extracted and subjected to QRT-PCR for various markers representative of the germ layers as indicated in the schematic presented in panel B. The strongest responses occurred within markers representative of the mesoderm and endoderm lineages.

FIG. 8 shows differential effects of lung-CM on the forward differentiation of hES versus hES derived definitive endoderm. Pluripotent (H1) cultures were seeded onto matrigel. Conditioned medium obtained from lung-specific mesenchymal cultures were then supplemented onto either pluripotent cultures or pluripotent cultures that were differentiated into a definitive endodermal culture. Conditions used to differentiate pluripotent cultures to a definitive endoderm cultures were as follows: first day cultures were supplemented with a RPMI based medium containing 100 ng/ml Activin A, 8 ng/ml bFGF, 20 ng/ml Wnt3a and 2% w/v BSA and on the following two days cultures were incubated in a RPMI based medium containing 100 ng/ml Activin A, 8 ng/ml bFGF, and 2% w/v BSA. RNA extractions were performed at the beginning of the experiment and at 1, 2, 3 and 4 days after incubation with lung-CM for both set of conditions as indicated on the X-axis of the graphs (where ES stands for embryonic stem cultures and DE stands for definitive endoderm cultures). Real time qPCR was performed on RNA samples with primers designed to amplify the lung specific transcripts FoxP2 and Nkx2.1. All samples were normalized to the expression levels found in the pluripotent cultures.

FIG. 9 shows the effects of one week incubation with lung-CM. Pluripotent cultures (H1) were passaged to Matrigel followed by one week long incubation with lung-CM. Medium was changed daily and RNA was extracted after the seventh day of incubation. Transcript analysis for messages specific to lung (Nkx2.1 and Cldn18), endoderm (Gata6, FoxA1 and FoxA2), intestines (Cdx2), liver (Hhex) and stomach (Fxyd3) are shown with the relative expression of each being compared to hES (H1) transcript levels.

FIG. 10 (A-B) shows pancreas-specific mesenchymal cultures selectively enhances pancreatic features of forward differentiating cultures. To assay whether the observed effects of Pan-CM on the differentiation of hES cultures were specific to mesenchyme derived from the embryonic pancreas, pluripotent cultures were incubated in the presence of organ-specific mesenchymal cultures from three different developing organs; the pancreas, the lungs and the liver. Experimental cultures were incubated with the differing conditioned medium for three consecutive weeks with the respective mediums being replenished daily. RNA extractions were preformed at three weeks and qRT-PCR analysis specifically assayed for messages representative of endoderm generation (FoxA2 and Sox17), pancreatic development (Pdx1, Hnf6 and Glucagon), intestinal development (Cdx2), and liver development (AFP). Cultures which were incubated in the presence of Pan-CM displayed substantial increased expression of both Pdx1 and Glucagon over the cultures incubated with conditioned medium obtained from mesenchyme derived form either the lung or liver.

FIG. 11 (A-D) shows the effects of short-term incubation with Pan-CM. Pluripotent cells were seeded onto Matrigel and incubated for a week in the presence of either CM (conditioned medium obtained from MEF-feeders derived from E13.5 body trunks) or Pan-CM (Pancreas-specific mesenchymal culture conditioned medium). Panel A shows decreased expression levels of transcripts representative of pluripotency in cultures that were maintained in the presence of Pan-CM. Panel B shows increased expression of endodermal markers within cultures maintained in the presence of Pan-CM. Panels C & D show brightfield images (2× and 10× respectively) of hES cultures maintained in the presence of Pan-CM, notably these colonies typically show large regions of differentiation as compared to hES colonies which are maintained CM.

FIG. 12 (A-I) shows the prolonged Incubation with Pan-CM is very efficient at generating endoderm. The H1 cell line was seeded onto matrigel and maintained in the presence of MEF-CM for 3 days before switching to Pan-CM. Control cultures continued being supplemented with MEF-CM for an equivalent period of time. Panel A & B show immunostaining for the pluripotent marker Oct3/4 (Santa Cruz SC-5279) and the endodermal marker FoxA2 (Santa Cruz SC-6554) after a one week incubation with Pan-CM (panel A) or MEF-CM (panel B). Panels C-H show the expression of FoxA2 in both culture systems at 3 weeks of incubation. Panel I shows real time transcript analysis of the pluripotent marker Oct3/4 and the endodermal markers FoxA2, Sox17 and CXCR4 over a three week time course. As evident by the immunostaining in panels A-B and the corresponding RT-qPCR time course analysis there is a sudden loss of pluripotency maintenance when comparing the effects of the Pan-CM to the control MEF-CM cultures. In addition cultures supplemented with Pan-CM take on an increasingly endodermal characteristic over time.

FIG. 13 shows Pan-CM incubation decreases the proliferative capabilities of pluripotent cultures. To measure the effects of Pan-CM medium on the proliferative capabilities of pluripotent cells the hES H1 cell line was passaged to Matrigel followed by seven days of incubation in the presence of conditioned medium from MEFs (MEF-CM) or conditioned medium obtained from pancreas-specific mesenchymal cultures (Pan-CM). The number of cells present per well was calculated at the beginning of the experiment and compared as a percentage of this number at each day after the start of the experiment. Three independent wells for each of the two samples were counted daily up to the one week time point. The number of cells per well was calculated by treating the wells being counted with trypsin for five minutes to detach cells. Collected samples were then counted on a hemocytometer and total number of cells present was divided by the number of cells present per well at the beginning of the experiment. Cultures maintained in organ-specific mesenchymal conditioned medium always displayed decreased proliferative capabilities for at least a week after the initial incubation with said medium.

FIG. 14 (A-F) shows differential morphology between hES cultures supplemented with Pan-CM verses MEF-CM. The H1 cell line was seeded onto Matrigel and maintained in MEF-CM for 3 days before switching to E13.5 Pan-MEF-CM for an additional 3 weeks. Control cultures were continuously incubated in the presence of MEF-CM for an equivalent length of time. Cultures supplemented with Pan-CM formed several cyst-like structures which were absent from control cultures maintained in MEF-CM. Cyst-like structures were gently scrapped off of plates and prepared for sectioning. Briefly, Cysts were incubated overnight at 4° C. in 4% paraformaldehyde followed by a 24 hour incubation in a 30% sucrose solution. Cyst were placed in OCT and frozen for sectioning.

FIG. 15 (A-B) shows Pan-CM mediated forward differentiation of pluripotent cells. Pluripotent H1 cultures were maintained in the presence of either conditioned medium from MEF cultures (CM) or conditioned medium from pancreas-specific mesenchymal cultures (Pan-CM). Triplicate RNA extractions were performed at both one week and two weeks after initiation of the experiment followed by qRT-PCR transcript analysis. At the one week timepoint messengers representative of endoderm (FoxA2 and Sox17) and organ-specific derivatives of endoderm (Pdx1, Cdx2 and Fxyd3) were up regulated. At the two week timepoint several pancreas-specific transcripts were detected including substantial endocrine up regulation as evident by somatostatin and glucagon expression levels. Genes representative of intestinal and liver development, Cdx2 and albumin respectively, were weakly expressed by comparison.

FIG. 16 (A-B)—Reduction of serum levels increases endocrine formation. Pluripotent cultures were passaged onto matrigel and maintained in MEF-conditioned medium for three days before being induced to differentiate by exposure to Pan-CM. Three different conditions were used as outlined in panel A. In the first condition Pan-CM was supplemented with Wnt3a on the first day followed by a 0.2% concentration of FBS for the rest of the reaction. In the second condition Pan-CM was supplemented with 0.2% FBS for the second day on. In the third condition Pan-CM was supplemented with 2% KO serum replacer. Panel B shows the transcript analysis of select genes associated with the cellular fates of mesoderm (MEOX1), Pancreatic trunk progenitor (HNF6 and HNF1-B) and beta cell (Insulin).

FIG. 17 shows comparison between E13.5 & E15.5 Pan-CM. Pancreas specific mesenchymal cultures were established from both E13.5 and E15.5 litters and unconditioned medium (DMEM/F12/1% nonessential amino acids/1 mM L-glutamine/0.0007% Beta mercaptoethanol) was conditioned from passage 3 (6 days in culture) of both respective cultures. The H1 pluripotent cell line was seeded onto Matrigel and maintained in MEF-Conditioned medium for 3 days before being changed to either the E13.5 Pan-MEF-CM or the E15.5 Pan-MEF-CM. Triplicate RNA extractions were performed at days 0 (ES pluripotent culture), 3, 7, 10, 14 and 17 followed by real-time analysis for various transcripts as indicated in the figure. All samples were standardized to the starting ES transcript levels. While there were many similarities between the differentiation events induced by either cultures, notably significant up regulation of several pancreas-specific transcripts, some interesting differences were also noted of particular interest the E15.5 Pan-CM treated cultures were more efficient at generating glucagon expressing cells, while the E13.5 derived Pan-CM displayed a slightly increased insulin expression when compared to the E15.5 Pan-CM treated colonies.

FIG. 18 (A-G) shows Pdx1 expression is localized within cyst structures. The H1 cell line was grown in the presence of Pan-CM for three weeks at which time the cyst structures were hand picked off of the plate and incubated over night in a 4% paraformaldehyde solution. Cysts were moved to a 30% sucrose solution for an additional 24 hours before being embedded in OCT compound block for sectioning. Panels A-C show sections which were stained for FoxA2 (Santa Cruz SC-6554) and Pdx1 (R&D MAB2419). While panels D-F show a higher resolution of Pdx1 staining. Panel G shows a typical transcript analysis for Pdx1 expression over the course of the three week incubation.

FIG. 19 (A-I) shows FoxA2 expression is localized within cyst structures. The hES cell line H1 was grown in the presence of Pan-CM for three weeks and the resulting cyst structures were prepared for sectioning as previously described. Panels A-I demonstrate the widespread expression of FoxA2 (Santa Cruz SC-6554) within these cyst-like structures.

FIG. 20 (A-D) shows Pdx1 expression within Pan-CM induced cyst-structures localized near DBA+ regions. The hES cell line H1 was grown on matrigel in the presence of Pan-CM for three weeks and the resulting cyst-like structures were prepared for sectioning as previously described. Panels A-D show the expression of Pdx1 (Rabbit-a-Idx-1 Hm 253 from J. Habener) and it's proximity to the ductal marker fluorescein dolichos biflorus agglutinin (DBA) (Vector FL-1031).

FIG. 21 (A-I) shows cyst structures display wide-spread Hnf6 expression. The hES cell line H1 was grown in the presence of Pan-CM for three weeks followed by preparation for sectioning as previously described. Panels A-C shows the expression of Hnf6 (Santa Cruz SC-13050) within a cyst structure before sectioning. Panels D-I are Hnf6 stainings performed on cyst structures that were sectioned. In all cases Hnf6 is shown to be widely expressed throughout the resulting cyst-like structures that are generated in the presence of Pan-CM.

FIG. 22(A-D) shows insulin expression within pan-CM induced cyst-structures. The hES cell line H1 was grown on Matrigel in the presence of Pan-CM for three weeks before being prepared for sectioning as previously described. Panels A-D show region present within the cyst-like structures that is Insulin+ (Dako A0564) Glucagon− (Sigma G2654) demonstrating the ability of the Pan-CM to instruct the differentiation of H1 cultures towards a beta-cell like fate.

FIG. 23 (A-C) shows cyst structures display isolated regions that are Cdx2+. The pluripotent hES cell Line H1 was grown in the presence of Pan-CM for three weeks at which time the resulting cyst-like structures were prepared for immunostaining. Panels A-C show regions within Pan-CM derived cysts which display Cdx2 (BioGeneX MU392A-UC) expression.

FIG. 24 (A-B) shows removal of Pan-CM increases terminal differentiation. To ascertain the optimal length of time forward differentiating cultures needed to be exposed to Pan-CM for a commit to the pancreatic lineages to occur, a series of experiments designed to evaluate the effects of removal of Pan-CM at different time points was undertaken. The experiment was conducted as outlined in panel A with pluripotent cultures being exposed to Pan-CM for either 1, 2 or 3 weeks. In addition parallel cultures which were exposed to Pan-CM for 1 or 2 weeks were subsequently maintained in an unconditioned medium (DMEM/F12 supplemented with 1% non-essential amino acids, 0.5% L-glutamine and 10% knockout serum replacer, hence forward referred to as UCM) for an additional week (hence forward referred to as 1+1 and 2+1 cultures respectively) to assay the plasticity of the forward differentiating cultures. Panel B shows select transcript analysis of these differing experimental approaches. Notably the switch from Pan-CM to UCM always correlated with an increase in Krt19 (ductal marker) expression and in both cases the switch to UCM resulted in a significant increase in an endocrine fate, Somatostatin in the case of the 1+1 culture and Glucagon in the case of the 2+1 culture. While cultures incubated in the presence of Pan-CM for a single week loss most their Oct3/4 expression, suggesting a loss of pluripotency, strong evidence of a pancreatic characteristic within these cultures was not achieved until the second week, however this seems to occur regardless of whether the second week of incubation was performed with Pan-CM or UCM.

FIG. 25 (A-B) shows Pan-CM treated hES cultures are competent to form insulin producing cells. We have shown in previously examples that the continued incubation of pluripotent cultures with Pan-CM results in the generation of pancreatic phenotype in forward differentiating cultures. Notably significant up regulation of genes associated with a pancreatic precursor cell (FoxA2, Pdx1), ductal cells (Krt19, Ift88), endocrine precursor cells (Hnf6, Sox9) and endocrine subtypes (Somatostatin, Glucagon) have all been regularly observed and previous described in this application. This current example and ones that follow explore the capacity of a Pan-CM induced forward differentiating culture to adopt an endocrine beta-cell like fate. Panel A shows a schematic representation of the experiment preformed. Pluripotent cultures were incubated in the presence of Pan-CM for 10 days at which time culture conditions were changed to UCM supplemented with either Activin A or DAPT or both. We have previously shown that the late administration of Activin A in directed differentiation protocols greatly enhances insulin production and it is also known that the gamma secretase mediated inhibition of notch signaling increases the beta-cell endocrine subtype. Panel B shows triplicate transcript analysis for the different experimental conditions standardized to ES levels. Markers for the pluripotent state (Oct3/4), Endoderm (FoxA2, Sox17), pancreatic precursor (Pdx1, Ptf1a) and endocrine subtypes (Glucagon, Insulin) are shown. While absolute levels of insulin were not increased dramatically there was a pattern suggesting the switch to the different mediums had an effect of the endocrine subtypes being generated. Notably the inclusion of both Activin A and DAPT not only increased the occurrence of Insulin transcript, but dramatically decreased the occurrence of the glucagon transcript, suggesting that the combination of DAPT and Activin A successfully promoted an endocrine subtype switch.

FIG. 26 (A-G) shows Ngn3-mediated pancreatic progenitor depletion due to endocrine differentiation. A. Schematic outline of the experiment. B, C: Dissected mid-gut region, E18.5 of WT (B) and Ngn3 ON (C) pancreas/stomach/spleen/duodenum. Complete pancreatic agenesis is observed, including gastric/duodenal disjunction, with end-capping of antral, and anterior duodenum. D-G: immunofluorescence analysis, dorsal pancreatic region at E11.5, using markers shown. D, E: conversion of pancreatic Pdx1-expressing progenitor cells (D) into glucagon-expressing endocrine cells (E) is accompanied by mitotic arrest (pHH3, E). (F,G) Mucin 1 (Muc1) characterizes emerging apical polarity of carboxypeptidase A (CPA)-expressing pancreatic progenitor cells in WT (F), such are not observed in Ngn3 ON pancreas (G).

FIG. 27 (A-F) shows conditional activation of Ngn3 leads to early pancreatic progenitor depletion. A-D: Immunofluorescence analysis of dorsal pancreatic region of WT and Ngn3 ON (DTG) embryos at E11.5, using markers shown. Extensive Ngn3 expression is uniform in DTG (B), as compared to WT (A), in which only few Ngn3-expressing nuclei are observed. A concurrent loss of Sox9 expression is observed in DTG pancreas (D) when compared to the WT (C), but the trunk progenitor marker Nkx6.1 remains expressed (F).

FIG. 28 (A-F) shows the effects of conditional activation of Ngn3 on early pancreatic development. A: schematic outline of the experiment. B. qRT-PCR evaluation of the conditional activation of Ngn3 in individual DTG embryos at E10.5 and E11.5, compared to WT littermates. C, D: Pdx1/Glucagon immunostaining, E12.5. E, F: Sox9/DBA immunostaining, E12.5.

FIG. 29 (A-G) shows delayed activation of Ngn3 induces pancreatic endocrine and ductal cell formation. A. Schematic outline of the experiment. B, C: Dissected mid-gut region containing stomach (st), spleen (sp), dorsal pancreas (dp), ventral pancreas (vp) and duodenum (duo). D-G: immunofluorescence analysis of E14.5 WT pancreas and Ngn3 Delayed ON littermate using markers as shown.

FIG. 30 (H-N) shows delayed activation of Ngn3 induces pancreatic endocrine and ductal cell formation. H-M: immunofluorescence analysis of E14.5 WT pancreas and Ngn3 Delayed ON littermate using markers as shown. Insert in L shows the presence of mature Pdx1+/insulin+ cells in WT pancreas. N: morphometric analysis of relative cell areas of DBA, glucagon, amylase, insulin, compared to WT littermates (n=3/condition).

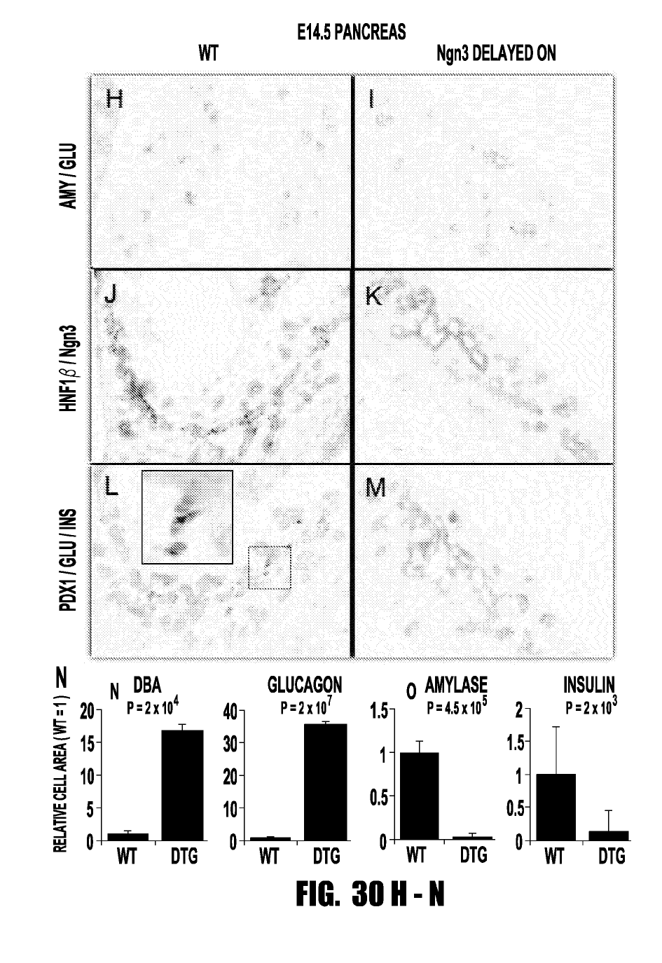

FIG. 31 (A-H) shows Ngn3 controls pancreatic patterning prior to the induction of terminal fates. A, B: immunofluorescence analysis of mucin and carboxypeptidase 1 (Cpa1) in WT and Ngn3 Delayed ON littermate at E12.5. C, D: similar, analysis of E-cadherin and Ptf1a. E, F: expression of Nkx6.1 in WT and Ngn3 Delayed ON E12.5 pancreas differs qualitatively by the presence of Nkx6.1 expressing cells at the epithelial/mesenchymal boundary (F). In WT pancreas, Nkx6.1 is at this time point excluded from distal cells (E). G: morphometric analysis of relative cell areas of Ptf1a and Nkx6.1, compared to WT littermates (n=3/condition). H. qRT-PCR analysis of select pancreatic genes.

FIG. 32 (A-L) shows a brief exposure to Ngn3 results in centralized patterning and ductal cell differentiation, disproportionately to induction of endocrine cell differentiation. A: schematic outline of the experiment. B-K: immunofluorescence analysis of E14.5 WT and Ngn3 Brief ON DTG pancreas with markers as outlined on the figure. L: morphometric analysis of relative cell areas of DBA, glucagon, Cpa1, insulin, compared to WT littermates (n=3/condition).

FIG. 33 (A-H) shows Notch/Hes1 controls Ngn3 protein stability. A-H: double immunofluorescence analysis of Sox9/Hes1 (A-D), and Ngn3/Hes1 (E-H) of WT pancreas from E11.5-E13.5 (A-C, E-G), and Ngn3 Delayed ON pancreas at E11.5 (D, H). White arrows in E, F denote Ngn3+ cells. The arrows in G denote Ngn3/Hes1-double negative epithelial-type cells.

FIG. 34 (I-L) shows Notch/Hes1 controls Ngn3 protein stability. I. Protein stability and half-life assessment of Ngn3 in HepG2 cells. HepG2 cells were transfected with pTRE2-Ngn3 and pCMV-rtTA, treated with Doxycycline for 24 hr, and on the subsequent day pre-exposed to the proteasomal inhibitor MG132 for 5 hr. At time t=0, protein synthesis was inhibited by cycloheximide (CHX). Western blot using anti-Ngn3 antibodies are shown; b-actin served as a control, and remains stable. MG132 treatment leads to Ngn3 protein accumulation at t=0, and strongly stabilizes the protein, which rapidly decays in non-treated cells. J. Experiment conducted similarly as in I, but CMV-driven versions of Notch1 and Notch2 intracellular domains (N1ICD, N2ICD respectively) (left), or dominant-negative mastermind-like1 (DN-MAML1) were co-transfected with pTRE2-Ngn3. The left-most blot is overexposed to reveal the lower level of accumulated Ngn3 protein in presence of N1ICD, N2ICD, and the loss of Ngn3 upon CHX treatment. The analysis was done 1 hr post CHX treatment. The right-most blot shows the relative increase in Ngn3 protein in cells expressing DN-MAML1; Ngn3 remains stable. K. Mouse pancreatic E14.5 explants were subjected to short-term treatment of CHX (1 hr) and MG132 (5 hr pre, and post-culture) as indicated. Total protein was isolated and subjected to anti-Ngn3 Western blotting. Levels of Ngn3 increase in presence of MG132, and decrease in response to CHX. Ngn3 protein levels increase correspondingly in presence of DAPT, a condition where the protein remains stable (CHX+DAPT lane). L. Stability measurements of Ngn3 in the presence of various cellular regulatory proteins. The experiment was conducted as in I and J. Decay scans (right) provide a measure of the decay rate of Ngn3. In all cases, the t=0 Ngn3 levels prior to CHX treatment is paralleled by the relative half-life of the protein. Destabilizing conditions include N1ICD, N2ICD, and Hes1. Stabilizing conditions include DN-MAML1 and DAPT-treatment. C-terminally truncated Hes1 (Hes1ΔC), and Skp2 presence do not affect Ngn3 stability. Hes1 is able to destabilize Ngn3 in the presence of DN-MAML1.

FIG. 35 (A-C) shows notch signaling inhibition promotes beta- and acinar-cell formation, at the expense of ductal cell development. A, B: mouse WT pancreatic explants (E12.5) were cultured for 4 days in the absence/presence of 100 mM DAPT. Sections were stained against Mucin1, carboxypeptidase1 (CPA) and glucagon (Glu). C. qRT-PCR analysis of explants performed as in A, B. Pancreatic marker genes were assessed and fold-changes to WT explants treated with DMSO were calculated by DDct method, using GAPDH mRNA as internal standard.

FIG. 36 (D-K) shows notch signaling inhibition promotes beta- and acinar-cell formation, at the expense of ductal cell development. D-K. Ngn3 Delayed ON pancreas (E12.5, doxycycline was provided at E7.5, E9.5 as in FIG. 2A) was isolated and subjected to 4 days of explant culture with and without DAPT as described in A,B. Immunofluorescence analyses of explant pancreas using select pancreatic markers, after 4 days in culture. D′: insert image showing the expression of glucagon.

FIG. 37 (A-B) shows differential effects of several compounds on endocrine development. As a means to optimize the differentiation of insulin producing cells a two stage protocol was used in which pluripotent cultures were incubated in the presence of Pan-CM for 8 days as a means of generating pancreatic precursors cellular population. The medium these cultures were incubated in were subsequently changed to a medium designed to enhance differentiation of a pancreatic precursor towards a beta-cell terminal fate. All second stage experiments were performed in a medium composed of DMEM/F12 containing 12 mM Glucose supplemented with 1% B27, 1% Pyruvate, 1 uM Alk5 Inh II, 500 ng/ml Noggin and 0.5 uM (1,25 OH2)-Vit D3. In addition MG132, DAPT, forskalin and sodium propionate were added in separately and in combinations as indicted in the schematic in panel A. Panel B shows the transcript analysis of markers important in endocrine differentiation.

FIG. 37C shows expression of relevant receptors for maturation medium signaling components in mouse pancreatic development, mouse insulinoma (bTC), mouse glucagonoma (aTC), adult mouse islets from CD1 strain, and mouse pancreatic adenocarcinoma (mPAC). Data were obtained from Affymetrix Microarray MOE-430 done RNA from isolated embryonic samples. Ffar2 (free fatty acid receptor 2), and Vdr (receptor for 1,25 (OH)2- Vitamin D3, Galr1 (Galanin receptor), Adra2a (GPCR family receptor for epinephrine/adrenaline, linked to adenylcyclase activity, and PKA activation.

FIG. 37D shows expression of SDF1/CXCL12 cognate receptors in mouse pancreatic development, mouse insulinoma (bTC), mouse glucagonoma (aTC), adult mouse islets from CD1 strain, and mouse pancreatic adenocarcinoma (mPAC). Data were obtained from Affymetrix Microarray MOE-430 done RNA from isolated embryonic samples.

FIG. 37E shows expression of EGFR family members in mouse pancreatic development. Data were obtained from Affymetrix Microarray MOE-430 done RNA from isolated embryonic samples.

FIG. 37F shows expression of IL6R (Gp1130 coupled) family members in mouse pancreatic development. Data were obtained from Affymetrix Microarray MOE-430 done RNA from isolated embryonic samples.

FIG. 38(A-B) shows maturation medium improves endocrine formation. To successfully differentiate an insulin producing cell a two stage approach was developed. Schematically represented in panel A, pluripotent cultures were treated with Pan-CM for two weeks followed by a switch to a maturation medium (abbreviated MM within panel B) composed of DMEM/F12 with 12 mM glucose supplemented with 5 uM DAPT, 0.5 uM Vit D3, 1% B27, 1 uM Alk 5 Inhibitor and 1 mM Sodium Propionate. In addition cultures were treated with the proteasome inhibitor MG132 at 10 uM for a 4 hour pulse daily for the length of the experiment. We have previously shown that MG132 mediated inhibition of protein degradation promotes sustained Ngn3 signaling resulting in increased endocrine differentiation. Panel B shows transcript analysis for several genes associated with endocrine development (Pdx1, Nkx6.1, Hnf6, Ngn3, Pax4, Mafa, Glucagon, Insulin and Slc30A8) as well as a few genes associated with the stray fates of intestinal (Cdx2, Gucy2c) and liver (AFP, Albumin) development. Substantial increases in several of the endocrine specific genes are observed.

FIG. 39A shows FGF and Tgfβ protein family expression patterns. FIG. 39A shows the expression pattern of the various members of the FGF and the TGF-beta families within ^{MEF}Pancreas as compared to mouse embryonic fibroblasts (MEF) derived from E13.5 mouse embryo body trucks. Triplicate RNA samples were collected from both MEFPancreas and MEF cultures for microarray analysis on Mouse Ref 6 Illumina microarray chips. Expression patterns were determined using GenomeStudio software. The FGF family members which were expressed within the pancreas specific MEF cultures were FGF13, though FGF10 expression was noted in other Pancreas-specific MEF cultures. The only TGF-β superfamily member which was shown to be expressed within pancreas-specific MEF cultures was Inhibin A (Activin A). Inhibin A was always found to be strongly expressed within these pancreatic derived primary cultures.

FIG. 39B shows the expression pattern of the various members of the DAN and EGF protein families within ^{MEF}Pancreas as compared to mouse embryonic fibroblasts (MEF) derived from E13.5 mouse embryo body trucks. Triplicate RNA samples were collected from both ^{MEF}Pancreas and MEF cultures for microarray analysis on Mouse Ref 6 Illumina microarray chips. Expression patterns were determined using GenomeStudio software. The only DAN protein family member that was expressed within the pancreas-specific mesenchyme was Nbl1. The EGF protein family member that was expressed the highest within the pancreas specific mesenchymal cultures was Hb-EGF.

FIG. 39C shows the expression pattern of the various members of the chemokine and interleukin protein families within ^{MEF}Pancreas as compared to mouse embryonic fibroblasts (MEF). Both MEF cultures were derived from E13.5 mouse embryos. Triplicate RNA samples were collected from both ^{MEF}Pancreas and MEF cultures for microarray analysis on Mouse Ref 6 Illumina microarray chips. Expression patterns were determined using GenomeStudio software and graphed using Prism graphing software. The only protein from the chemokine family that was significantly expressed within the pancreas-specific mesenchymal cultures was CXCL12. The interleukin protein family member that was expressed strongest within the pancreas-specific mesenchymal cultures was Ill-11.

FIG. 40 shows secreterome analysis of pancreas-specific mesenchyme conditioned medium. As a means of providing a complimentary and independent analysis of the proteins being expressed within pancreas-specific mesenchymal cultures, conditioned medium obtained from a 24-hour incubation on Pan-MEF culture was subjected to LC-MS analysis at the Case Western Reserve Center for Proteomics and Bioinformatics. Two experimental approaches were performed bottom up protein identification approach on the non-fractioned sample and a bottom up SDS-PAGE fractionated approach. FIG. 40 shows the secreted or membrane bound proteins detected through the non-fractionated approach as analyzed using Ingenuity™ software. The raw data used to perform this analysis is presented elsewhere in this patent.

FIG. 41(A-B) shows defined medium replicates effects of Pan-CM. To determine the functional components present within Pan-CM, microarray data was analyzed using GenomeStudio software as shown in example 12. The proteinous components which were differentially expressed within the pancreas specific mesenchymal cultures were assayed in combination and compared to the differentiation effects observed using Pan-CM. Pluripotent cultures were either supplemented with Pan-CM or a DMEM/F12 based medium supplemented with 10 ng/ml Hb-EGF, 100 ng/ml SDF1, 100 ng/ml ActA, 50 ng/ml FGF10, 100 ng/ml Il11, 100 ng/ml Noggin, 2 uM RA and 2% Knock Out serum replacer, the latter mixture is referred to as defined medium (DM) in FIG. 40B. Differentiation experiments were performed in triplicate and ran for 5 days at which time RNA extractions were evaluated by qRT-PCR. Panel B shows the similarities between the two reactions with emphasis on pancreas-specific transcripts.

FIG. 42 (A-C)—Nbl1 inhibits BMPs. Pluripotent cells were incubated in the presence of Bmp4 alone or in combination with Nbl1 or Noggin for 24 hours. The following day cultures were assayed for downstream targets of Bmp4 signaling. Panel A shows a Western blot analysis of Bmp4 induced phospho smad 1/5/8 activation in the presence of Nbl1 or Noggin from representative protein extractions. Note the reduced expression of the phosphorylated smads for both Nbl1 and Noggin. Panel B shows the expression of the phosphorylated smad 1/5/8 and the loss of its expression in cultures treated with Nbl1 or Noggin. In addition the down stream activation of AFP was loss in the cultures treated with Nbl1 and Noggin. Panel C shows the RNA analysis of ID factors which are the direct targets of Phospho Smad 1/5/8 and demonstrates a loss of their activation in the presence of Nbl1 or noggin.

FIG. 43(A-H) shows inclusion of defined medium components enhance the generation of pancreatic endoderm in a directed differentiation protocol. To assay the individual protein components within the cell conditioned medium defined by the microarray analysis, the individual components were included throughout stages 2 and 3 of a directed differentiation protocol. Panel A shows a schematic outlining the experiment. Panels B-G show immunohistochemical stainings for Pdx1 and Hnf1-beta for the control reaction (panel B, D and F) and the reaction performed with the addition of Hb-EGF (panels C, E and G). Panel H shows qRT-PCR analysis of the various experiments performed with the added protein component indicated on the X-axis. The control reactions are stage3 cultures that did not have any change in the protocol and all reactions were compared to transcript levels present within undifferentiated hES cultures. Notably all components tested increased Pdx1 levels according to transcript analysis, however this effect was the greatest when Hb-EGF or Fgf10 were included in the reactions.

FIG. 44 (A-B)—Cell conditioned medium improves over the Pan-CM induction of pluripotent cells. Pluripotent cultures were passaged onto matrigel and maintained in the presence of MEF conditioned medium for 3 days to maintain pluripotency. They were next subjected to either Pan-CM or our cell conditioned medium supplemented with either 2% KO serum replacer or low concentrations of FBS as defined in panel A. The addition of Wnt3a on the initial day of differentiation was also assayed. Panel A shows a schematic of the experiments performed and compares three sets of conditions using either cell conditioned medium as described in panel A or Pan-C. Panel B shows the transcript analysis of several key genes associated with the fates of endoderm (FOXA2 and SOX17), pancreatic progenitors (PTF1A, PDX1 and NKX6.1), and endocrine cells (GLUCAGON). Note the occurrence of the trunk progenitor cell is best defined with the loss of NKX6.1.

FIG. 45 (A-C)—Generation of pancreatic endoderm using informed cell conditioned medium. Pluripotent cultures were maintained either in the presence of the cell conditioned medium (as defined in panel A) or a basal medium supplemented with 2% KO for three weeks followed by an immunohistochemical analysis (as described previously) of markers for pancreatic endoderm. It was noted that widespread expression of the endodermal marker FOXA2 was observed in cultures maintained in the presence of the cell conditioned medium were FOXA2 expression was extremely limited in cultures maintained in the basal medium supplemented with 2% KO. Panel A shows a schematic outlining the conditions used throughout the experiment. Panel B shows a representative staining of FOXA2 expression throughout both cultures with a region of PDX1+ expression shown. Panel C shows another representative region of the cultured maintained in the presence of the cell conditioned medium with a higher magnification of the FOXA2 expression.

FIG. 46 (A-B)—Stage 4 administration of defined components. Pluripotent cultures were passaged onto matrigel and maintained in the presence of MEF-conditioned medium for three days before being subjected to a directed differentiation protocol. At the end of stage 3 the individual components of the cell conditioned medium were supplemented into the cultures. After 4 days RNA was extracted from the various cultures and subjected to RT-qPCR analysis for key markers. Panel A shows a schematic outlining the directed differentiation protocol used throughout this set of experiments. Panel B shows the transcript analysis of key pancreas-specific factors.

FIG. 47(A-J) shows notch signaling is required for endocrine differentiation. (A) Schematic representation of the Notch transcriptional complex and truncated mastermind like-1 leading to a dominant negative effect. (B) Schematic representation of transgenic overexpression of dnMAML1. Pdx1 promoter driven expression of tTA results in transcriptional activation of the dnMAML1-IRES-nGFP mRNA. Presence of doxycycline prevents tTA mediated expression of the transgene. (C) mid-gut dissection of wild type (Wt) and dnMAML1;tTA double transgenic embryo (DTG) at E18.5. (D) Image in panel C under fluorescent light. Notice the fluorescence of the DTG pancreas. (E-H) Immunofluorescence staining for pancreatic differentiation markers in Wt (E, G) and DTG pancreas (F, H) at E18.5. Morphometric quantification of the exocrine tissue based on amylase (I), the endocrine compartment based on endocrine specific gene expression and the duct cells marked by the duct specific lectin DBA (J). Values are presented as mean±S.D. *: p<0.05; **: p<0.005; n.s.: not significant. N=3 for all analyses in (I) and (J). Scale bar: 50 μm.

FIG. 48(A-E) shows Acinar cell specific expression of dnMAML1 transgene. Immunofluorescence staining of amylase and insulin in E18.5 wild type (A) and DTG (B) as well as the ductal markers HNF1β and DBA in wild type (C) and DTG pancreas (D). In B and D, the expression of EGFP depicts distribution of transgene expression relative to pancreatic differentiation makers. Relative expression of EGFP to differentiation markers are: GFP/Amylase—0.69±0.10; GFP/Insulin—0.048±0.031; GFP/HNF1β—0. Quantification of proliferation rates of E13.5 Pdx¹⁺ Wt and DTG; Nkx6.¹⁺ Wt and DTG; and transgene negative (GFP−) or positive (GFP+) epithelial cells in the DTG pancreas based on pHH3 immunofluorescence staining (E). n=3. Values are presented as mean±S.D. Scale bar: 50 μm. n.s.: p-value is not statistically significant.

FIG. 49 (A-S) shows the effect of dnMAML1 on pancreatic epithelial patterning. Fluorescence visualization of transgene derived nEGFP expression relative Immunofluorescence staining of Ptf1a (A-C), Nkx6.1 (E-G), HNF1β (I-K) and Sox9 (M-O). Quantitative analysis of the percentage of nEGFP cells that are positive or negative for Ptf1a (D), Nkx6.1 (H), HNF1β (L) and Sox9 (P). Schematic representation of isolation of E13.5 pancreatic EGFP positive and EGFP negative epithelial cells by flow cytometry (Q). Dissociated embryonic pancreas tissue was stained with APC-conjugated anti-E-cadherin prior to sorting. x-axis (FITC) indicates GFP intensity and y-axis (APC-A) indicates intensity of epithelial staining by APC-conjugated E-cadherin (R). (S) qRT-PCR analysis of pancreatic progenitor markers in EGFP positive epithelial cells expressed as fold increase or decrease relative to non-EGFP epithelial cells. EGFP⁺ cells have 790.8±71.6 fold dnMAML1 over EGFP− cells. White arrows indicate GFP cells that are negative for a given marker gene; arrowheads indicate EGFP+ cells that are positive for pancreatic marker genes; black arrow indicate EGFP⁺ cells that express medium level of Sox9. Values are presented as mean±S.D. Scale bar: 50 μm.

FIG. 50(A-T) shows suppression of Nkx6.1 expression by dnMAML1 precedes its effect on Ptf1a. Fluorescence visualization of the transgene derived nEGFP relative to immunofluorescence staining of Hes1 (A-C), Ptf1a (E-G), Nkx6.1 (I-K), HNF1β (M-O) and Sox9 (Q-S). Quantitative analysis of the percentage of EGFP⁺ positive cells that are positive or negative for Hes1 (D), Ptf1a (H), Nkx6.1 (L), HNF1b (P) and Sox9 (T). White arrows indicate EGFP+ cells that are negative, and arrowheads indicate EGFP⁺ cells that are positive for a given pancreatic maker gene. Values are presented as mean±S.D. Scale bar: 50 μm.

FIG. 51 (A-E) shows Nkx6.1 is a direct target of Notch. Immunofluorescence staining of Nkx6.1 (A) and Hes1 (B) in E12.5 wild type pancreas. (C) Overlay of panel A and B. (D-E) ChIP-Seq analysis on the Nkx6.1 locus (D) and Hes1 locus (E) with: anti-RBP-jk antibodies on E12.5, E15.5 and E17.5 pancreatic chromatin; anti-Ptf1a antibody on E15.5 pancreatic chromatin; anti-RNA Polymerase-2 antibody on E15.5 pancreatic chromatin as well as input tracks. Scale bar: 50 μm.

FIG. 52 (A-H) shows suppression of Notch signaling through dnMAML1 does not lead to premature endocrine differentiation. Immunofluorescence staining of Pdx1 and insulin in E11.5 (A) and E12.5 wild type pancreas and in the presence of transgene derived nEGFP expression in E11.5 (B) and E12.5 (D) DTG embryos. Immunofluorescence staining of Pdx1 and Glucagon in E11.5 (E) and E12.5 (G) wild type pancreas and relative to transgene derived nEGFP in DTG embryos at E11.5 (F) and (E12.5). Scale bar: 50 μm.

FIG. 53 (A-C) shows the model of Notch mediated patterning of multipotent pancreatic progenitor cells into tip and trunk domains. At E12.5, the pancreatic epithelium is organized into tip and trunk domains, while the expression of Pdx1 is throughout all pancreatic cells; Ptf1a is restricted to the tip domain marking pro-acinar cells. While the majority of Notch-deficient cells are localized to the tip domain, a few remain in the trunk (arrows) (A). By E14.5 almost all Notch-deficient cells have resolved into the tip domain (B). This suggests a dynamic process of Notch-mediated patterning of pancreatic progenitor cells in which the loss of Notch within a multipotent progenitor leads to a tip fate and localization (C). Scale bar: 50 μm.

FIG. 54 (A-F) shows postnatal dnMAML1 overexpressing animals are impaired in blood glucose clearance. A. Intraperitoneal glucose tolerance test of 1 month old dnMAML1 DTG animals and wild type litter mates. Animals were fasted overnight followed by intraperitoneal glucose injection (2 mg/g body weight). Immunofluorescence staining of insulin on E14.5 pancreas indicates lack of insulin in dnMAML1 expressing cells (B-D). Postnatal day 18 pancreas express dnMAML1 in insulin producing cells as indicated by EGFP (F). Compared to wild type islets which have glucagon producing cells on the periphery of insulin⁺ cells (E), dnMAML1 animals have disrupted islet architecture, with glucagon+ cells intermixed with insulin+ cells (F). Scale bars: 50 μm.

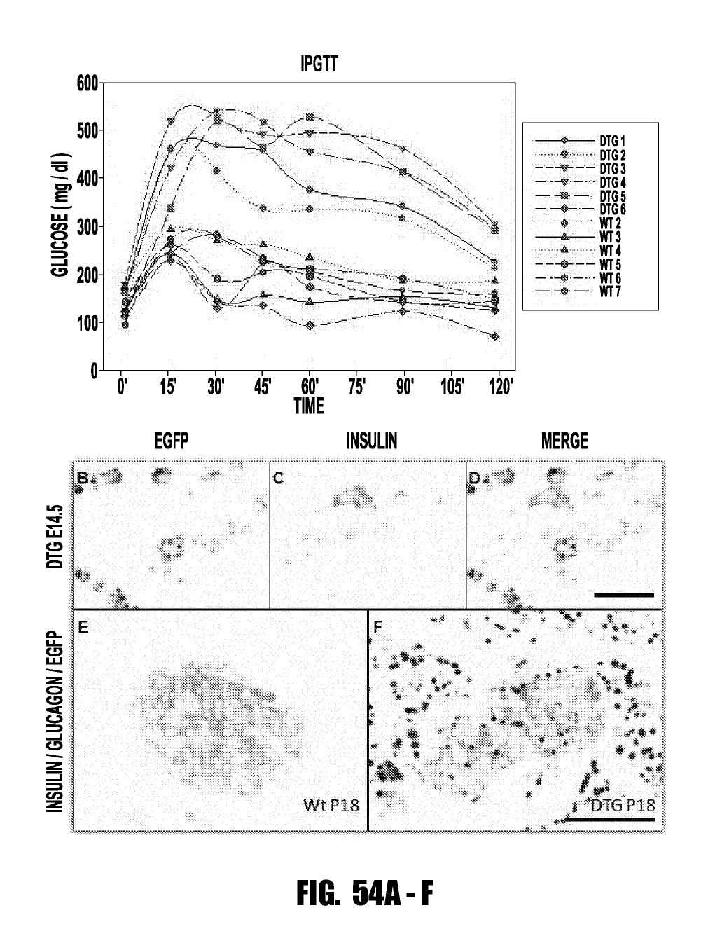

FIG. 55 (A-C) shows whole body and pancreatic mass of E18.5 Wt and DTG embryos. (A) Whole body mass of E18.5 Wt and DTG embryos. (B) Mass of Wt and DTG pancreas at E18.5. (C) Ratio of E18.5 pancreas to whole body mass in Wt and DTG embryos. Values are presented as mean±S.D.*: p<0.05; **: p<0.005; ***p: <0.0005. Wt: N=19; DTG: N=7.

FIG. 56 (A-L) shows random mosaic expression of dnMAML1 in early pancreatic epithelium. Expression of dnMAML1 as indicated by EGFP is mosaic within the pancreatic epithelium, marked by Pdx1, at all stages analyzed: (A-C) E10.5; (D-F) E11.5; (G-I) E12.5; (J-L) E13.5. Though dnMAML1 seems to be expressed in a few Pdx1 negative cells at E10.5 and E11.5, this is due to non-homogenous staining of the Pdx1 antibody which we observe in Wt pancreas as well (FIG. 6 A, E). Scale bar: 50 μm.

FIG. 57 (A-L) shows dnMAML1 differentiate into acinar fate following a similar time course as in wild type pancreas. Fluorescence visualization of EGFP expression and immunofluorescence staining of amylase in wild type E12.5 (A-C) and E14.5 (G-I) compared to DTG E12.5 (D-F) and E14.5 (J-L) pancreas. Scale bar: 50 μm.

FIG. 58 (A-L) shows the effect of dnMAML1 on pancreatic progenitor markers at E12.5. Immunofluorescence staining of Ptf1a and Pdx1 in Wt (A) and DTG pancreas (B); Nkx6.1 and Pdx1 in Wt (D) and DTG (E); Hnf1b and Pdx1 in Wt (G) and DTG (H); Sox9 and Pdx1 in Wt (J) and DTG (K). Quantification of the ratio of: Ptf1a to Pdx1 in Wt and DTG (C); Nkx6.1 to Pdx1 in Wt and DTG (F); Hnf113 to Pdx1 in Wt and DTG (I); Sox9 to Pdx1 in Wt and DTG. Values are presented as mean±S.D. **: p<0.001; n.s.: p value is not significant. Scale bar: 50 μm.

FIG. 59 (A-B) shows Nkx6.1 is a Notch target gene (A) Chromatin immunoprecipitation on E13.5 pancreas with anti-RBP-jk antibody. GAPDH promoter primer readout indicates effective ChIP with anti-RNA Polymerase II. (B) The location of qPCR products containing putative RBP-jk sites in (A) on the Nkx6.1 gene. Values are presented as mean±S.D.

FIG. 60 (A-B) shows organ Domain Patterning. A: Double in situ hybridization for insulin and amylase, performed at the secondary transition. B: Pseudo-colored schematic of the trunk and the tip fields.

FIG. 61 (A-D) shows TrPC and TipPC markers and Notch signaling A, B: E12.5 pancreas, Nkx6.1 is expressed in TrPCs, overlapping with the Notch target, Hes1 (B, insert). C, D: E14.5, cells inhibited in Notch signaling (DN-MAML1, here detected as nEGFP) are sorted to Tips. Such cells co-express the TipPC marker Ptf1a (D), but not the TrPC marker Hnf1b (C).

FIG. 62 (A-B) shows inclusion of a Canonical Wnt Signal at Stage 4 Enhances De Novo Insulin Generation. Panel A—Schematic representation of the two different directed differentiation protocols used throughout this series of experiments. The only difference between the two different differentiation protocols is the inclusion of Wnt3a at stage 4 of the directed differentiation protocol as illustrated. Control Cultures and cultures which had Wnt3a administrated at stage 4 were incubated an additional 4 days in maturation medium before RNA extractions and immunohistochemical analysis were performed. Panel B—An RT-qPCR analysis of the two differing conditions. Transcripts assayed are the endodermal marker FoxA2, the pancreatic progenitor marker Pdx1, the TrPC marker Nkx6.1 and the two endocrine markers Glucagon and Insulin. Note two different primer sets were used for insulin.

FIG. 62 (C-J) shows inclusion of a Canonical Wnt Signal at Stage 4 Enhances Insulin Generation. Panel C-J—Immunohistochemical analysis of stage 5 (day 4) cultures differentiated in the presence and absence of Wnt3a at stage 4. Panels CD and E-F compare the relative levels of C-peptide and Pdx1 respectively between the two differing differentiation protocols. Panels G-J show the combined expression of C-peptide and Pdx1 at both 10× magnification (panels G-H) and 20× Magnification (panels I-J).

FIG. 63 (A-B) shows comparison of Pan-CM Mediated Differentiation to “Gold Standard” Protocol. Panel A—show schematic representations of the three different differentiation reactions being compared throughout this series of experiments. The reaction labeled stage 4 is a directed differentiation protocol by Kroon et al. The culture condition labeled Day 14 throughout the figure is pluripotent cultures which were incubated in the presence of Pan-CM for 14 days and the reaction condition labeled as CM is pluripotent cultures which were incubated in the presence of conditioned medium from embryonic body-trunk derived feeders. Panel B shows select RT-qPCR analysis for the pancreatic progenitor marker Pdx1, the alpha cell specific endocrine marker Glucagon, the beta-cell specific endocrine markers Insulin and Mafa and the hepatocyte specific marker Albumin.

FIG. 64 (A-I)—Formation of trunk Progenitors. Pluripotent cultures were subjected to a directed differentiation protocol as outlined in panel A. Stage 4 cultures were then treated with collagenase and incubated at 37 degrees for 4 minutes at which times cultures were gently scrapped and collected by centrifugation at 1000 RPM for 3 minutes. Cultures were grown overnight as a suspension culture supplemented with maturation medium. The following day cells aggregates were collected through centrifugation (1000 RPM for 3 minutes) and placed in 4% PFA solution for 15 minutes. This was changed to a 4% sucrose solution for an hour at which time cultures were placed into OCT solution and prepared for sectioning as previously described. Panel A shows the protocol used. Panels B-E shows an example of the differentiated culture stained for the pancreatic marker Pdx1 and the endodermal marker FoxA2. Prime letters are higher magnification images from the region indicated by the box in panel C. Panels F-I shows the same culture stained for the pancreatic marker Nkx6.1 and the endodermal marker FoxA2. Prime letters are higher magnification images from the region indicated by the box in panel F.

FIG. 65 (A-B) shows maturation Medium Improves Differentiation Towards Insulin Producing Cells. Panel A shows a schematic outline of the two different reactions performed. Note the only difference between the two reactions are the incubation conditions which occur at stage 5-6, namely reaction 2 is incubated in the presence of our maturation medium as compared to the published conditions used in reaction 1. Panel B shows the relative expression levels between the two different reaction conditions for the genes indicated in the graphs and the relative ratio between the improvements observed between the endocrine genes insulin and glucagon.

FIG. 65 (C-J) shows Cultures Treated with Maturation Medium Display Higher Percentage of Ins+Glu− Cells. Panels C-J show representative immunohistochemical stainings corresponding to the reactions shown in panel A. Panels C-F show stainings of the two endocrine markers C-peptide and Glucagon under both sets of conditions. Panels G-H show the coexpression of C-peptide and glucagon at 10× magnification (Panels G-H). Panels I-J show a close of the indicated regions with emphasis on monohormonal cells: the red arrows show C-Pep+/Glu− cells where as the arrows indicate Glu+/C-pep− cells.

FIG. 66 (A-B) illustrates a mutational series of Ngn3, designed to stabilize the protein. A. listing of site directed point mutations. B. Testing of five substitution mutants.

FIG. 67 (A-D) shows Wnt-induced formation of TrPCs. Expression of Nkx6.1 and Pdx1 was assessed at E13.5 in Wnt7b gain of function embryos (Wnt7B ON, fig. B, D) and WT embryos (A, B). The TrPC determinant Nkx.6.1 us widely expressed in presence of Wnt7b, but is gradually lost in tip-regions of the WT pancreas.

FIG. 68 (A-F) shows gain-of-function model of canonical Wnt signaling in pancreas (pTRE2-Wnt7b-IRES-EGFP; Pdx1-tTA). A, B: stereoimage of DTG and WT. EGFP reporter activity seen in B. D, F: DTG pancreas consists of pancreatic ductal cells, expressing Hnf6 (D), and DBA (F). A general loss of endocrine (Single-positive Pdx1+/Hnf6− (C), INS+ cells and acinar cells is observed in DTGs (D,F).

FIG. 69 shows In-situ hybridization of the notch target gene Hes1 mRNA, E13.5 in WT and Wnt7b overexpressing pancreas.

### DETAILED DESCRIPTION

The methods and techniques described herein are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### DEFINITIONS

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced potency to achieve the effects described in this application. Following release from storage, and prior to administration to the subject, it may be preferable to again assay the cells for potency. This can be done using any of the assays, direct or indirect, described in this application or otherwise known in the art. Then cells having the desired potency can then be administered to the subject for treatment. Banks can be made using cells derived from the individual to be treated (from their pre-natal tissues such as placenta, umbilical cord blood, or umbilical cord matrix or expanded from the individual at any time after birth). Or banks can contain cells for allogeneic uses.

A “cell growth medium,” as defined herein, is an aqueous media containing the factors and nutrients suitable for supporting the growth of multipotent or pluripotent cells, in particular human multipotent or pluripotent cells. For maintenance of multipotent or pluripotent cells, the preferred basic medium is DMEM-F12 (Gibco, Invitrogen cell culture, USA Cat. No. 11320-033) supplemented with 10% KOSR (Life Technologies, Catalog Number 10828-028), non-essential amino acids (1%, Gibco, Catalog Number, 11140-050), 2 ng/ml bFGF (Invitrogen, 13256-029), and L-Glutamine (1% v/v, Gibco, Catalog Number, 25030-081). In some embodiments, such as those pertaining to stage-wise directed differentiation, the basic medium in supporting the first stage of stem cells differentiating into somatic cells from pluripotency is RPMI1640 supplemented with Bovine Serum Albumin (2%, Sigma). Most preferably the present invention makes use of DMEM-F12 as the basic medium (i.e. the basic media consists essentially of DMEM-F12) for direct induction of pancreatic fate as well as during stage-wise directed differentiation of multipotent or pluripotent cells (except for induction of stage 1, as aforementioned). For most embodiments during directed differentiation, the preferred culture medium is supplemented with 1% B27 supplement, except when excluded as noted in examples. The cell culture media applied are well known in the art of cell cultures and all are commercially available.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning, as does the word “includes.”

“Conditioned medium” (CM) refers to any cell culture growth medium which is exposed to a cellular source for 24 hours before being used on a second cellular source as a growth medium. Throughout this application the cellular source that will be used to condition the medium will be mesenchyme or fibroblasts of an embryonic origin. Specifically these cellular sources used to condition medium will be either MEFs or organ-specific MEFs. The secondary cell with which conditioned medium will be used as a supportive growth or differentiation inducing medium will be pluripotent cultures. Within this application the term unconditioned medium (UCM) is also used, UCM specifically refers to a defined medium mixture which is intended to be supplemented onto either MEF or organ-specific MEF cultures to create CM or organ-specific CM.

“Defined medium” refers to a cell culture growth medium to which specific, indicated factors have been added.

“Differentiated cells” or “differentiation of cells” relates to the acquisition of specific abilities of cells, and an ensuing display of specific markers. In the terminal differentiated state, of which the pancreatic beta cell represents one such, it is generally assumed that such are not competent for adoption of other cell fates. Any cell state that existed prior to the formation of the terminally differentiated cells, in terms of the historical events occurring in cells that existed before the terminal cell fate was adopted and if such cells could be traced to give rise to the terminal differentiated one, are said to be of a progenitor cell type.

“Directed differentiation” or “DD” protocols, refer to the stage-wise induction of specific fates, using particular compositions of matter applied to the forward differentiating pluripotent culture in order to guide its fate. The terminology of Directed Differentiation and its application is covered by prior art, (US patents referrals), and remains largely the current state-of-the-art in methods to derive particular fates from pluripotent cells.

“Direct induction” or “DI” protocols, refer to the one-step induction of a particular fate, in this case pancreatic, or lung, dependent on the specific DI method described. In contrast to the DD protocols, the DI protocol does not claim to involve the serial progression through embryonic stages mimicking normal development, such as for instance the definitive endoderm, the primitive gut tube, the segmented gut, the organ-patterned evaginations of the gut, and the defined organ state. Rather, the DI method refers to a method for which pluripotent cells, exposed to a particular matter composition adopts a state such as the MPC, or the TrPC state, as described within. Therefore, in contrast to conventional differentiation strategies which rely on the sequential use of specific factors, generally for 2-4 days at a time, to induce a stage-wise specific differentiation response within hES cultures, the method describe herein relies on the continued exposure of the same differentiating inducing medium over prolonged periods of time resulting in one embodiment in the generation of pancreatic endoderm. In another embodiment, this technology leads to the generation of cells having pulmonary identity.

“Effective amount” generally means an amount which provides the desired local or systemic effect, e.g., effective to ameliorate undesirable effects of inflammation, including achieving the specific desired effects described in this application. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“Embryonic stem cells (ESCs)” refers to cells that have unlimited self-renewal and multipotent differentiation potential and are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

“Endocrine Progenitor Cell” or “EnPC” refers to a subset of the trunk-progenitor cell (TrPC) pool, in which expression of the pro-endocrine factor Neurogenin3 is initiated.

“Exogenously added,” compounds such as growth factors, differentiation factors, and the like, in the context of cultures or conditioned media, refers to growth factors that are added to the cultures or media to supplement any compounds or growth factors that may already be present in the culture or media. For example, in some embodiments, cells cultures and or cell populations do not include an exogenously-added retinoid.

“Induced pluripotent stem cells (IPSC or IPS cells)” are somatic cells that have been reprogrammed, for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

“Insulin producing cell” refers to a cell differentiated from a pancreatic progenitor which secretes insulin. An insulin producing cell includes pancreatic β-cells as that term is described herein, as well as pancreatic β-like cells that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner. A population of insulin producing cells, e.g., produced by differentiating to pancreatic progenitors and then subsequent differentiation into insulin producing cells according to the methods described herein can be pancreatic β-cells or β-like cells (e.g., cells that have at least two characteristics of an endogenous β-cell).

“Pancreatic β-like cell” as used herein refers to a cell produced by the methods as disclosed herein which expresses at least 15% of the amount of insulin expressed by an endogenous pancreatic β-cell, or at least about 20% or at least about 30%, or at least about 40%, or at least about 50%, or at least about 60%, or at least about 70%, or at least about 80%, or at least about 90%, or at least about 100% or greater than 100%, such as at least about 1.5-fold, or at least about 2-fold, or at least about 2.5-fold, or at least about 3-fold, or at least about 4-fold or at least about 5-fold or more than about 5-fold the amount of the insulin secreted by an endogenous pancreatic beta-cell, or alternatively exhibits at least one, or at least two characteristics of an endogenous pancreatic β-cell, for example, but not limited to, secretion of insulin in response to glucose, and expression of β-cell markers, such as for example, c-peptide, Pdx1 and glut-2. In one embodiment, the pancreatic β-like cell is not an immortalized cell (e.g., proliferate indefinitely in culture). In one embodiment, the pancreatic β-like cell is not a transformed cell, e.g., a cell that exhibits a transformation property, such as growth in soft agar, or absence of contact inhibition.

“β-cell marker” refers to, without limitation, proteins, peptides, nucleic acids, polymorphism of proteins and nucleic acids, splice variants, fragments of proteins or nucleic acids, elements, and other analytes which are specifically expressed or present in pancreatic β-cells. Exemplary β-cell markers include, but are not limited to, pancreatic and duodenal homeobox 1 (PDX-1) polypeptide, insulin, c-peptide, amylin, E-cadherin, Hnf3β, PC1/3, BETA2/NEUROD, NKX2.2, NKX6.1, GLUT2, PC2, ZnT8/SLC30A8, and those described in Zhang et al., Diabetes. 50(10):2231-6 (2001). In some embodiments, the β-cell marker definition is provided as a composite expression of three nuclear marker determinants, belonging to the above group of markers. In some embodiments, the β-cell marker is PDX-1 expressed together with Insulin, or PDX1 expressed together with NKX6.1, or PDX1 expressed together with Insulin, and NKX6.1. Those skilled in the art will understand that cellular phenotype maturity is defined by co-expression of select marker genes. As a corollary, the definition criteria for mature insulin producing cells is directly related to the number of markers in the above exemplary list, being co-expressed in the said beta cell.

“Non-insulin producing cell” as used herein means any cell of endoderm origin that does not naturally synthesize, express, or secrete insulin constitutively or by induction. Thus, the term “non-insulin producing cells” as used herein excludes pancreatic β cells. Examples of non-insulin producing cells that can be used in the methods include pancreatic non-β cells, such as amylase producing cells, acinar cells, cells of ductal adenocarcinoma cell lines (e.g., CD18, CD11, and Capan-I cells (see Busik et al., 1997; Schaffert et al. 1997). Non-pancreatic cells of endoderm origin could also be used, for example, non-pancreatic stem cells and cells of other endocrine or exocrine organs, including, for example, liver cells, thymus cells, thyroid cells, intestine cells, lung cells and pituitary cells. In some embodiments, the non-insulin producing endodermal cells can be mammalian cells or, even more specifically, human cells. Examples of the present method using mammalian pancreatic non-islet, pancreatic amylase producing cells, pancreatic acinar cells are provided herein.

“Isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture. However, as used herein, the term “isolated” does not indicate the presence of only the cells described herein. Rather, the term “isolated” indicates that the cells described herein are removed from their natural tissue environment and are present at a higher concentration as compared to the normal tissue environment. Accordingly, an “isolated” cell population may further include cell types in addition to the cells described herein cells and may include additional tissue components. This also can be expressed in terms of cell doublings, for example. A cell may have undergone 10, 20, 30, 40 or more doublings in vitro or ex vivo to become enriched compared to its original numbers in vivo or in its original tissue environment (e.g., bone marrow, peripheral blood, placenta, umbilical cord, umbilical cord blood, adipose tissue, etc.).

“Maturation medium” (MM) refers to a defined medium composition designed to enhance the generation of insulin producing cells from a forward differentiating culture containing a population of MPCs or TrPCs.

The term “MEF” is an acronym which refers to mouse embryonic fibroblasts and when it is stated without a prefix in this application it specifically refers to primary cell cultures derived from the body trunks of mouse embryos 13.5 days (E13.5) into their development. Throughout this application the term organ-specific-MEFs refer to primary mesenchyme cultures derived from embryonic mouse organs. The organ of origin will be named as a prefix to the acronym MEF (i.e., Pan-MEF implies a primary mesenchyme culture derived from mouse embryonic pancreas) and in all cases, unless explicitly stated otherwise in a figure or example, the age of the embryo from which the organ-specific MEFs were derived was E13.5 mouse embryos.

“Mesenchymal stem cells” or “MSCs” are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

“Multipotent” or “multipotent cell” refers to a cell type that can give rise to a limited number of other particular cell types. Multipotent cells are committed to one or more embryonic cell fates, and thus, in contrast to pluripotent cells, cannot give rise to each of the three embryonic cell lineages as well as extraembryonic cells.

“Multipotent pancreatic progenitor cells” or “MPCs” refers to multipotent progenitor cells characterized by co-expression of several markers defining the pancreatic state, thus distinguishing this from other non-pancreatic endodermal derivative states such as, but not limited to, liver, intestine, gastric, and pulmonary states. The MPC state is defined as being multipotent for all terminal fates of endodermal descendancy in the pancreas. Such include, but may not be limited to, endocrine cells of the insulin, glucagon, ghrelin, somatostatin, or Pancreatic Polypeptide expressing states, the pancreatic ductal cells, the pancreatic centroacinar cells, the pancreatic acinar cells. The multipotent pancreatic progenitor cells is defined as pancreatic state for which no organ domain patterning, and/or loss of specific competence for development into any of the aforementioned differentiation states has occurred. The MPC state can be described by co-expression of the following markers, Ptf1a, Pdx1, Nkx6.1, Nkx2.2, Hnf1b, Sox9, FoxA2. In the developing mouse, and developing human embryo the MPC state is induced at organ formation from the definitive endoderm at early somite stages, and MPC formation precedes formation of the pancreatic beta cell complement.

“Multi-stage differentiation” is a process wherein cells transition from low, or non-existing potency, gradually to increasing potency. Those skilled in the art will understand that the selection processes and differentiation inducing methods as described previously, can be sequentially applied, with a combined effect of increasing potency of the original cell population. During Multi-stage differentiation, the transition of cells of origin will proceed through a process going, as an example, from pluripotency, to multipotency, with a gradual restriction of multipotency.

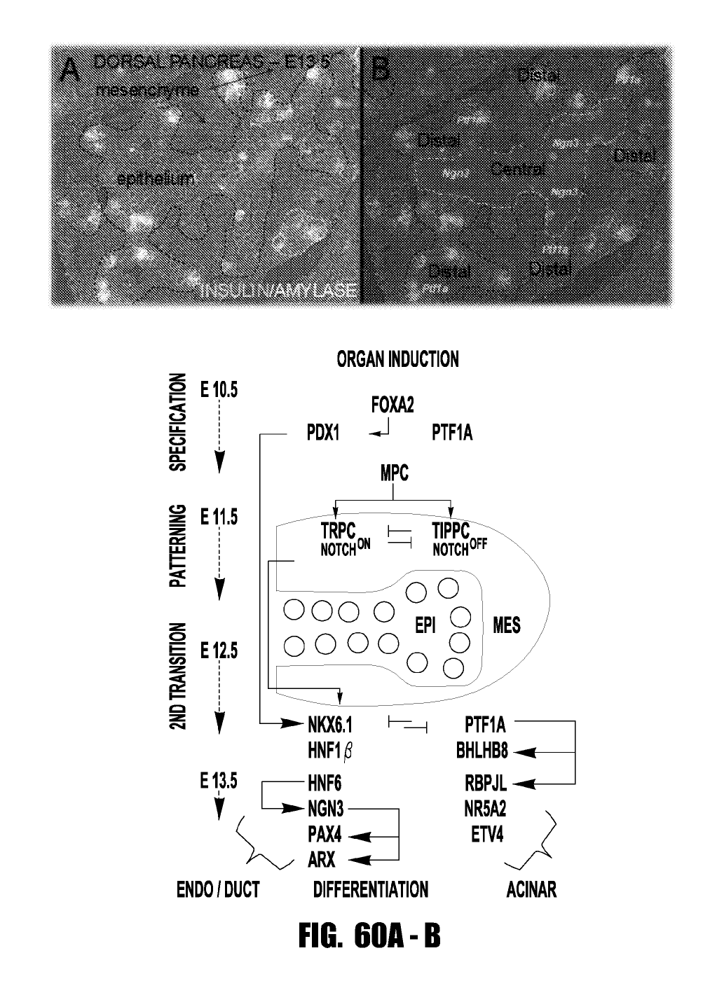

“Organ domain patterning” refers to the process of splitting descendant fate competencies in the MPC pool, and is visible by the formation of regionalized domains of gene expression as demonstrated in FIG. 60 (A-B). The events involving organ domain patterning occur subsequently to the formation of the organ itself at the early budding stages, and is initiated as the MPC-definition set of markers segregate into at least two specific cell populations characterized by co-expression of particular marker genes. The organ domain patterning event precedes formation of the pancreatic beta cell complement. The two predominant cell populations arising from the organ domain patterning event are tip-progenitor cells (TipPCs) and trunk-progenitor cells (TrPC).

“Pancreatic endoderm” refers to a progenitor cell population which is capable of differentiating into all of the cellular sub-types present in the adult organ, and this multipotent in nature. The continued expansion of the two buds brings the first defining differences within the pancreatic endoderm as it begins to partition different expression patterns in a regionalized fashion. This is later referred to as organ domain patterning, and is a process involving changes in cell fate competencies among pancreatic progenitor cells. Pancreatic endoderm initially forms as an outgrowth of the gut tube at two distinct regions destined to become the dorsal and ventral pancreatic buds. The initial emergency of the pancreatic program is visualized by regionalized expressed of specific marker genes within the primitive gut tube at somite stage 6-10 in both mice and humans. While there are some defining differences between the expression patterns of the dorsal and ventral pancreatic buds for practical purposes the cells present within these buds are very homologous in expression patterns and share most of the defining markers, and both tissues are able to give rise to all terminal pancreatic cell fates. The marker genes that define the pancreatic state in the early forming pancreas are those described in the definition of the MPC.

When used in connection with cell cultures and/or cell populations, the term “portion” means any non-zero amount of the cell culture or cell population, which ranges from a single cell to the entirety of the cell culture or cells population. In preferred embodiments, the term “portion” means at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94% or at least 95% of the cell culture or cell population.

The term “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pluripotent” is meant that the cell can give rise to each of the three embryonic cell lineages as well as extraembryonic cells. Pluripotency as used herein is limited to the inner cell mass state, and is not used at any point to describe lineage restricted progenitors that form during development. The pluripotent cell state is a natural one, but can be propagated in-vitro under specific conditions known to those skilled in the art. In such culture conditions, perpetual maintenance of the stem cell, or pluripotent state, occurs. The defining qualities of the pluripotent cells are not limited to their functional characteristic, but can also be described through gene expression patterns and consequently the identification of pluripotent cells can be accomplished through staining patterns for markers they exhibit. The presence of the protein transcription factors OCT3/4, SOX2 and NANOG represent a group of core transcription factors involved in maintaining the pluripotent state and the down regulation of these factors is a prerequisite for forward differentiation. These three transcription factors have all been shown to interact with one another and there is a great deal of overlap in the genes that are under their transcriptional control. In addition to the transcription factors responsible for pluripotent maintenance are often identified through the use of several surface markers present on them, the most common ones being Stage Specific Embryonic Antigens 3 and 4 (SSEA3 and SSEA4), Tra-1-61 and Tra-1-81. Pluripotent cells, such as embryonic stem cells, can also exhibit alkaline phosphatase activity which is often used in the identification of pluripotent cells.

“Progenitor cell” refers to a normal cellular state, at any point, which represented a direct lineage ancestor for the terminally differentiated cell. By definition progenitors are not irreversibly destined to adopt the said terminal fate, but may be multipotent for more than one terminal state. As such, they display competence for differentiation into more than a single descendant fate. As development proceeds in any organism, the gradual loss of competency, and thus loss of multipotency, is the hall mark of differentiation and the temporally defined formation of tissues and organs.

“Self-renewal” of a stem cell refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation, and this process essentially describes a gain in multipotency. Stem cells also may be derived from abnormal tissue, such as a teratocarcinoma and some other sources such as embryoid bodies (although these can be considered embryonic stem cells in that they are derived from embryonic tissue, although not directly from the inner cell mass). The pluripotent stem cell state may also be created by introducing genes associated with stem cell function into a non-stem cell, such as an induced pluripotent stem cell.

A “subject”, as used therein, can be a vertebrate or a mammal. Examples of subjects include livestock, test animals, and pets, such as ovine, bovine, porcine, canine, feline and murine mammals, as well as reptiles, birds and fish. The terms, “patient” and “subject” are used interchangeably herein. Preferably, the subject is a mammal. The mammal can be a human, non-human primate, mouse, rat, dog, cat, horse, or cow, but are not limited to these examples.

With respect to cells in cell cultures or in cell populations, the term “substantially free of” means that the specified cell type of which the cell culture or cell population is free, is present in an amount of less than about 10%, less than about 9%, less than about 8%, less than about 7%, less than about 6%, less than about 5%, less than about 4%, less than about 3%, less than about 2% or less than about 1% of the total number of cells present in the cell culture or cell population.

“Tip-progenitor cell” or “TipPC” refers to the tip-progenitor cell descending from the MPC state, being of pancreatic origin. The TipPC state or phenotype is transient in nature. The TipPC state is positionally defined by the location of the distal-most tips of the developing pancreas, and is characterized by a defined set of marker genes, including, but not limited to, PDX1, PTF1A, bHLHb8. The TipPC is also defined by absence of expression HNF1b, HNF6, SOX9, HES1, and NKX6.1. The TipPC does not initiate Ngn3 expression, and to the understanding of the inventors, does not, following organ domain patterning, contribute significantly to the endocrine complement of the pancreas, including the pancreatic beta cell population. The descendants of the TipPC, as described within is predominantly acinar cells of the pancreas, expressing multiple zymogen-encoding genes, including, but not limited to, pancreatic-expressed versions of gene encoding amylase, carboxypeptidases, trypsin, chymotrypsin, RNAse and lipases.

“Trunk-progenitor cell” or “TrPC” refers to the trunk-progenitor cell descending from the MPC state. The TrPC state or phenotype is transient in nature. The TrPC state is positionally defined by the location of the central mass of the developing pancreas, and is characterized by a defined set of marker genes, including, but not limited to, PDX1, HNF1b, HNF6, SOX9, HES1, and NKX6.1. The TrPC is also defined by absence of expression PTF1a, and bHLHb8.

“Therapeutically effective amount” refers to the amount of an agent determined to produce any therapeutic response in a mammal. For example, effective anti-inflammatory therapeutic agents may prolong the survivability of the patient, and/or inhibit overt clinical symptoms. For example, effective anti-diabetic therapeutic treatments could include a biologic material able to produce the insulin polypeptide hormone. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of inflammation.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy. In various embodiments, the symptoms of a disease or disorder are alleviated by at least 5%, at least 10%, at least 20%, at least 30%, at least 40%, or at least 50%.

“Validate” means to confirm. In the context of the invention, one confirms that a cell is an expressor with a desired potency. This is so that one can then use that cell (in treatment, banking, drug screening, etc.) with a reasonable expectation of efficacy. Accordingly, to validate means to confirm that the cells, having been originally found to have/established as having the desired activity, in fact, retain that activity. Thus, validation is a verification event in a two-event process involving the original determination and the follow-up determination. The second event is referred to herein as “validation.”

Induction of Pancreatic Fates from Multipotent and Pluripotent Cells

Embodiments described herein relate to the induction of pancreatic fates, pancreatic precursor cells, pancreatic insulin producing cells, enriched populations of pancreatic precursor cells, or enriched populations of pancreatic insulin producing cells from multipotent and pluripotent cells. The pancreatic fates can include those of the pancreatic endocrine compartment, and specifically include enriched populations of pancreatic progenitor cells, such as Trunk progenitor cells (TrPCs), as well as enriched populations of pancreatic insulin producing cells that are differentiated from the pancreatic progenitor cells or TrPCs. The pancreatic fates, pancreatic precursor cells, pancreatic insulin producing cells, enriched populations of pancreatic precursor cells, or enriched populations of pancreatic insulin producing cells can be induced from the multipotent and pluripotent cells using a pancreas-specific mesenchymal stem cell conditioned medium or a defined medium based on characterization of the transcriptome and secretome of the pancreas-specific mesenchymal stem cell conditioned medium.

During development, mesenchyme forms the connective tissue between and within the developing tissues and organs. Further, mesenchyme has been implicated in secreting signals needed for the growth and differentiation of developing organs. No better example of the importance of mesenchymal cells in development can be offered than the stem cell niche. The niche is the environment which helps sustain stem cells in an undifferentiated state; it is the surrounding microenvironment that stem cells reside in which is composed of by extracellular components as well as adjacent cellular components. While the existence of organ-specific stem cells is debated depending on the organ in question, there are some well characterized stem-cell niches within mammalian systems including the hematopoietic stem cell niche and the intestinal crypt stem cell niche. Both cases have a common theme between them being the association of stromal or mesenchymal cells respectively within these niches. These associated cells have been implicated in the regulation of cell activation, proliferation and differentiation of the actual stem cells making them an integral functional component of the niches.

Mesenchymal cells are characterized by their ability to produce and secrete structural molecules commonly found within the extra cellular matrix (ECM), though it is well established that mesenchymal cells also produces an array of different signaling molecules which can function in both the differentiation of cells (as in the case of the developing organ) as well as the maintenance of stemness, as seen in the mesenchymal components of the intestinal stem-cell niche. The expression patterns of mesenchymal cells have been shown to be vastly different dependent on the site of origin, though some general markers expressed in multiple mesenchymal lineages are vimentin, fibronectin and various forms of collagen. In contrast to the cells of the epithelial components of a developing organ mesenchyme is marked by a migrating capability. We have shown herein that embryonic and adult organ-specific mesenchymal cells when cultured ex vivo retain enough of their initial signaling properties to drive an increased percentage of pluripotent cells to differentiate toward specific organ fates.

There are two broad categories of mammalian stem cells, embryonic stem cells and adult stem cells. Embryonic stem cells refer to stem cells derived from the inner cell mass of blastula stage embryos, while adult stem cells reside in specialized regions, niches, within the different organs or tissues of the adult organism. While the defining difference between the two categories is based on the site of origin of the stem cell, these differences lead to important functional differences. Most important of the fundamental differences deals with the competence of the stem cells to generate different kinds of tissues or cell types. Adult stem cells reside within specialized niches of the adult organs and are multipotent meaning that they are capable of differentiating into a limited number of cell types, usually representative of the different cell types present within the organ of origin. Embryonic stem cells are pluripotent cells capable of differentiating into all the cell types of the adult organisms. This capability is reflective of their natural role in development. The migration of the inner cell mass through the primitive streak during gastrulation establishes the formation of the three embryonic germ layers: ectoderm, mesoderm and endoderm. In turn these three germs layers will develop into all of the tissues and organs present within the body. Ectoderm is destined to develop into the nervous system and the epidermis. Mesoderm forms the hematopoietic system, connective tissue, heart, kidneys, muscular and skeletal systems. While the endoderm will give rise to the gut tube and all of the organs that are derived from it including; the trachea, lungs, thyroid, stomach, liver, pancreas, intestines and colon.

While embryonic stem cells have a clear theoretical definition based on developmental time and position in the embryo, the in vitro cultivation of hES cells have a slightly different set of criteria. Unlimited growth potential means that embryonic stem cells are capable of replicating indefinitely and the progeny of this replication are capable of taking on the characteristics of the pluripotent parent cell. One of the reasons ES cells are capable of escaping growth crisis, a characteristic of ex vivo cultured somatic cells which limits the number of progeny capable of forming from a parent cell, is through the use of telomerase activity. Telomeres are regions present at the end of chromosomes composed of by a known repeating nucleotide sequence which does not encode for a gene. The length of the telomere is shortened progressively during each replication cycle until it is essentially gone. The loss of the telomeres corresponds to the limited growth potential observed in ex vivo cultured primary cells. The telomerase activity observed in ES cells enables the lengthening of the telomeres and thus the number of replications an ES cell is capable of undergoing is not limited.

In addition to replicating additional copies of themselves ES cells also possess the capacity to differentiate into any cell-type of the adult body including unique cells representative of embryonic developmental intermediates as well as multipotent stem cells or progenitors normally found in the adult organism. ES cells have been successfully differentiating into cells representative of all three germ layers, in fact the ability to do so is a defining property of an ES cell demonstrating their capacity to differentiate into progenitors of multiple lineages.

The defining qualities of the embryonic stem cell are not limited to its functional characteristic, but can also be described through ES expression patterns and consequently the identification of pluripotent cells can be accomplished through the staining patterns they exhibit. The presence of the protein transcription factors Oct3/4, Sox2 and Nanog represent a group of core transcription factors involved in maintaining the pluripotent state and the down regulation of these factors is a prerequisite for forward differentiation. These three transcription factors have all been shown to interact with one another and there is a great deal of overlap in the genes that are under their transcriptional control. In addition to the transcription factors responsible for pluripotent maintenance ES cells are often identified through the use of several surface markers present on them, the most common ones being Stage Specific Embryonic Antigens 3 and 4 (SSEA3 and SSEA4), Tra-1-61 and Tra-1-81. ES cells also exhibit alkaline phosphatase activity which is often used in the identification of pluripotent cells.

Sources of pluripotent cells include cultures derived from the inner cell mass of blastula staged embryos as well as induced pluripotent stem cells (IPS). The latter being cells which are transformed into expressing key transcription factors involved in pluripotent maintenance, the most commonly used combination of factors being

Pancreatic endoderm initially forms as an outgrowth of the gut tube at two distinct regions destined to become the dorsal and ventral pancreatic buds. While there are some defining differences between the expression patterns of the dorsal and ventral pancreatic buds for practical purposes the cells present within these buds are very homologous in expression patterns and share most of the defining markers. Pancreatic endoderm is a progenitor pool which is responsible for differentiating into all of the cellular sub-types present in the adult organ. It is best described as a population of cells that coexpress the transcription factors FoxA2, Pdx1, Hnf1-β, Nkx.6.1, Ptf1a. The continued expansion of these buds brings the first defining differences within the pancreatic endoderm as it begins to partition different expression patterns in a regionalized fashion. Notably the expression of Ptf1a becomes restricted to the peripheral region of the developing pancreas while Nkx.6.1 and Pdx1 stays centralized. This split in the expression patterns of selective pancreatic endoderm markers has considerable developmental consequences.

While the majority of these organs are composed of cells derived from the endoderm, the associated mesenchyme is derived from the mesoderm.

Most of the work up to the present that has tried to generate pancreatic β-cells from an ES cell has focused on the directed differentiation of the ES cells. The directed differentiation of a hES culture tries to imitate the natural development of the desired cell (in this case the pancreatic β-cell) through directing the differentiation of the intermediate cell populations which normally occur during development. Since endoderm is the germ layer that gives rise to the pancreas, hES cultures first have to be prompted to form definitive endoderm. In contrast to conventional differentiation strategies which rely on the sequential use of specific factors, generally for 2-4 days at a time, to induce a stage-wise specific differentiation response within hES cultures, the method described herein relies on the continued exposure of the same differentiating inducing medium over prolonged periods of time resulting in the generation of pancreatic endoderm.

Accordingly, embodiments described herein relate to the functional components present within the cell conditioned medium and defined medium and their use in the direct induction of pluripotent cells toward pancreatic fates, pancreatic precursor cells, pancreatic insulin producing cells, enriched populations of pancreatic precursor cells, or enriched populations of pancreatic insulin producing cells.

Other embodiments relate to the direct induction of a novel pancreatic precursor cell state, herein named trunk progenitor cell (TrPC), or an enriched population of TrPCs from multipotent and pluripotent cells. It was found that the TrPC state is a requisite state for the generation of pancreatic insulin producing cells and pancreatic ductal cells. As described above, this novel pancreatic precursor cell state is transient in nature and is characterized by a defined set of marker genes, including, but not limited to, Pdx1, Hnf1b, Hnf6, Sox9, Hes1, and Nkx6.1. The TrPC is also defined by absence of expression Ptf1a, and bHLHb8. This pancreatic precursor cell state, TrPC state, is distinguished from other pancreatic precursor cells states, such a Tip-progenitor cell state, which is defined above.

Still other embodiments relate to the use of Notch signaling and Wnt signaling induction as a means to induce the TrPC state.

Yet other embodiments relate to the formulation of a specific media composition that enhances the formation of pancreatic insulin producing cells from pancreatic progenitor cells, or an enriched population of TrPC cells, or other stem cell sources.

In some methods described herein, human multipotent or pluripotent cells are induced to pancreatic progenitor cells that comprise an enriched population of TrPCs by continued exposure to the same differentiating inducing medium. The TrPC state is a requisite state for the generation of pancreatic insulin producing cells and pancreatic ductal cells.

The human multipotent or pluripotent cells that are induced by the method described herein can include human embryonic stem cells (hESCs) or induced pluripotent stem cells. Such pluripotent cells can be cells that originate from the morula, embryonic inner cell mass or those obtained from embryonic gonadal ridges. The human embryonic stem cells can express SSEA3, SSEA4, Tra-1-60, Tra-1-80, OCT3/4, SOX2 and Nanog.

The hESCs can be maintained in culture in a pluripotent state without substantial differentiation using methods that are known in the art. Such methods are described, for example, in U.S. Pat. Nos. 5,453,357, 5,670,372, 5,690,926 5,843,780, 6,200,806 and 6,251,671 the disclosures of which are incorporated herein by reference.

In some methods, hESCs are maintained on a feeder layer. In such methods, any feeder layer, which allows hESCs to be maintained in a pluripotent state can be used. One commonly used feeder layer for the cultivation of human embryonic stem cells is a layer of mouse fibroblasts. More recently, human fibroblast feeder layers have been developed for use in the cultivation of hESCs (see US Patent Publication No. 2002/0072117, the disclosure of which is incorporated herein by reference). Alternative methods permit the maintenance of pluripotent hESC without the use of a feeder layer. Methods of maintaining pluripotent hESCs under feeder-free conditions have been described in U.S. Pat. No. 7,413,902, the disclosure of which is incorporated herein by reference.

The hESCs used herein can be maintained in culture either with or without serum. In some embryonic stem cell maintenance procedures, serum replacement is used. In others, serum free culture techniques, such as those described in U.S. Pat. No. 7,217,569, the disclosure of which is incorporated herein by reference, are used. Stem cells, including hESCs, can be maintained in culture in a pluripotent state by routine passage until it is desired that they be differentiated into the TrPCs and then ultimately to pancreatic ductal and/or pancreatic insulin producing cells.

Use of Agents, Proteins, Growth Factors, and/or Cytokines to Induce Pancreatic Progenitor Cells

The human multipotent or pluripotent cells can be induced to the pancreatic progenitor cells by providing a culture or a cell population comprising the multipotent or pluripotent cells and then providing to, contacting with, or culturing with the multipotent or pluripotent cells agents, proteins, growth factors, and/or cytokines that include at least three of, at least four of, at least five of, or at least six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of an enriched population of TrPCs from the human multipotent or pluripotent cells.

In some embodiments, the pancreatic progenitor cells are induced from multipotent or pluripotent cells that are contacted with a defined medium consisting essentially of a cell growth medium and at least three of, at least four of, at least five of, or at least six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor. The inventors have determined that while certain agonists and other factors are useful for stimulating the differentiation of cells, the presence of other agonists and/or factors can lead to the formation of other types of cells, thereby reducing conversion efficiency.

In some embodiments, the CXCR4 agonist can include at least one of CXCL12 or SDF-1, an SDF-1 peptide analogue, or a CXCR4 stimulating molecule. Stromal cell derived factor one (SDF-1) is a member of the CXC family of chemokines that has been found to be constitutively secreted from the bone marrow stroma (Tashiro, (1993) Science 261, 600-602). The native amino acid sequences of SDF-1α and SDF-1β are known, as are the genomic sequences encoding these proteins (see U.S. Pat. No. 5,563,048 and U.S. Pat. No. 5,756,084). In some embodiments, the CXCR4 agonists may be substantially purified peptide fragments, modified peptide fragments, analogues or pharmacologically acceptable salts of either SDF-1α or SDF-1β. SDF-1 derived peptide agonists of CXCR4 may be identified by known biological assays and a variety of techniques such as the aforementioned or as discussed in Crump et al., 1997, The EMBO Journal 16(23) 6996-7007; and Heveker et al., 1998, Current Biology 8(7): 369-376; each of which are incorporated herein by reference.

In other embodiments, the EGFR agonist comprises at least one agent that stimulates the PI3K signaling pathway and/or the MAPK/MEK/ERK signaling pathway. Examples of EGFR agonists that that stimulate the PI3K signaling pathway and/or the MAPK/MEK/ERK signaling pathway can include EGF, HB-EGF, TGF-α, heregulin, amphiregulin, betacellulin, epigen, epiregulin, and/or neuregulin 2.

In some embodiments, the FGFR agonist can include an FGFR2b-IIIC agonist that stimulates the PI3K signaling pathway and/or the MAPK/MEK/ERK signaling pathway. The FGFR2b-IIIC agonist can include an FGF growth factor, such as FGF10 and/or FGF7.

In other embodiments, the Activin receptor agonist or agent that stimulates SMAD3 can include at least one of Activin A, nodal, TGFβ1, TGFβ2, TGFβ3, GDF8, or GDF11. In certain embodiments, the Activin receptor agonist can include Activin A.

In still other embodiments, the IL11R agonist or IL6R agonist can include an agent that stimulates the JAK/STAT3 signaling pathway. The agent can include at least on one of IL11 or IL6. In certain embodiments, the agent is IL11.

In some embodiments, the notch agonist can be a canonical or a non-canonical notch agonist. The canonical notch agonist can include at least one of Dll1, Jag1, Jag2, Dlk1, or pref1/Dlk1. In certain embodiments, the non-canonical notch agonist can be Mfap5.

In other embodiments, the BMP inhibitor can be a member of the DAN family proteins. Members of the DAN family proteins that act as BMP inhibitors can include at least one of Nbl1, chordin,chordin-like proteins, DAN, sclerostin, decorin, gremlin 1, gremlin2, cerberus, or Dand5.

In still other embodiments, the RXR agonist and/or RAR agonist can be retinoic acid, a retinoid, or a derivative thereof.

The at least three of, four of, five of, or six of (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor can be provided in cell growth medium or cell conditioned medium that is administered to, provided to, contacted with, or cultured with the cells at concentrations sufficient to promote differentiation of at least a portion of the pluripotent or multipotent cells to TrPCs. In some embodiments, the medium that includes the at least three of, four of, five of, or six of Activin A, Hb-EGF, CXCL12, an FGF growth factor, IL-11, retinoic acid, or a BMP inhibitor can be a cell conditioned medium that is be prepared by exposing a cell growth medium to isolated pancreas specific mesenchymal stem cells to provide the at least three of, four of, five of, or six of Activin A, Hb-EGF, CXCL12, an FGF growth factor, IL-11, retinoic acid, or a BMP inhibitor in the cell conditioned medium at a concentration to induce differentiation of the multipotent or pluripotent cells.

The pancreas specific mesenchymal stem cells can include mouse pancreas specific mesenchymal stem cells. The mouse pancreas specific mesenchymal stem cells can be isolated and expanded from pancreas specific mouse mesenchyme of mouse embryos. The mouse embryos can be about 5 days, about 6 days, about 7 days, about 8 days, about 9 days, about 10 days, about 11 days, about 12 days, about 13 days, about 13.5 days, about 14 days, about 15 days, about 16 days, about 17 days, about 18 days, about 19 days, about 20 days or more into development. The mouse pancreas specific mesenchymal stem cells can express CD90 but not PECAM and CD105. In other embodiments, the mouse pancreas specific mesenchymal stem cells do not express InhbA and can be isolated and expanded from adult mouse pancreas.

In other embodiments, the (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor can be provided in a defined medium that is prepared by adding the at least three of, four of, five of, or six of the (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor (e.g., at least three of, four of, five of, or six of Activin A, Hb-EGF, CXCL12, FGF growth factor, IL-11, retinoic acid, BMP inhibitor, and non-canonical notch activator) to a cell growth medium. The at least three of, four of, five of, or six of the (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor (e.g., at least three of, four of, five of, or six of Activin A, Hb-EGF, CXCL12, FGF growth factor, IL-11, retinoic acid, BMP inhibitor, and non-canonical notch activator) can be present in the medium that is administered to the cell culture at a concentration, for example, of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 1000 ng/ml, at least about 2000 ng/ml, at least about 3000 ng/ml, at least about 4000 ng/ml, at least about 5000 ng/ml or more than about 5000 ng/ml.

In some embodiments, Activin A can be provided in the medium at a concentration of at least about 10 pg/ml, at least about 25 pg/ml, at least about 50 pg/ml, at least about 100 pg/ml, at least about 200 pg/ml, at least about 500 pg/ml, at least about 750 pg/ml, at least about 1 ng/ml, at least about 5 ng/ml, or at least about 10 ng/ml. In certain embodiments, Activin A is provided in the medium at a concentration of about 10 pg/ml to about 10 ng/ml.

In other embodiments, Hb-EGF can be provided in the medium at a concentration of at least about 1 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, Hb-EGF is provided in the medium at a concentration of about 1 ng/ml to about 100 ng/ml.

In still other embodiments, SDF-1 or CXCL12 can be provided in the medium at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, CXCL12 is provided in the medium at a concentration of about 10 ng/ml to about 1 μg/ml.

In other embodiments, FGF-10 or FGF7 can be provided in the medium at a concentration of at least about 1 ng/ml, at least about 2 ng/ml, at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, FGF-10 is provided in the medium at a concentration of about 5 ng/ml to about 500 ng/ml.

In some embodiments, IL-11 can be provided in the medium at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, IL-11 is provided in the medium at a concentration of about 10 ng/ml to about 1 μg/ml.

In other embodiments, the retinoic acid can be provided in the medium at a concentration of at least about 1 nM, at least about 0.01 μM, at least about 0.02 μM, at least about 0.04 μM, at least about 0.08 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.1 μM, at least about 1.2 μM, at least about 1.3 μM, at least about 1.4 μM, at least about 1.5 μM, at least about 1.6 μM, at least about 1.7 μM, at least about 1.8 μM, at least about 1.9 μM, at least about 2 μM, at least about 2.1 μM, at least about 2.2 μM, at least about 2.3 μM, at least about 2.4 μM, at least about 2.5 μM, at least about 2.6 μM, at least about 2.7 μM, at least about 2.8 μM, at least about 2.9 μM, at least about 3 μM, at least about 3.5 μM, at least about 4 μM, at least about 4.5 μM, at least about 5 μM, at least about 10 μM, at least about 20 μM, at least about 30 μM, at least about 40 μM or at least about 50 μM. In certain embodiments, the retinoic acid can be provided in the medium at a concentration of about about 1 nM to about 10 μM.

In other embodiments, the BMP inhibitor, such as Noggin and/or Nbl1, can be provided in the medium at a concentration of at least about 1 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, the BMP inhibitor can be provided in the defined medium at a concentration of about 1 ng/ml to about 100 μg/ml

In still other embodiments, a non-canonical notch activator, such as Mfap5, can be provided in the medium at a concentration of at least about 1 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments, the non-canonical notch activator can be provided in the defined medium at a concentration of about 1 ng/ml to about 100 μg/ml

In certain embodiments, at least one of the Activin A can be provided in the defined medium at a concentration of about 10 pg/ml to about 100 ng/ml, the Hb-EGF can be provided in the medium at a concentration of about 1 ng/ml to about 100 ng/ml, the CXCL12 can be provided in the medium at a concentration of about 10 ng/ml to about 1 μg/ml, the FGF growth factor can be provided in the medium at a concentration of about 5 ng/ml to about 500 ng/ml, the IL-11 can be provided in the medium at a concentration of about 10 ng/ml to about 1 μg/ml, the retinoic acid can be provided in the medium at a concentration of about 1 nM to about 10 μM, or the BMP inhibitor can be provided in the medium at a concentration of about 10 ng/ml to about 100 μg/ml.

In some embodiments, the cell population can be provided with the at least three of, four of, five of, or six of the (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor (e.g., at least three of, four of, five of, or six of Activin A, Hb-EGF, CXCL12, FGF growth factor, IL-11, retinoic acid, BMP inhibitor, and non-canonical notch activator) at about three days, at about four days, at about five days, at about six days, at about seven days, at about eight days, at about nine days, at about ten days, at about 11 days, at about 12, at about 13 days, at about 14 days, at about 15 days, at about 16 days, at about 17, at about 18, at about 19 day, at about 20 days, at about 21 day or greater. In certain embodiments, the cell population can be provided with the at least three of, four of, five of, or six of (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor (e.g., at least three of, four of, five of, or six of Activin A, Hb-EGF, CXCL12, FGF growth factor, IL-11, retinoic acid, BMP inhibitor, and non-canonical notch activator) for about 5 days to about 3 weeks, or more particularly a about 7 days to about 2 weeks.

Production of Trunk Precursor Cells (TrPCs) from Multipotent or Pluripotent Cells

Cultures of TrPCs can be produced from multipotent or pluripotent cells, such as embryonic stem cells, in medium containing reduced serum or no serum. Under certain culture conditions, serum concentrations can range from about 0.05% v/v to about 20% v/v. For example, in some differentiation processes, the serum concentration of the medium can be less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v), less than about 10% (v/v), less than about 15% (v/v) or less than about 20% (v/v). In some embodiments, TrPCs are grown without serum or without serum replacement.

In some embodiments for differentiating human TrPCs from hESCs, differentiation is initiated in the absence of serum and in the absence of insulin and/or insulin-like growth factor. During the course of differentiation, the serum concentration may be gradually increased in order to promote adequate cell survival. In other embodiments, differentiation of hESCs to TrPCs is initiated in the absence of serum

Cell compositions produced by the above-described methods include cell cultures comprising TrPCs and cell populations enriched in other pancreatic precursor cells. For example, cell cultures and/or cell populations that comprise TrPCs can be produced, wherein at least about 50-99% of the cells in the cell culture and/or cell population are definitive TrPCs. Because the efficiency of the differentiation process can be adjusted by modifying certain parameters, which include but are not limited to, cell growth conditions, growth factor concentrations and the timing of culture steps, the differentiation procedures described herein can result in about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, about 99% or greater than about 99% conversion of pluripotent or multipotent cells to TrPCs. In methods in which isolation of TrPCs is employed, for example, by using an affinity reagent that binds to a receptor of the TrPCs, a substantially pure TrPC population can be recovered. In embodiments where the cell cultures or cell populations comprise human feeder cells, the above percentages are calculated without respect to the human feeder cells in the cell cultures or cell populations.

The progression of the pluripotent or multipotent cells to TrPCs can be monitored by determining the expression of markers characteristic of TrPCs. In some embodiments, the expression of certain markers is determined by detecting the presence or absence of the marker. Alternatively, the expression of certain markers can be determined by measuring the level at which the marker is present in the cells of the cell culture or cell population. In such processes, the measurement of marker expression can be qualitative or quantitative. One method of quantitating the expression of markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. In certain processes, the expression of marker genes characteristic of TrPCs as well as the lack of significant expression of marker genes characteristic of hESCs, extraembryonic endoderm, mesoderm, ectoderm, pancreatic insulin producing cells and/or other cell types is determined.

As described above and further in the Examples below, markers of TrPCs are Hnf6, Nkx6.1, and Hnf1b. Other markers of TrPCs cells are Sox9, Pdx1, and FoxA2. It will be appreciated that Hnf6, Nkx6.1, and/or Hnf1b marker expression is induced over a range of different levels in TrPCs depending on the differentiation conditions. As such, in some embodiments described herein, the expression of the Hnf6, Nkx6.1, and/or Hnf1b marker in TrPCs or cell populations is at least about 2-fold higher to at least about 10,000-fold higher than the expression of the Hnf6, Nkx6.1, and/or Hnf1b marker in non-TrPCs or other cell populations, for example pluripotent stem cells, PDX1-positive foregut endoderm cells pancreatic MPCs. In other embodiments, the expression of the Hnf6, Nkx6.1, and/or Hnf1b marker in TrPCs or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the Hnf6, Nkx6.1, and/or Hnf1b marker in non-TrPCs or cell populations, for example pluripotent stem cells, and/or pancreatic MPCs.

The TrPCs can be enriched, isolated and/or purified. In some embodiments, cell populations enriched, isolated and/or purified for TrPCs are produced by isolating such cells from cell cultures. TrPCs produced by any of the method described herein can be enriched, isolated and/or purified by using an affinity tag that is specific for such cells. Examples of affinity tags specific for TrPCs are antibodies, antibody fragments, ligands or other binding agents that are specific to a marker molecule, such as a polypeptide, that is present on the cell surface of TrPCs but which is not substantially present on other cell types that would be found in a cell culture produced by the methods described herein.

Methods for making antibodies and using them for cell isolation are known in the art and such methods can be implemented for use with the antibodies and TrPCs described herein. In one process, an antibody which binds to marker expressed by the TrPCs can be attached to a magnetic bead and then allowed to bind to TrPCs in a cell culture which has been enzymatically treated to reduce intercellular and substrate adhesion. The cell/antibody/bead complexes are then exposed to a movable magnetic field which is used to separate bead-bound TrPCs from unbound cells. Once the TrPCs are physically separated from other cells in culture, the antibody binding is disrupted and the cells are replated in appropriate tissue culture medium. If desired, the isolated cell compositions can be further purified by using an alternate affinity-based method or by additional rounds of enrichment using the same or different markers that are specific for TrPCs.

In some embodiments of the processes described herein, a nucleic acid encoding green fluorescent protein (GFP) or another nucleic acid encoding an expressible fluorescent marker gene (e.g., yellow fluorescent protein (YFP), luciferase or the like) is used to label TrPCs. For example, in some embodiments, at least one copy of a nucleic acid encoding GFP or a biologically active fragment thereof is introduced into a pluripotent or multipotent cell, preferably a human embryonic stem cell, downstream of a marker that is expressed by the TrPC, or the promoter of any TrPC-specific gene such that the expression of the GFP gene product or biologically active fragment thereof is under control of the promoter. It is possible to utilize a combination of two-, or more, TrPC-specific gene promoters to activate independently trackable, viable fluorescent reporters by this method, as a means to more specifically label and track, TrPCs, than otherwise achieved using a single-marker strategy.

Fluorescently marked cells, such as the above-described pluripotent or multipotent cells, are differentiated to TrPCs as described herein. Because TrPCs express the fluorescent marker gene, whereas other cell types do not, TrPCs can be separated from the other cell types. In some embodiments, cell suspensions comprising a mixture of fluorescently-labeled TrPCs and unlabeled non-TrPCs are sorted using a FACS. TrPCs are collected separately from non-fluorescing cells, thereby resulting in the isolation of TrPCs. If desired, the isolated cell compositions can be further purified by additional rounds of sorting using the same or different markers that are specific for TrPCs.

In certain embodiments, TrPCs are enriched, isolated and/or purified from other non-TrPCs after pluripotent or multipotent cells are induced to differentiate towards the TrPCs. It will be appreciated that the above-described enrichment, isolation and purification procedures can be used with such cultures at any stage of differentiation.

In addition to the procedures just described, TrPCs may also be isolated by other techniques for cell isolation. Additionally, TrPCs may also be enriched or isolated by methods of serial subculture in growth conditions which promote the selective survival or selective expansion of the TrPCs.

Using the methods described herein, cell populations or cell cultures can be enriched in TrPC content by at least about 2- to about 1000-fold as compared to untreated cell populations or cell cultures. In some embodiments, TrPCs can be enriched by at least about 5- to about 500-fold as compared to untreated cell populations or cell cultures. In other embodiments, TrPCs can be enriched from at least about 10- to about 200-fold as compared to untreated cell populations or cell cultures. In still other embodiments, TrPCs can be enriched from at least about 20- to about 100-fold as compared to untreated cell populations or cell cultures. In yet other embodiments, TrPCs can be enriched from at least about 40- to about 80-fold as compared to untreated cell populations or cell cultures. In certain embodiments, TrPCs can be enriched from at least about 2- to about 20-fold as compared to untreated cell populations or cell cultures.

Some embodiments described herein relate to cell compositions, such as cell cultures or cell populations, comprising TrPCs, wherein the TrPCs are multipotent cells that can differentiate into cells of the endocrine system, such as pancreatic islet insulin producing cells. Other embodiments described herein relate to compositions, such as cell cultures or cell populations, comprising TrPCs and cells that are less specifically differentiated than TrPCs. In such embodiments, cells that are less specifically differentiated than TrPCs comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the culture.

Other embodiments relate to compositions, such as cell cultures or cell populations, comprising TrPCs and cells that are more specifically differentiated than TrPCs, such as immature pancreatic islet hormone-expressing cells and/or mature pancreatic islet hormone-expressing cells. In such embodiments, cells that are more specifically differentiated than TrPCs comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the culture.

Additional embodiments relate to compositions, such as cell cultures or cell populations, produced by the processes described herein and which comprise TrPCs as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% TrPCs. In preferred embodiments, the cells of the cell cultures or cell populations comprise human cells. In other embodiments, the processes described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about 11%, at least about 10%, at least about 9%, at least about 8%, at least about 7%, at least about 6%, at least about 5%, at least about 4%, at least about 3%, at least about 2% or at least about 1% TrPCs. In preferred embodiments, the cells of the cell cultures or cell populations comprise human cells. In some embodiments, the percentage of TrPCs in the cell cultures or populations is calculated without regard to cells remaining in the culture.

Producing an Enriched Population of Pancreatic Insulin Producing Cells from Multipotent or Pluripotent Cells

Embodiments described herein further relate to methods of producing an enriched population of pancreatic insulin producing cells starting from multipotent or pluripotent cells, such as hESCs. Pancreatic insulin producing cells can be produced by first differentiating multipotent or pluripotent cells, such as hESCs, to produce an enriched population of TrPCs. In some embodiments, the process is continued by allowing the TrPCs to further differentiate to pancreatic insulin producing cells by removing the cell condition medium or defined medium or placing the TrPCs in a cell growth medium free of the (i) CXCR4 agonist, (ii) EGFR agonist, (iii) FGFR agonist, (iv) Activin receptor agonist or an agent that stimulates SMAD3, (v) IL11R agonist or IL6R agonist, (vi) notch agonist, (vii) RXR agonist or RAR agonist, or (viii) BMP inhibitor (e.g., at least three of, four of, five of, or six of Activin A, Hb-EGF, CXCL12, FGF growth factor, IL-11, retinoic acid, BMP inhibitor, and non-canonical notch activator).

In some embodiments, the pancreatic insulin producing cells are induced from multipotent or pluripotent cells that are contacted with a defined medium consisting essentially of a cell growth medium that is substantially free of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor. In further embodiments, the cell growth medium is a maturation medium, and further consists essentially of an Ngn3 stabilizer or any other factor or agent described herein as being present in some embodiments of the maturation medium. The inventors have determined that while certain agonists and other factors are useful for stimulating the differentiation of cells, the presence of other agonists and/or factors can lead to the formation of other types of cells, thereby reducing conversion efficiency.

In some embodiments, differentiation from TrPCs to pancreatic insulin producing cells proceeds by continuing the incubation of a culture of TrPCs with a maturation medium that includes an agent, which increases the generation or stabilization of Ngn3 in the pancreas precursor cells and permits the TrPCs to become competent to express at least one pancreatic cell hormone, such as insulin. An example of an agent that increases the generation or stabilization of Ngn3 in the TrPCs is MG132 (benzyl (S)-4-methyl-1-((S)-4-methyl-1-((S)-4-methyl-1-oxopentan-2-ylamino)-1-oxopentan-2-ylamino)-1-oxopentan-2-ylcarbamate). The maturation medium can also include an agent that inhibits notching signaling of the TrPCs. In some embodiments, the agent that inhibits Notch signaling of the TrPCs can be N—[N-(3,5-Diflurophenacetyl-L-alanyl)]-S-phenylglycine t-Butyl Ester (DAPT). DAPT is a specific inhibitor of the g-secretase enzyme. G-secretase enzymatic activity is a requirement for Notch activation, and is the enzyme that liberates the Notch-receptor intracellular domain (Notch1, Notch2, Notch3, Notch4), allowing the intracellular domain to enter the nucleus and activated Notch-regulated gene target promoters. DAPT is an example of a useful g-secretase inhibitor. Other specific inhibitors to the g-secretase enzyme are known to those skilled in the art.

In other embodiments, the maturation medium can include at least one of (a) an agent that promotes the generation of intracellular cAMP, (b) an a Ffar2 agonist, (c) a VDR agonist, or (d) glucose. An agent that promotes the generation of intracellular cAMP can include at least one of 8-Br-cAMP, Forskolin, an Adra2a agonist, epinephrine, adrenaline, Galanin, Galr1 activators, Glp1R agonists, Glp1, or exendin. The Ffar2 agonist can include at least one of a propionate or butyrate, such as sodium propionate or sodium butyrate. The VDR agonist can include Vitamin D3 or metabolites thereof.

In still other embodiments, the maturation medium can include DAPT and/or MG132 and at least one of, two of, or three of (a) an agent that promotes the generation of intracellular cAMP, (b) an a Ffar2 agonist, (c) a VDR agonist, or (d) glucose. The DAPT and/or MG132 and at least one of, two of, or three of (a) an agent that promotes the generation of intracellular cAMP, (b) an a Ffar2 agonist, (c) a VDR agonist, or (d) glucose can be present in the medium that is administered to the cell culture at a concentration, for example, of at least about 1 ng/ml at least about 5 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 65 ng/ml, at least about 70 ng/ml, at least about 75 ng/ml, at least about 80 ng/ml, at least about 85 ng/ml, at least about 90 ng/ml, at least about 95 ng/ml, at least about 100 ng/ml, at least about 110 ng/ml, at least about 120 ng/ml, at least about 130 ng/ml, at least about 140 ng/ml, at least about 150 ng/ml, at least about 160 ng/ml, at least about 170 ng/ml, at least about 180 ng/ml, at least about 190 ng/ml, at least about 200 ng/ml, at least about 250 ng/ml, at least about 300 ng/ml, at least about 350 ng/ml, at least about 400 ng/ml, at least about 450 ng/ml, at least about 500 ng/ml, at least about 750 ng/ml, or at least about 1000 ng/ml. In a preferred embodiment, the maturation medium can also include B27 and Dulbecco's Modified Eagle's Medium (DMEM).

In some embodiments, the maturation medium that includes an agent that increases the generation or stabilization of Ngn3. Such an agent can be provided in the maturation medium with the TrPCs about 1 day, about 2 days, about 3 days, about 4 days, about 5 days, about 6 days, about 7 days, about 8 days, about 9 days, about 10 days or more than about 10 days after the induction of TrPCs. In other embodiments, the maturation medium that includes an agent, which increases the generation or stabilization of Ngn3 can be provided in intermittently with a duration of about 1 h, about 2 h, about 3 h, about 4 h, followed by rest of the culture, of about 1 h, about 2 h, about 3 h, about 4 h, about 8 h, about 24 h, after which a reiteration of the administration of the stabilizing agent is provided again.

In some embodiments, the maturation medium includes an agent, which inhibits notch signaling can be provided in a maturation medium with the TrPCs about 1 day, about 2 days, about 3 days, about 4 days, about 5 days, about 6 days, about 7 days, about 8 days, about 9 days, about 10 days or more than about 10 days after the induction of TrPCs.

In certain processes for producing pancreatic insulin producing cells as described herein, one or more of the above-mentioned differentiation factors are removed from the cell culture or cell population subsequent to their addition. For example, glucose can be removed within about one day, about two days, about three days, about four days, about five days, about six days, about seven days, about eight days, about nine days or about ten days after the addition. Additionally, the glucose after removal can then be added to the differentiation medium. In some embodiments, the glucose removal and addition to the maturation medium can be used to simulate increase and decreases of blood glucose levels during embryo development.

Cultures of pancreatic insulin producing cells can be produced in medium containing reduced serum or no serum. Under certain culture conditions, serum concentrations can range from about 0.05% v/v to about 20% v/v. For example, in some differentiation processes, the serum concentration of the medium can be less than about 0.05% (v/v), less than about 0.1% (v/v), less than about 0.2% (v/v), less than about 0.3% (v/v), less than about 0.4% (v/v), less than about 0.5% (v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8% (v/v), less than about 9% (v/v), less than about 10% (v/v), less than about 15% (v/v) or less than about 20% (v/v). In some processes, pancreatic insulin producing cells are grown without serum, without serum replacement and/or without any supplement containing insulin or insulin-like growth factor.

Optionally, prior to culturing the enriched population of TrPCs in the maturation medium, the cell growth medium or defined medium can be removed from the enriched population of TrPCs and the TrPCs can be cultured in a cell grown medium that includes a Wnt signaling pathway activation agent. Culturing the enriched population of TrPCs in a cell growth medium comprising the Wnt signaling pathway activation agent prior to culturing the TrPCs in the maturation medium can enhance the differentiation of the TrPCs to pancreatic insulin producing cells.

The Wnt genes belong to a family of proto-oncogenes and encode over 20 cysteine-rich secreted glycoproteins that activate the Wnt signaling pathway by binding to Frizzled (Fz) receptors found on target cells. Binding of Wnt ligands to Fz receptors activates the Dishevelled (Dsh/Dvll) protein, allowing it to inhibit the activity of the multiprotein complex comprising beta-catenin, axin-adenomatous polyposis coli (APC) and glycogen synthase kinase (GSK)-3beta. Inhibition of the beta-catenin/APC/GSK-3beta complex prevents phosphorylation of beta-catenin by GSK-3beta. Phosphorylated beta-catenin is targeted for ubiquitin mediated degradation by the proteosome. Therefore, Wnt binding to the Fz receptor results in beta-catenin accumulation in the cytoplasm.

In some embodiments, the Wnt signaling pathway activation agent can include a Wnt receptor ligand or agonist, such as a Frizzled receptor agonist. Examples of Wnt ligands include Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11 and Wnt16. Such Wnt ligands, as well as their accession numbers, are described in U.S. Pat. No. 8,460,928, which is herein incorporate by reference. Any one or more of these may be employed to activate Wnt signalling in the TrPCs.

Wnt ligands may be obtained from R&D Systems (Minnesota, USA) and from PeproTech, Inc (New Jersey, USA).

In some embodiments, the Wnt ligand comprises Wnt1 or Wnt3A, such as Wnt3A. The Wnt ligand may comprise Human WNT1 (PAL1) (ATCC 57198/57199), Human WNT1 (MGC 30915522), Human WNT3 (pHP1) (ATCC MBA-174), Mouse Wnt3 (ATCC MBA-175) or Mouse Wnt3A (ATCC MBA-176).

In addition, the Norrin ligand (Xu et al., 2004, Cell 116(6):883-95), which binds to Frizzled with high affinity, may be used to activate the Wnt signalling pathway. The R-spondin2 protein (Kazanskaya et al (2004) Dev Cell. 7(4):525-34 and Kim et al., (2005) Science 309(5738):1256-9) also binds to the Frizzled receptors and may similarly be used in the methods and compositions described here.

In other embodiments, pharmacological inhibitors of GSK-3β can be used as direct intracellular activators of the canonical Wnt pathway. In ES cells, the stability of beta-catenin, the operative molecule of the canonical Wnt pathway, is controlled by glycogen synthase kinase 3β (GSK-3beta) via phosphorylation and subsequent degradation. Upon Wnt pathway activation, GSK-3beta is inhibited, and the non-phosphorylated beta-catenin is stabilized and enters the nucleus to activate transcription of Wnt-regulated genes. (See Gregorieff et al., (2005) Genes Dev. 19, 877-890). GSK-3beta inhibitors (also referred to herein as GSK3 inhibitors) include, but are not limited to, commercially available GSK-3β inhibitors, for example, those inhibitors available through EMD Biosciences, Madison, Wis., including 1-Azakepaullone, Aloisine, Alsterpaullone, FRATtide (188-225 amino acids of FRAT1), GSK-3beta Inhibitor No. IX, X, XIII, XIV, XV, and GSK-3beta Inhibitor I, II, III, VI, VII, VIII, IX, XI, XII, Peptide Inhibitor (Cat. 361545 and 361546), Indirubin-3′-monoxime, Indirubin-3′-monoxime 5-lodo, Indirubin-3′-monoxime-5-sulfonic Acid, and Kenpaullone and functional derivatives thereof. Other known GSK-3beta inhibitors include small molecules, such as lithium, bivalent zinc, beryllium, aloisines, hymenialdisine, indirubins, maleimides, muscarinic agonists, pyridazinone derivatives, e.g., pyrazolo[3,4-b]quinoxalines, 5-aryl-pyrazolo[3,4-b]pyridazines, and functional derivatives thereof.

In some embodiments, the Wnt signaling pathway activation agent can be present in the medium that is administered to the enriched population of TrPCs at a concentration, for example, of at least about 1 ng/ml at least about 5 ng/ml, at least about 10 ng/ml, at least about 15 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 65 ng/ml, at least about 70 ng/ml, at least about 75 ng/ml, at least about 80 ng/ml, at least about 85 ng/ml, at least about 90 ng/ml, at least about 95 ng/ml, at least about 100 ng/ml, at least about 110 ng/ml, at least about 120 ng/ml, at least about 130 ng/ml, at least about 140 ng/ml, at least about 150 ng/ml, at least about 160 ng/ml, at least about 170 ng/ml, at least about 180 ng/ml, at least about 190 ng/ml, at least about 200 ng/ml, at least about 250 ng/ml, at least about 300 ng/ml, at least about 350 ng/ml, at least about 400 ng/ml, at least about 450 ng/ml, at least about 500 ng/ml, at least about 750 ng/ml, or at least about 1000 ng/ml. In a preferred embodiment, the cell growth medium can also include Dulbecco's Modified Eagle's Medium (DMEM).

In other embodiments, the cell growth medium that includes the Wnt signaling pathway activation agent can be provided with the TrPCs about 1 day, about 2 days, about 3 days, about 4 days, about 5 days, about 6 days, about 7 days, about 8 days, about 9 days, about 10 days or more than about 10 days after the induction of TrPCs.

### Monitoring the Progression of TrPCs to Mature Pancreatic Insulin Producing Cells by Determining the Expression of Markers Characteristic of Pancreatic Insulin Producing Cells

In some embodiments, the progression of TrPCs to mature pancreatic insulin producing cells can be monitored by determining the expression of markers characteristic of pancreatic insulin producing cells. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of mature pancreatic insulin producing cells as well as the lack of significant expression of markers characteristic of hESCs, TrPCs and/or other cell types is determined.

As described in connection with monitoring the production of the TrPCs, qualitative or semi-quantitative techniques, such as blot transfer methods and immunocytochemistry, can be used to measure marker expression. Alternatively, marker expression can be accurately quantitated through the use of technique such as Q-PCR. Additionally, it will be appreciated that at the polypeptide level, many of the markers of pancreatic insulin producing cells are secreted proteins. As such, techniques for measuring extracellular marker content, such as ELISA, may be utilized.

Markers of pancreatic insulin producing cells include, but are not limited to, islet amyloid polypeptide (IAPP), insulin (INS), NKX6 transcription factor related, locus 1 (NKX6.1), glucokinase, (GCK), and/or connecting peptide (C-peptide). The mature pancreatic islet hormone-expressing cells produced by the processes described herein express one or more of the above-listed markers, thereby producing the corresponding gene products. However, it will be appreciated that pancreatic insulin producing cells need not express all of the above-described markers. For example, pancreatic insulin producing cells from hESCs do not co-express INS and GHRL. This pattern of gene expression is consistent with the expression of these genes in human fetal pancreas.

Because pancreatic insulin producing cells do not substantially express the endocrine precursor cell markers NGN3, transition of TrPCs to mature pancreatic islet hormone-expressing cells can be validated by monitoring the decrease in expression of NGN3 while monitoring the increase in expression of one or more of IAPP, INS, NKX6.1, PP, GCK, and/or C-peptide. In addition to monitoring the increase and/or decrease in expression of one or more the above-described markers, in some processes, the diminished expression of genes indicative hESCs and TrPCs is also monitored.

It will be appreciated that IAPP, INS, NKX6.1, GCK, and/or C-peptide marker expression is induced over a range of different levels in mature pancreatic insulin producing cells depending on the differentiation conditions. As such, in some embodiments described herein, the expression of IAPP, INS, NKX6.1, GCK, and/or C-peptide markers in mature pancreatic insulin producing cells or cell populations is at least about 2-fold higher to at least about 10,000-fold higher than the expression of IAPP, INS, NKX6.1, GCK, and/or C-peptide markers in non-pancreatic insulin producing cells or cell populations, for example pluripotent stem cells and/or TrPCs. In other embodiments, the expression of the IAPP, INS, NKX6.1, GCK, and/or C-peptide markers in pancreatic insulin producing cells or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about I0,000-fold higher than the expression of the IAPP, INS, NKX6.1, GCK, and/or C-peptide markers in non-pancreatic insulin producing cells or cell populations, for example pluripotent stem cells and TrPCs.

In some embodiments of the processes described herein, the amount of hormone release from cells and/or cell populations can be determined. For example, the amount of insulin release, glucagon release, somatostatin release and/or ghrelin release can be monitored. In a preferred embodiment, the amount of insulin secreted in response to glucose (GSIS) is measured. In still other embodiments, secreted breakdown or by-products produced by the mature pancreatic islet hormone-expressing cells, such as c-peptide and islet amyloid protein, can be monitored.

It will be appreciated that methods of measuring the expression of secreted proteins are well known in the art. For example, an antibody against one or more hormones produced by islet cells can be used in ELISA assays.

Enrichment, Isolation, and/or Purification of Pancreatic Insulin Producing Cells

Pancreatic insulin producing cells produced by the above-described processes can be enriched, isolated and/or purified by using an affinity tag that is specific for such cells. Examples of affinity tags specific for pancreatic insulin producing cells are antibodies, ligands or other binding agents that are specific to a marker molecule, such as a polypeptide, that is present on the cell surface of mature pancreatic islet hormone-expressing cells but which is not substantially present on other cell types that would be found in a cell culture produced by the methods described herein. In some processes, an antibody which binds to a cell surface antigen on human pancreatic islet cells is used as an affinity tag for the enrichment, isolation or purification of mature pancreatic islet hormone-expressing cells produced by in vitro methods, such as the methods described herein. Such antibodies are known and commercially available. For example, a monoclonal antibody that is highly specific for a cell surface marker on human islet cells is available from USBiological, Swampscott, Mass. (Catalog Number P2999-40). Other examples include the highly specific monoclonal antibodies to glycoproteins located on the pancreatic islet cell surface, which have been described by Srikanta, et al., (1987) Endocrinology, 120:2240-2244, the disclosure of which is incorporated herein by reference in its entirety.

In some embodiments, a Betacam-derived polypeptide, a Betacam reacting antibody, or a small engineered molecule, such as described in U.S. Patent Publication No. 2012/0070847, can be contacted to crude fractions of differentiated pancreatic insulin producing cells to isolate the pancreatic insulin producing cells. If the Betacam-derived polypeptide; Betacam reacting antibody; or a small engineered molecule was previously conjugated or otherwise stably connected to a ligand, affinity-Tag moiety, or fusion protein domain which allows binding to a support material (e.g., plastic dish, plastic tube, sutures, membranes, ultra thin films, bioreactors, microparticles) or suitable matrix (e.g., polymeric matrix), cellular fractional enrichment either through centrifugal spinning, gravitational force, magnetic bead cell adhesion, flow-sorting or other fluid-pressure methodologies, enrichment of the Betacam-expressing cell population, including pancreatic β cells can be achieved.

In some embodiments of the processes described herein, pancreatic insulin producing cells are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label mature pancreatic islet hormone-expressing cells using the methods described above. For example, in some embodiments, at least one copy of a nucleic acid encoding GFP or a biologically active fragment thereof is introduced into a pluripotent cell, preferably a human embryonic stem cell, downstream of the NKX6.1 promoter such that the expression of the GFP gene product or biologically active fragment thereof is under control of the NKX6.1 promoter. In some embodiments, the entire coding region of the nucleic acid, which encodes NKX6.1, is replaced by a nucleic acid encoding GFP or a biologically active fragment thereof. In other embodiments, the nucleic acid encoding GFP or a biologically active fragment thereof is fused in frame with at least a portion of the nucleic acid encoding NKX6. 1, thereby generating a fusion protein. In such embodiments, the fusion protein retains a fluorescent activity similar to GFP.

Fluorescently marked cells, such as the above-described pluripotent cells, are differentiated to pancreatic insulin producing cells as described previously above. Because pancreatic insulin producing cells express the fluorescent marker gene, whereas other cell types do not, pancreatic insulin producing cells can be separated from the other cell types. In some embodiments, cell suspensions comprising a mixture of fluorescently-labeled pancreatic insulin producing cells and unlabeled non-pancreatic insulin producing cells are sorted using a FACS. Mature pancreatic insulin producing cells are collected separately from non-fluorescing cells, thereby resulting in the isolation of pancreatic insulin producing cells. If desired, the isolated cell compositions can be further purified by additional rounds of sorting using the same or different markers that are specific for pancreatic insulin producing cells.

In addition to the procedures just described, pancreatic insulin producing cells may also be isolated by other techniques for cell isolation. Additionally, pancreatic insulin producing cells may also be enriched or isolated by methods of serial subculture in growth conditions which promote the selective survival or selective expansion of the pancreatic insulin producing cells.

Using the methods described herein, enriched, isolated and/or purified populations of pancreatic insulin producing cells and or tissues can be produced in vitro from multipotent or pluripotent cell cultures or cell populations, such as stem cell cultures or populations, which have undergone sufficient differentiation to produce at least some pancreatic insulin producing cells. In a preferred method, the cells are directed to differentiate primarily into pancreatic insulin producing cells. Some preferred enrichment, isolation and/or purification methods relate to the in vitro production of pancreatic insulin producing cells from human embryonic stem cells.

Using the methods described herein, cell populations or cell cultures can be enriched in pancreatic insulin producing cells content by at least about 2- to about 1000-fold as compared to untreated or less specifically differentiated cell populations or cell cultures. In some embodiments, pancreatic insulin producing cells can be enriched by at least about 5- to about 500-fold as compared to untreated or less specifically differentiated cell populations or cell cultures. In other embodiments, pancreatic insulin producing cells can be enriched from at least about 10- to about 200-fold as compared to untreated or less specifically differentiated cell populations or cell cultures. In still other embodiments, pancreatic insulin producing cells can be enriched from at least about 20- to about 100-fold as compared to untreated or less specifically differentiated cell populations or cell cultures. In yet other embodiments, pancreatic insulin producing cells can be enriched from at least about 40- to about 80-fold as compared to untreated or less specifically differentiated cell populations or cell cultures. In certain embodiments, pancreatic insulin producing cells can be enriched from at least about 2- to about 20-fold as compared to untreated or less specifically differentiated cell populations or cell cultures.

Some embodiments described herein relate to cell compositions, such as cell cultures or cell populations, comprising pancreatic insulin producing cells, wherein the pancreatic insulin producing cells are cells, which have been derived from human pluripotent or multipotent cells in vitro, which express one or more pancreatic hormones and which have at least some of the functions of human pancreatic islet cells. In accordance with certain embodiments, the pancreatic insulin producing cells are mammalian cells, and in a preferred embodiment, such cells are human cells.

Other embodiments described herein relate to compositions, such as cell cultures or cell populations, comprising pancreatic insulin producing cells and cells that are less specifically differentiated than pancreatic insulin producing cells. In such embodiments, cells that are less specifically differentiated than pancreatic insulin producing cells comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the culture.

Certain other embodiments described herein relate to compositions, such as cell cultures or cell populations, comprising pancreatic insulin producing cells and cells of one or more cell types selected from the group consisting of pancreatic duct cells and pancreatic acinar cells.

### Administering a Stabilized Form of Ngn3 to Pancreatic Precursor Cells

Still other embodiments relate to a method of inducing or enriching the formation of pancreatic insulin producing cells in a subject by administering a stabilized form of Ngn3 to pancreatic precursor cells. Ngn3 is normally destabilized through the proteasomal system, and this is mediated by Notch, as well as Hes1. Proteasomal degradation relies on ubiquitination of key lysine residues in proteins targeted for destruction. The addition of ubiquitin occurs due to the activity of an appropriate E3 ubiquitin ligase that is able to specifically bind the target protein and perform Ub-conjugation. Several bHLH factors are known to be regulated by ubiquitin-mediated turn-over, including close homologues to Ngn3, such as Ngn1 and MASH1. It was found that it is possible to stabilize the bHLH target upon specific mutation by conversion of Lysine to Arginine, preserving the net positive charge in the chain.

Accordingly, stabilized Ngn3 (Ngn3^{ST}) can include at least one Lysine to Arginine substitution that is refractory to Notch/Hes1 mediated destabilization. This Ngn3^{ST} can be administered to pancreatic precursor cells to promote induction or enrichment of the formation of insulin producing cells. In some embodiments, the pancreatic precursor cells can include TrPCs. In other embodiments, the pancreatic precursor cells can be derived from, or reside in the adult pancreas.

The Ngn3^{ST} can also include other substitutions and/or modifications. For example, the T120 residue in the KLTK loop can be substituted to a Glutamic acid (E) to mimic a possible stabilizing phosphorylation event. The Ngn3^{ST} can be further modified by natural processes, such as post-translational processing, and/or by chemical modification techniques, which are known in the art. Modifications may occur anywhere in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to flavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Other type of peptide modifications may include for example, amino acid insertion (i.e., addition), deletion and substitution (i.e., replacement), either conservative or non-conservative (e.g., D-amino acids) in the polypeptide sequence where such changes do not substantially alter the overall competitive inhibitor ability of the polypeptide.

The Ngn3^{ST} may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may promote axonal growth (without being restricted to the present examples).

The Ngn3^{ST} may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the Ngn3^{ST} may be done by affinity methods, ion exchange chromatography, size exclusion chromatography, hydrophobicity or any other purification technique typically used for protein purification. The purification step can be performed under non-denaturating conditions. On the other hand, if a denaturating step is required, the protein may be renatured using techniques known in the art.

In some embodiments, the Ngn3^{ST} is administered to the pancreatic precursor cells by direct protein delivery to the pancreas in-vivo. The direct protein delivery to the pancreas in-vivo can be in a manner where the protein is designed to cross the plasma membrane.

In some embodiments, the Ngn3^{ST} can be in the form of a conjugate protein or drug delivery construct having at least a transport subdomain(s) or moiety(ies) (i.e., transport moieties). The transport moieties can facilitate uptake of the Ngn3^{ST} into a mammalian (i.e., human or animal) tissue cell. The transport moieties can be covalently linked to a peptides and/or proteins. The covalent link can include a peptide bond or a labile bond (e.g., a bond readily cleavable or subject to chemical change in the interior target cell environment). Additionally, the transport moieties can be cross-linked (e.g., chemically cross-linked, UV cross-linked) to the Ngn3^{ST}.

The transport moieties can be repeated more than once in the Ngn3^{ST}. The repetition of a transport moiety may affect (e.g., increase) the uptake of the peptides and/or proteins by a desired cell. The transport moiety may also be located either at the amino-terminal region of an active agent or at its carboxy-terminal region or at both regions.

In an aspect of the invention, the transport moiety can include at least one transport peptide sequence that allows the Ngn3^{ST} to penetrate into a pancreatic precursor cell by a receptor-independent mechanism.

Additional examples of transport sequences that can be used in accordance with the present invention include a Tat-mediated protein delivery sequence (Vives (1997) 272: 16010-16017), polyargine sequences (Wender et al. 2000, PNAS 24: 13003-13008) and antennapedia (Derossi (1996) J. Biol. Chem. 271: 18188-18193). Other examples of known transport moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, (conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No.: 2,301,157 (Crisanti et al,) incorporated by reference in their entirety). Similarly, HIV Tat protein was shown to be able to cross cellular membranes (Frankel A. D. et al., Cell, 55: 1189).

In addition, the transport moiety(ies) can include polypeptides having a basic amino acid rich region covalently linked to an active agent moiety (e.g., pro-PrP regulating polypeptide). As used herein, the term “basic amino acid rich region” relates to a region of a protein with a high content of the basic amino acids such as arginine, histidine, asparagine, glutamine, lysine. A “basic amino acid rich region” may have, for example 15% or more of basic amino acid. In some instance, a “basic amino acid rich region” may have less than 15% of basic amino acids and still function as a transport agent region. More preferably, a basic amino acid region will have 30% or more of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of the present invention can function as a transport agent region.

In some embodiments, the Ngn3^{ST} can be non-covalently linked to a transfection agent. An example of a non-covalently linked polypeptide transfection agent is the Chariot protein delivery system (See U.S. Pat. No. 6,841,535; Morris et al. (1999) J. Biol. Chem. 274(35):24941-24946; and Morris et al. (2001) Nature Biotech. 19:1173-1176), all herein incorporated by reference in their entirety.

The Chariot protein delivery system includes a peptide transfection agent that can non-covalently complex with the Ngn3^{ST}. Upon cellular internalization, the transfection agent dissociates and the Ngn3^{ST} is free to function. The complex of the Chariot transfection peptide and the Ngn3^{ST} can be delivered to and internalized by mammalian cells allowing for higher dosages of therapeutics to be delivered to the site of pathology. A molar excess of peptide transfection agent relative to the Ngn3^{ST} to be delivered can be employed to accomplish peptide transfection.

In other embodiments, the Ngn3^{ST} can be administered to pancreatic precursor cells by introducing the agent into a pancreatic precursor that causes, increase, upregulates, or promotes expression of Ngn3^{ST} in the pancreatic precursor cells in vivo.

### Inducing Expression of Ngn3^{ST} in a Pancreatic Precursor Cell

One method of introducing the agent into a target cell involves using gene therapy. Gene therapy in some embodiments of the application can be used to express Ngn3^{ST} protein from a pancreatic precursor cell.

In some embodiments, the gene therapy can use a vector including a nucleotide encoding Ngn3^{ST}. A “vector” (sometimes referred to as gene delivery or gene transfer “vehicle”) refers to a macromolecule or complex of molecules comprising a polynucleotide to be delivered to a target cell, either in vitro or in vivo. The polynucleotide to be delivered may comprise a coding sequence of interest in gene therapy. Vectors include, for example, viral vectors (such as adenoviruses (‘Ad’), adeno-associated viruses (AAV), and retroviruses), non-viral vectors, liposomes, and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use herein include viral vectors, lipid based vectors and other non-viral vectors that are capable of delivering a nucleotide to the cells of weakened region, ischemic region, and/or peri-infarct region of the myocardial tissue. The vector can be a targeted vector, especially a targeted vector that preferentially binds to the cells of weakened region, ischemic region, and/or peri-infarct region of the myocardial tissue. Viral vectors for use in the methods herein can include those that exhibit low toxicity to the cells of pancreas and induce production of therapeutically useful quantities of Ngn3^{ST} in a tissue-specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the Ngn3^{ST} and is replication-defective in humans.

Other viral vectors that can be use in accordance with method of the application include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in some embodiments of the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and an Ngn3^{ST} nucleic acid. In methods of delivery to cells proximate the wound, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells.

Lentiviral vectors for use in the methods herein may be derived from human and non-human (including SIV) lentiviruses. Examples of lentiviral vectors include nucleic acid sequences required for vector propagation as well as a tissue-specific promoter operably linked to a Ngn3^{ST} gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used herein. Use of alphaviruses is described in Lundstrom, K., Intervirology 43:247-257, 2000 and Perri et al., Journal of Virology 74:9802-9807, 2000.

In many of the viral vectors compatible with methods of the application, more than one promoter can be included in the vector to allow more than one heterologous gene to be expressed by the vector. Further, the vector can comprise a sequence which encodes a signal peptide or other moiety which facilitates the expression of a Ngn3^{ST} gene product from the target cell.

To combine advantageous properties of two viral vector systems, hybrid viral vectors may be used to deliver a nucleic acid encoding Ngn3^{ST} to a target tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable Ngn3^{ST} gene expression.

Other nucleotide sequence elements which facilitate expression of the Ngn3^{ST} gene and cloning of the vector are further contemplated. For example, the presence of enhancers upstream of the promoter or terminators downstream of the coding region, for example, can facilitate expression.

In addition to viral vector-based methods, non-viral methods may also be used to introduce a Ngn3^{ST} nucleic acid into a target cell. A review of non-viral methods of gene delivery is provided in Nishikawa and Huang, Human Gene Ther. 12:861-870, 2001. An example of a non-viral gene delivery method according to the invention employs plasmid DNA to introduce an Ngn3^{ST} nucleic acid into a cell.

Optionally, a synthetic gene transfer molecules can be designed to form multimolecular aggregates with plasmid Ngn3^{ST} DNA. These aggregates can be designed to bind to cells of weakened region, ischemic region, and/or peri-infarct region of the myocardial tissue. Cationic amphiphiles, including lipopolyamines and cationic lipids, may be used to provide receptor-independent Ngn3^{ST} nucleic acid transfer into target cells (e.g., cardiomyocytes). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Additionally, the Ngn3^{ST} nucleic acid can be introduced into the target cell by transfecting the target cells using electroporation techniques. Electroporation techniques are well known and can be used to facilitate transfection of cells using plasmid DNA.

Vectors that encode the expression of Ngn3^{ST} can be delivered to the target cell in the form of an injectable preparation containing pharmaceutically acceptable carrier, such as saline, as necessary. Other pharmaceutical carriers, formulations and dosages can also be used in accordance with the present invention.

In some embodiments, the Ngn3^{ST} or a vector encoding the Ngn3^{ST} can be provided in a pharmaceutical composition. The pharmaceutical compositions can include a pharmaceutically effective amount of a Ngn3^{ST} described above and a pharmaceutically acceptable diluent or carrier.

In certain embodiments, the Ngn3^{ST} or vector encoding the Ngn3^{ST} can be delivered to cells by site-specific means. Cell-type-specific delivery can be provided by conjugating a therapeutic agent to a targeting molecule, for example, one that selectively binds to the affected cells. Methods for targeting include conjugates, such as those described in U.S. Pat. No. 5,391,723. Targeting vehicles, such as liposomes, can be used to deliver a compound, for example, by encapsulating the compound in a liposome containing a cell-specific targeting molecule. Methods for targeted delivery of compounds to particular cell types are well-known to those skilled in the art.

Methods of Using Trunk Precursor Cells and/or Pancreatic Insulin Producing Cells

In another aspect, methods of use of an isolated population of TrPCs and/or pancreatic insulin producing cells are described herein. In one embodiment, a population of TrPCs and/or pancreatic insulin producing cells may be used for the production of a pharmaceutical composition for the use in transplantation into subjects in need of treatment, e.g., a subject that has, or is at risk of developing diabetes, for example but not limited to subjects with congenital and acquired diabetes. In one embodiment, a population of TrPCs and/or pancreatic insulin producing cells may be genetically modified. In another aspect, the subject may have or be at risk of diabetes and/or metabolic disorder.

In some embodiments, compositions comprising a population of TrPCs and/or pancreatic insulin producing cells as disclosed herein have a variety of uses in clinical therapy, research, development, and commercial purposes. For therapeutic purposes, for example, a population of TrPCs and/or pancreatic insulin producing cells as disclosed herein may be administered to enhance insulin production in response to increase in blood glucose level for any perceived need, such as an inborn error in metabolic function, the effect of a disease condition (e.g., diabetes), or the result of significant trauma (i.e., damage to the pancreas or loss or damage to islet β-cells). In some embodiments, a population of TrPCs and/or pancreatic insulin producing cells as disclosed herein are administered to the subject not only help restore function to damaged or otherwise unhealthy tissues, but also facilitate remodeling of the damaged tissues.

In some embodiments, a method of treating diabetes or a metabolic disorder in a subject is provided that includes administering an effective amount of a composition comprising a population of TrPCs and/or pancreatic insulin producing cells as disclosed herein to a subject with diabetes and/or a metabolic disorder. In a further embodiment, a method for treating diabetes is provided comprising administering a composition comprising a population of TrPCs and/or pancreatic insulin producing cells as disclosed herein to a subject that has, or has increased risk of developing diabetes in an effective amount sufficient to produce insulin in response to increased blood glucose levels.

In one embodiment of the above methods, the subject is a human and the population of TrPCs and/or pancreatic insulin producing cells that is used comprises human cells. In some embodiments, the invention contemplates that a population of TrPCs and/or pancreatic insulin producing cells as disclosed herein are administered directly to the pancreas of a subject, or is administered systemically. In some embodiments, a population of TrPCs and/or pancreatic insulin producing cells as disclosed herein can be administered to any suitable location in the subject, for example in a capsule in the blood vessel or the liver or any suitable site where administered population of TrPCs and/or pancreatic insulin producing cells can differentiate into insulin producing cells and can secrete insulin in response to increased glucose levels in the subject.

In some embodiments, a population of TrPCs and/or pancreatic insulin producing cells as disclosed herein may be used for tissue reconstitution or regeneration in a human patient or other subject in need of such treatment. In some embodiments compositions of populations of TrPCs and/or pancreatic insulin producing cells can be administered in a manner that permits them to graft or migrate to the intended tissue site and reconstitute or regenerate the functionally deficient area. Special devices are available that are adapted for administering cells capable of reconstituting a population of β-cells in the pancreas or at an alternative desired location. Accordingly, the TrPCs and/or pancreatic insulin producing cells may be administered to a recipient subject's pancreas by injection, or administered by intramuscular injection.

In some embodiments, the cells described herein, e.g., a population of TrPCs and/or pancreatic insulin producing cells are transplantable, e.g., a population of TrPCs and/or pancreatic insulin producing cells can be administered to a subject. In some embodiments, the subject is suffering from diabetes, such as type I diabetes, or is a normal subject. For example, the cells for transplantation (e.g., a composition comprising a population of TrPCs and/or pancreatic insulin producing cells) can be in a form suitable for transplantation, e.g., organ transplantation. In some embodiments, the TrPCs and/or pancreatic insulin producing cells may be autologous and/or allogenic.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

In one aspect, a population of TrPCs and/or pancreatic insulin producing cells as disclosed herein are suitable for administering systemically or to a target anatomical site. A population of definitive TrPCs and/or pancreatic insulin producing cells can be grafted into or nearby a subject's pancreas, for example, or may be administered systemically, such as, but not limited to, intra-arterial or intravenous administration. In alternative embodiments, a population of TrPCs and/or pancreatic insulin producing cells can be administered in various ways as would be appropriate to implant in the pancreatic or secretory system, including but not limited to parenteral, including intravenous and intraarterial administration, intrathecal administration, intraventricular administration, intraparenchymal, intracranial, intracisternal, intrastriatal, and intranigral administration. Optionally, a population of TrPCs and/or pancreatic insulin producing cells are administered in conjunction with an immunosuppressive agent.

A composition comprising a population of TrPCs and/or pancreatic insulin producing cells can be administered to a subject using an implantable device. Implantable devices and related technology are known in the art and are useful as delivery systems where a continuous or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Pharmaceutical compositions comprising effective amounts of a population of definitive TrPCs and/or pancreatic insulin producing cells are also contemplated by the present invention. These compositions comprise an effective number of definitive TrPCs and/or pancreatic insulin producing cells, optionally, in combination with a pharmaceutically acceptable carrier, additive or excipient. In certain aspects, a population of definitive TrPCs and/or pancreatic insulin producing cells are administered to the subject in need of a transplant in sterile saline. In other aspects, a population of definitive TrPCs and/or pancreatic insulin producing cells are administered in Hanks Balanced Salt Solution (HBSS) or Isolyte S, pH 7.4. Other approaches may also be used, including the use of serum free cellular media. In one embodiment, a population of definitive TrPCs and/or pancreatic insulin producing cells are administered in plasma or fetal bovine serum, and DMSO. Systemic administration of a population of definitive TrPCs and/or pancreatic insulin producing cells to the subject may be preferred in certain indications, whereas direct administration at the site of or in proximity to the diseased and/or damaged tissue may be preferred in other indications.

In some embodiments, a population of definitive TrPCs and/or pancreatic insulin producing cells can optionally be packaged in a suitable container with written instructions for a desired purpose, such as the reconstitution or thawing (if frozen) of a population of definitive TrPCs and/or pancreatic insulin producing cells prior to administration to a subject.

For administration to a subject, cell populations produced by the methods as disclosed herein, e.g., a population of TrPCs and/or pancreatic insulin producing cells, can be administered to a subject, for example in a pharmaceutically acceptable compositions. These pharmaceutically acceptable compositions comprise a therapeutically-effective amount of a population of TrPCs and/or pancreatic insulin producing cells as described above, formulated together with one or more pharmaceutically acceptable carriers (additives) and/or diluents.

In some embodiments, an amount of a population of TrPCs and/or pancreatic insulin producing cells are administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

In some embodiments, a population of TrPCs and/or pancreatic insulin producing cells as disclosed herein may be administered in any physiologically acceptable excipient, where the TrPCs and/or pancreatic insulin producing cells may find an appropriate site for regeneration and differentiation. In some embodiments, a population of TrPCs and/or pancreatic insulin producing cells as disclosed herein can be introduced by injection, catheter, or the like. In some embodiments, a population of TrPCs and/or pancreatic insulin producing cells as disclosed herein can be frozen at liquid nitrogen temperatures and stored for long periods of time, being capable of use on thawing. If frozen, a population of TrPCs and/or pancreatic insulin producing cells will usually be stored in a 10% DMSO, 50% FCS, 40% RPMI 1640 medium. Once thawed, the cells may be expanded by use of growth factors and/or feeder cells associated with culturing TrPCs and/or pancreatic insulin producing cells as disclosed herein.

In other embodiments, a population of TrPCs and/or pancreatic insulin producing cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of TrPCs and/or pancreatic insulin producing cells as disclosed herein will be adapted in accordance with the route and device used for administration. In some embodiments, a composition comprising a population of TrPCs and/or pancreatic insulin producing cells can also comprise or be accompanied with one or more other ingredients that facilitate the engraftment or functional mobilization of the TrPCs and/or pancreatic insulin producing cells. Suitable ingredients include matrix proteins that support or promote adhesion of the TrPCs and/or pancreatic insulin producing cells, or complementary cell types, especially endothelial cells. In another embodiment, the composition may comprise resorbable or biodegradable matrix scaffolds.

A composition comprising a population of TrPCs and/or pancreatic insulin producing cells can be administrated to the subject in the same time, of different times as the administration of a composition comprising pancreatic insulin producing cells. When administrated at different times, the compositions comprising a population of TrPCs and/or pancreatic insulin producing cells for administration to a subject can be administered within 5 minutes, 10 minutes, 20 minutes, 60 minutes, 2 hours, 3 hours, 4, hours, 8 hours, 12 hours, 24 hours of administration of the other. When compositions comprising a population of TrPCs and/or pancreatic insulin producing cells are administered in different pharmaceutical compositions, routes of administration can be different. In some embodiments, a subject is administered a composition comprising TrPCs. In other embodiments, a subject is administered a composition comprising pancreatic insulin producing cells. In another embodiment, a subject is administered a compositions comprising a population of TrPCs mixed with pancreatic insulin producing cells. In another embodiment, a subject is administered a composition comprising a population of TrPCs and/or a population of pancreatic insulin producing cells, where administration is substantially at the same time, or subsequent to each other.

In other embodiments, a population of TrPCs and/or pancreatic insulin producing cells is stored for later implantation/infusion. A population of TrPCs and/or pancreatic insulin producing cells may be divided into more than one aliquot or unit such that part of a population of TrPCs and/or pancreatic insulin producing cells is retained for later application while part is applied immediately to the subject. Moderate to long-term storage of all or part of the cells in a cell bank is also within the scope of this invention, as disclosed in U.S. Patent Publication No. 2003/0054331, which is incorporated herein by reference. At the end of processing, the concentrated cells may be loaded into a delivery device, such as a syringe, for placement into the recipient by any means known to one of ordinary skill in the art.

Subjects Diagnosed with or Identified as Having Diabetes

In certain embodiments, the subject is a mammal, e.g., a primate, e.g., a human. Mammals other than humans can be advantageously used as subjects that represent animal models of Type 1 diabetes, Type 2 Diabetes Mellitus, or pre-diabetic conditions. A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

As used herein, the phrase “subject in need of a population of TrPCs and/or pancreatic insulin producing cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of TrPCs and/or pancreatic insulin producing cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods described herein further comprise selecting a subject identified as being in need of a population of TrPCs and/or pancreatic insulin producing cells. A subject in need of a population of TrPCs and/or pancreatic insulin producing cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

### Co-Administration of a Pharmaceutically Active Agent

In some embodiments, a composition comprising a population of TrPCs and/or pancreatic insulin producing cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g., Pramlintide), Sodium-dependent glucose cotransporter T2 (SGLT T2) inhibitors (e.g., Dapgliflozin, Remogliflozin and Sergliflozin) and others (e.g., Benfluorex and Tolrestat).

In type 1 diabetes, β-cells are undesirably destroyed by continued autoimmune response. This autoimmune response may also destroy a population of TrPCs and/or pancreatic insulin producing cells implanted into a subject. Thus, this autoimmune response can be attenuated by use of compounds that inhibit or block such an autoimmune response. In some embodiments, a composition comprising a population of TrPCs and/or pancreatic insulin producing cells for administration to a subject can further comprise a pharmaceutically active agent which is an immune response modulator. As used herein, the term “immune response modulator” refers to compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, but are not limited to, members of the group consisting of Lisofylline (LSF) and the LSF analogs and derivatives described in U.S. Pat. No. 6,774,130, contents of which are herein incorporated by reference. Another example of achieving tolerance to TrPC, and/or pancreatic insulin-producing cells, is via thymocyte depletion, achieved, as example by anti-CD3 antibody administration. Other forms of more cell-type selective immune ablation may involve anti-CD4, anti-CD8, and other similar cell-type specific reagents, generally developed towards autoimmune disease prevention and therapy.

In another embodiment, an isolated population of TrPCs and/or pancreatic insulin producing cells as disclosed herein are administered with a differentiation agent. In one embodiment, the definitive TrPCs and/or pancreatic insulin producing cells are combined with the differentiation agent to administration into the subject. In another embodiment, the cells are administered separately to the subject from the differentiation agent. Optionally, if the cells are administered separately from the differentiation agent, there is a temporal separation in the administration of the cells and the differentiation agent. Examples of differentiation agents include retinoids (such as all-trans-retinoic acid (ATRA), 9-cis retinoic acid, 13-cis-retinoic acid (13-cRA) and 4-hydroxy-phenretinamide (4-HPR)); arsenic trioxide; histone deacetylase inhibitors HDACs (such as azacytidine (Vidaza) and butyrates (e.g., sodium phenylbutyrate)); hybrid polar compounds (such as hexamethylene bisacetamide ((HMBA)); vitamin D; and cytokines (such as colony-stimulating factors including G-CSF and GM-CSF, and interferons). The temporal separation may range from about less than a minute in time, to about hours or days in time. The determination of the optimal timing and order of administration is readily and routinely determined by one of ordinary skill in the art.

Effective Dosage Levels of TrPCs and/or Pancreatic Insulin Producing Cells

In some embodiments, a population of TrPCs and/or pancreatic insulin producing cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of TrPCs and/or pancreatic insulin producing cells can be administered to a subject the following locations: clinic, clinical office, emergency department, hospital ward, intensive care unit, operating room, catheterization suites, and radiologic suites.

Toxicity and therapeutic efficacy of administration of a compositions comprising a population of TrPCs and/or pancreatic insulin producing cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of TrPCs and/or pancreatic insulin producing cells that exhibit large therapeutic indices are preferred.

The amount of a composition comprising a population of TrPCs and/or pancreatic insulin producing cells can be tested using several well-established animal models.

The non-obese diabetic (NOD) mouse carries a genetic defect that results in insulitis showing at several weeks of age (Yoshida et al., Rev. Immunogenet. 2:140, 2000). 60-90% of the females develop overt diabetes by 20-30 weeks. The immune-related pathology appears to be similar to that in human Type I diabetes. Other models of Type I diabetes are mice with transgene and knockout mutations (Wong et al., Immunol. Rev. 169:93, 1999). A rat model for spontaneous Type I diabetes was recently reported by Lenzen et al. (Diabetologia 44:1189, 2001). Hyperglycemia can also be induced in mice (>500 mg glucose/dL) by way of a single intraperitoneal injection of streptozotocin (Soria et al., Diabetes 49:157, 2000), or by sequential low doses of streptozotocin (Ito et al., Environ. Toxicol. Pharmacol. 9:71, 2001). To test the efficacy of implanted islet cells, the mice are monitored for return of glucose to normal levels (<200 mg/dL).

Larger animals provide a good model for following the sequalae of chronic hyperglycemia. Dogs can be rendered insulin-dependent by removing the pancreas (J. Endocrinol. 158:49, 2001), or by feeding galactose (Kador et al., Arch. Opthalmol. 113:352, 1995). There is also an inherited model for Type I diabetes in keeshond dogs (Am. J. Pathol. 105:194, 1981).

By way of illustration, a pilot study can be conducted by implanting a population of TrPCs and/or pancreatic insulin producing cells into the following animals: a) non-diabetic nude (T-cell deficient) mice; b) nude mice rendered diabetic by streptozotocin treatment; and c) nude mice in the process of regenerating islets following partial pancreatectomy. The number of cells transplanted is equivalent to about 1000-2000 normal human islets implanted under the kidney capsule, in the liver, or in the pancreas. For non-diabetic mice, the endpoints of can be assessment of graft survival (histological examination) and determination of insulin production by biochemical analysis, RIA, ELISA, and immunohistochemistry. Streptozotocin treated and partially pancreatectomized animals can also be evaluated for survival, metabolic control (blood glucose) and weight gain.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of TrPCs and/or pancreatic insulin producing cells can also be estimated initially from cell culture assays. A dose may be formulated in animal models in vivo to achieve a secretion of insulin at a concentration which is appropriate in response to circulating glucose in the plasma. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

To determine the suitability of cell compositions for therapeutic administration, the TrPCs and/or pancreatic insulin producing cells can first be tested in a suitable animal model. At one level, cells are assessed for their ability to survive and maintain their phenotype in vivo. Cell compositions comprising TrPCs and/or pancreatic insulin producing cells can be administered to immunodeficient animals (such as nude mice, or animals rendered immunodeficient chemically or by irradiation). Tissues are harvested after a period of regrowth, and assessed as to whether the administered cells or progeny thereof are still present.

This can be performed by administering cells that express a detectable label (such as green fluorescent protein, or β-galactosidase); that have been prelabeled (for example, with BrdU or [³H] thymidine), or by subsequent detection of a constitutive cell marker (for example, using human-specific antibody). The presence and phenotype of the administered population of TrPCs and/or pancreatic insulin producing cells can be assessed by immunohistochemistry or ELISA using human-specific antibody, or by RT-PCR analysis using primers and hybridization conditions that cause amplification to be specific for human polynucleotides, according to published sequence data.

A number of animal models for testing diabetes are available for such testing, and are commonly known in the art, for example as disclosed in U.S. Pat. No. 6,187,991 which is incorporated herein by reference, as well as rodent models; NOD (non-obese mouse), BB_DB mice, KDP rat and TCR mice, and other animal models of diabetes as described in Rees et al, Diabet Med. 2005 April; 22(4):359-70; Srinivasan K, et al., Indian J Med. Res. 2007 March; 125(3):451-7; Chatzigeorgiou A, et al., In vivo. 2009 March-April; 23(2):245-58, which are incorporated herein by reference.

Efficacy of treatment of a subject administered a composition comprising a population of TrPCs and/or pancreatic insulin producing cells can be monitored by clinically accepted criteria and tests, which include for example, (i) Glycated hemoglobin (A1C) test, which indicates a subjects average blood sugar level for the past two to three months, by measuring the percentage of blood sugar attached to hemoglobin, the oxygen-carrying protein in red blood cells. The higher your blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of TrPCs and/or pancreatic insulin producing cells as disclosed herein to a subject in need thereof is associated with improved exercise tolerance or other quality of life measures, and decreased mortality. The effects of cellular therapy with TrPCs and/or pancreatic insulin producing cells can be evident over the course of days to weeks after the procedure. However, beneficial effects may be observed as early as several hours after the procedure, and may persist for several years. Successful treatment can also be determined by treatment that results in fewer patients having complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

Genetic Alteration of TrPCs and/or Pancreatic Insulin Producing Cells

In some embodiments, a population of TrPCs and/or pancreatic insulin producing cells as disclosed herein may be genetically altered in order to introduce genes useful in differentiated progeny, e.g., genes useful in insulin producing cells such as pancreatic β-cells, e.g., repair of a genetic defect in an individual, selectable marker, etc., or genes useful in selection against non-insulin producing cells differentiated from a TrPC and/or pancreatic insulin producing cell or for the selective suicide of implanted TrPCs and/or pancreatic insulin producing cells. In some embodiments, a population of TrPCs and/or pancreatic insulin producing cells can also be genetically modified to enhance survival, control proliferation, and the like. In some embodiments, a population of TrPCs and/or pancreatic insulin producing cells as disclosed herein can be genetically altered by transfection or transduction with a suitable vector, homologous recombination, or other appropriate technique, so that they express a gene of interest. In one embodiment, a TrPCs and/or pancreatic insulin producing cells is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of TrPCs and/or pancreatic insulin producing cells. In some embodiments, a population of TrPCs and/or pancreatic insulin producing cells may be genetically altered using vector containing supernatants over an 8-16 h period, and then exchanged into growth medium for 1-2 days. Genetically altered TrPCs and/or pancreatic insulin producing cells can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (i.e., prevent rejection).

In an alternative embodiment, a population of TrPCs and/or pancreatic insulin producing cells as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types, such as somatostatin, glucagon, and other factors.

Many vectors useful for transferring exogenous genes into target TrPCs and/or pancreatic insulin producing cells as disclosed herein are available. The vectors may be episomal, e.g., plasmids, virus derived vectors such as cytomegalovirus, adenovirus, etc., or may be integrated into the target cell genome, through homologous recombination or random integration, e.g., retrovirus derived vectors such MMLV, HIV-1, ALV, etc. In some embodiments, combinations of retroviruses and an appropriate packaging cell line may also find use, where the capsid proteins will be functional for infecting the TrPCs and/or pancreatic insulin producing cells as disclosed herein. Usually, TrPCs and/or pancreatic insulin producing cells and virus will be incubated for at least about 24 hours in the culture medium. In some embodiments, the TrPCs and/or pancreatic insulin producing cells s are then allowed to grow in the culture medium for short intervals in some applications, e.g., 24-73 hours, or for at least two weeks, and may be allowed to grow for five weeks or more, before analysis. Commonly used retroviral vectors are “defective”, i.e., unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g., MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g., 4070A, are capable of infecting most mammalian cell types, including human, dog and mouse. Amphotropic packaging cell lines include PA12 (Miller et al. (1985) Mol. Cell. Biol. 5:431-437); PA317 (Miller et al. (1986) Mol. Cell. Biol. 6:2895-2902) GRIP (Danos et al. (1988) PNAS 85:6460-6464). Retroviruses packaged with xenotropic envelope protein, e.g., AKR env, are capable of infecting most mammalian cell types, except murine cells. In some embodiments, the vectors may include genes that must later be removed, e.g., using a recombinase system such as Cre/Lox, or the cells that express them destroyed, e.g., by including genes that allow selective toxicity such as herpesvirus TK, Bcl-Xs, etc.

Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold. Various promoters are known that are induced in different cell types.

In some embodiments, a population of TrPCs and/or pancreatic insulin producing cells can be applied alone or in combination with other cells, tissue, tissue fragments, growth factors, such as VEGF and other known angiogenic or arteriogenic growth factors, biologically active or inert compounds, resorbable plastic scaffolds, or other additive intended to enhance the delivery, efficacy, tolerability, or function of the population. In some embodiments, a population of TrPCs and/or pancreatic insulin producing cells may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, and may include viral transfection techniques, and more specifically, adeno-associated virus gene transfer techniques. Non-viral based techniques may also be performed.

In another aspect, a population of TrPCs and/or pancreatic insulin producing cells could be combined with a gene encoding pro-angiogenic growth factor(s). Genes encoding anti-apoptotic factors or agents could also be applied. Addition of the gene (or combination of genes) could be by any technology known in the art including but not limited to adenoviral transduction, “gene guns,” liposome-mediated transduction, and retrovirus or lentivirus-mediated transduction, plasmid adeno-associated virus. Cells could be implanted along with a carrier material bearing gene delivery vehicle capable of releasing and/or presenting genes to the cells over time such that transduction can continue or be initiated. Particularly when the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Pat. No. 7,094,874, which is incorporated herein by reference. In one embodiment, an immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the cardiovascular stem cells of the invention.

Use of TrPCs and/or Pancreatic Insulin Producing Cells in a Medical Device

In some embodiments, compositions comprising populations of TrPCs and/or pancreatic insulin producing cells can also be used as the functional component in a mechanical device designed to produce one or more of the endocrine polypeptides of pancreatic islet cells. In its simplest form, the device contains a population of TrPCs and/or pancreatic insulin producing cells behind a semipermeable membrane or immunoprotective barrier that prevents passage of the cell population, retaining them in the device, but permits passage of insulin, glucagon, or somatostatin secreted by the cell population. This includes populations of definitive TrPCs and/or pancreatic insulin producing cells that are microencapsulated, typically in the form of cell clusters to permit the cell interaction that inhibits dedifferentiation. For example, U.S. Pat. No. 4,391,909 describe islet cells encapsulated in a spheroid semipermeable membrane made up of polysaccharide polymers >3,000 mol. wt. that are cross-linked so that it is permeable to proteins the size of insulin, but impermeable to molecules over 100,000 mol. wt. U.S. Pat. No. 6,023,009 describes islet cells encapsulated in a semipermeable membrane made of agarose and agaropectin. Microcapsules of this nature are adapted for administration into the body cavity of a diabetic patient, and are thought to have certain advantages in reducing histocompatibility problems or susceptibility to bacteria.

In some embodiments, compositions comprising populations of TrPCs and/or pancreatic insulin producing cells can also be used as the functional component in a mechanical device designed to produce one or more of the endocrine polypeptides of pancreatic islet cells, with the added provision of oxygen. In its simplest form, the device contains a population of TrPCs and/or pancreatic insulin producing cells behind a semipermeable membrane or immunoprotective barrier that prevents passage of the cell population, retaining them in the device, but permits passage of insulin, glucagon, or somatostatin secreted by the cell population, where the device is furnished with a secondary chamber system that provides for oxygen delivery. This includes populations of definitive TrPCs and/or pancreatic insulin producing cells that are encapsulated, typically in the form of cell clusters to permit the cell interaction that inhibits dedifferentiation. For example, U.S. Pat. No. 8,043,271 describes islet cells encapsulated in an apparatus that includes a housing configured for insertion into a body of a subject. The apparatus includes functional islet cells coupled to the housing and a source of oxygen configured to supply oxygen to the functional cells. The apparatus further includes an external oxygen delivery interface configured to receive oxygen from the source of oxygen, and to facilitate passage of the oxygen to the functional cells, while the housing is implanted within the body of the subject.

More elaborate devices are also contemplated for use to comprise a population of TrPCs and/or pancreatic insulin producing cells, either for implantation into diabetic patients, or for extracorporeal therapy. U.S. Pat. No. 4,378,016 describes an artificial endocrine gland containing an extracorporeal segment, a subcutaneous segment, and a replaceable envelope containing the hormone-producing cells. U.S. Pat. No. 5,674,289 describes a bioartificial pancreas having an islet chamber, separated by a semipermeable membrane to one or more vascularizing chambers open to surrounding tissue. Useful devices typically have a chamber adapted to contain the islet cells, and a chamber separated from the islet cells by a semipermeable membrane which collects the secreted proteins from the islet cells, and which may also permit signaling back to the islet cells, for example, of the circulating glucose level.

Use of TrPCs and/or Pancreatic Insulin Producing Cells as Models or for Screening Potential Therapeutic Compositions and Regulatory Factors

In another embodiment, the TrPCs and/or pancreatic insulin producing cells can be cultured in vitro can be used for the screening of potential therapeutic compositions, such as diabetic therapeutic agents, obesity related therapeutic agents, and/or metabolic syndrome therapeutic agents. These compositions can be applied to cells in culture at varying dosages, and the response of the cells monitored for various time periods. Physical characteristics of the cells can be analyzed by observing cell and neurite growth with microscopy. The induction of expression of new or increased levels of proteins such as enzymes, receptors and other cell surface molecules, or of amino acids, neuropeptides and biogenic amines can be analyzed with any technique known in the art which can identify the alteration of the level of such molecules. These techniques include immunohistochemistry using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radioimmune assays (RIA). Nucleic acid analysis such as Northern blots can be used to examine the levels of mRNA coding for these molecules, or for enzymes which synthesize these molecules.

Alternatively, TrPCs and/or pancreatic insulin producing cells treated with these pharmaceutical compositions can be transplanted into an animal, and their survival, ability to treat diabetes, produce insulin, and biochemical and immunological characteristics examined in an in vivo environment.

In some embodiments, the TrPCs and/or pancreatic insulin producing cells may be genetically engineered to comprise markers operatively linked to promoters that are expressed when a marker is expressed or secreted, for example, a marker can be operatively linked to an insulin promoter. In some embodiments, a population of TrPCs and/or pancreatic insulin producing cells can be used as a model for studying the differentiation pathway of cells which differentiate into islet β-cells or pancreatic β-like cells.

Another embodiment of the present invention includes the use of TrPCs and/or pancreatic insulin producing cells as pancreatic cell models. In another embodiment, an isolated population of TrPCs and/or pancreatic insulin producing cells can be used as models for studying properties for the differentiation into insulin producing cells or pathways of development of cells of endoderm origin into pancreatic β-cells. For the preparation of pancreatic cell models, TrPCs and/or pancreatic insulin producing cells may be proliferated using the methods described above.

The TrPCs and/or pancreatic insulin producing cells can also be used in methods of determining the effect of a biological agent or agents thereon. The term “biological agent” refers to any agent, such as a virus, protein, peptide, amino acid, lipid, carbohydrate, nucleic acid, nucleotide, drug, pro-drug or other substance that may have an effect on the TrPCs and/or pancreatic insulin producing cells whether such effect is harmful, beneficial, or otherwise. The ability of various biological agents to increase, decrease or modify in some other way the number and nature of the TrPCs and/or pancreatic insulin producing cells proliferated in the presence of a proliferative factor can be screened on cells proliferated by various methods, such as those described above. Using an assay identical to or similar to the one described above, for example, it is possible to screen for biological agents that increase the proliferative ability of TrPCs and/or pancreatic insulin producing cells, which would be useful for generating large numbers of cells for transplantation purposes. It is also possible to screen for biological agents, which inhibit neural stem cell or progenitor cell proliferation and/or differentiation. Changes in proliferation may be observed by an increase or decrease in the number of TrPCs and/or pancreatic insulin producing cells that form.

The proliferation and/or differentiation of the TrPCs and/or pancreatic insulin producing cells can be used to identify regulatory factors. The term “regulatory factor” is used herein to refer to a biological factor that has a regulatory effect on the proliferation of the TrPCs and/or pancreatic insulin producing cells. For example, a biological factor would be considered a “regulatory factor” if it increases or decreases the number of stem cells that proliferate in vitro in response to a proliferation-inducing growth factor. Alternatively, the number TrPCs and/or pancreatic insulin producing cells that respond to proliferation-inducing factors may remain the same, but addition of the regulatory factor affects the rate at which the stem cell and stem cell progeny proliferate. A proliferative factor may act as a regulatory factor when used in combination with another proliferative factor.

Using these screening methods, it is possible to screen for potential drug side-effects on TrPCs and/or pancreatic insulin producing cells by testing for the effects of the biological agents on cell and progenitor cell proliferation, and on progenitor cell differentiation or the survival and function of TrPCs and/or pancreatic insulin producing cells. If it is desired to test the effect of the biological agent on a particular differentiated cell type or a given make-up of cells, the ratio of desired cell types obtained after differentiation can be manipulated by separating the different types of cells by, for example, the use of antibodies known to bind and thus select for a particular cell type.

The effects of the biological agents are identified on the basis of significant differences relative to control cultures with respect to criteria such as the ratios of expressed phenotypes, cell viability and alterations in gene expression. Physical characteristics of the cells can be analyzed by observing cell morphology and growth with microscopy. The induction of expression of new or increased levels of proteins such as enzymes, receptors and other cell surface molecules, or, amino acids, neuropeptides and biogenic amines can be analyzed with any technique known in the art which can identify the alteration of the level of such molecules.

The factors involved in the proliferation of stem cells and the proliferation, differentiation and survival of stem cell progeny, and/or their responses to biological agents can be isolated by constructing cDNA libraries from stem cells or stem cell progeny at different stages of their development using techniques known in the art. The libraries from cells at one developmental stage are compared with those of cells at different stages of development to determine the sequence of gene expression during development and to reveal the effects of various biological agents or to reveal new biological agents that alter gene expression in TrPCs and/or pancreatic insulin producing cells. When the libraries are prepared from dysfunctional tissue, genetic factors may be identified that play a role in the cause of dysfunction by comparing the libraries from the dysfunctional tissue with those from normal tissue. This information can be used in the design of therapies to treat the disorders. Additionally, probes can be identified for use in the diagnosis of various genetic disorders or for use in identifying TrPCs and/or pancreatic insulin producing cells at a particular stage in development.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Isolation of Pancreatic-Specific Embryonic Mesenchymal Cells (Pan-MEF Cells)

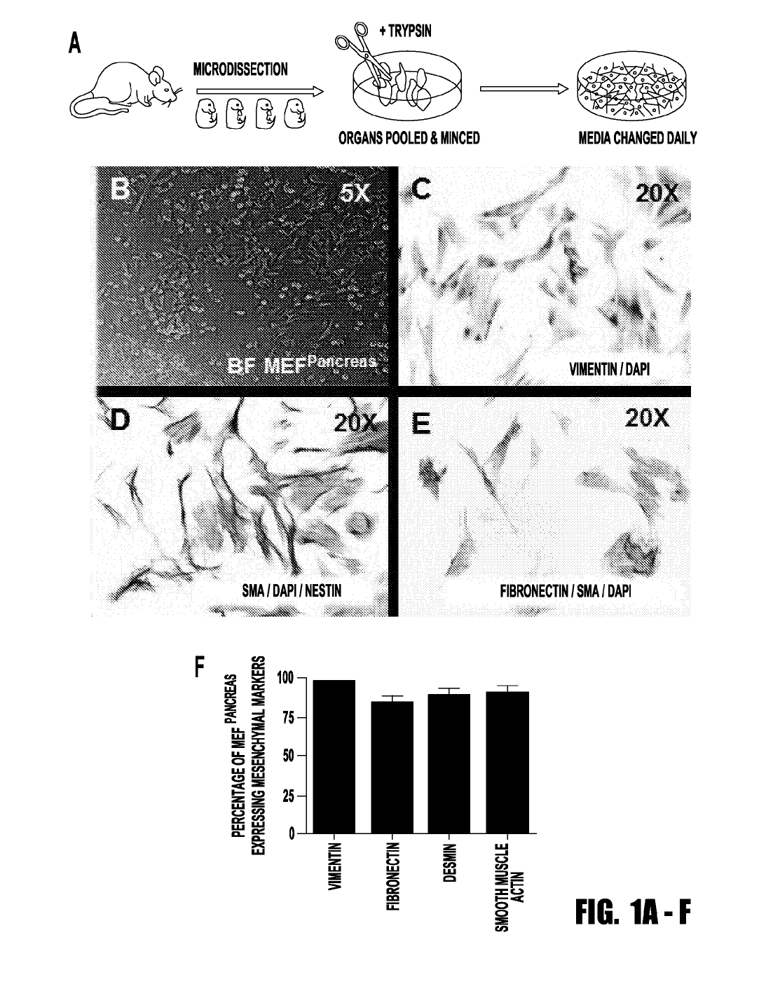

The derivation of pancreas-specific mesenchymal cells was accomplished through a combination of mechanical and enzymatic digestions. The timing of CD-1 pregnant mice was accomplished through the daily inspection for vaginal plugs, when vaginal plugs were detected it was assumed to be E0.5 (0.5 days of embryonic development). Culture derivation was accomplished by asphyxiating repugnant dams of E13.5 embryos in a carbon dioxide chamber followed by cervical dislocation. The abdomen was thoroughly washed with ethanol before creating an incision to expose the uterine horns for removal. Embryos were removed from embryo sacs and separated from the placenta followed by decapitation. Dissections were performed under a microscope and organs from an entire litter pooled. Pooled pancreatic tissue was minced for five minutes followed by a 15 minute trypsin digestion performed at 37°. The enzymatic digestion was diluted five-fold in a MEF medium preparation to stop the trypsin digestion. Primary cultures were established best when two timed pregnancies were used for establishing a culture within a single well of a six-well plate. Cultures were washed daily in PBS and passaged at a ratio of one to three every other day. The continued washing and passaging of primary cultures was found to successfully remove any epithelial components from cultures. Cultures were consistently used on the sixth day of ex vivo culture for the conditioning of medium. Cultures did not proliferate well in the absence of FBS and consequently cultures were grown in the presence of serum, but were used to condition medium lacking serum. Cultures were used for conditioning medium for three consecutive days before being discarded. A representative established culture is shown in panel B of FIG. 1.

To evaluate the composition of Pan-MEF cultures with regard to purity an immuno-histochemical analysis of several mesenchymal markers was used. The intermediate filament Vimentin, a well accepted marker for mesenchymal cells, was found present within over 98% of the cells present in Pan-MEF cultures (FIG. 1 Panels C and F). While other mesenchymal markers assayed had lower percent expression within Pan-MEF cultures; Smooth Muscle Actin (denoted SMA in panel D and E of FIG. 1) was present in a little more than 90% of the culture, Desmin (FIG. 1 panel F) was found in slightly less than 90% of the culture and Fibronectin was present at the lowest percentage only being expressed within approximately 84% of the cells within the culture (FIG. 1 panels E and F). When considering mesenchymal markers present in 90% or less of the culture, it is important to note that perfect co-expression for all the mesenchymal markers assayed was not observed, for instance the occurrence of Fibronectin⁺/SMA⁻ cells are shown in FIG. 1 panel E. In addition FACS analysis of several different Pan-MEF cultures have demonstrated that between 92-98% of the culture is PCAM⁻ (data not shown), further evidence that the endothelial components of the embryonic pancreatic pool do not get established in the ex vivo culture system described.

### Example 2

### Isolation of Endodermal Organ Specific Mesenchymal Cells and their Characterization

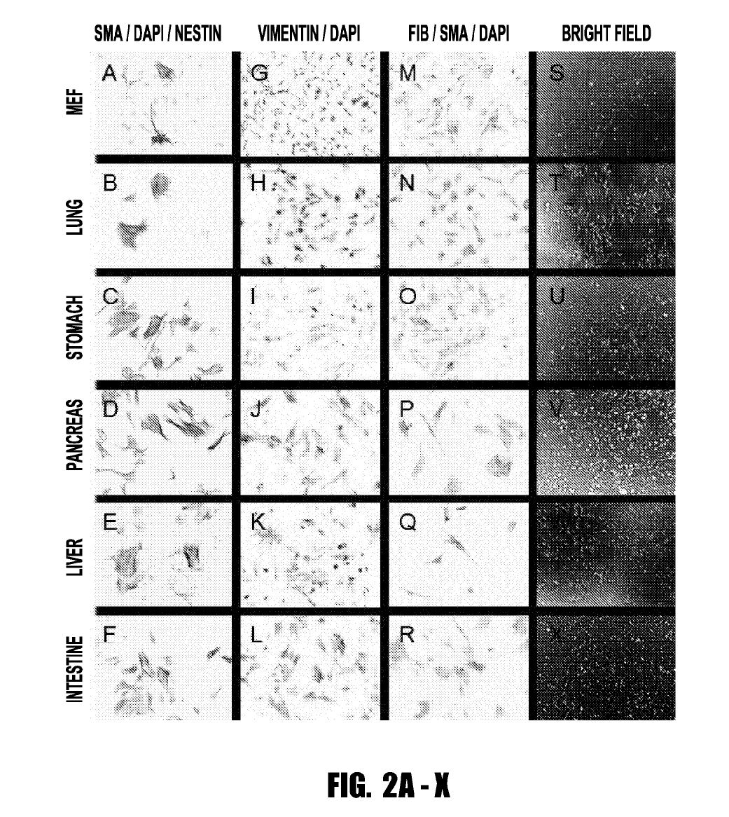

As a means of evaluating the unique phenotype present within the Pan-MEF cultures several organ-specific mesenchymal cultures were established form different endodermal derived embryonic organs. While the methodologies used to establish the different organ-specific MEF cultures were the same as explained within Example 1, important differences with respect to the seeding density needed to obtain a proliferating culture were noted. Cultures derived from embryonic intestines and embryonic stomachs both required initial seeding in a single well of a six-well plate for proper seeding density. Lung-MEF cultures were easily established in a single 8 cm culture plate, while Liver-MEFs required a T-175 flask to successfully be established. In addition Liver-MEFs required far more washings and one to one ratio passages (non-expansion) to properly set up a culture consisting of mesenchymal cells. However once established the various organ-specific mesenchymal cultures displayed similar morphology between cultures established from different organs to the extent that cells of fibroblastic appearance were derived. Minor differences were noted as to the overall cell size and growth pattern between cultures. All cultures could be passaged, and were taken through three passage events prior to RNA isolation. FIG. 2 shows representative cultures of the various established organ-specific mesenchymal cultures as compared to a generic mouse embryonic fibroblast culture (feeder culture).

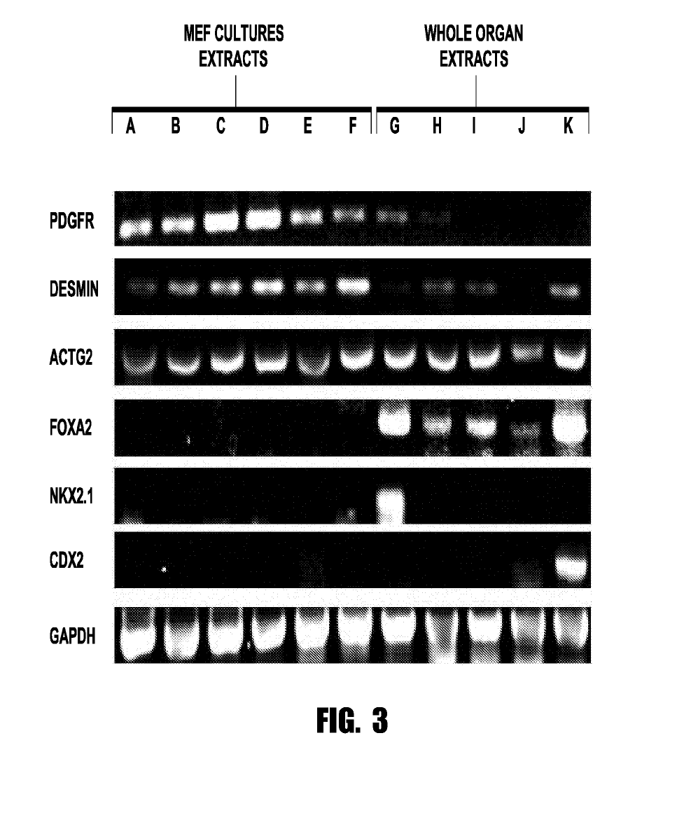

Bright field images of all of the organ-specific cultures (FIG. 2 panels S-X) demonstrate highly homogenous cultures composed of by cells displaying a fibroblast morphology. Immunohistochemical analysis of these same cultures agrees with this qualitative assessment. The vast majority of all cells present within all the different organ-specific primary cultures analyzed displayed Vimentin expression (FIG. 2 panels G-L), Fibronectin expression (FIG. 2 panels M-R) and Nestin expression (FIG. 2 panels A-F). The greatest degree of variability in expression patterns were noted in the Smooth Muscle Actin expression between the different cultures, Liver-MEF cultures displayed a sizable population of SMA⁻ cells and MEFs derived from embryonic body trunks displayed few SMA⁺ cells. In addition the generic MEF culture also displayed a much smaller percentage of Nestin⁺ cells when compared to all of the organ-specific cultures. As a means to specifically ensure mesenchymal homology throughout the differing cultures RNA extracts were analyzed for the presence of epithelial/endodermal markers using RT-PCR. FIG. 3 compares the expression patterns of several mesenchymal and epithelial markers between the various organ-specific cultures and the respective whole embryonic organs. While the mesenchymal markers assayed all have higher expression within the RNA samples derived from the organ-specific MEF cultures, signifying an enrichment in the cellular components responsible for these markers, the endothelial markers assayed were completely absent from these samples. The general endodermal marker FoxA2 was found expressed within all the whole embryonic organ RNA extracts and missing from all established cultures clearly demonstrating the loss of epithelial components from these primary cultures. Further the tissue specific markers for lung and intestines, Nkx2.1 and Cdx2 respectively, were found only in the whole embryonic organ extracts isolated from these two organs. All together this demonstrates that the derivation process and following culture conditions used in the establishment of the organ-specific MEF cultures successfully removes any epithelial components present in the crude embryonic organ extracts used to establish the various cultures.

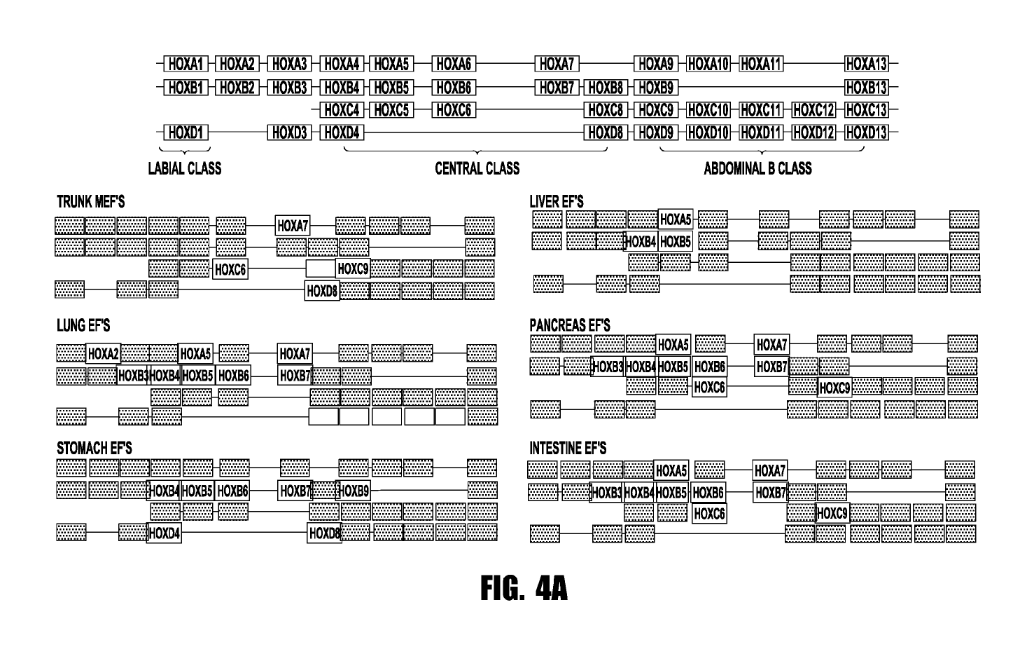

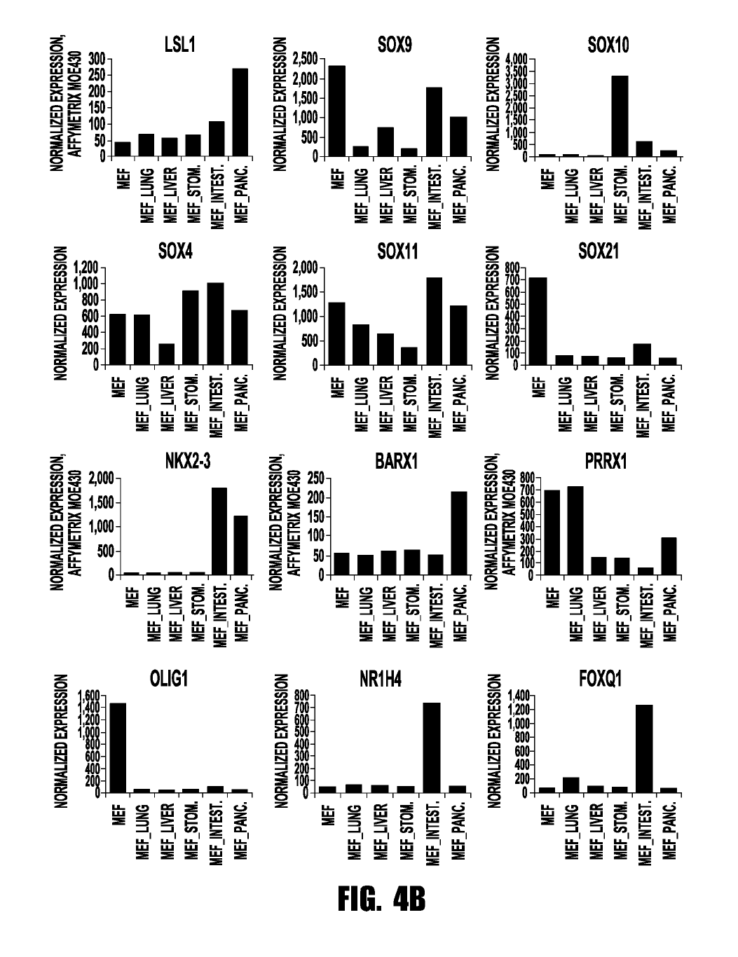

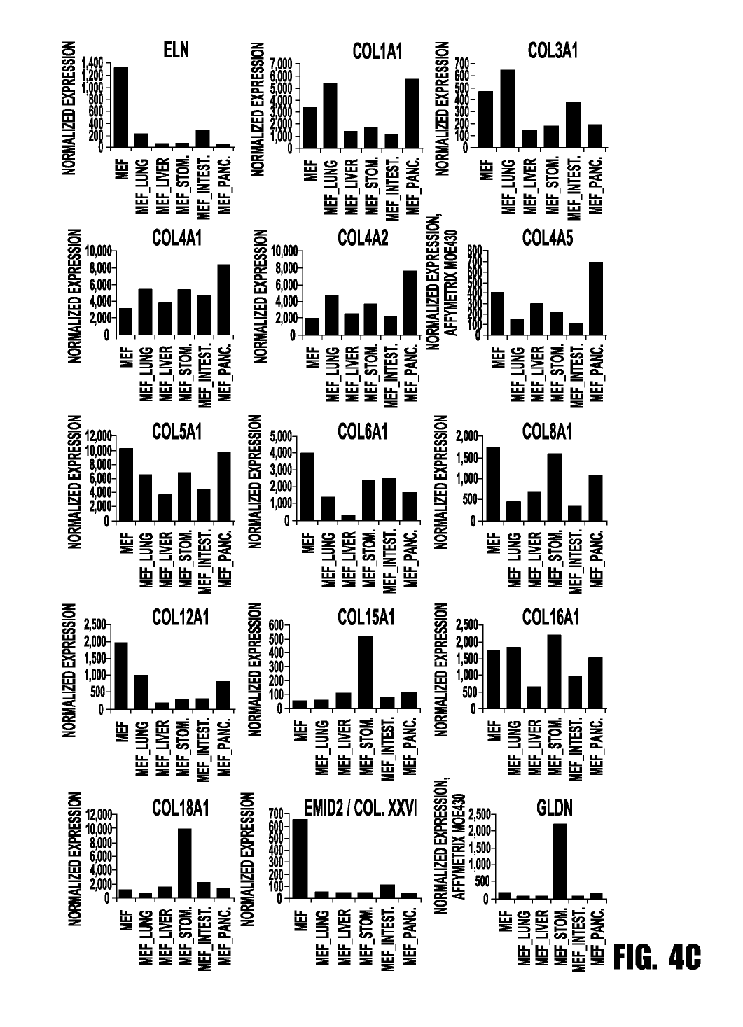

While all of the noted differences in expression patterns above imply variances between the different organ-specific mesenchymal cultures, microarray analysis of these different cultures confirms vast phenotypic differences. FIG. 4A shows the differential expression of Hox genes between the various organ-specific MEF cultures. While the Hoxb4 and Hoxb5 genes are expressed throughout all of the organ-specific MEF cultures, many of the detected Hox genes are segregated into fewer cultures and many are unique to individual cultures being assayed. In terms of least utilized Hox codes both the MEFs derived from the body trunk and the Liver-MEF cultures have few Hox genes expressed. While the MEFs derived from the other organ compartments (lung, stomach, pancreas and intestines) share a higher similarity in Hox gene expression patterns, there are many differences between cultures. These four cultures commonly express the Hoxb4, Hoxb5, Hoxb6 and Hoxb7 gene cluster, suggesting a greater similarity between these cultures when compared to the MEFs and Liver-MEF. All together this clearly demonstrates a unique hox code utilization between the different cultures and this is indicative of drastic changes in the gene expression patterns among the different cultures.

While the paralogous HOX-family members are useful to identify different spatial origins of the MEF cells along anterior/posterior and dorsal/ventral axes of the embryo, it is also clear that further inspection of cell fate determining genes, encoding specific transcription factors belonging to several different families are differentially expressed in the MEF cultures (FIG. 4B). These include Sox-family members, Sox4, 9, 11, which are all variably expressed among the cultures, and Sox10 highly selectively expressed in Stomach-MEF, and Sox21 highly specific for Trunk-MEF. Nkx2.3 is in contrast highly selective for intestinal-MEF and pancreas-MEF, but Olig1 is exclusive to Trunk-MEFs. Of note, the bile-acid nuclear receptor Nr1h4 is exclusive for intestine-MEF, as is the FOX-family member, Foxq1, encoding a factor that is involved in suppressing smooth muscle fates.

The differential expression of cell intrinsic regulators of phenotype, as provided by the investigations of the gene regulatory factors above would indicate dramatic differences in gene expression of terminal, downstream targets. To evaluate a relevant gene family, we focused on the collagen-domain containing protein class. In mice, a total of 109 different genes encode Collagen-Domain (IPR008160)-containing proteins. Collagens are among the most abundantly expressed genes in mesenchymal stem cells, as these represent one of the primary components of the extracellular matrix (ECM) produced by mesenchymal cells. Collagens are almost exclusively expressed by mesenchymal-type cells, and the family of collagens include proteins that are providing elastic support, such as elastin (Eln), and ECM-type collagens (such as Col4- and Col6-family members. The individual properties of the collagen defines the tensive strength of the ECM, or specific ligamentous structures in which the protein participates. Inspecting the collagen family between the MEF-type cultures, we noted extreme variation in Col-family expression, telling about significant functional differences of MEFs in relation to their tissue of origin. For instance, the Col4 family members were expressed by all MEF cultures, although most abundantly in Pancreas-MEF. Not surprisingly, the Elastin collagen was only abundant on Trunk-MEFs. Some collagens were quite specific for a single MEF-type cells, such as Col15a1, Col18a1, and Gliomedin (gldn), which all were abundantly expressed in stomach-MEF. Similar to Elastin, Emid2 (Collagen XXVI) was selective for trunk-MEFs.

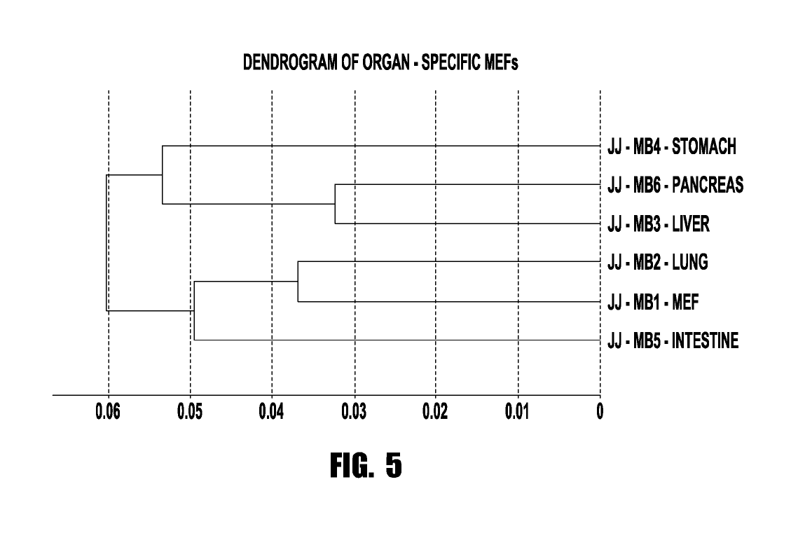

The above analyses were supported by the provided dendrogram seen in FIG. 5, which provides a complete comparison of phenotypes segregation of MEF-type cells. The dendogram shows that the samples segregate into two distinct branches; one composed of the samples derived from the Stomach-MEF, Pancreas-MEF and Liver-MEF cultures and a second branch composed of by the samples from the Lung-MEF, Intestine-MEF and body trunk-MEF cultures. Indicating that the greatest similarities between the organ-specific cultures occur between the mesenchyme derived from embryonic stomachs, pancreas and liver, while the embryonic lung and intestinal derived MEF cultures were more similar to the MEF cultures derived from the embryonic body trunks. In addition wide variance between the morphogens expression pattern present within the different cultures was observed (not shown).

### Example 3

### Organ-Specific MEF-Cells do not Support Pluripotency Maintenance

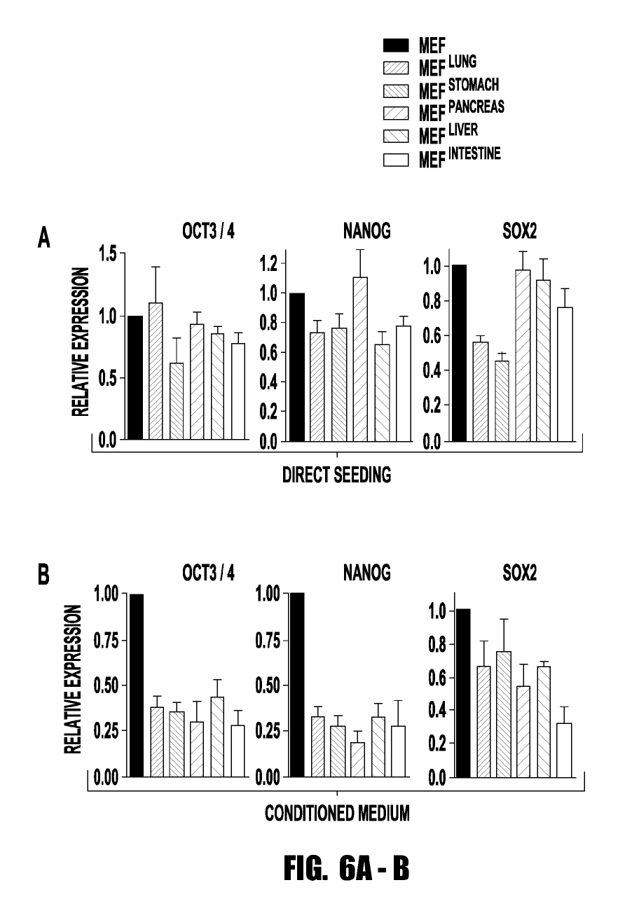

Since it has been established that there are vast phenotypic variance between the different organ-specific MEF cultures and that these differences were also seen in the morphogens compliment associated with the different cultures, we next assayed for the capacity of the different cultures to sustain pluripotency within a hES culture. Most of the organ-specific MEF cultures were impaired to various degrees in their capacity to sustain pluripotency when the H1 hES cell line were directly seeding onto them. Only the pancreas specific MEF culture was shown to be capable of sustaining pluripotency to the same efficiency as the MEF controls (FIG. 6 Panel A). The liver and intestinal specific MEF cultures only displayed a slight impairment in pluripotent maintenance as inferred through the 70-90% expression levels of the pluripotent markers Oct3/4, Nanog and Sox2. While the lung and stomach derived MEF cultures were the most impaired in their ability to sustain pluripotency when the H1 hES cell culture was directly seeded onto them as is evident through the fact that the expression levels of the three markers assayed reached levels as low as 50% of the controls. It should be noted that the lung-specific MEF cultures did show a slight increase in Oct3/4 expression, though this could be attributed to a forward differentiation event since Oct3/4 expression is not exclusively limited to the inner mass cells during development.

While direct seeding onto the various organ-specific cultures failed to demonstrate a strong tendency for the pluripotent cultures to differentiate, when the various organ-specific MEF cultures were used to condition medium which then was supplemented onto pluripotent cultures any ability to successfully sustain pluripotency by these cultures was lost (FIG. 6 panel B). Regardless of the organ of origin, when organ-specific conditioned medium was used on feeder-free pluripotent cultures, both Oct3/4 and Nanog expression levels ranged from 20-50% of the levels found within control cultures, indicating a significant loss in pluripotency maintenance. While Sox2 levels were found to be higher, within the range of 30-80% of the control cultures, its lone maintenance cannot account for sustained pluripotency of the cultures. All together this shows that any capacity of the organ-specific MEFs to sustain pluripotency relies on the direct contact of hES with the organ-specific MEFs in a coculture system reminiscent of feeders and cannot be attributed solely to the factors being secreted from said cultures.

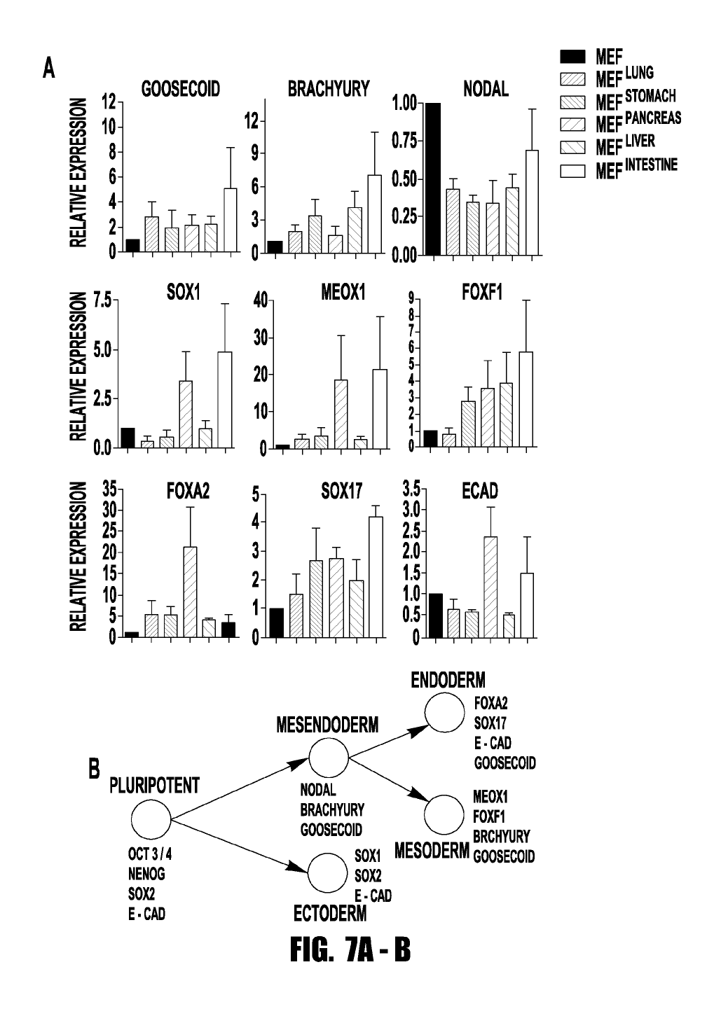

Since we have shown that the pluripotency of hES cultures collapses when said cultures are maintained in organ-specific MEF condition medium, the feeder-free system was used to assay the ability of these primary mesenchymal cultures to induce differentiation upon a pluripotent culture. As a means of identifying the effects the various organ-specific MEF cultures had on forward differentiating hES cultures an array of different markers representative of the different germ layers were investigated as outlined in FIG. 7 panel B. In all cases the primitive streak markers Goosecoid and Brachyury were up regulated over control reactions (hES cultures supplemented with conditioned medium derived from MEF-feeder cultures), though this up-regulation was moderate ranging from approximately 2-6 fold over controls. Interestingly the primitive streak marker Nodal was not up regulated in any of the samples. Only two of the samples, the hES cultures which were supplemented with the pancreas and intestinal specific conditioned medium, showed any increase in the ectodermal marker Sox1. While all of the samples showed an increased expression in markers representative of the mesodermal and endodermal lineages. Interestingly, the hES culture which was supplemented with pancreas-specific conditioned medium demonstrated a substantial 20-fold increase in FoxA2 expression over the control culture condition (FIG. 7 panel A). All together this demonstrates that the organ-specific mediated forward differentiation of pluripotent cultures proceeds mainly through a mesoendodermal intermediate and generates cells of both the endodermal and mesodermal lineages.

### Example 4

### Lung-Specific MEF Cells Induce Pulmonary Identity within Pluripotent Cultures

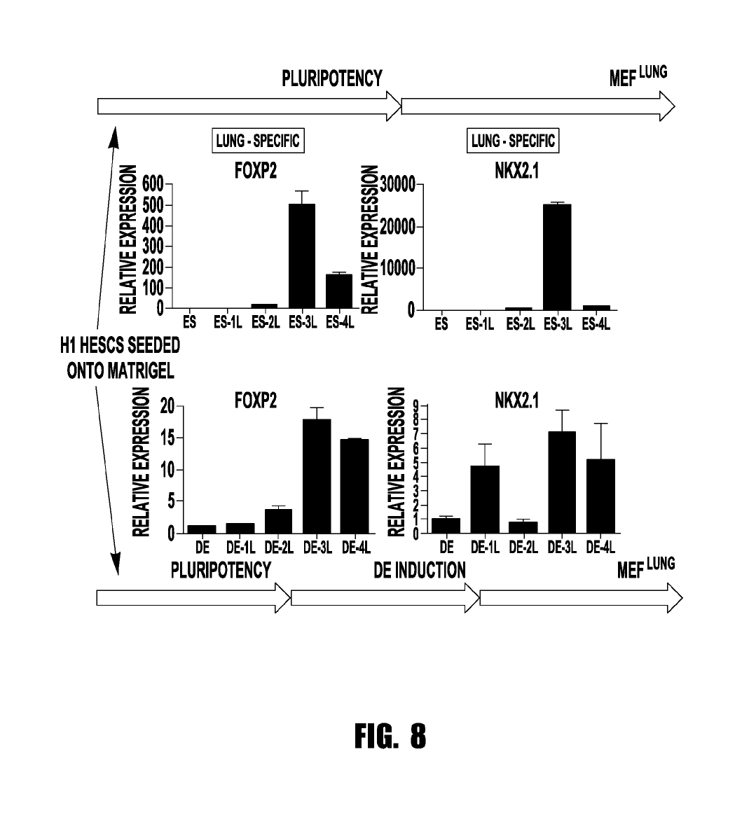

Since we have seen evidence of the different organ-specific cultures ability to drive the differentiation of pluripotent cultures towards endodermal fates, we next investigated weather the endodermal fates derived from the organ-specific conditioned medium had organ-specific characteristics. Specifically we assayed for the ability of lung-specific MEF conditioned medium (Lung-CM) to inform lung characteristics on pluripotent cultures. Pluripotent cultures were passaged onto Matrigel and grown in MEF conditioned medium for 4 days to produce a confluent pluripotent plate, at which time these cultures were supplemented with Lung-CM for an additional 4 days. Daily transcript analysis for the lung specific genes FoxP2 and Nkx2.1 (FIG. 8) demonstrated a substantial up-regulation of both genes peaking at 3 days and decreasing sharply thereafter. In a parallel experiment pluripotent cultures were directed to differentiate to definitive endoderm before the cultures were supplemented with Lung-CM In this scenario the relative expression of both FoxP2 and Nkx2.1 again peaked at day 3, however the overall levels of these two transcripts were much lower. The maximum relative fold increases for FoxP2 and Nkx2.1 when the lung-specific MEF conditioned medium was supplemented directly onto pluripotent cultures were 500 and 25,000 respectively as compared to 20 and 7 fold increases when hES cultures were taken to a definitive endoderm intermediate before supplementation of the lung-specific conditioned medium. All together this demonstrates that the ability of Lung-CM to inform lung identity within a pluripotent culture was greatly impaired when the pluripotent culture was differentiated to a definitive endodermal culture before being incubated in the presence of Lung-CM.

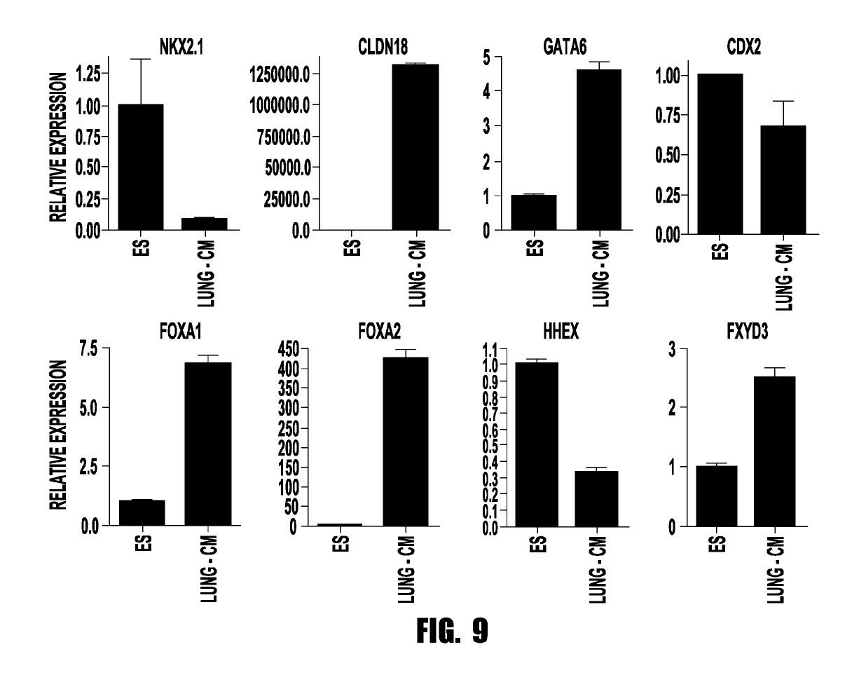

We next addressed the expression patterns of pluripotent cultures which were maintained for an entire week in the presence of Lung-CM (FIG. 9). While the expression of the lung-specific homeobox gene Nkx2.1 was almost entirely lost by this point, several endodermal genes known to play important roles in lung development were abundantly expressed. Both FoxA1 and Gata6 were moderately up regulated with relative fold increases of 6 and 4.5 fold respectively. The endodermal gene FoxA2 displayed a fold increase 425 times higher than the control and the lung-specific gene Cldn18 was up regulated over 1,250,000 fold. Additionally a number of genes known to be specific to other organs were assayed, the intestinal gene Cdx2, the liver-specific gene Hex and a stomach specific gene Fxyd3, and none of these genes were found to be significantly up-regulated over the control conditions. All together this shows that the lung specific mesenchymal cultures secrete factors which are at least partially capable of driving the differentiation of pluripotent cells toward endodermal and lung specific fates.

### Example 5

### Organ-Specific Fate Induction of Pluripotent Cells Using Organ Specific MEF Cells

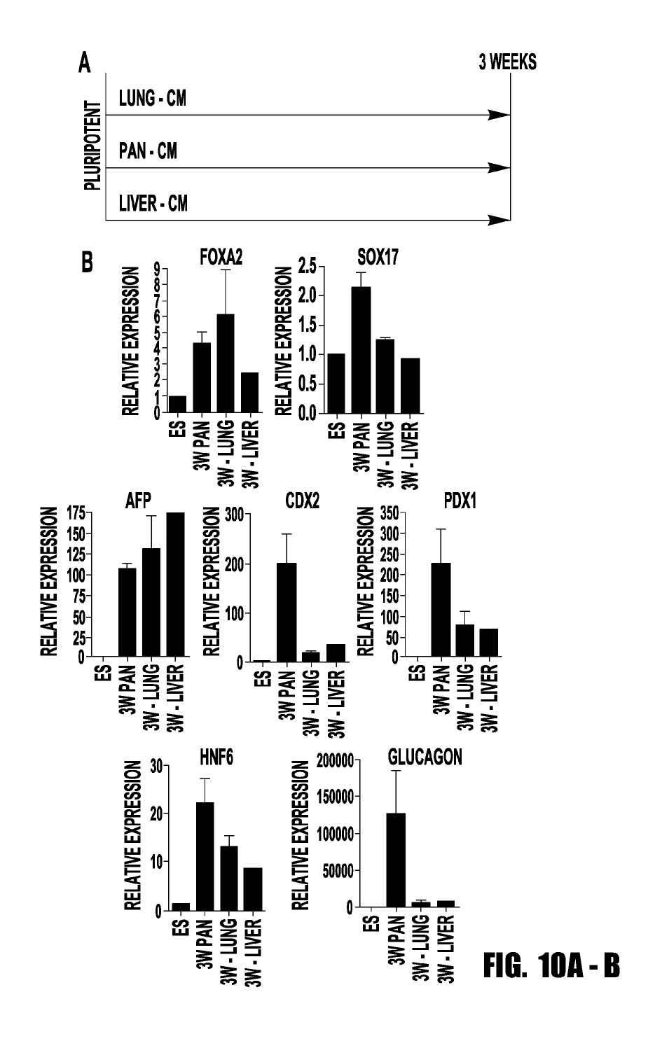

We next assayed the ability of different organ-specific MEF cultures to drive the differentiation of pluripotent cells toward their own specific organ fates. Our main objective in doing this was to specifically test weather the organ fates being induced in the pluripotent cultures were specific to the organ identity of the MEF cultures used to drive their differentiation and not the effects of a random forward differentiation event which could occur to a loss of pluripotent maintenance. Conditioned medium from three different organ-specific MEF cultures, pancreas, intestines and lung, were used to condition medium which in turn was supplemented onto feeder free pluripotent cultures for a three week period of time. All three experimental conditions showed evidence of endoderm formation with the relative values for FoxA2 expression ranging from a 2.5 fold increase, as seen in the sample being supplemented with Liver-CM, to a 6 fold increase in the pluripotent sample grown in the presence of Lung-CM (FIG. 10 panel B). Sox17 expression was lower ranging from no fold increase, as seen in the sample supplemented with Liver-CM, to a two fold increase reached with the sample incubated in the presence of Pan-CM.

More importantly a differential expression pattern was observed with all of the organ-specific transcripts analyzed. AFP expression was highest in the sample supplemented with Liver-CM, though it's the relative expression was not too much greater than the pluripotent cultures supplemented with either Pan-CM or Lung-CM. The pancreatic specific progenitor transcript Pdx1 expression was substantially higher within the pluripotent sample supplemented with Pan-CM when compared to the other samples. Relative expression of Pdx1 within cultures supplemented with Pan-CM was ˜225 fold increased over hES levels as compared to ˜75 fold induction observed in the Lung-CM sample or 65 fold induction observed in the sample supplemented with Liver-CM. In addition the pancreatic endocrine marker Glucagon supported the notion that the pluripotent sample exposed to Pan-CM had a greater pancreatic phenotype than the other samples. Relative Glucagon levels were increased ˜125,000 fold over hES levels in pluripotent samples supplemented with Pan-CM. This level of induction far exceeded the relative expression levels of Glucagon observed in the other samples which ranged from a ˜6000 fold increase in the pluripotent culture supplemented with Lung-CM or the ˜7500 fold increase observed in the pluripotent sample supplemented with Liver-CM.

All together this demonstrates that the Pan-CM successfully increase pancreatic characteristics found within forward differentiating pluripotent cultures. However this pair-wise induction observed through the different organ-specific MEF cultures is not absolute as Cdx2 and AFP expressions are increased in the pluripotent samples exposed to Pan-CM and some Pdx1 and Glucagon expression is also observed within the pluripotent cultures which were supplemented with either Lung-CM or Liver-CM. It is more reasonable to conclude that the different organ-specific MEF cultures selectively enhances endodermal fates within forward differentiating cultures with the strongest endodermal fate being dictated by the organ the different MEFs were derived from, however this induction is not absolute in that other endodermal fates are permitted, but occur to a lesser degree.

### Example 6

### Pancreatic Specific MEFs Efficiently Induce Definitive Endoderm from Pluripotent Cells

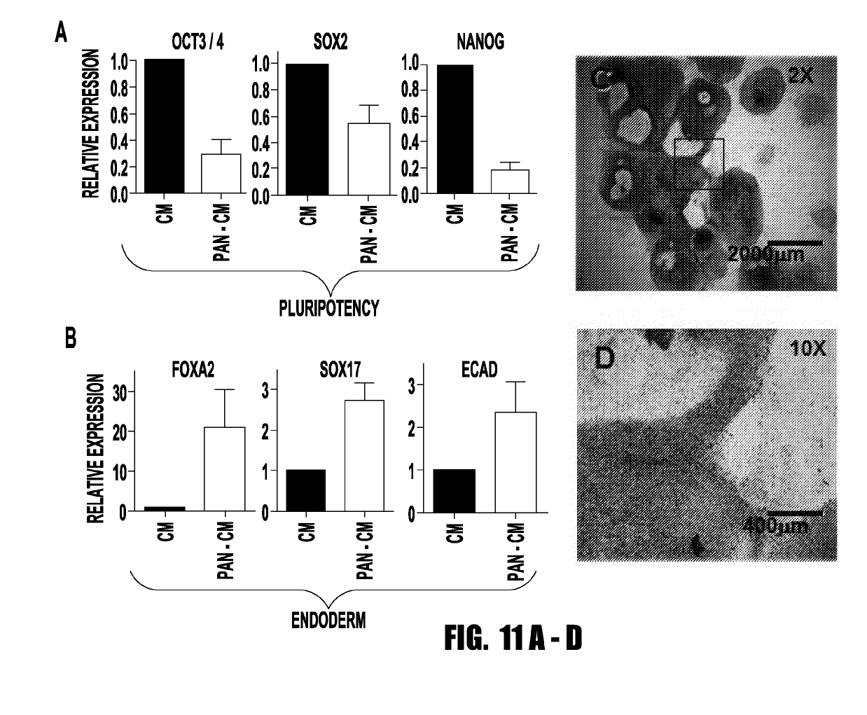

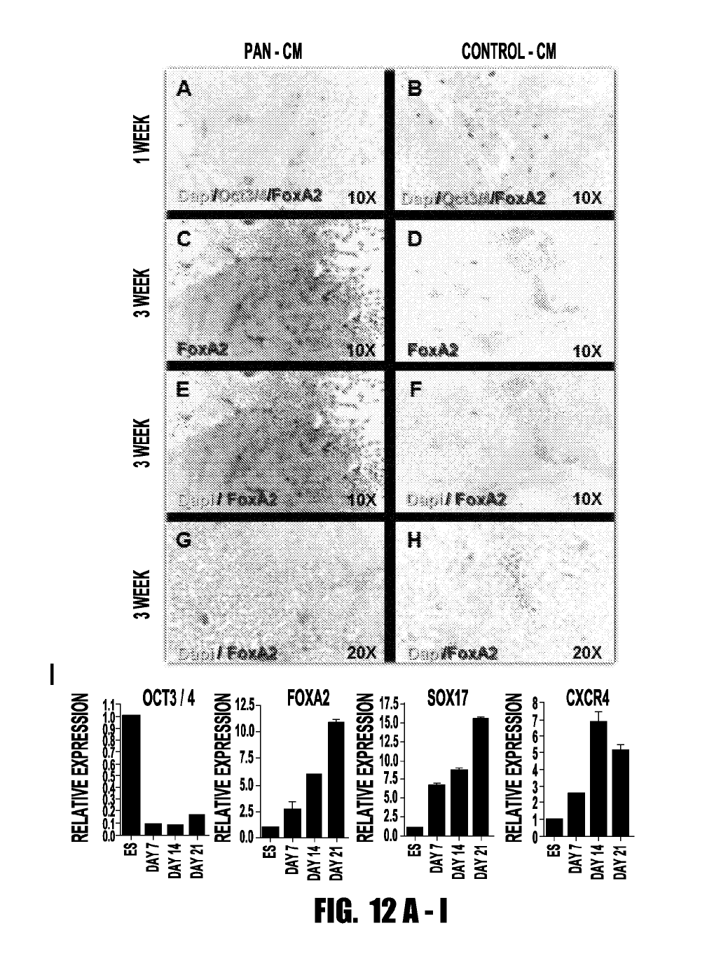

To establish the extent with which pluripotent cultures supplemented with Pan-CM begin to differentiate towards a pancreatic fate, we more carefully addressed the effects at the first week of incubation. As described previously, pancreas-specific MEF cultures derived from E13.5 timed embryos were used to condition medium which in turn was supplemented onto the H1 pluripotent hES cell line. Within the first week of incubation there was a significant down-regulation in genes involved in pluripotency maintenance. Nanog, Oct3/4 and Sox2 relative transcript levels were all severely diminished ranging from 20%, 30% and 50% of control levels respectively (FIG. 11 panel A). This diminished pluripotent transcript level was correlated to a substantial increase in the relative levels of several endodermal transcripts. Notably FoxA2 levels increased ˜20 fold over controls (FIG. 11 panel B), though Sox17 and Ecad levels were only moderately increased during the first week of differentiation with relative increases of both being ˜2.5 fold over controls (FIG. 11 panel B). While the relative level endodermal transcripts were increased varied between experiments, possibly because of slight variances between different pancreas-specific MEF preparations, endodermal genes were consistently up-regulated over controls and the levels with which these genes were up-regulated always increased over time. Assaying the relative transcript level of various endodermal genes at weekly time intervals demonstrated a continued increase in the expression of several endodermal markers. FoxA2 and Sox17 levels were shown to continuously increase over three weeks time course (FIG. 12 panel I) with relative levels reaching a 10-fold and 15-fold increase over controls respectively. Cxcr4 transcript levels displayed a similar pattern, though its relative expression did decrease between the second and third week of Pan-CM incubation.

Immunohistochemical analysis of FoxA2 expression within a pluripotent culture supplemented with Pan-CM as compared to a pluripotent culture which was supplemented with MEF conditioned medium confirmed the expression patterns seen in the transcript analysis. After a single week of incubating a pluripotent culture in the presence of either Pan-CM or MEF-CM Oct3/4 expression was greatly decreased within the culture supplemented with Pan-CM (FIG. 12 panel A) while control cultures still expressed high levels of the pluripotent marker (FIG. 12 panel B). Interestingly neither of these cultures showed strong evidence of FoxA2 expression after the first week of incubation with the respective mediums. However by the third week of incubation, the endodermal gene FoxA2 has widespread expression within the forward differentiating culture grown in the presence of the pancreas-specific MEF culture (FIG. 12 panels C, E and G), while the continued incubation of pluripotent cultures in the presence of embryonic body trunk derived MEF conditioned medium only showed limited FoxA2 expression (FIG. 12 panels D, F H). All together this clearly shows a very efficient and preferential definitive endoderm formation occurring within the pluripotent cultures that are maintained in the presence of the Pan-CM.

### Example 7

### Differential Effects Between Pluripotent Cultures Maintained with Pan-CM as Compared to Body Trunk Derived MEF-CM

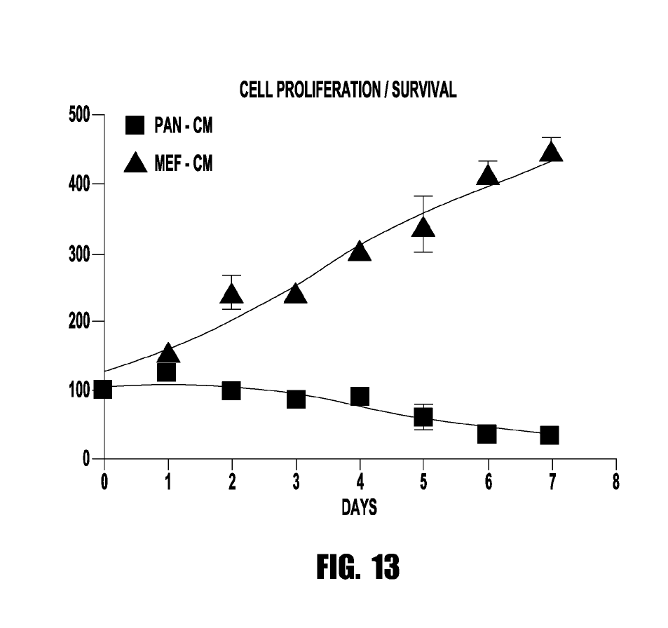

Not only are there significant differences between the expression patterns displayed in pluripotent cultures grown in the presence of Pan-CM when compared to pluripotent cultures sustained in body-trunk derived MEF-CM, but drastic differences were also noted in both the proliferation and morphology as a result of these differing culture conditions. Pluripotent cultures which were maintained in conditioned medium derived from regular feeder MEFs continued to be proliferative until the culture was completely overgrown. This was in complete contrast to what was observed in pluripotent cultures which were maintained in Pan-CM, a large percentage of these cultures died off within days after being exposed to the Pan-CM. Growth only occurred within discrete regions of these cultures and these regions would latter form cyst-like structures. Cultures maintained in regular MEF conditioned medium never formed these cyst-like structures nor was the growth of the cultures restricted or limited in any fashion instead all colonies grow equally well. Within the first week of culturing using the respective conditions colonies grown in the presence of regular MEF conditioned medium grow to approximately ˜450% of the starting number of cells. This was a large increase when compared to the colonies maintained within the Pan-CM which after the first week was ˜33% the starting number of the starting conditions (FIG. 13). It is important to note that this was not the result of pluripotent cultures maintained in the presence of the Pan-CM not growing, but was a result of only a limited portion of these pluripotent cultures actively expanding while the majority of the culture died off. This implies that only a limited percentage of the initial pluripotent culture was capable of surviving within the Pan-CM and it is this sub-population which in turn would grow into cyst-like structures.

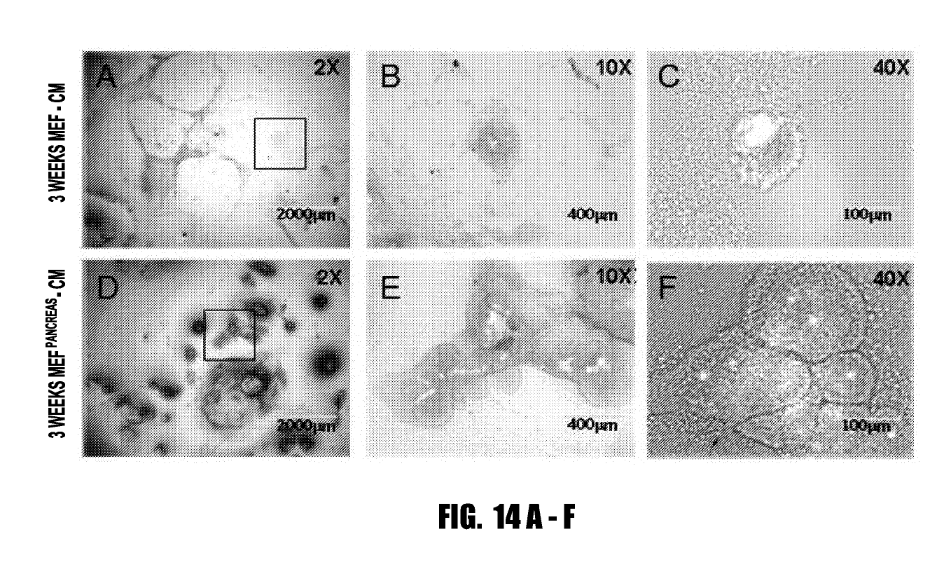

The continued incubation of pluripotent culture in the presence of Pan-CM resulted in the formation of several cyst-like structures forming per well. These Cyst-like structures generally started appearing by the second week of continued Pan-CM incubation and by the third week these structures were predominant and displayed complex morphologies. Examples of several hES derived cyst-like structures grown in Pan-CM are shown in FIG. 14 panels D-F. Pluripotent colonies that were exposed to regular MEF conditioned medium for the same 3 week period are also shown for comparison (FIG. 14 panels A-C), though no cyst-like structures are formed under these conditions.

### Example 8

### Direct Induction Pancreatic Identity, and Terminal Pancreatic Cell Fates, from Pluripotent Cells Using Pancreatic Specific MEF Cells

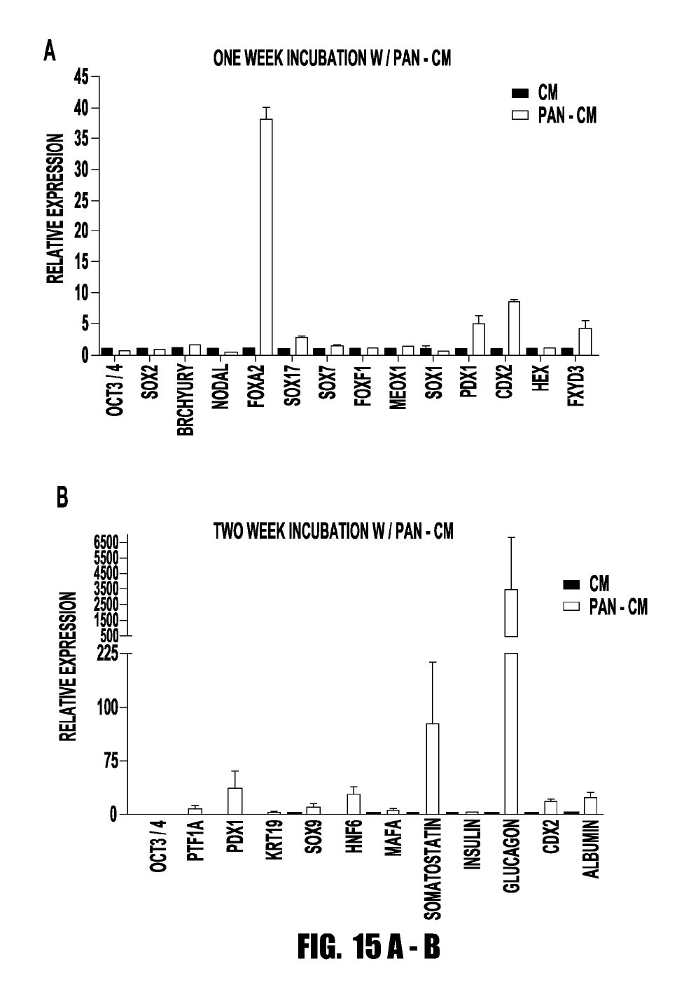

Since we have established that pluripotent cultures grown in the presence of Pan-CM display a stronger tendency to differentiate towards pancreatic fates, we next investigated the extent of this pancreatic induction. First we examined the relative expression levels of several lineages markers for early fate decisions within pluripotent cultures maintained for a week in either Pan-CM or regular MEF conditioned medium (FIG. 15 panel A). Significant decreases in the expression levels of the pluripotency markers Oct3/4 and Sox2 were observed in cultures maintained in Pan-CM. The mesendodermal marker Brachyury showed a slight increase in relative expression values while the mesendodermal marker Nodal showed was decreased as compared to hES level. Endodermal genes were up-regulated in Pan-CM stimulated colonies with relative FoxA2 expression levels reaching ˜38 fold over controls and a modest ˜3 fold increase was observed in Sox17 transcript levels. Even with the low levels of Sox17 this is indicative of definitive endoderm when considering that the visceral endodermal gene Sox 7 was not increased. There was no significant change in the relative transcript levels of either ectodermal makers (Sox1 and Sox2) or mesodermal markers (FoxF1 and Meox1). However homeobox genes expressed during the patterning of the gut tube during organogenesis of the pancreas (Pdx1), the intestines (Cdx2) and the stomach (Fxyd3) were all found to be increased in pluripotent cultures supplemented with Pan-CM, though the liver specific gene Hex failed to increase. Not surprisingly none of these markers were detected to any significant levels in the control cultures maintained in regular MEF conditioned medium, since this medium is generally used to maintain pluripotency. All together this demonstrates a strong tendency of the Pan-CM to direct the differentiation of pluripotent cultures toward endodermal fates as well as the fates which normally arise from the endoderm.

Since the genes being detected after the first week of Pan-CM treatment tended to be representative of early fate decisions, pluripotent cultures were next assayed at the second week of incubation in Pan-CM with the transcript analysis focusing on pancreas-specific fates (FIG. 15 panel B). Both the early pancreatic progenitor genes Ptf1a and Pdx1 were up-regulated over the control cultures however Pdx1 levels were consistently higher (˜35 fold increase over control in this example). In addition transcripts required for normal in vivo endocrine differentiation were also consistently up-regulated over controls. Both Sox9 and Hnf6 transcript levels increased ˜10 and ˜30 fold respectively over controls and transcripts representative of the endocrine subtypes of the alpha and delta cells were extremely abundant. The relative transcript levels for Somatostatin and Glucagon increased ˜125 fold and ˜3500 fold respectively over controls. Insulin expression levels did not increase significantly only reaching an ˜2.8 fold increase over controls. The intestinal specific and liver specific genes Cdx2 and Albumin respectively both were increased over the levels found in control differentiation experiments, however their relative transcript levels were less than the pancreas specific genes shown with Cdx2 levels reaching ˜18 fold and Albumin reaching ˜24 fold levels. All together this demonstrates that the pluripotent cultures which were sustained in the presence of Pan-CM displayed a greatly increased pancreatic phenotype over hES cultures maintained in conditioned medium derived from body-trunk MEFs.

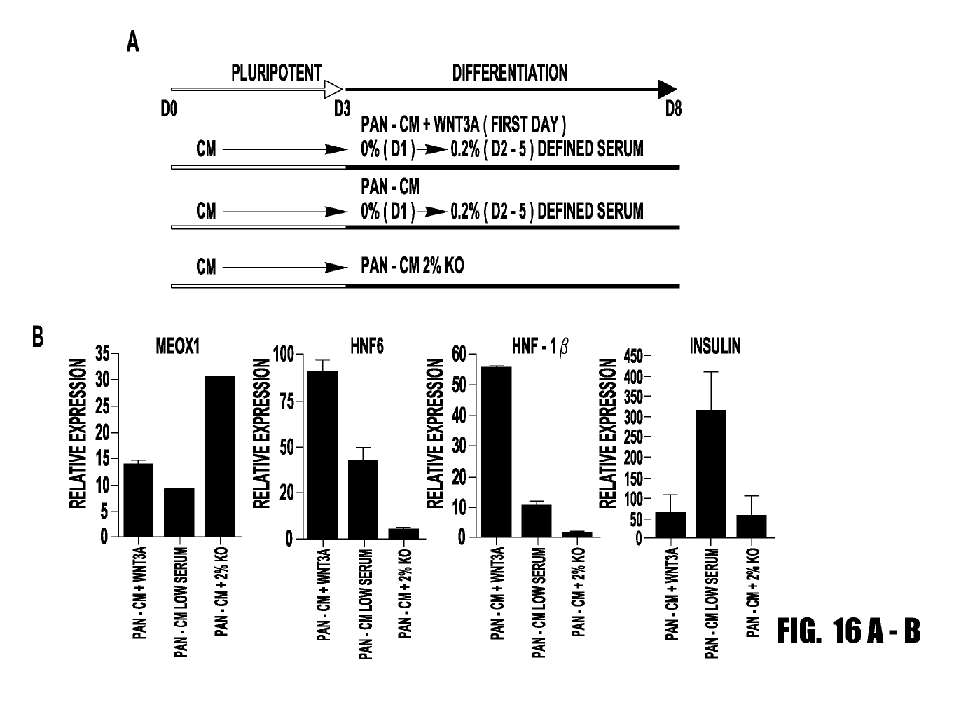

The Pan-CM mediated induction of insulin producing cells from the H1 hES cultures is improved when low concentrations of serum is used. Panel A of FIG. 16 shows a schematic outlining the three conditions used throughout this series of experiments in which the addition of Wnt3a into Pan-CM and low levels of defined serum are used in place of the KO serum replacement. Panel B of FIG. 16 shows transcript analysis of two markers of the trunk progenitor state (HNF6 and HNF1-B) which are up regulated in response to low serum levels (˜40× and ˜10× respectively) and further by the addition of Wnt3A on the first day of differentiation (˜85× and ˜55× respectively). Interestingly, insulin transcript levels are only significantly up regulated in the presence of low serum levels (˜300 fold), when Wnt3a is used on the first day of the differentiation experiment levels are comparable to the Pan-CM induction with 2% KO serum replacer. Finally it was noted that lower levels of serum reduced to levels of MEOX1 (a gene associated with the mesodermal lineage) observed in these cultures.

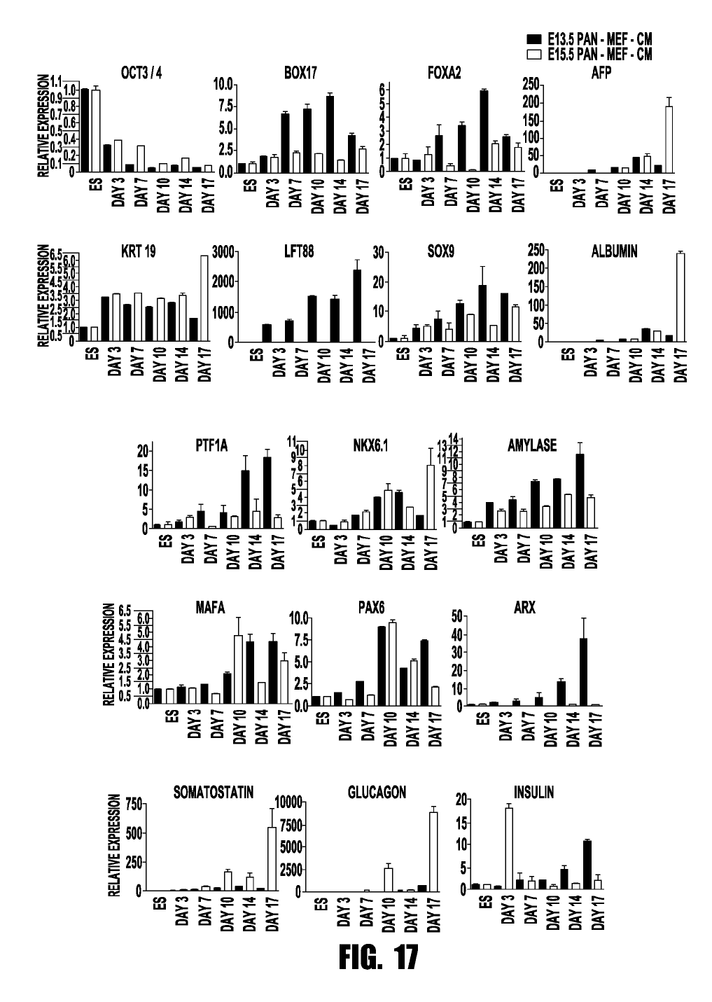

All differentiation experiments up till this point were performed using organ-specific MEF cultures derived from E13.5 timed embryos. We next compared the differentiation inducing potential of pancreatic specific MEF cultures derived from E13.5 timed embryos as compared to cultures derived from E15.5 timed embryos to establish whether the observed effects were specific to the stage of development that the organ-specific MEF cultures were derived from or if they were general effects that could occur from pancreas-specific mesenchyme regardless of age. While many of the genes assayed in this series of experiments displayed similar expression patterns between pluripotent cultures maintained in the presence of Pan-CM derived from either E13.5 or E15.5 embryos some striking differential expression patterns were noted. First, genes representative of definitive endoderm tended to have a greater up-regulation when the Pan-CM was derived from E13.5 timed embryos (FIG. 17). Both Sox17 and FoxA2 relative transcript levels were higher in pluripotent cultures maintained in E13.5 derived Pan-CM with maximal up-regulated levels reaching ˜8.5 fold and ˜6 fold over hES levels respectively. Maintaining pluripotent cultures in Pan-CM derived from E13.5 embryos greatly increased the ductal phenotype over what was observed in cultures maintained in E15.5 derived Pan-CM. This is evident since the intraflagellar transport protein 88 homolog (Ift88), a protein involved in cilia formation which has been found to be crucial for proper pancreatic duct formation, was found to be preferentially up-regulated in Pan-CM derived from E13.5 embryos when compared to the effects of the Pan-CM derived from E15.5 embryos. Relative transcript levels of Ift88 were shown to increase ˜2500 fold over hES levels, this substantial increase did not occur in cultures grown in the presence of E15.5 derived Pan-CM indicating that the Pan-MEF cultures derived from an earlier developmental time were better at generating ductal phenotypes within forward differentiating pluripotent cultures. Finally the alpha-cell specific gene Arx was far more prevalent within forward differentiating cultures exposed to Pan-CM derived from E13.5 embryos with relative expression levels reaching a ˜40 fold increase over cultures maintained in Pan-CM derived from E15.5 embryos. Interestingly the only pancreas-specific fate which was significantly increased in pluripotent cultures maintained in Pan-CM derived from E15.5 embryos as compared to Pan-CM derived from E13.5 embryos was Glucagon. Glucagon levels reached an ˜8500 fold increase in reactions maintained in the E15.5-Pan-CM while only ˜700 fold increase was observed in cultures sustained in E13.5 Pan-CM. The E15.5 derived Pan-CM was also found to have a greater presence of liver specific transcripts with AFP levels being increased ˜200 fold and Albumin transcript levels increasing ˜250 fold.

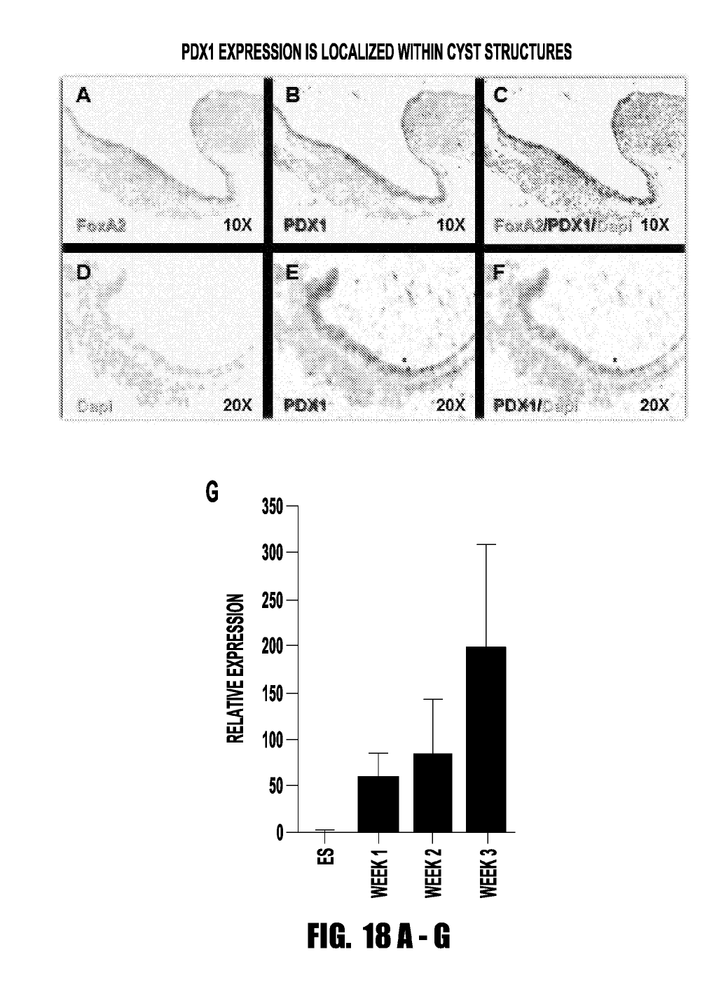

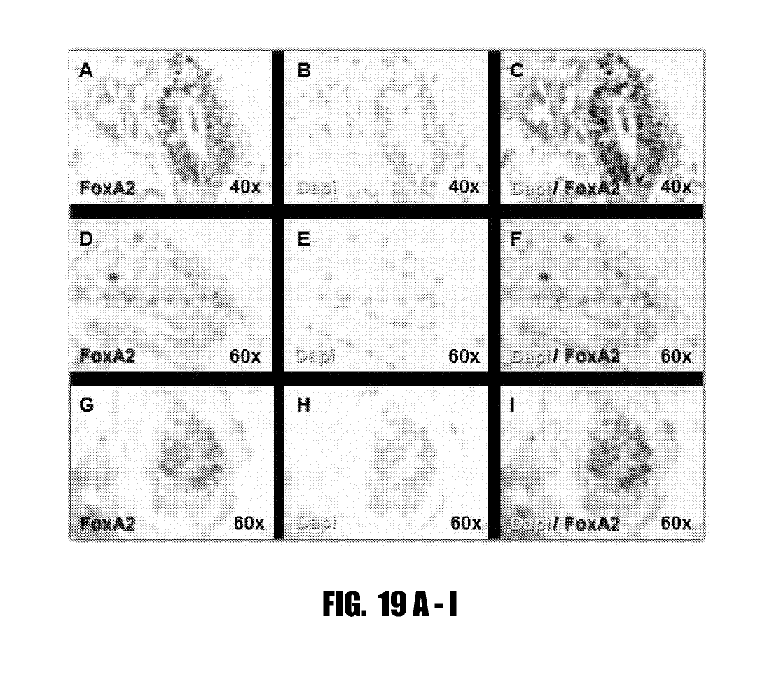

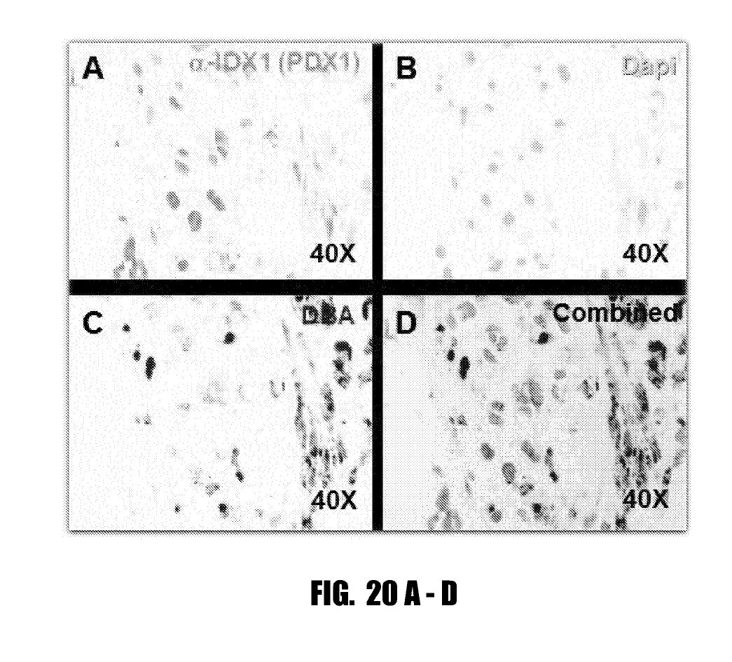

Next we used histology to collaborate the expression patterns observed through our transcript analysis of pluripotent cultures maintained in the presence of Pan-CM. The cyst-like structures which form during the continued exposure of Pan-CM to forward differentiating cultures were prepared and sectioned followed by immunostaining for several pancreas-specific proteins. The pancreatic progenitor Pdx1 was found to be widely expressed within discrete regions all throughout the cyst-like structures. FIG. 18 shows some typical cyst sections demonstrating the widespread expression of Pdx1. Pdx1 was commonly found to be expressed within FoxA2 positive regions (FIG. 18 panels A-C), however FoxA2 expression was not limited to Pdx1⁺ cells (FIG. 19 panels A-I). In addition FoxA2 expression was commonly found within surviving monolayer regions present in experimental plates that did not generate the cyst-like outgrowths (FIG. 12 panels C, E and G), while Pdx1 expression was always limited to the cyst-like outgrowths. In addition Pdx1 positive regions were commonly found adjacent to dolichos biflorus agglutinin⁺ (DBA) regions (FIG. 20 panel A-D). DBA is a commonly accepted marker of pancreatic ducts and this observed spatial expression pattern of DBA to Pdx1 is analogous to what is found during the development of the pancreas in utero. All together this suggests that the widespread generation of FoxA2 regions occurs during the continued exposure of a forward differentiating culture to Pan-CM. In turn only a limited number of these FoxA2+ regions are capable of responding to the Pan-CM medium by growing cyst-like structures and it is these cyst-like culture outgrowths which display increased pancreatic phenotypes.

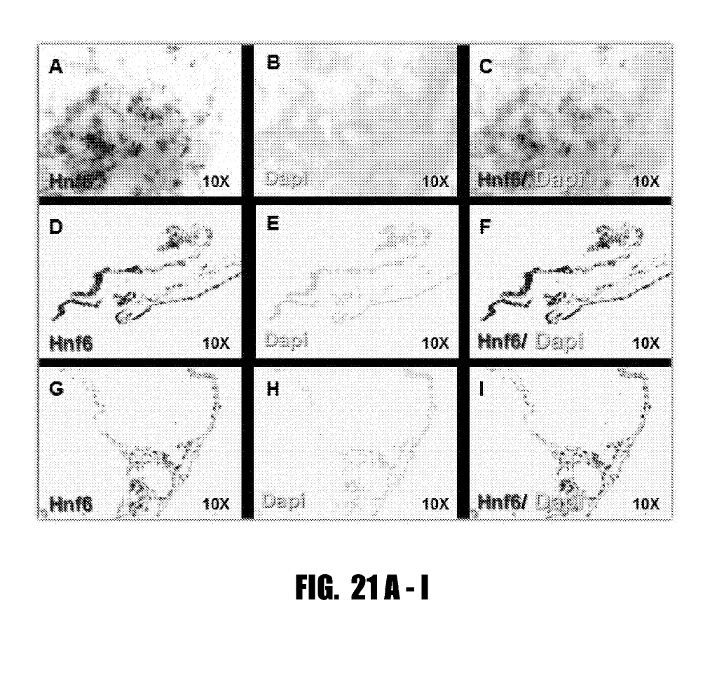

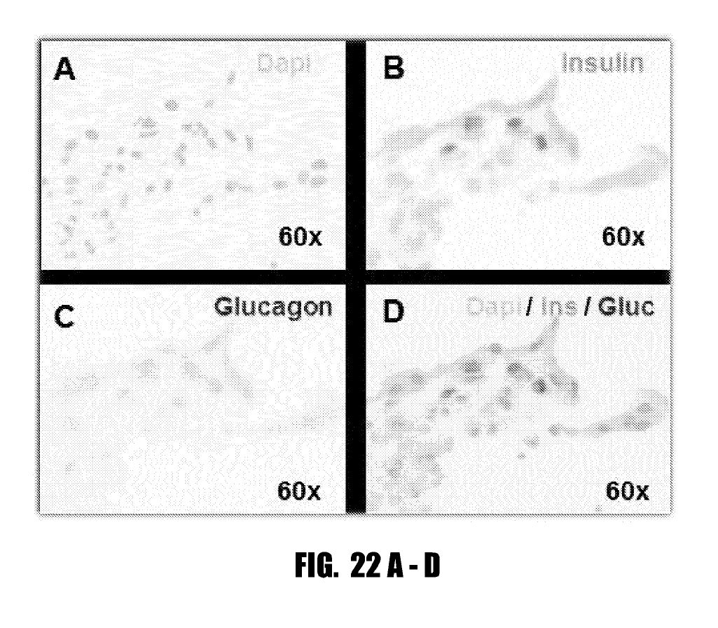

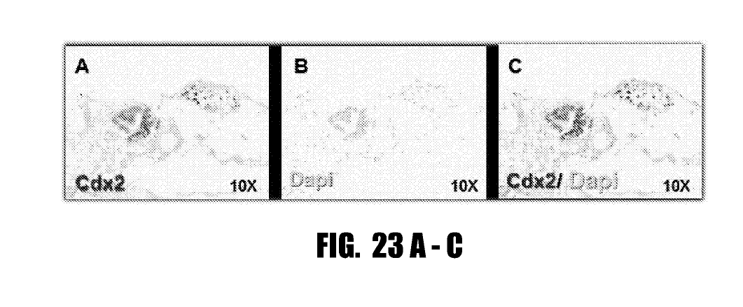

The proendocrine transcription factor hepatic nuclear factor 6 (Hnf6) also displayed widespread expression within pluripotent cultures maintained in Pan-CM, however its expression was also restricted to the hES derived cysts. FIG. 21 panels A-C shows Hnf6 expression within a forward differentiating culture which was maintained in Pan-CM for 3 weeks. Note Hnf6 expression is restricted to the areas where cyst-like outgrowths are occurring and is abundant within these regions. Hnf6 expression was also noted in immunostained cyst sections (FIG. 21 panels D-I) and suggests a large portion of the cyst-like formations may have proendocrine phenotypes though the generation of endocrine markers in cysts continuously maintained in Pan-CM may be hindered. While glucagon expression within these hES-derived cysts is common, Insulin expression is severely limited in comparison; though occasional Insulin⁺ cells are observed (FIG. 22 panels A-D). Finally the pancreatic phenotype within these structures is not absolute and expression patterns of markers of other endodermal fates and mesenchymal fates have been observed. An example of the expression of the intestinal marker Cdx2 is given in FIG. 23 panels A-C.

All together this shows that pluripotent cultures maintained in the continued presence of Pan-CM had a strong tendency to differentiate towards pancreatic lineages with an especially efficient differentiation towards ductal, proendocrine and endocrine lineages, though the preferred endocrine sub-type fate under the set of conditions provided tended to be an alpha-like Glucagon expressing cell with only modest up-regulations in insulin producing cells. Evaluating forward differentiating cultures at different time points over the course of three weeks demonstrated that endodermal transcripts were up-regulated within a few days of incubation in the presence of Pan-CM. Up-regulation of early pancreas-specific genes tended to take longer with significant levels of Pdx1 and limited Ptf1a levels usually occurring between one to two weeks time. Endocrine fates were only seen after 2 to 3 weeks of incubation. Since endodermal genes are observed before early pancreatic progenitor genes which in turn precede the up-regulation of terminal pancreatic genes, this would argue that the forward differentiating cultures are developing through normal developmental stages and that the Pan-CM may simply provide the optimal conditions for the development of pancreatic phenotypes with an emphasis on the glucagon expressing cell-type.

### Example 9

### A Two-Step Induction Protocol of Terminal Pancreatic Endocrine Fates

Since we observed the continuous up-regulation of markers for both the pancreatic and endocrine precursors we next began to speculate that the Pan-CM was informing a centralized/trunk progenitor pancreatic fate. Since both of these fates are normally transient in nature we next tested how forward differentiating cultures would respond to the removal of the Pan-CM environment once these fate have been established. To establish optimal parameters a 3 week time course was analyzed with an analysis of the effects of the removal of Pan-CM at the 1 week and 2 week time points. By switching the culture conditions from the Pan-CM to an unconditioned medium at these time points we were able to establish the minimal time requirements for the Pan-CM to establish a pancreatic phenotype in forward differentiating cultures. Pluripotent cultures which were maintained for a single week in the presence of Pan-CM did not show any significant increase in any of the pancreas-specific transcripts analyzed. However cultures maintained in the presence of Pan-CM for a week followed by an additional week of culturing in the presence of unconditioned medium displayed a greater relative increase in the terminal endocrine marker Somatostatin than cultures maintained the entire two weeks in Pan-CM. The relative expression levels of Somatostatin for these two conditions were ˜500 fold increase and a ˜200 fold increase respectively. However this effect only occurred with Somatostatin, cultures maintained a single week in Pan-CM followed by an additional week in an unconditioned medium loss displayed a greatly reduced relative level of Glucagon expressing cells only reaching an ˜750 fold increase over hES levels while cultures maintained the full two weeks in Pan-CM expressed a relative Glucagon transcript level ˜10,000 fold over control levels. This suggests that a partial commitment towards a pancreatic phenotype has already occurred within a week's time and the removal of the Pan-CM does result in greater terminal differentiation of Somatostatin expressing cells, however other fates were lost in this shortened incubation time.

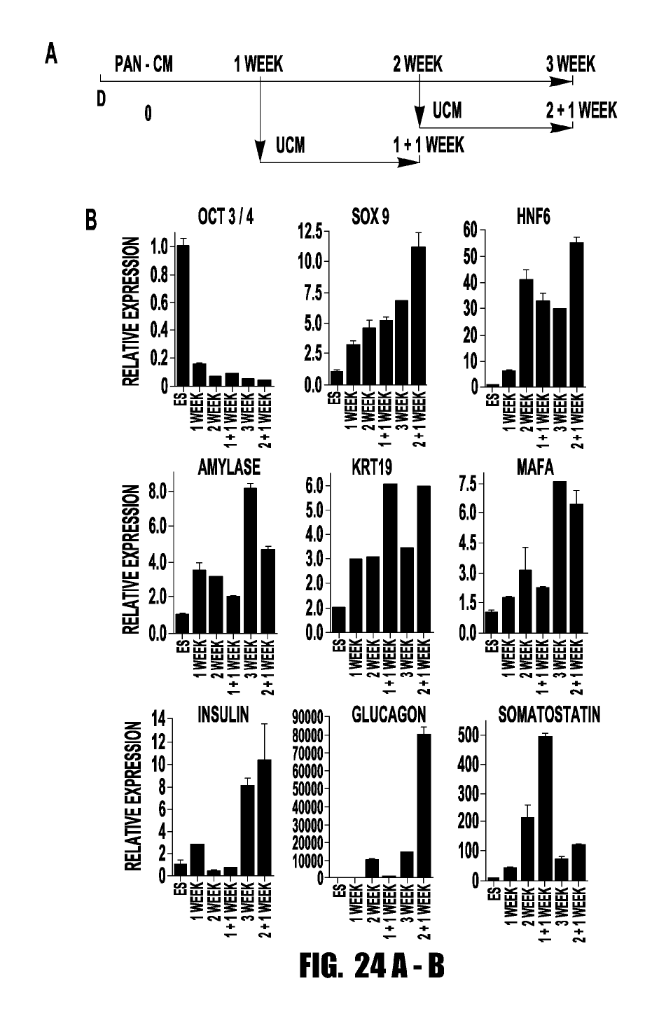

While the relative levels of Glucagon expression were greatly decreased when Pan-CM was removed after the first week of incubation, Glucagon transcript levels were greatly accelerated when the Pan-CM was removed after the second week of incubation. Removal of Pan-CM after the second week of incubation resulted in an almost ˜6 fold increase over the relative levels reached during the full three week incubation in Pan-CM and an ˜80,000 fold increase over hES glucagon transcript levels (FIG. 24 panel B). In addition the removal of the Pan-CM at both the first and second week resulted in an increase in the ductal fate as indicated by cytokeratin 19 (Krt19) relative expression levels whereas relative expression levels of the progenitor markers (Sox9 and Hnf6) assayed throughout this series of experiments tended not to be effected by the Pan-CM withdrawal. This implies that Pan-CM mediated hES-derived pancreatic phenotype is maintained at least partially in a progenitor state and full terminal differentiation is inhibited in its presence. In addition this establishes that the initial push towards a pancreatic expressing phenotype occurs within the first to second week of incubation in Pan-CM at which time the endogenous regulation occurring in the forward differentiating cultures can propagate this pancreatic phenotype and drive it towards terminal differentiated fates.

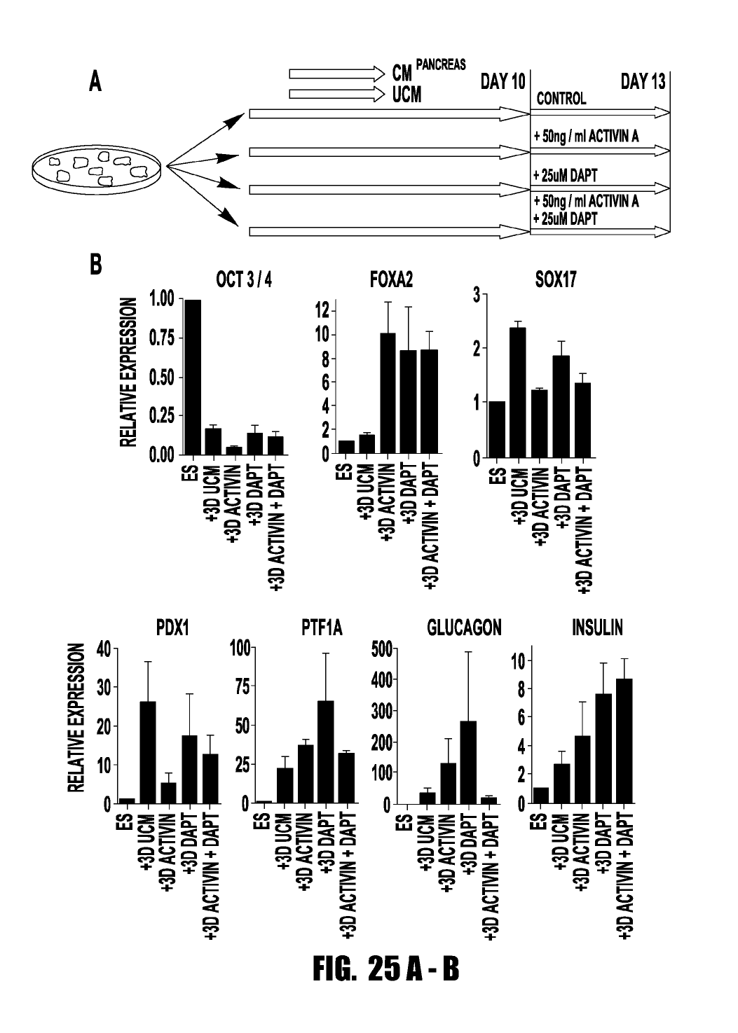

Since we have established that the continued culturing of pluripotent cells in the presence of Pan-CM results in an increased differentiation towards pancreatic fates and that there is an especially strong tendency for these cells to either adapt a ductal or endocrine precursor fate, which in turn has a strong tendency to become a Glucagon⁺ cell, we next wanted to establish weather we could influence the respective endocrine subtype fate choice. Specifically we tested weather changing culture conditions after the formation of pancreatic and endocrine precursor had occurred, but before complete terminal commitment towards the glucagon expressing fate occurs, could increase the tendency of these forward differentiating cultures to adopt an insulin producing fate. This effort was explored using proteins and small molecules additives to influence the terminal endocrine fate decision. Our initial attempts at specifically influencing the glucagon/insulin subtype fate assignment concentrated on the use of the gamma secretase inhibitor, DAPT, since it is well established that Notch inhibition is central in this endocrine subtype assignment and Activin A which has previously been shown to greatly assist in the de novo generation of Insulin producing cells in a directed differentiation context. Pluripotent cultures were treated 10 days with Pan-CM at which time the growth medium was switched to an unconditioned medium (UCM) or an UCM supplemented with Activin A or DAPT or both (as outlined in FIG. 25 panel A). In all cases switching to the UCM increased terminal differentiation towards an insulin producing cell, however the greatest effects were observed when DAPT was used in combination with Activin A (FIG. 25 panel B). This latter condition had a synergistic effect, not only was a 10 fold increase in insulin transcript levels observed, but a complimentary decrease in Glucagon transcript levels occurred as compared to cultures which were treated with only DAPT or Activin A. All together this implies that Pan-CM treated cultures are competent to differentiate towards other pancreas specific fates if the culture conditions are changed as to favor the other terminal fates.

### Example 10

### Studies Defining the Molecular Mechanism of Endocrine Cell Fate Allocation in the Developing Embryo

While the ability of pancreas-MEF cells was shown to potently induce pancreatic cells from pluripotent cells, further descendants of the pancreatic cells were ineffectively differentiating into pancreatic insulin-producing cells. To facilitate and devise studies for the improvement of formation of pancreatic insulin producing cells we investigated the basic fundamental principles of beta cell formation in the context of the normal developing embryo. This was done via specific studies of murine pancreatic development, and in particular the construction of novel murine transgenic models that query critical pathways in the endocrine differentiation program. These studies were used by us to define the TrPC state, and the pathway control of such cells, subsequently allowing modification of the forward differentiation program of human pluripotent stem cells.

### Example 10 (i)

### Ectopic Ngn3 Expression Leads to Accelerated Endocrine Differentiation at the Expense of Pancreatic Progenitors

We generated a conditional gain-of-function model of the pro-endocrine gene Ngn3, in order to understand its ability to induce endocrine fates from pancreatic progenitor cells. pTRE2-Ngn3 mice were generated by pro-nuclear injections with a linearized DNA construct. Nineteen pTRE2-Ngn3 founders were identified from 3 independent injections. Of these, ten founder animals transmitted the transgene to F1 offspring. All the transmitting lines displayed normal behavior and did not show any detectable phenotype as single transgene (STG) carriers. Multiple lines were tested and validated for doxycycline (Dox)-dependent Ngn3 expression. Throughout this study, we used a Pdx1-tTA knock-in line (Pdx1-tTA^{KI}) to evaluate the effect of ectopic Ngn3 expression throughout the Pdx1 domain in the developing foregut Holland A M, Hale M A, Kagami H, Hammer R E, MacDonald R J., Proc Natl Acad Sci USA. 2002 Sep. 17; 99(19):12236-41. In control experiments with continuous Dox administration to pregnant females, Pdx1-tTA^{KI}; pTRE2-Ngn3 crosses revealed complete inactivity of the transgene at all ages studied.

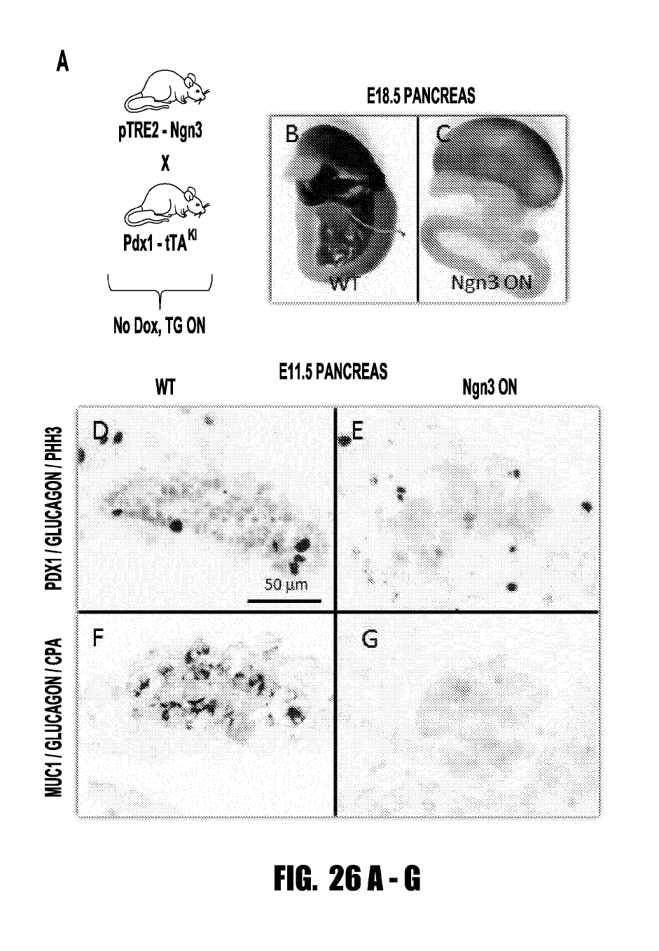

Ectopic Ngn3 gene induction was achieved by omitting Dox in the drinking water (FIG. 26A). Embryos were harvested at gestational day 18.5 (E18.5), as this would resample previous findings of ectopic expression of Ngn3 in the Pdx1 domain Hart A W, Baeza N, Apelqvist A, Edlund H., Nature. 2000 Dec. 14; 408(6814):864-8. Double-transgenic Pdx1-tTA^{KI}; pTRE2-Ngn3 embryos (Ngn3 ON) displayed significant pancreatic hypoplasia (FIG. 26C) and a loss of gut continuity at the antral stomach/duodenal level, comparing with the wild type (WT) (FIG. 26B). In this study, we focus on the effect of Ngn3 overexpression on the developing pancreas whereas the causal role of Ngn3 in endocrine cell conversion and patterning of the lateral duodenal wall and developing stomach will be described elsewhere.

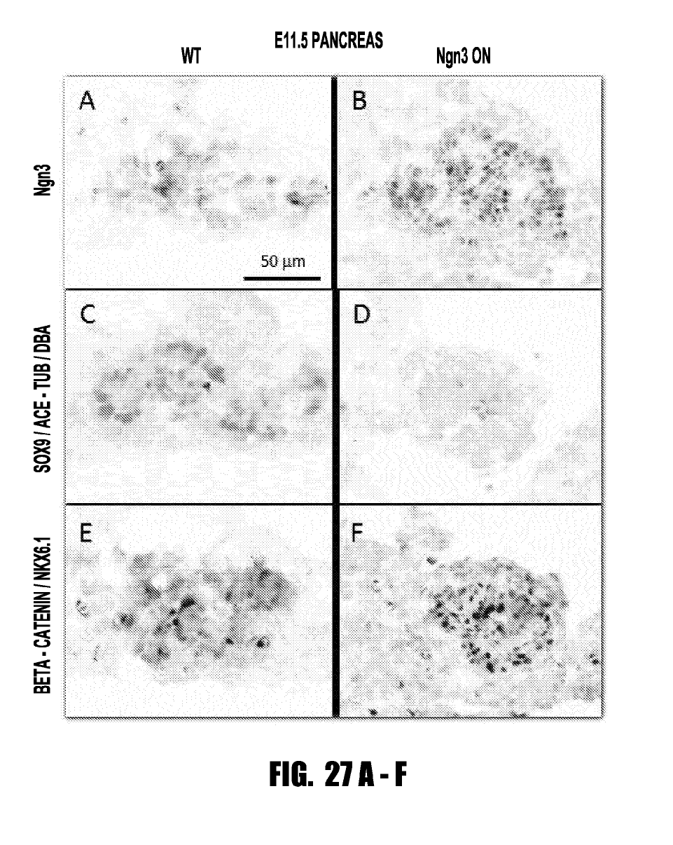

Ngn3-mediated pancreatic organ hypoplasia could be explained by progenitor depletion through accelerated differentiation. We analyzed E11.5 Ngn3 ON and WT pancreas and observed the presence of glucagon-positive (Glu+) cells, most of which expressed Pdx1 at low to intermediate levels (FIG. 26E) in Ngn3 ON pancreas. Such cells were not proliferative as judged by absence of M-phase activity (pHH3 staining, FIG. 26E). The WT pancreas displayed ubiquitous Pdx1 expression and cells were proliferating (FIG. 26D). We found no evidence of Mucin 1 (Muc1) expression in Ngn3 ON pancreas (FIG. 26G). Mucin1 expression reveals the initial emergence of tubulogenesis through microlumen formation and signifies epithelial apical polarity (FIG. 26F, and as described by Kesavan et al., Cell. 2009 Nov. 13; 139(4):791-80). A complete loss of the progenitor and later acinar marker carboxypeptidase A1 (CPA1) was prominent in Ngn3 ON pancreas (FIG. 26G). Dramatically increased Ngn3 expression was detected in the Ngn3 ON pancreas. Sox9, a progenitor marker, was essentially abrogated in the Ngn3 ON pancreas (FIG. 27D). Dolichos Biflorus agglutinin (DBA), a marker of pancreatic ductal cells, was not expressed in either WT or Ngn3 ON pancreas (FIG. 27C,D). The trunk progenitor marker Nkx6.1 remained expressed in Ngn3 ON pancreas (FIG. 27F).

### Example 10 (ii)

### Overexpression of Ngn3 Prior to the Secondary Transition Results in the Induction of Pancreatic Duct Cells

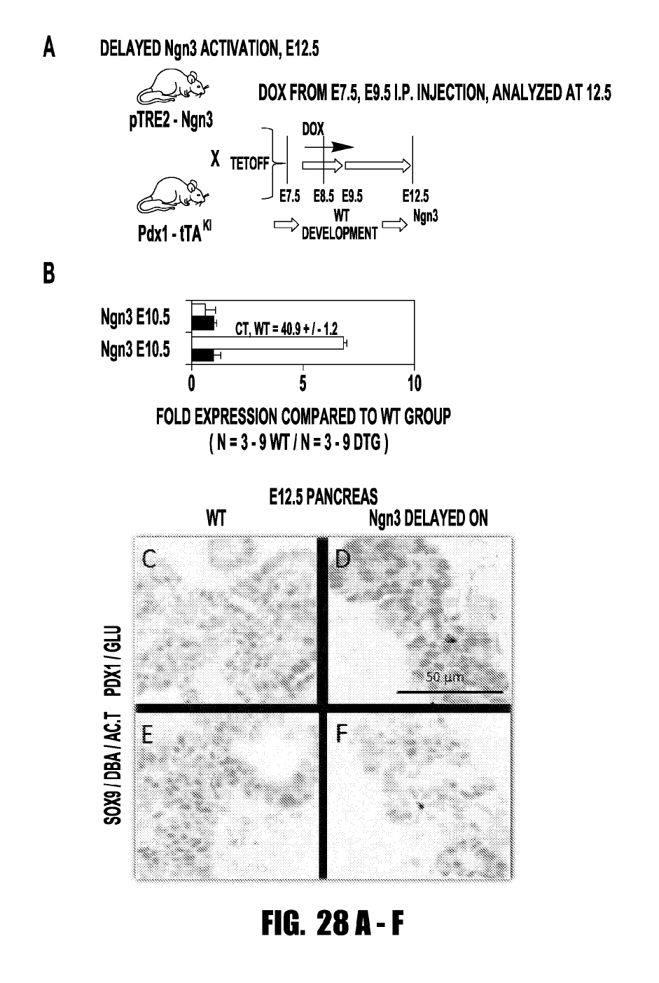

Our initial investigations corroborated the published role of Ngn3 in the induction of the pancreatic Glu+ endocrine fate. However, the primary goal of this study was to apply Ngn3 for the induction of insulin-producing (insulin+) cells, and we surmised that because Ngn3 was expressed since the initiation of pancreatic budding this explained the predominant Glu+ cell formation. To circumvent this, and to facilitate the formation of insulin+ cells, we used Dox to bypass the initial Ngn3-induced effects observed in the Ngn3 ON model. We administered Dox through intraperitoneal (i.p.) injections into the pregnant mouse when the embryos reached gestational day 7.5 (E7.5) and E9.5, and isolated embryos at E10.5, E11.5 and E12.5 (FIG. 28A). The kinetics of Ngn3 induction was assessed by qRT-PCR at E10.5 and E11.5. We did not observe an increase in total Ngn3 mRNA at 24 hr post-Dox administration (E10.5), but at 48 hr (E11.5), total levels of Ngn3 mRNA was 6.8-fold increased, compared to WT (FIG. 28B). These Pdx1-tTA^{KI}; pTRE2-Ngn3 DTG embryos receiving Dox at E7.5 and E9.5 are in the following referred to as Ngn3 Delayed ON embryos. All Ngn3 Delayed ON embryos, at all stages analyzed, successfully bypassed the apancreatic phenotype of the Ngn3 ON condition (example shown in FIG. 28C). Using this strategy, we then sought to establish how Ngn3 would affect gene expression and patterning of pancreatic multipotent progenitors while they became committed to terminal fates, so we analyzed E12.5 Ngn3 Delayed ON pancreas in more detail. Ngn3 mRNA expression was on average 9.1-fold increased in the Ngn3 Delayed ON group compared to WT littermates (data not shown, n=6 WT, n=5 DTG). The histology of E12.5 Ngn3 Delayed ON pancreas resembled that of WT pancreas, revealing the presence of a stratified, plexus-type epithelial tissue, expressing Pdx1 and Sox9 in a uniform pattern (FIG. 28D, F). Epithelial cells were proliferative in both WT and Ngn3 Delayed ON pancreas as assessed by BrdU incorporation (data not shown). The excessive formation of Glu+ clusters causing progenitor depletion observed in Ngn3 ON embryos was not prevalent (FIG. 28D). Furthermore, DBA+ expressing ductal cells was not found in the E12.5 Ngn3 Delayed ON pancreas (FIG. 28F).

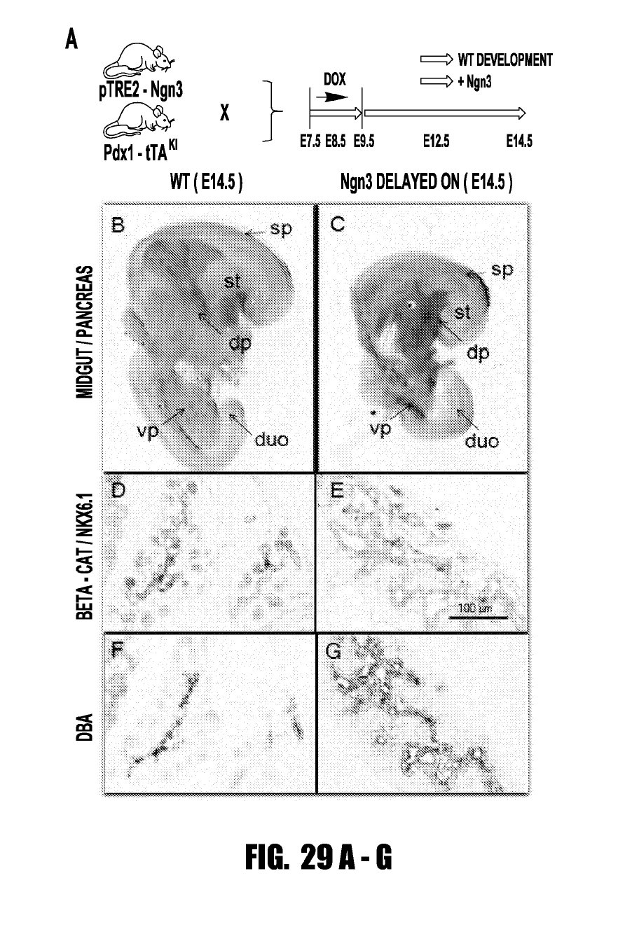

We were interested in the differentiation abilities of Ngn3 Delayed ON pancreas. Were such able to exclusively generate endocrine cells that were predominantly insulin+. To address this, we analyzed E14.5 Ngn3 Delayed ON pancreas (FIG. 29A, C). At E14.5, Ngn3 Delayed ON embryos developed significant pancreatic tissue, of both dorsal and ventral origin (FIG. 29C). Surprisingly, histological analysis revealed an expanded tubular network of β-catenin-expressing cells, interspersed with few cells expressing Nkx6.1 (FIG. 29E). There was a noticeable lack of epithelial branching and a general absence of forming acini at epithelial tips (compare FIG. 29D, E). The reduction of Nkx6.1 indicated that the epithelial cells were not of a progenitor type, and had undergone differentiation in the two-day period following E12.5. This was confirmed by using markers of terminally differentiated cells, as the epithelial-derived pancreatic tissue of Ngn3 Delayed ON (E14.5) consisted of mainly two types of cells: glucagon expressing endocrine cells (FIG. 29I, M) and DBA-expressing ductal cells (FIG. 29G). The dramatic increase in relative cell areas of Glu+ and DBA+ cells were offset by reductions in acinar and insulin+ cells (FIG. 29I, M, N), and agreed with the marked decrease in Nkx.6.1 expression when compared to E12.5 Ngn3 Delayed ON embryos. Acinar cells (amylase+, Amy+) were largely absent (FIG. 29 I, N), but present in WT (FIG. 29H); and insulin+ cells were similarly almost completely absent (FIG. 29 M, N) but present in WT (FIG. 29L, N).

The 17-fold increase in ductal cell differentiation (FIG. 29N) was surprising, and the DBA+ cell population outnumbered that of the endocrine cells (compare FIG. 29 G, I). To further clarify the phenotypic state of the ductal cells induced by Ngn3, we analyzed the expression of Hnf1β, encoded by Tcf2. Hnf1β initially overlaps with Nkx6.1 and Pdx1 within pancreatic progenitors but only ductal cells continue to express Hnf1β following differentiation. In Ngn3 Delayed ON E14.5 pancreas, we observed that the cuboidal-epithelial DBA+ expressing cells were Hnf1β+, and ciliated as noted by presence of ciliary basal bodies positive for acetylated tubulin (data not shown). This cellular phenotype is identical to that of normal pancreatic ductal cells. These ductal cells did not express Ngn3 (FIG. 29K).

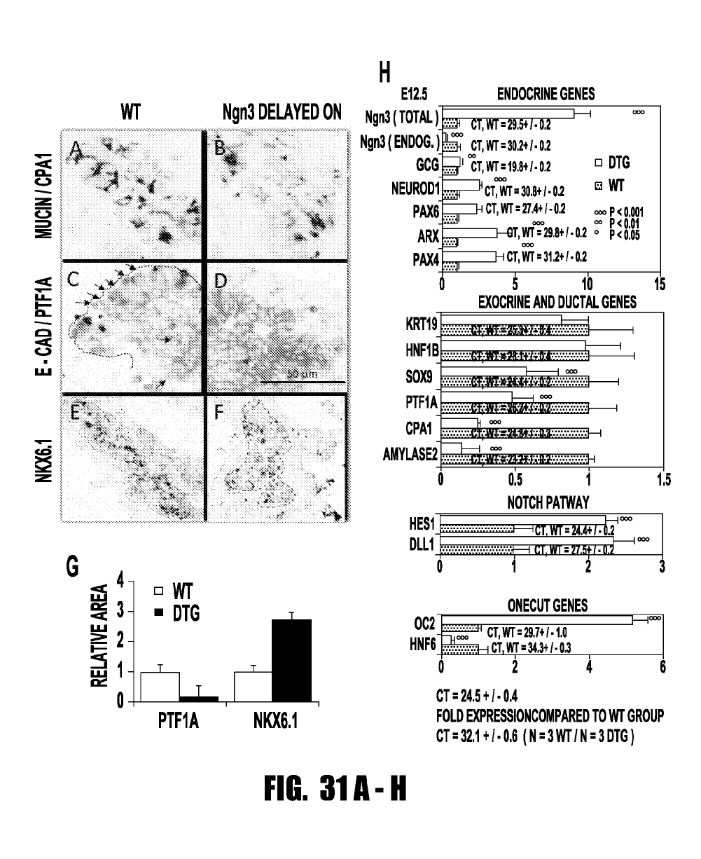

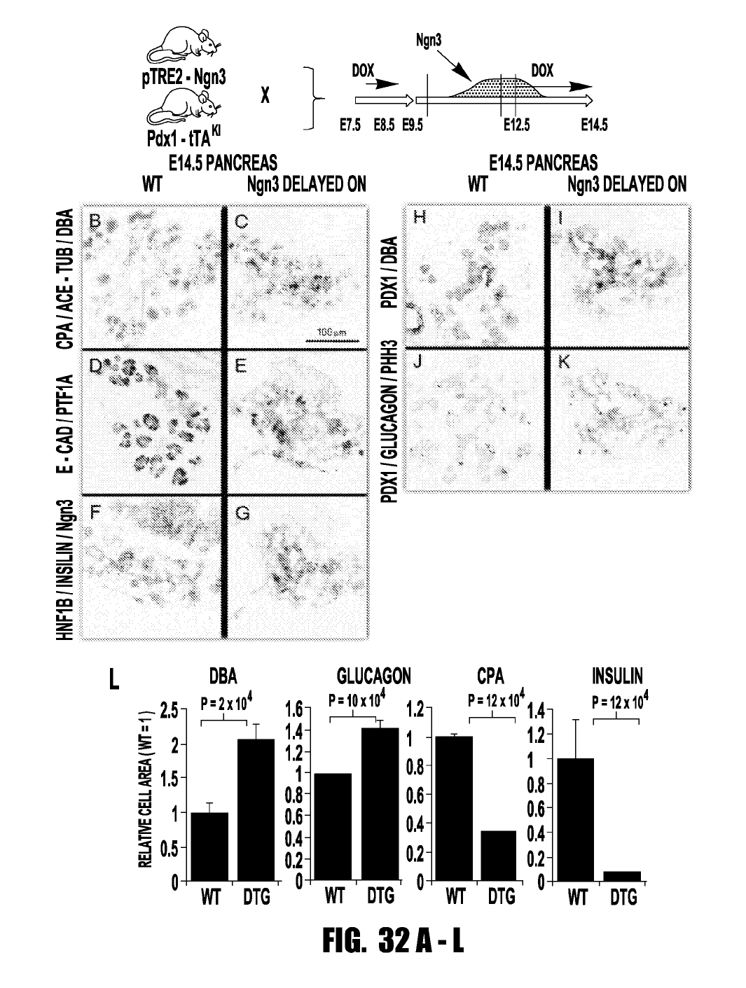

The degree of ductal cell differentiation in the model was both unexpected and undesired. This could be a consequence of the prolonged exposure to Ngn3 protein. For that reason, we sought to test the effects on pancreatic development of a temporal burst of exogenous Ngn3 expression prior to the time of the secondary transition. To do this, we administered Dox to Pdx1-tTA^{KI}; pTRE2-Ngn3 pregnant females at E7.5, E9.5 as well as at E12.5, followed by analysis at E14.5 (FIG. 32A). The time-window for additional Ngn3 expression would thus be limited to a brief period between E11.5 and E12.5. The embryos are in the following referred to as Ngn3 Brief ON, (FIG. 32B-K) We were initially surprised to see that the formation of pancreatic endocrine cells was not prevalent, however, we noted a reduction in acinar cell formation (CPA1+, Ptf1a+, FIGS. 31C, E, L), and increased ductal formation (DBA+, Hnf1β+ (FIG. 32C,G,I,L). Glu+ cells were only modestly increased in density (1.4 fold) (FIG. 32K, L), compared to the approximate 35-fold induction observed in the Ngn3 Delayed ON E14.5 pancreas (FIG. 29N). Also, only rare insulin+ cells were detected (FIG. 32G, L). Expanded trunk regions expressing Pdx1 was found (FIG. 321, K). Based on morphometrical assessment, ductal induction was found to be disproportionately higher than the induction of Glu+ cells under these conditions (FIG. 32L). We conclude that a brief exposure of Ngn3 expression is ineffective in the induction of endocrine fate, and that induction of ductal cells does not require continued Ngn3 expression.

### Example 10 (iii)

### Ngn3 is Capable of Promoting the Pancreatic Trunk Field Prior to Onset of Terminal Ductal and Endocrine Differentiation

As the results of the Ngn3 Brief ON experiments could indicate that an early change to pancreatic progenitor patterning had resulted from the elevated Ngn3 expression, we carried out a detailed analysis of the Ngn3 Delayed ON pancreas at E12.5. qRT-PCR revealed a modest (˜1.2-fold) increase in glucagon (Gcg) mRNA expression and a reduction (˜40%) in Sox9 mRNA expression (FIG. 31H). Insulin (Ins) was not expressed at significant levels in WT, or DTG pancreas at this stage. Attesting to the functional ability of ectopic Ngn3 protein, we noted a significant activation of Ngn3 target genes, including NeuroD1, Pax4, Pax6 and Arx (FIG. 31H). Endogenous Ngn3 expression was attenuated to ˜20% of WT levels. A possible explanation for the reduced expression of endogenous Ngn3 could be increased activity of Notch signaling. We addressed expression of the Dll1 gene, which has been identified as a target of Ngn3. Dll1 expression was significantly increased (2.4 fold compared to WT) (FIG. 31H). A similar increase was noted in the Notch target gene Hes1, which is critical for pancreatic development, whereas expression of multiple other hairy/enhancer-of-split family members were unchanged (data not shown).

The increased endocrine progenitor marker expression was paralleled by a commensurate decrease in acinar markers. Expression of the Ptf1a gene was reduced to ˜50% of WT levels, and the initiation of terminal differentiation of acinar cells was severely blunted as revealed by a lowered expression of Cpa1, Amylase2 (FIG. 31H), and Cpa2 (data not shown) to ˜20-30% of WT levels. This was contrasted by a maintained expression of the, at this time, epithelial progenitor markers Krt19 and Tcf2 genes. Although we did not find evidence of increased terminal ductal differentiation, a significant increase in expression of the Onecut-family member 2 (Oc2) was evident (FIG. 31H), and the normal epithelial apical restriction of Mucin1 was observed (31B). Interestingly, expression of the homologous gene Oc1 (encoding Hnf6, a known regulator of Ngn3 expression), was significantly reduced to ˜20% of WT levels (FIG. 31H).

The observed changes in gene expression argued for a profound change in organ compartmentalization. We addressed the creation of the pancreatic pro-acinar field. Ptf1a is a useful marker, at E12.5 being confined to distal tip cells contacting the outer basal lamina of the epithelium (FIG. 31C). The nuclear staining for Ptf1a was almost abolished in Ngn3 Delayed ON pancreas (FIG. 31D). This loss was proportionate with a reduction in staining for CPA1 (FIG. 31B), as well as amylase (not shown). Next, we addressed the creation of the trunk progenitor field. Nkx6.1 is a useful marker, as this becomes localized to the trunk field prior to E12.5 and is excluded from the distal tip field (FIG. 31E). Nkx6.1 has been shown to operate antagonistically to Ptf1a, as described by Schaffer et al., Dev Cell. 2010 Jun. 15; 18(6):1022-9. Nkx6.1 was widely distributed throughout the E12.5 Ngn3 Delayed ON pancreas (FIG. 31F); expressed in cells analogous to a tip-position, at the epithelial/mesenchymal border. Morphometrical analysis confirmed the relative shift between the Nkx6.1 and Ptf1a trunk/tip markers (FIG. 31G). We conclude that ectopic Ngn3 expression elicited a shift towards Nkx6.1 expression, with a concomitant loss of Ptf1a expression, and hereby distorts the tip/trunk pattern that normally segregates multipotent pancreatic progenitors prior to the secondary transition.

### Example 10 (iv)

### Preservation of the Dynamics of Hes1/Ngn3 Expression, and Consequently Lateral Inhibition, During Ectopic Ngn3 Expression

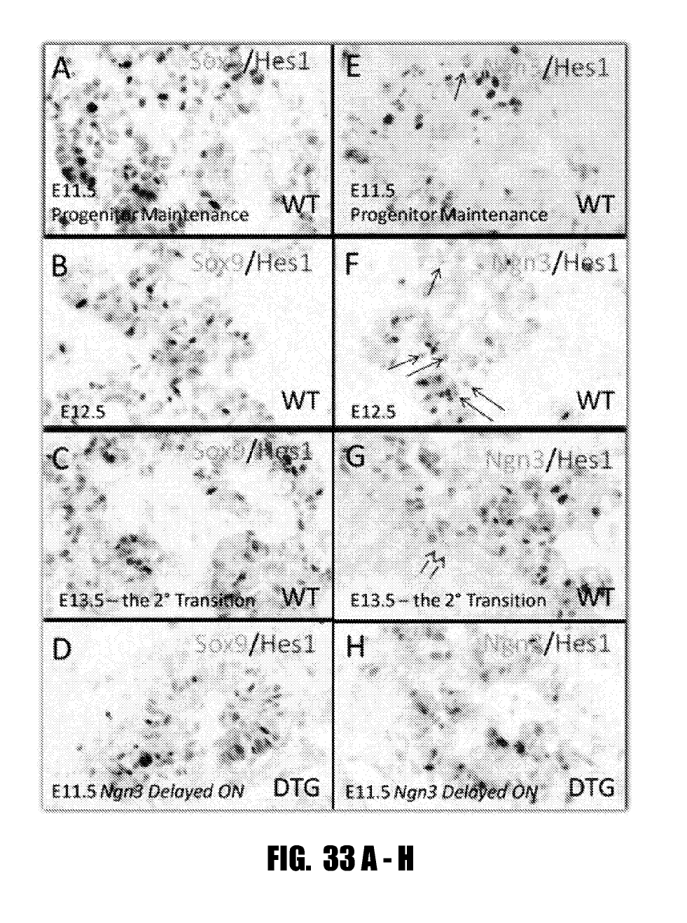

The effect of Ngn3 on Dll1 and Hes1 expression prompted us to examine if lateral inhibition was prematurely engaged in the Ngn3 Delayed ON pancreas, as if so, this could help explain the dichotomy of the trunk-field resolution into both endocrine and ductal cells. To do this, we carried out co-immunostaining of Ngn3 and the oscillatory Notch-target Hes1 and also related the expression of Hes1 to the progenitor marker Sox9. In WT pancreas, Hes1 protein is expressed heterogeneously, but widely, throughout the progenitor pool at E11.5 (FIG. 33A). This expression always overlaps with Sox9 (FIG. 33A), although Sox9 is more homogenously expressed, in agreement with Sox9 being upstream of Hes1. This pattern remains qualitatively intact between E11.5-E13.5 (FIG. 33A-C), and exists prior to the emergence of Ngn3+ cells (FIG. 33E), arguing that Hes1 is expressed dynamically in individual progenitor cells prior to Ngn3>Dll1>Notch lateral signaling. This observation implies that Notch signaling is engaged prior to normal onset of Ngn3 expression at the secondary transition at E13.5. In WT, the numbers of Ngn3+ cells gradually increase over E11.5-E13.5 (FIG. 33E-G), decreasing thereafter (not shown). At these time points, Ngn3 is always reciprocally expressed with Hes1, agreeing with the mechanism of lateral inhibition. At E13.5, Hes1-negative/Ngn3-negative cells are found, likely corresponding to differentiated cells (FIG. 33G, yellow arrows). We anticipated that the transgenic Ngn3 model would result in Ngn3+/Hes1+ double-positive cells, as the driving promoter (Pdx1) for the transgenic system should be refractory to lateral inhibition. We also expected to see a gradual loss of the progenitor cell population, reflected by loss of Sox9 when the cells would enter the trunk-field. But, this was not found. Analyzing Ngn3 Delayed ON pancreas at E11.5, the Sox9/Hes1 expression pattern remained qualitatively intact compared to WT (FIG. 33D). Yet, the number of Ngn3+ cells (which are mainly detectable due to the TG Ngn3 production, as the endogenous Ngn3 mRNA is effectively reduced, FIG. 33H) was markedly increased at E11.5 as compared to WT pancreas as expected. Ngn3 and Hes1 remained reciprocally expressed (FIG. 33H), qualitatively similar to WT at E13.5 (FIG. 33G). This would signify the premature engagement of lateral inhibition.

### Example 10 (v)

### Notch Signaling Controls Ngn3 Protein Stability

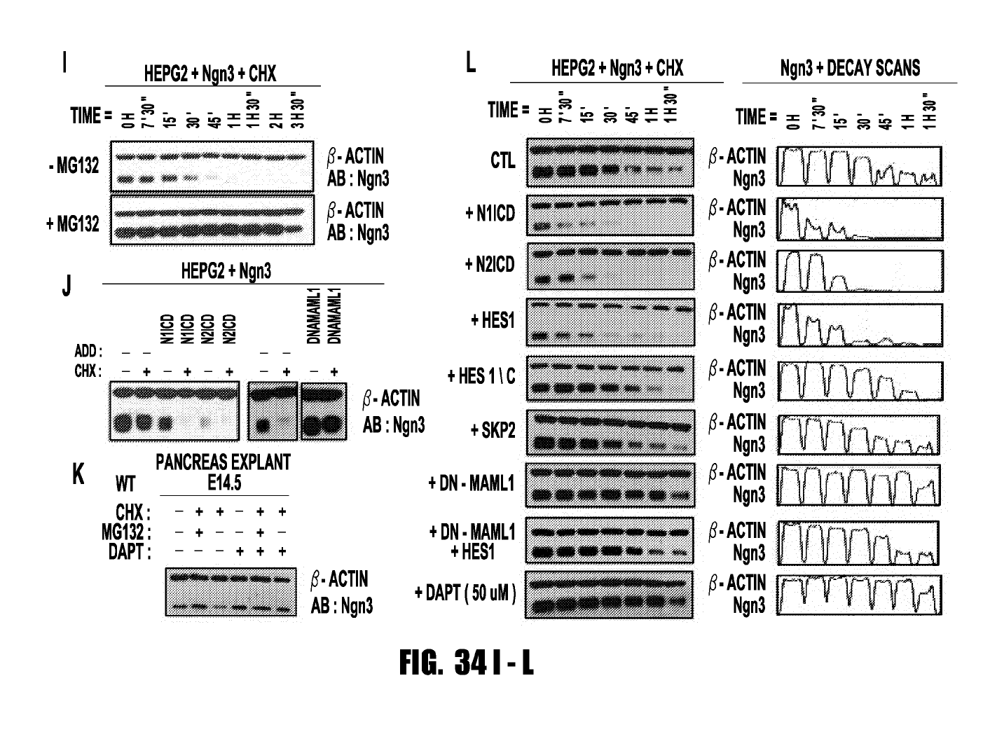

While Hes1 is known to directly repress Ngn3 expression, this would be an insufficient explanation for the observed results in which Ngn3 was driven by a Pdx1-tTA;tetO governed system. Therefore, the lack of homogenous Ngn3 presence in the DTG pancreas strongly suggested that Notch-mediated lateral signaling negatively controls Ngn3 protein. As destabilization of other bHLH factors, including NGN, and hASH1 has been observed, which has also implicated Notch, we then tested if a similar mechanism might operate in pancreas. As a means to mechanistically address this question, we first determined if Ngn3 protein half-life was regulated by proteasomal activity. Using three different heterologous cell lines (HepG2, Hek293, and CHO) conditionally expressing Ngn3, we observed a rapid turnover of Ngn3 protein after cycloheximide (CHX) addition; conditions where the control protein, beta-Actin, remained stable (FIG. 34I and data not shown). As Notch signaling pathway is involved in growth control of HepG2 cells, and because these cells express Delta1, Jagged1, Notch3 ICD, and Hes1, we chose the HepG2 cell line for more detailed studies. Through CHX time course experiments, we defined the half-life of Ngn3 to be very short (T_{1/2}˜30′) (FIG. 34I), comparable to T_{1/2} values for Ngn1, and Mash1, as previously reported. Exposure of Ngn3-expressing HepG2 cells to the proteasomal inhibitor MG132 stabilized the protein, T_{1/2}>3 hr (FIG. 34I). We then tested if Notch signaling controlled Ngn3 protein accumulation and Ngn3 degradation in HepG2 cells by co-expression of the Notch1, or Notch2 intracellular domains (N1ICD, N2ICD, FIG. 34J, L). In presence of Notch, Ngn3 protein failed to accumulate (FIG. 34J), and the measured T₁₁₂ of Ngn3 was extremely short (T_{1/2}<15′, FIG. 34L). Investigating if transcriptional regulation was required downstream of Notch processing, we co-transfected a dominant negative version of the prerequisite Notch signaling co-activator Mastermind-like-1 (DN-MAML1). This completely abrogated the ability of Notch in destabilizing Ngn3, and furthermore led to a marked increase in total amounts of Ngn3 produced in the HepG2 cells (FIG. 34J, L). We conclude that the basal turnover of Ngn3 in the HepG2 line was mainly conferred by the ongoing Notch signaling in those cells; a finding confirmed by an increase in total Ngn3 protein levels—with a commensurate protein stabilizing effect—in the presence of the Notch inhibitor, N—[N-(3,5-difluorophenacetyl)-1-alanyl]-S-phenylglycine t-butyl ester (DAPT) at 50 μM (FIG. 34L, and data not shown). It could be argued that the mechanistic insight derived from the HepG2 studies was cell culture specific, but E14.5 mouse pancreatic explant studies (WT) using CHX and DAPT revealed that endogenous Ngn3 indeed was rapidly turning over following exposure to CHX, and that this loss was abrogated by exposure to MG132 (FIG. 34K). Also, MG132 led to a significant increase in total Ngn3 protein in presence of CHX. The turnover of Ngn3 protein in pancreas was not only dependent on the proteasomal system, but Notch as well, as exposure of explants to DAPT over the same time course led to similar levels of Ngn3 accumulating as compared to MG132-treated pancreas (FIG. 34K). The DAPT-induced increase in Ngn3 protein was not reduced upon CHX presence (FIG. 34K). As the total level of DAPT-induced Ngn3 protein was comparable to that of MG132 treated pancreas, we conclude that Notch signaling is the predominant regulator of Ngn3 protein stability in the E14.5 normal mouse pancreas, requiring proteasomal function.

While the mechanism linking Notch signaling to Ngn3 degradation is unknown, we noted that overexpression of Hes1 protein also led to the destabilization of Ngn3, with almost identical kinetics as observed with N1ICD, or N2ICD (FIG. 34L). Deletion of the C-terminal domain of Hes1 abrogated this effect (FIG. 34L). To ascertain if Hes1 was able to control Ngn3 turnover in absence of transcriptional events downstream of active Notch, we overexpressed DN-MAML1 in the presence of Hes1. While DN-MAML1 led to a marked increase in total Ngn3 protein as expected, Hes1 remained able to destabilize Ngn3 (FIG. 34L). Consequently, Hes1 is—independently of other transcriptional events elicited by Notch—able to control Ngn3 degradation.

### Example 10 (vi)

### Induction of Notch Signaling is the Underlying Molecular Mechanism for Ngn3-Mediated Ductal Cell Differentiation

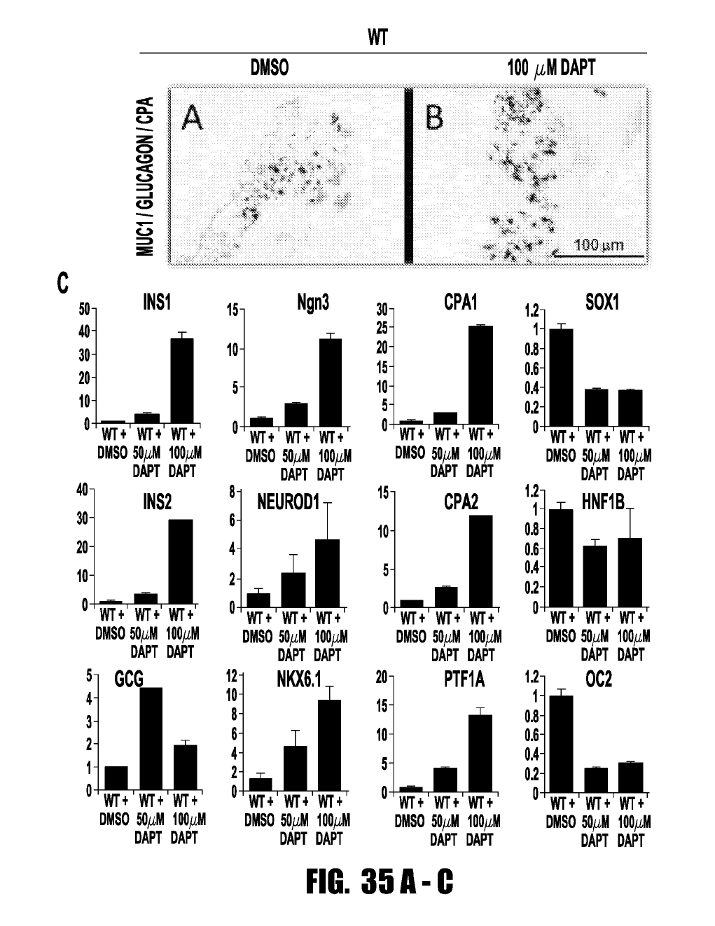

While implying a contributory role of Notch signaling, our previous data did not allow us to formally establish that this molecular mechanism is the underlying reason for ductal cell differentiation during elevated Ngn3 presence. Given that pancreatic ductal cells form in Ngn3 null mice, and because ductal genes have not been identified as direct Ngn3 targets, we set forth to determine if the cell-extrinsic mechanism involving Notch indeed was causal for the Ngn3 Delayed ON phenotype. We first tested if pancreatic ductal cell differentiation was dependent on Notch. We cultured WT pancreatic explants in the presence of DAPT (FIG. 35A, B, C). These experiments were based on E12.5 pancreas, as at that age, the majority of tip/trunk patterning has occurred, and cells are actively undergoing terminal fate decisions. These cultures developed significant numbers of Muc1+ ductal cells during culture in absence of DAPT (FIG. 35A), with a density exceeding that found in-vivo. Acinar cell development (CPA1+) proceeded in WT (FIG. 35A), albeit at a much reduced level compared to normal development. In the presence of 25-50 μM DAPT, ductal cell development was significantly reduced, and fully abolished at 100 μM DAPT addition (data not shown, and FIG. 35B). Given that Notch processing inhibition strongly affected cellular differentiation, we investigated this further by performing qRT-PCR on similar-type explants (FIG. 35C). As shown by others, Notch signaling inhibition led to quite dramatic increases in endocrine cell differentiation (dose-dependent increases observed for Ngn3, NeuroD1, Nkx6.1), and it also strongly promoted acinar gene expression (Cpa1, Cpa2, Ptf1a). However, variable effects on endocrine subtypes was noted, where at high levels of DAPT (100 μM), Gcg expression was reduced, while Ins1, Ins2 was further enhanced. In agreement with a dependency on Notch signaling for pancreatic progenitor maintenance, Sox9 expression was reduced and ductal-marker expression (Hnf1β, Oc2) was also substantially decreased.

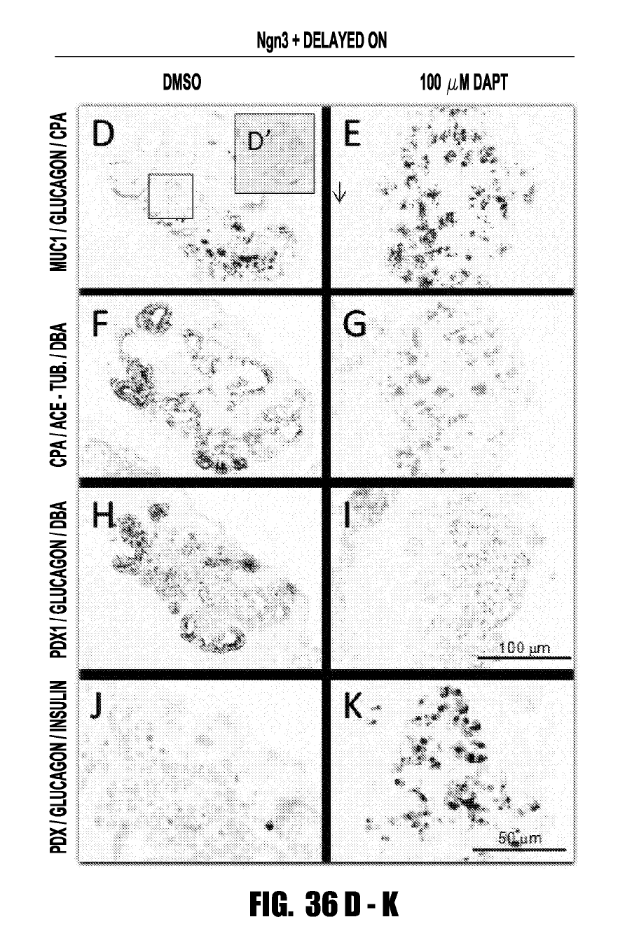

Given that Dll1 and Hes1 were >2-fold increased in the E12.5 Ngn3 Delayed ON pancreas, we next asked to what degree Notch processing was also required for the enhanced Ngn3-mediated ductal cell formation in that model. Similar to above, microdissected E12.5 Ngn3 Delayed ON pancreas was cultured for four days ex-vivo with and without DAPT (FIG. 36D-K). In contrast to WT explants, Ngn3 Delayed ON pancreas developed in a quite similar manner as observed in-vivo; in addition to widespread ductal formation, glucagon-producing clusters were observed, and extremely few acinar cells were found (FIG. 36D, F, H). Similar results were obtained using staining for Hnf1β (data not shown). As observed for WT pancreas, acinar density (CPA1+ cells) was much increased upon DAPT treatment (FIG. 36E, G). The Ngn3-mediated ductal cell formation was abrogated by DAPT treatment (FIG. 35E, G, I). Of note, numerous Pdx1+/Ins+ endocrine cells were observed upon DAPT treatment (FIG. 36K), while glucagon-expressing cells (normally encountered at significant numbers in non-DAPT treated Ngn3 Delayed ON pancreas, FIG. 36D, H, J) were correspondingly reduced (FIG. 36E,I,K). We conclude that Notch signaling is required for both normal, as well as Ngn3-mediated, pancreatic ductal cell development, and that blocking this pathway promotes acinar and endocrine fates at the expense of ductal cells. Of note, at high levels of DAPT treatment, γ-secretase inhibition impacts endocrine subtype determination as well, and if a concomitant increase in Ngn3 protein is provided, the organ regains capacity for significant insulin-cell differentiation.

### Example 11

### The Effect of a Maturation Media Additive Set on the Generation of Pancreatic Endocrine Fates from Directly Induced Pancreatic Progenitors Derived from Pluripotent Cells

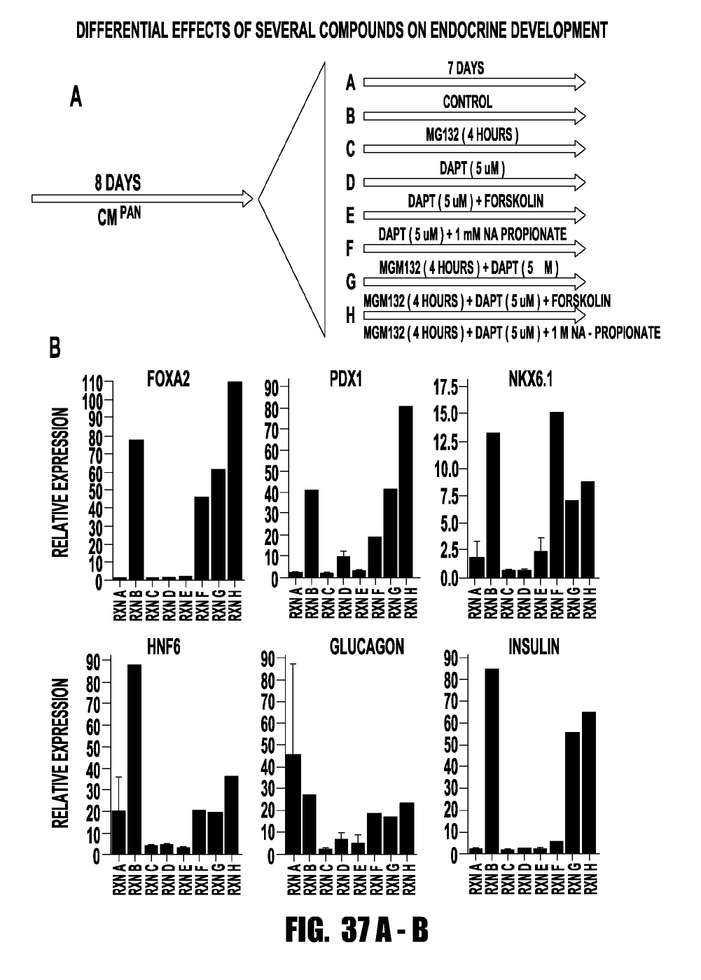

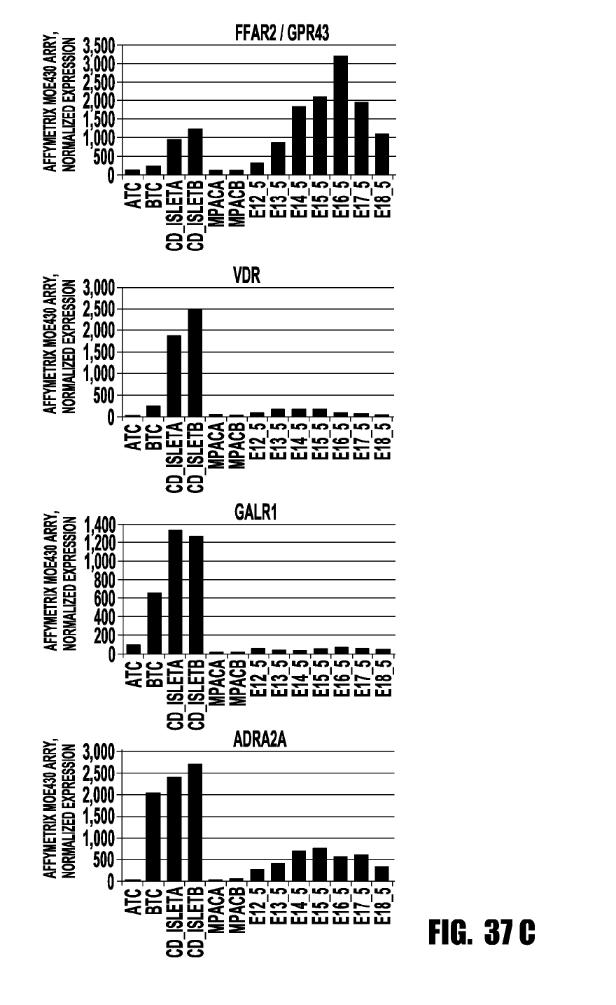

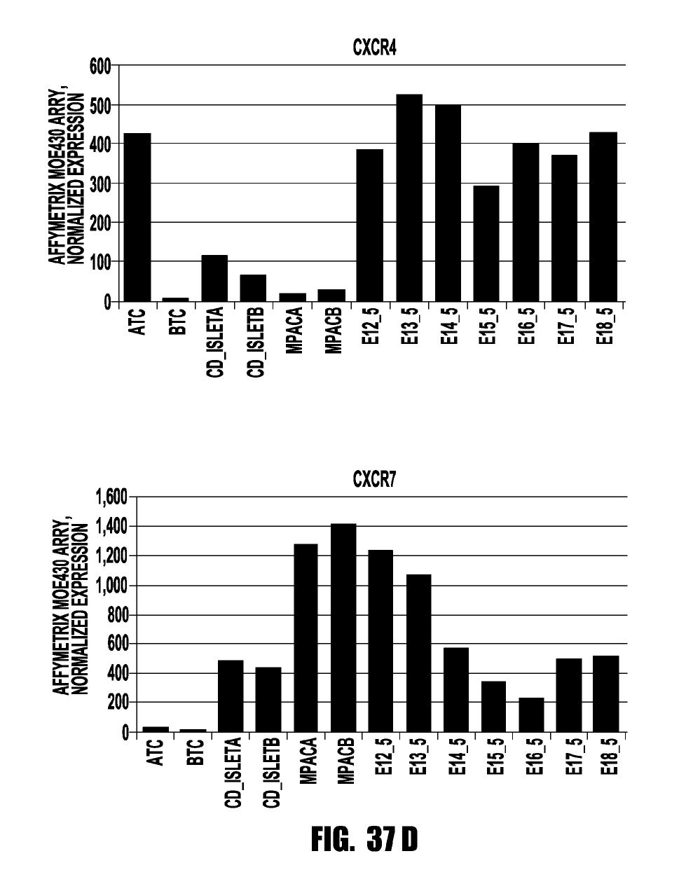

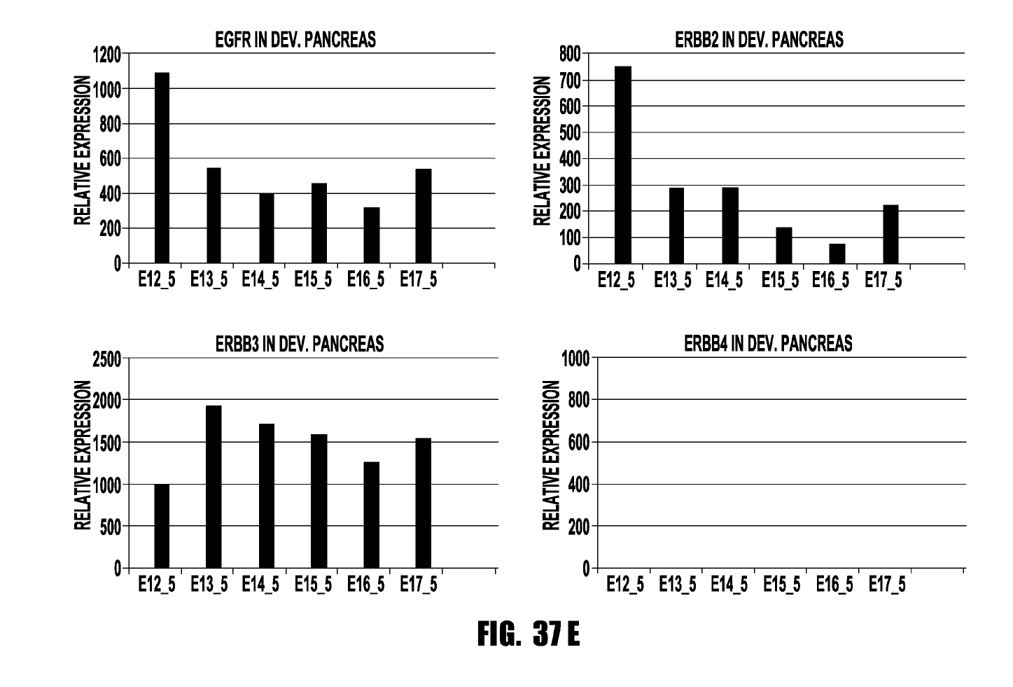

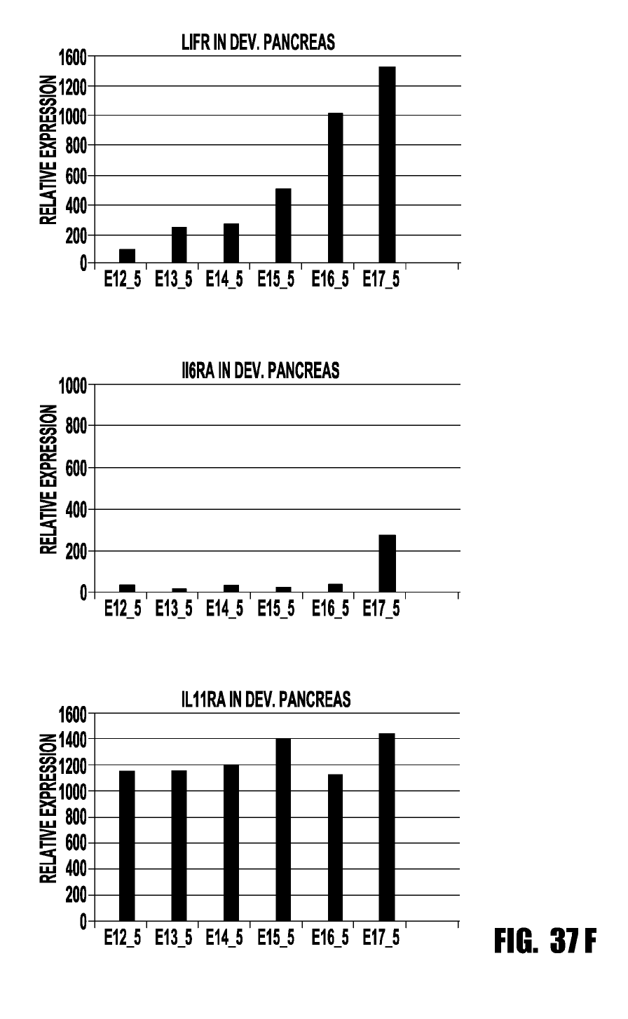

Since our initial attempts at redirecting the terminal fates realized in a pluripotent culture maintained in Pan-CM successfully demonstrated a competence of these forward differentiating cultures to respond to exogenous signals allowing for an increased formation of insulin producing cells, we next screened a number of compounds previous indicated to improve beta cell differentiation, by us and others, for their ability to promote differentiation towards an insulin producing cell. Among the compounds assayed for the ability to increase insulin production in a forward differentiating culture was the proteosomal inhibitor MG132 which we used in the previous example to stabilize Ngn3 protein. We also included the gamma secretase inhibitor DAPT, which was shown in the previous example to greatly increase beta-cell formation in the developing embryonic pancreatic explant study. We also included the short fatty acid salt sodium propionate, which acts as an agonist of short-chain fatty acid receptors. We also included the adenylyl cyclase activator forskolin. We also included the nuclear hormone receptor agonist 1,25 (OH)2)-Vit D3, which binds to, and activates the VDR protein. We also used intermediate concentrations of glucose, shown by others to increase formation of insulin producing cells from pluripotent derivatives. These compounds were used individually or in combination (as outlined in FIG. 37 panel A) to increase insulin-producing endocrine cell formation in a short term Pan-CM treated forward differentiating hES culture system.

The rationale for the administration of these compounds were based on considerations to beta cell physiology, and in particular our demonstration that cognate receptor types, and signaling mediates for these components are present selectively, or with high specificity, in pancreatic beta cells, and/or developing pancreas at the time when beta cells form (E13.5-E14.5 in mice). Supportive data for these pathway agonists are shown in FIG. 37. FIG. 37 demonstrates expression of Ffar2/GPR43, which is a short chain fatty acid receptor that responds to propionate, and butyrate. It is expressed at very high levels in pancreatic islets, and strongly at time of beta cell development in the mouse embryo. Vdr encodes the nuclear hormone receptor for the active derivative of vitamin D3, 1,25 (OH)₂-Vit D3. It is selectively expressed in mature islets, and is enriched in pancreatic insulin producing tumor cells, compared to glucagon-expressing tumor cells, arguing that Vdr is beta cell specific among islet cells. Vdr is autoregulated as shown by others, and is involved in controlling Ca²⁺ secondary messenger pathway genes; a pathway that is critical for adult beta cell function, in consideration of the physiological role of Ca²⁺ in physically mediating insulin granule release via binding to synaptotagmin present at the granule, and it is also the main parameter that responds to membrane depolarization, which occurs upon glucose-uptake and metabolism in the beta cell. The process of ATP-generation causes ATP-sensitive potassium channels to close, subsequently eliciting a depolarization of the cell membrane. This event triggers opening of voltage-dependent calcium channels, and the influx of extracellular calcium. The calcium uptake triggers insulin release. The maturation of a physiologically response Ca²⁺ pathway, is therefore of crucial importance in normal beta cell physiology. Here, we also demonstrate expression of Galr1, galanin-receptor 1, another GPCR family member, being selectively expressed by pancreatic islets (FIG. 37). Here we also demonstrate the specific expression of Adra2a (FIG. 37), the GPCR involved in binding, and transmitting, effects of epinephrine/adrenaline. Adra2a involves adenylate cyclase activity, and is specifically expressed in pancreatic insulinoma, but not glucagonoma cells. It is abundantly expressed in islets, being the most abundant GPCR member (out of >1200 such genes) expressed in islets. Its expression in embryonic pancreas is consistent with possible epinephrine/adrenaline response competency at time of beta cell formation at E13.5-E14.5.

While our investigations provide data on the functional relevance of several of these pathways, those skilled in the art will recognize that each of the identified pathways may be modulated by alternative means than those described here. In particular, the use of forskolin, a PKA agonist, can be mimicked by 8-Br-cAMP, and the generation of intracellular cAMP can be achieved by extracellular agonists that mediate activation of GPCR family members that connect with the adenylate cyclase enzyme resulting in the generation of intracellular cAMP. Examples of such are represented by, as per example, provision of Adra2a agonists, such as, but not limited to, epinephrine/adrenaline, or alternatively Galanin, or derivatives of Galanin, or small molecules that activate the Galr1, or alternatively, Glp1R agonists, such as Glp1, or exendin4, or alternatively, pharmaceutically developed and stabilized Glp1-protein derivatives, or alternatively, small molecule agonists. An alternative stimulation may occur via the GIPR, and thus include GIP, or small molecules activating the GIPR. It is known to those skilled in the art that both GIPR are GLP1R are both expressed with reasonable selectivity on the surface of pancreatic beta cells.

Our studies also include the provision of an agonist of the Free Fatty Acid Receptor family. In particular, we include short-chain free fatty acids, which can be represented by propionate, or alternatively, butyrate. The rationale for their application is related to the presence of the Ffar2 on beta cells; a receptor that mimics but is not identical to, the functions of the medium/long-chain free fatty acid receptor (GPR40), which operates in adult beta cells to regulate insulin secretion. Ffar2, however, is specifically binding short-chain free fatty acids. The mechanism by which these receptors mediate intracellular signaling involve potentiation of the Ca²⁺ response in the beta cell, and suggest that they may work alongside Vdr signaling.

The overarching rationale for the combinatorial use of the signaling pathways described above is therefore to provide maximal stimulation of embryonic-expressed pathways that may lead to appropriate development of key signaling pathways in the adult beta cells, notably that of glucose-dependent metabolism and adaption; the emergence of an incretin response, mediated by the responsiveness to the second messenger cAMP; the emergence of a Ca²⁺ response, maximized in relation to membrane depolarization and granule exocytosis, and finally, the maximal stabilization of the key endocrine inducing factor, Ngn3, in order to circumvent alternative fate adoptions of TrPC cells present in the culture, otherwise able to adopt, in particular, undesired ductal fates.

All cases in which forward differentiating cultures were treated with MG132 cultures showed increased relative transcript levels for all of the pancreas-specific markers assayed. The TrPC markers Pdx1, Nkx6.1 and Hnf6 were increased ˜40, ˜12.5 and ˜90 fold respectively (FIG. 37 panel B), while the effects of this compound on transcript levels of the respective terminal endocrine genes varied with Glucagon transcript levels remaining approximately the same as controls and Insulin transcript levels increasing ˜80 fold over controls. This suggests that the stabilization of Ngn3 specifically enhances the differentiation towards Insulin producing cells in the hES model system. The effects observed from the small molecules sodium propionate and forskolin were minor in comparison, though inclusion of both these compounds with Mg132 resulted in increases in FoxA2 and Pdx1 transcript levels over reactions which were solely treated with MG132.

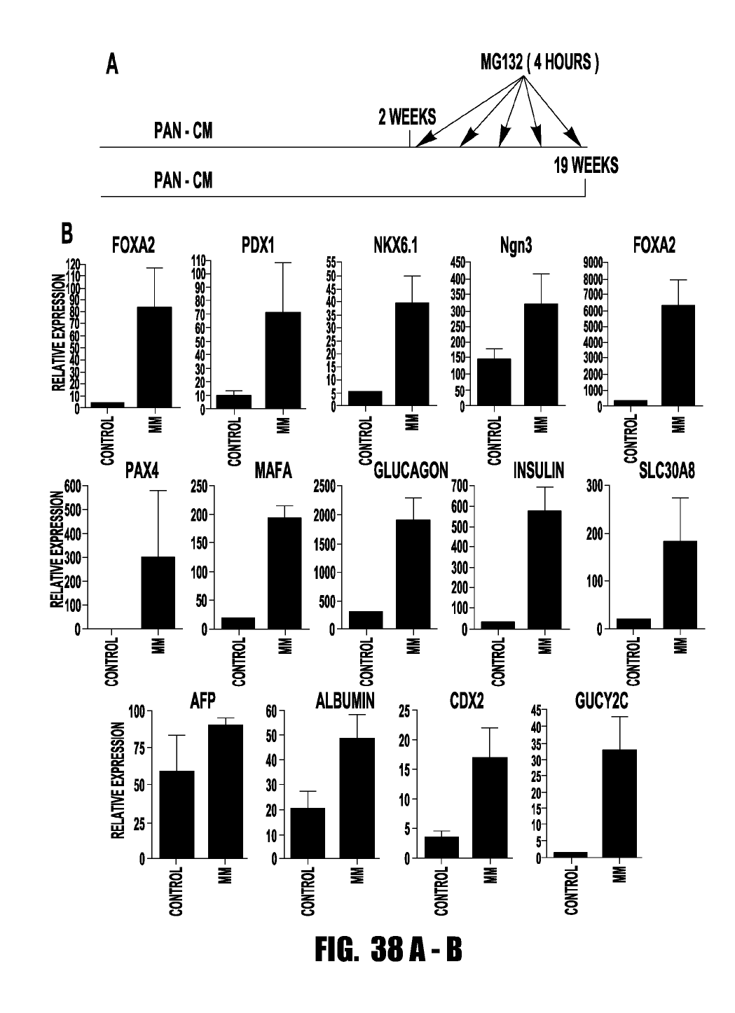

Since our initial attempts at increasing the in vitro generation of insulin producing cells through the MG132 mediated inhibition of protein degradation demonstrated a significant enhancement in insulin production, we next took a closer look at the effects of MG123 administration on several beta-cell specific transcripts. Forward differentiating hES cultures were incubated for two weeks in the presence of Pan-CM at which time culture conditions were changed to a defined maturation medium in addition to being treated daily with MG132. Control cultures remained within Pan-CM for the entire duration of the experiment. Forward differentiating cultures which were treated with MG132 showed increased expression for several genes associated with a pancreatic trunk progenitor including Pdx1, Nkx6.1 and Hnf6 (FIG. 38 panel B). These transcript levels were significantly increased over both hES levels as well as the transcript levels associated with hES cultures maintained in Pan-CM for an equivalent amount of time. In addition several endocrine and beta-cell specific transcripts were also significantly increased. Transcript levels for Pax4 and Mafa, transcription factors associated with the differentiation of beta-cells during embryonic development, showed an ˜300 fold and an ˜200 fold relative increase respectively over control conditions. Transcript levels of Slc30A8, a gene associated with the transport of zinc during the processing of insulin, were increased ˜200 fold when forward differentiating cultures were exposed to MG132. Both the endocrine markers Glucagon and Insulin, alpha cell and beta cell specific genes, were significantly increased over controls with absolute relative levels increased ˜2000 and ˜600 fold respectively over hES levels. It is important to note that while the absolute Glucagon transcript level fold increase was greater than that of insulin, when comparing the effects of forward differentiating cultures treated with Mg132 as to those which were solely treated with Pan-CM there was an ˜5 fold greater increase in relative Glucagon transcript levels as compared to an ˜24 fold greater increase in Insulin transcript levels. This indicates that there was a preferential increase in Insulin transcript levels over that of relative Glucagon transcript levels. Finally, while all of the pancreatic transcripts assayed through this series of experiments showed significant high fold increases over the relative levels obtained in forward differentiating cultures maintained in Pan-CM without the MG132 treatment, similar increases were not noted in non-pancreatic organ-specific transcripts. The liver specific transcripts levels of AFP and Albumin, an ˜85 and ˜50 fold increase respectively, were only minimally up-regulated. A similar low increase was also noted for the intestinal specific transcripts Cdx2 and Gucy2c genes, with only an ˜15 and ˜30 fold increase over hES transcript levels.

All together this demonstrates that the proteasomal inhibitor MG132 is capable of successfully increasing insulin production within a forward differentiating that has an increased pancreatic phenotype, however long incubations of this compound within a forward differentiating culture tended to have an overall detrimental effect on the culture and it was found within this study that shorter and fewer incubation times was needed to successfully establish terminal differentiation towards beta cell-like fates. Also it is important to note that while all of the transcripts assayed throughout this series of experiments were increased in the cultures treated with MG132 the increase in pancreas specific transcripts was always far greater than the increases observed in non-pancreas specific transcripts (i.e., transcripts representative of the liver and intestines). More importantly beta-cell specific transcripts always increased several hundred-fold over controls while the intestinal and liver genes assayed generally increased substantially less than a hundred fold indicating a preferential effect on endocrine cell development.

### Example 12

### Defining the Pan-MEF Secretome

To help determine the critical components present in the Pan-CM needed to induce a pancreatic phenotype within a forward differentiating hES culture a combined analysis of the corresponding transcriptome (microarray approach) and the secretome was undertaken. Triplicate RNA extractions from passage 3 (6 days in culture) pancreas-specific MEF cultures were subjected to microarray analysis and compared to MEFs derived from the body trunk of the same litter of embryos. The analysis of the microarray data focused on the morphogens and signaling molecules being expressed uniquely in the pancreas-specific MEF cultures. The expression profiles of several protein families are shown in FIG. 38. Only limited expression of members of the FGF protein family was observed. The strongest expression of FGF molecules were FGF7 within the MEF culture derived from the bodies trunk of E13.5 embryos and FGF13 for the pancreas-specific mesenchymal cultures, though neither were significantly expressed with arbitrary levels reaching ˜125 and ˜120 respectively. However, it is important to note that FGF10 expression was noted in different Pan-MEF preparations (data not shown) suggesting that FGF10 expression can occur within the Pan-MEF population.

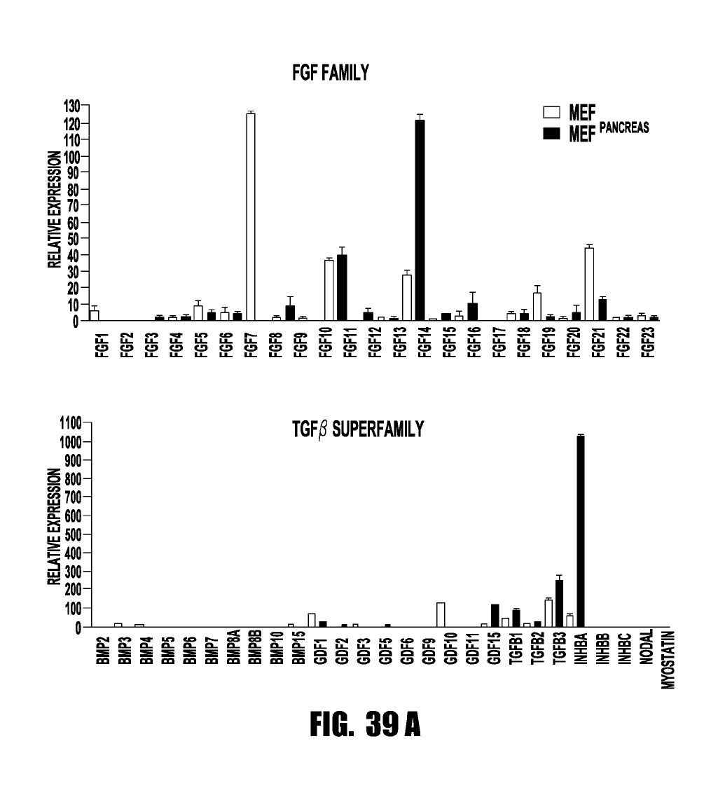

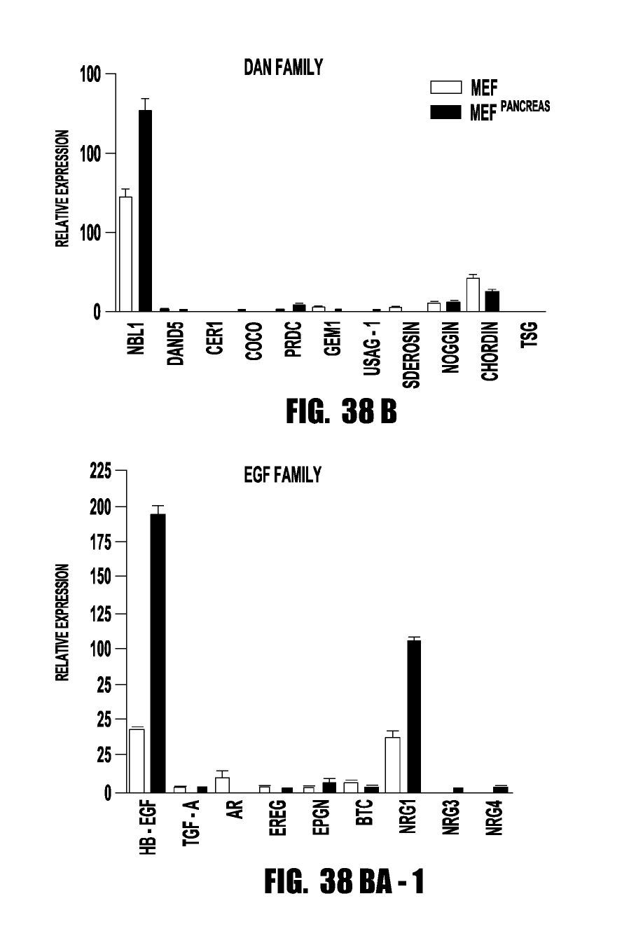

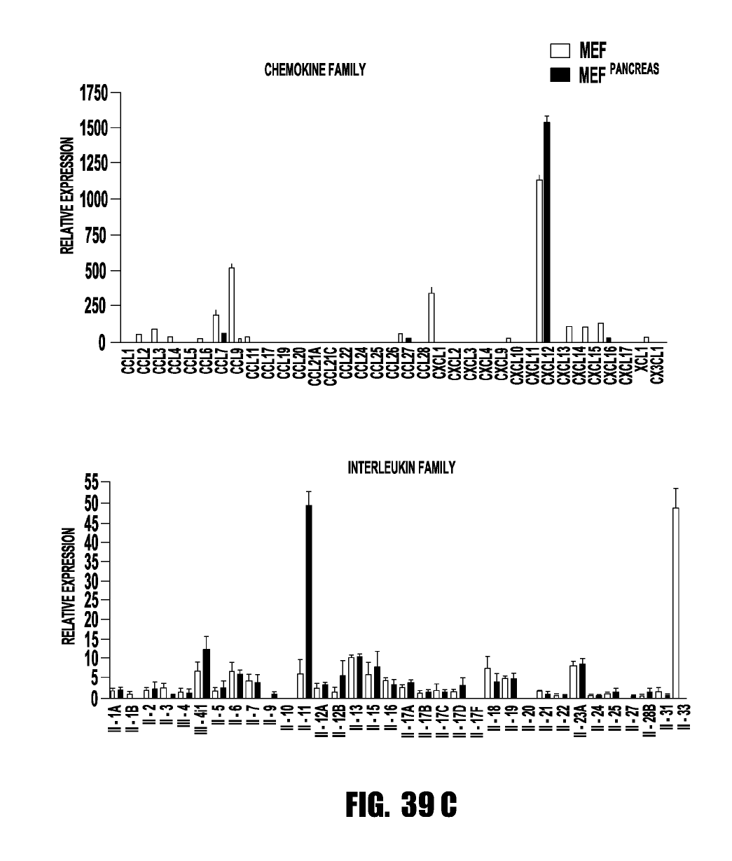

Next we examined the expression pattern for proteins of the Tgfβ superfamily (FIG. 39A). While the Pan-MEF expression of Gdf15, Tgfb1 and Tgfb3 were low to moderate with arbitrary levels ranging from ˜115, ˜90 and ˜250 respectively, the expression of Inhba was high with arbitrary levels reaching a little more than ˜1000. This high level of Inhba was consistent and Inhba expression was observed in all Pan-MEF preparations that were assayed for its presence. It is well established that pluripotent cells are capable of responding to Inhba stimulation, further it is commonly administered to pluripotent cultures during stage 1 of directed differentiation protocols as a means of pushing cultures through an in vitro gastrulation event towards endodermal lineages. It has also been shown to be extremely beneficial later in pancreatic directed differentiation protocols increasing the percentage of endocrine progenitors during stage 4 administration. All together this suggests that Inhba is probably a crucial functional component of the Pan-CM since it plays such a crucial role in all directed differentiation protocols designed to generate pancreatic lineages.

We next examined the expression pattern of the DAN protein family members and the EGF protein family (FIG. 39B). While the majority of the DNA protein family members were not expressed in either the Pan-MEF cultures or the MEF cultures derived from embryonic body trunks, Nbl1 was shown to be expressed in both cultures with the higher expression occurring in the Pan-MEF cultures. Arbitrary expression levels reaching ˜250 and ˜150 for the Pan-MEF cultures and the MEF cultures derived from embryonic body trunks respectively. Expression of EGF protein family members was also limited with only Hb-EGF and modest levels of Nrg1 being detected within the Pan-MEF cultures. Pan-MEF cultures showed the level of Hb-EGF reaching ˜200 and the level of Nrg1 reaching ˜100. Both of these levels were significantly increased over the respective levels reached by these proteins in the MEF cultures derived from the embryonic body trunks.

We next assayed the expression patterns of the members of the chemokine and the interleukin protein families (FIG. 39C). While the body-trunk derived MEF cultures displayed several chemokines being expressed to different levels the Pan-MEF cultures showed only CXCL12 expression. Assessment of the body-trunk derived MEF cultures showed the expression of CCL7, CCL9, CXCL1, CXCL12, CXCL14, CXCL15 and CXCL16 at the respective arbitrary levels ˜190, ˜520, ˜340, ˜1100, ˜100, ˜100 and ˜125. This widespread expression of several different chemokines was contrasted with the Pan-MEF cultures which clearly only expressed high levels of CXCL12 (levels reaching ˜1500). This high level of CXCL12 expression within the Pan-MEF cultures was greater than the expression noted in the MEF cultures derived from the body trunk. Analysis of the interleukin protein family between the two different cultures showed little to no expression of any members of the protein family in either culture. The highest expression was observed with IL-11 in the Pan-MEF culture and IL-33 within the MEF culture, though expression levels of these two proteins were low in both cases. The relative expression of the IL-11 within the Pan-MEF culture was ˜50 and the expression of IL-33 within the MEF cultures was also ˜50. This indicates that interleukin signaling is probably not crucial for the observed differentiating inducing effects of Pan-CM.

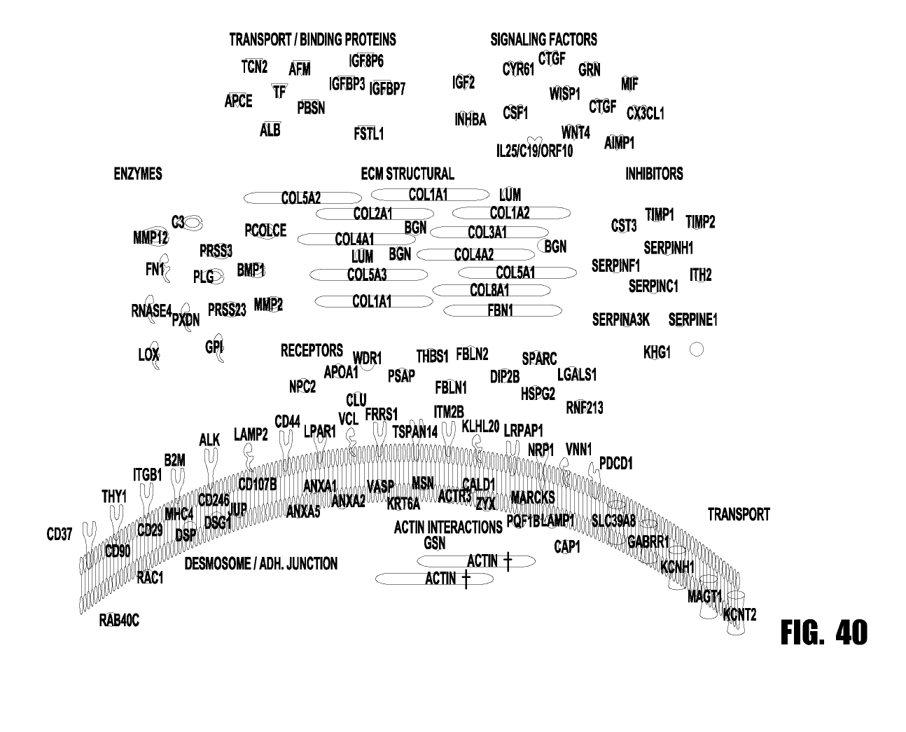

A complementary secretome analysis of the Pan-CM produced an independent analysis focused on the protein molecules present within the conditioned medium. While the bulk of the proteins detected through this methodology tended to be structural adhesion molecules, which would be expected from a mesenchymal culture, several signaling molecules were also detected. FIG. 40 present the data obtained through this methodology with a focus limiting the protein presented to those that are either solvent exposed or secreted (i.e., at the surface of the cell or secreted from the cell). The protein molecules detected are assembled into different groups using ingenuity software; the different groupings of molecules are indicated in the figure. Several ECM molecules were detected and the actual number of detections of the individual ECM molecules (i.e., hits on the MS) indicate that these molecules are by far the most abundantly expressed molecules. The secretome analysis also indicated that there was a number of signaling molecules present within the Pan-CM including: CTGF, CYR61, GRN, WISP1 MIF, IGF2, CSF1, MANF, CX3CL1, AIMP1, IL25/C19orf10, Wnt4 and Inhba. While this portion of the secretome analysis does not agree in its entirety with the molecules indicated through the array analysis there is at least a partial overlap between the two complimentary approaches. To allow for further analysis of the secretome data and completion of the analysis the entire data set from one of the secretome trials is presented in Table 1.

TABLE 1

| | prot_acc | prot_desc | prot_score | prot_mass |
|---|---|---|---|---|
| 1 | IPI00329872 | Tax_Id = 10090 Gene_Symbol = Col1a1 Isoform 1 of | 3495 | 137948 |
| | | Collagen alpha-1(I) chain | | |
| 2 | IPI00222188 | Tax_Id = 10090 Gene_Symbol = Col1a2 Collagen alpha- | 3356 | 129478 |
| | | 2(I) chain | | |
| 3 | IPI00110588 | Tax_Id = 10090 Gene_Symbol = Msn Moesin | 1666 | 67725 |
| 4 | IPI00131695 | Tax_Id = 10090 Gene_Symbol = Alb Serum albumin | 1591 | 68648 |
| 5 | IPI00230395 | Tax_Id = 10090 Gene_Symbol = Anxa1 Annexin A1 | 1504 | 38710 |
| 6 | IPI00323592 | Tax_Id = 10090 Gene_Symbol = Mdh2 Malate | 1444 | 35589 |
| | | dehydrogenase, mitochondrial | | |
| 7 | IPI00139301 | Tax_Id = 10090 Gene_Symbol = Krt5 Keratin, type II | 1434 | 61729 |
| | | cytoskeletal 5 | | |
| 8 | IPI00230108 | Tax_Id = 10090 Gene_Symbol = Pdia3 Protein disulfide- | 1175 | 56643 |
| | | isomerase A3 | | |
| 9 | IPI00131368 | Tax_Id = 10090 Gene_Symbol = Krt6a Keratin, type II | 1132 | 59299 |
| | | cytoskeletal 6A | | |
| 10 | IPI00323357 | Tax_Id = 10090 Gene_Symbol = Hspa8 Heat shock cognate | 1097 | 70827 |
| | | 71 kDa protein | | |
| 11 | IPI00221797 | Tax_Id = 10090 Gene_Symbol = Krt75 Keratin, type II | 1070 | 59704 |
| | | cytoskeletal 75 | | |
| 12 | IPI00407130 | Tax_Id = 10090 Gene_Symbol = Pkm2 Isoform M2 of | 1060 | 57808 |
| | | Pyruvate kinase isozymes M1/M2 | | |
| 13 | IPI00131547 | Tax_Id = 10090 Gene_Symbol = Serpine1 Plasminogen | 1036 | 45141 |
| | | activator inhibitor 1 | | |
| 14 | IPI00110850 | Tax_Id = 10090 Gene_Symbol = Actb Actin, cytoplasmic 1 | 993 | 41710 |
| 15 | IPI00845840 | Tax_Id = 10090 Gene_Symbol = Pkm2 Isoform M1 of | 960 | 57948 |
| | | Pyruvate kinase isozymes M1/M2 | | |
| 16 | IPI00462072 | Tax_Id = 10090 | 886 | 47111 |
| | | Gene_Symbol = Eno1; LOC100044223; Gm5506 Alpha- | | |
| | | enolase | | |
| 17 | IPI00330480 | Tax_Id = 10090 Gene_Symbol = −35 kDa protein | 872 | 35144 |
| 18 | IPI00555069 | Tax_Id = 10090 Gene_Symbol = Pgk1 Phosphoglycerate | 825 | 44522 |
| | | kinase 1 | | |
| 19 | IPI00556878 | Tax_Id = 10090 Gene_Symbol = Fstl1 Putative | 758 | 34532 |
| | | uncharacterized protein | | |
| 20 | IPI00121120 | Tax_Id = 10090 Gene_Symbol = Col5a2 Collagen alpha- | 718 | 144929 |
| | | 2(V) chain | | |
| 21 | IPI00126343 | Tax_Id = 10090 Gene_Symbol = Sparc SPARC | 708 | 34428 |
| 22 | IPI00230365 | Tax_Id = 10090 Gene_Symbol = Krt17 Keratin, type I | 699 | 48132 |
| | | cytoskeletal 17 | | |
| 23 | IPI00227299 | Tax_Id = 10090 Gene_Symbol = Vim Vimentin | 679 | 53655 |
| 24 | IPI00227140 | Tax_Id = 10090 Gene_Symbol = Krt14 Keratin, type I | 661 | 52834 |
| | | cytoskeletal 14 | | |
| 25 | IPI00625729 | Tax_Id = 10090 Gene_Symbol = Krt1 Keratin, type II | 648 | 65565 |
| | | cytoskeletal 1 | | |
| 26 | IPI00135686 | Tax_Id = 10090 Gene_Symbol = Ppib Peptidyl-prolyl cis- | 618 | 23699 |
| | | trans isomerase B | | |
| 27 | IPI00317309 | Tax_Id = 10090 Gene_Symbol = Anxa5 Annexin A5 | 613 | 35730 |
| 28 | IPI00127417 | Tax_Id = 10090 Gene_Symbol = Nme2 Nucleoside | 602 | 17352 |
| | | diphosphate kinase B | | |
| 29 | IPI00468696 | Tax_Id = 10090 Gene_Symbol = Krt42 Keratin, type I | 591 | 50102 |
| | | cytoskeletal 42 | | |
| 30 | IPI00314748 | Tax_Id = 10090 Gene_Symbol = Wdr1 WD repeat- | 591 | 66365 |
| | | containing protein 1 | | |
| 31 | IPI00322513 | Tax_Id = 10090 Gene_Symbol = Igfbp7 Igfbp7 protein | 575 | 27141 |
| | | (Fragment) | | |
| 32 | IPI00331088 | Tax_Id = 10090 Gene_Symbol = Serpinf1 Pigment | 556 | 46205 |
| | | epithelium-derived factor | | |
| 33 | IPI00319994 | Tax_Id = 10090 Gene_Symbol = Ldha L-lactate | 553 | 36475 |
| | | dehydrogenase A chain | | |
| 34 | IPI00226515 | Tax_Id = 10090 Gene_Symbol = Tagln Transgelin | 549 | 22561 |
| 35 | IPI00221402 | Tax_Id = 10090 Gene_Symbol = Aldoa Fructose- | 540 | 39331 |
| | | bisphosphate aldolase A | | |
| 36 | IPI00124499 | Tax_Id = 10090 Gene_Symbol = Krt79 Keratin, type II | 536 | 57517 |
| | | cytoskeletal 79 | | |
| 37 | IPI00109588 | Tax_Id = 10090 Gene_Symbol = Col4a1 Collagen alpha- | 533 | 160579 |
| | | 1(IV) chain | | |
| 38 | IPI00228633 | Tax_Id = 10090 Gene_Symbol = Gpi1 Glucose-6-phosphate | 525 | 62727 |
| | | isomerase | | |
| 39 | IPI00113539 | Tax_Id = 10090 Gene_Symbol = Fn1 Fibronectin | 514 | 272319 |
| 40 | IPI00755181 | Tax_Id = 10090 Gene_Symbol = Krt10 keratin, type I | 496 | 57007 |
| | | cytoskeletal 10 | | |
| 41 | IPI00515360 | Tax_Id = 10090 Gene_Symbol = Hspg2 basement | 490 | 469486 |
| | | membrane-specific heparan sulfate proteoglycan core | | |
| | | protein | | |
| 42 | IPI00405227 | Tax_Id = 10090 Gene_Symbol = Vcl Vinculin | 483 | 116644 |
| 43 | IPI00890117 | Tax_Id = 10090 Gene_Symbol = Cfl1 Cofilin-1 | 480 | 18548 |
| 44 | IPI00553419 | Tax_Id = 10090 Gene_Symbol = Dsp desmoplakin | 476 | 332706 |
| 45 | IPI00554989 | Tax_Id = 10090 Gene_Symbol = Ppia Peptidyl-prolyl cis- | 475 | 18302 |
| | | trans isomerase | | |
| 46 | IPI00468203 | Tax_Id = 10090 Gene_Symbol = Anxa2 Annexin A2 | 473 | 38652 |
| 47 | IPI00400300 | Tax_Id = 10090 Gene_Symbol = Lmna Isoform C of | 458 | 65407 |
| | | Lamin-A/C | | |
| 48 | IPI00322312 | Tax_Id = 10090 Gene_Symbol = Arhgdia Rho GDP- | 444 | 23393 |
| | | dissociation inhibitor 1 | | |
| 49 | IPI00347110 | Tax_Id = 10090 Gene_Symbol = Krt73 Keratin, type II | 435 | 58875 |
| | | cytoskeletal 73 | | |
| 50 | IPI00467833 | Tax_Id = 10090 Gene_Symbol = Tpi1 triosephosphate | 432 | 32171 |
| | | isomerase | | |
| 51 | IPI00229475 | Tax_Id = 10090 Gene_Symbol = Jup Junction plakoglobin | 432 | 81749 |
| 52 | IPI00348328 | Tax_Id = 10090 Gene_Symbol = Krt78 keratin Kb40 | 419 | 112194 |
| 53 | IPI00123891 | Tax_Id = 10090 Gene_Symbol = Csrp1 Cysteine and | 413 | 20570 |
| | | glycine-rich protein 1 | | |
| 54 | IPI00227835 | Tax_Id = 10090 Gene_Symbol = Tpm1 Isoform 2 of | 404 | 32689 |
| | | Tropomyosin alpha-1 chain | | |
| 55 | IPI00307837 | Tax_Id = 10090 Gene_Symbol = Eef1a1 Elongation factor | 401 | 50082 |
| | | 1-alpha 1 | | |
| 56 | IPI00462140 | Tax_Id = 10090 Gene_Symbol = Krt77 Keratin, type II | 387 | 61322 |
| | | cytoskeletal 1b | | |
| 57 | IPI00131209 | Tax_Id = 10090 Gene_Symbol = Krt16 Keratin | 383 | 52021 |
| | | intermediate filament 16a | | |
| 58 | IPI00110827 | Tax_Id = 10090 Gene_Symbol = Acta1 Actin, alpha | 380 | 42024 |
| | | skeletal muscle | | |
| 59 | IPI00122565 | Tax_Id = 10090 Gene_Symbol = Gdi2 Isoform 1 of Rab | 354 | 50505 |
| | | GDP dissociation inhibitor beta | | |
| 60 | IPI00266188 | Tax_Id = 10090 Gene_Symbol = Cfl2 Cofilin-2 | 341 | 18698 |
| 61 | IPI00116498 | Tax_Id = 10090 Gene_Symbol = Ywhaz 14-3-3 protein | 340 | 27754 |
| | | zeta/delta | | |
| 62 | IPI00346834 | Tax_Id = 10090 Gene_Symbol = Krt76 Keratin, type II | 331 | 62806 |
| | | cytoskeletal 2 oral | | |
| 63 | IPI00319992 | Tax_Id = 10090 Gene_Symbol = Hspa5 78 kDa glucose- | 325 | 72377 |
| | | regulated protein | | |
| 64 | IPI00269076 | Tax_Id = 10090 Gene_Symbol = Ak2 Isoform 2 of | 325 | 25589 |
| | | Adenylate kinase 2, mitochondrial | | |
| 65 | IPI00128154 | Tax_Id = 10090 Gene_Symbol = Ctsl Cathepsin L1 | 318 | 37523 |
| 66 | IPI00123319 | Tax_Id = 10090 Gene_Symbol = Tpm2 Isoform 1 of | 312 | 32817 |
| | | Tropomyosin beta chain | | |
| 67 | IPI00137409 | Tax_Id = 10090 Gene_Symbol = Tkt Transketolase | 310 | 67588 |
| 68 | IPI00131459 | Tax_Id = 10090 Gene_Symbol = Nme1; LOC100046344 | 302 | 17197 |
| | | Nucleoside diphosphate kinase A | | |
| 69 | IPI00336324 | Tax_Id = 10090 Gene_Symbol = Mdh1 Malate | 294 | 36488 |
| | | dehydrogenase, cytoplasmic | | |
| 70 | IPI00125778 | Tax_Id = 10090 Gene_Symbol = Tagln2 Transgelin-2 | 284 | 22381 |
| 71 | IPI00130240 | Tax_Id = 10090 Gene_Symbol = Ppic Peptidyl-prolyl cis- | 280 | 22780 |
| | | trans isomerase C | | |
| 72 | IPI00338452 | Tax_Id = 10090 Gene_Symbol = Col4a2 Collagen alpha- | 269 | 167220 |
| | | 2(IV) chain | | |
| 73 | IPI00309997 | Tax_Id = 10090 Gene_Symbol = Fhl1 Isoform 1 of Four | 266 | 31867 |
| | | and a half LIM domains protein 1 | | |
| 74 | IPI00224740 | Tax_Id = 10090 Gene_Symbol = Pfn1 Profilin-1 | 264 | 14948 |
| 75 | IPI00129571 | Tax_Id = 10090 Gene_Symbol = Col3a1 Collagen alpha- | 258 | 138858 |
| | | 1(III) chain | | |
| 76 | IPI00353563 | Tax_Id = 10090 Gene_Symbol = Fscn1 Fascin | 243 | 54474 |
| 77 | IPI00120045 | Tax_Id = 10090 Gene_Symbol = Hspe1-rs1 CPN10-like | 237 | 10971 |
| | | protein | | |
| 78 | IPI00131138 | Tax_Id = 10090 Gene_Symbol = Flna Isoform 1 of Filamin- | 229 | 281018 |
| | | A | | |
| 79 | IPI00107952 | Tax_Id = 10090 Gene_Symbol = Lyz2 Lysozyme C-2 | 228 | 18301 |
| 80 | IPI00119202 | Tax_Id = 10090 Gene_Symbol = S100a11 Protein S100- | 224 | 11075 |
| | | A11 | | |
| 81 | IPI00759948 | Tax_Id = 10090 Gene_Symbol = Gsn Isoform 2 of Gelsolin | 221 | 80712 |
| 82 | IPI00622240 | Tax_Id = 10090 Gene_Symbol = Krt2 Keratin, type II | 218 | 70880 |
| | | cytoskeletal 2 epidermal | | |
| 83 | IPI00130102 | Tax_Id = 10090 Gene_Symbol = Des Desmin | 207 | 53465 |
| 84 | IPI00130391 | Tax_Id = 10090 Gene_Symbol = Tcrb-V20; Prss3; Prss1 | 206 | 26118 |
| | | protease, serine, 1 | | |
| 85 | IPI00137331 | Tax_Id = 10090 Gene_Symbol = Cap1 Adenylyl cyclase- | 204 | 51542 |
| | | associated protein 1 | | |
| 86 | IPI00120176 | Tax_Id = 10090 Gene_Symbol = Pcolce Procollagen C- | 203 | 50136 |
| | | endopeptidase enhancer 1 | | |
| 87 | IPI00114733 | Tax_Id = 10090 Gene_Symbol = Serpinh1 Serpin H1 | 196 | 46560 |
| 88 | IPI00229517 | Tax_Id = 10090 Gene_Symbol = Lgals1 Galectin-1 | 194 | 14856 |
| 89 | IPI00121471 | Tax_Id = 10090 Gene_Symbol = Serpinb6a Serpin B6 | 192 | 42571 |
| 90 | IPI00132722 | Tax_Id = 10090 Gene_Symbol = Anxa3 Annexin A3 | 189 | 36348 |
| 91 | IPI00132950 | Tax_Id = 10090 Gene_Symbol = Rps21 40S ribosomal | 188 | 9136 |
| | | protein S21 | | |
| 92 | IPI00136642 | Tax_Id = 10090 Gene_Symbol = Serpinc1 Antithrombin-III | 181 | 51971 |
| 93 | IPI00331394 | Tax_Id = 10090 Gene_Symbol = Dnpep aspartyl | 177 | 52433 |
| | | aminopeptidase isoform a | | |
| 94 | IPI00663627 | Tax_Id = 10090 Gene_Symbol = Flnb Filamin-B | 174 | 277579 |
| 95 | IPI00553777 | Tax_Id = 10090 Gene_Symbol = Hnrnpa1 Putative | 172 | 38780 |
| | | uncharacterized protein | | |
| 96 | IPI00121788 | Tax_Id = 10090 Gene_Symbol = Prdx1 Peroxiredoxin-1 | 171 | 22162 |
| 97 | IPI00118899 | Tax_Id = 10090 Gene_Symbol = Actn4 Alpha-actinin-4 | 168 | 104911 |
| 98 | IPI00230707 | Tax_Id = 10090 Gene_Symbol = Ywhag 14-3-3 protein | 168 | 28285 |
| | | gamma | | |
| 99 | IPI00354819 | Tax_Id = 10090 Gene_Symbol = Myl6 Isoform Smooth | 167 | 16950 |
| | | muscle of Myosin light polypeptide 6 | | |
| 100 | IPI00114285 | Tax_Id = 10090 Gene_Symbol = Gsto1 Glutathione S- | 161 | 27480 |
| | | transferase omega-1 | | |
| 101 | IPI00139788 | Tax_Id = 10090 Gene_Symbol = Trf Serotransferrin | 161 | 76674 |
| 102 | IPI00122450 | Tax_Id = 10090 Gene_Symbol = Cald1 caldesmon 1 | 160 | 60417 |
| 103 | IPI00114403 | Tax_Id = 10090 Gene_Symbol = Timp1 Metalloproteinase | 160 | 22613 |
| | | inhibitor 1 | | |
| 104 | IPI00230204 | Tax_Id = 10090 Gene_Symbol = Got1 Aspartate | 157 | 46202 |
| | | aminotransferase, cytoplasmic | | |
| 105 | IPI00466128 | Tax_Id = 10090 Gene_Symbol = Akr1a4 Alcohol | 152 | 36564 |
| | | dehydrogenase [NADP+] | | |
| 106 | IPI00230682 | Tax_Id = 10090 Gene_Symbol = Ywhab Isoform Long of | 152 | 28069 |
| | | 14-3-3 protein beta/alpha | | |
| 107 | IPI00117312 | Tax_Id = 10090 Gene_Symbol = Got2 Aspartate | 149 | 47381 |
| | | aminotransferase, mitochondrial | | |
| 108 | IPI00118384 | Tax_Id = 10090 Gene_Symbol = Ywhae 14-3-3 protein | 147 | 29155 |
| | | epsilon | | |
| 109 | IPI00112904 | Tax_Id = 10090 Gene_Symbol = Mmp2 72 kDa type IV | 141 | 74055 |
| | | collagenase | | |
| 110 | IPI00323624 | Tax_Id = 10090 Gene_Symbol = C3 Isoform Long of | 141 | 186365 |
| | | Complement C3 (Fragment) | | |
| 111 | IPI00113517 | Tax_Id = 10090 Gene_Symbol = Ctsb Cathepsin B | 141 | 37256 |
| 112 | IPI00117910 | Tax_Id = 10090 Gene_Symbol = Prdx2 Peroxiredoxin-2 | 139 | 21765 |
| 113 | IPI00420261 | Tax_Id = 10090 Gene_Symbol = Hmgb1; Gm15387 High | 138 | 24878 |
| | | mobility group protein B1 | | |
| 114 | IPI00111013 | Tax_Id = 10090 Gene_Symbol = Ctsd Cathepsin D | 135 | 44925 |
| 115 | IPI00124692 | Tax_Id = 10090 Gene_Symbol = Taldo1 Transaldolase | 133 | 37363 |
| 116 | IPI00469251 | Tax_Id = 10090 Gene_Symbol = Txnrd1 Isoform 2 of | 133 | 54463 |
| | | Thioredoxin reductase 1, cytoplasmic | | |
| 117 | IPI00138892 | Tax_Id = 10090 Gene_Symbol = 2810422J05Rik; Uba52 | 133 | 14719 |
| | | Ubiquitin A-52 residue ribosomal protein fusion product 1 | | |
| 118 | IPI00123176 | Tax_Id = 10090 Gene_Symbol = - Putative uncharacterized | 132 | 36519 |
| | | protein ENSMUSP00000100794 | | |
| 119 | IPI00113141 | Tax_Id = 10090 Gene_Symbol = Cs Citrate synthase, | 132 | 51703 |
| | | mitochondrial | | |
| 120 | IPI00271951 | Tax_Id = 10090 Gene_Symbol = Pdia4 protein disulfide- | 131 | 72325 |
| | | isomerase A4 | | |
| 121 | IPI00129178 | Tax_Id = 10090 Gene_Symbol = Oat Ornithine | 130 | 48324 |
| | | aminotransferase, mitochondrial | | |
| 122 | IPI00227834 | Tax_Id = 10090 Gene_Symbol = Itih2 inter-alpha-trypsin | 130 | 106295 |
| | | inhibitor heavy chain H2 | | |
| 123 | IPI00466069 | Tax_Id = 10090 Gene_Symbol = Eef2 Elongation factor 2 | 129 | 95253 |
| 124 | IPI00229510 | Tax_Id = 10090 Gene_Symbol = Ldhb L-lactate | 128 | 36549 |
| | | dehydrogenase B chain | | |
| 125 | IPI00125091 | Tax_Id = 10090 Gene_Symbol = Lasp1 LIM and SH3 | 128 | 29975 |
| | | domain protein 1 | | |
| 126 | IPI00828528 | Tax_Id = 10090 Gene_Symbol = Krt14 Type I epidermal | 126 | 10655 |
| | | keratin (Fragment) | | |
| 127 | IPI00124979 | Tax_Id = 10090 Gene_Symbol = Rbmx RNA binding motif | 124 | 42275 |
| | | protein, X-linked | | |
| 128 | IPI00113427 | Tax_Id = 10090 Gene_Symbol = Lyz1 Lysozyme C-1 | 122 | 18406 |
| 129 | IPI00125220 | Tax_Id = 10090 Gene_Symbol = Ctsz Cathepsin Z | 120 | 34153 |
| 130 | IPI00222496 | Tax_Id = 10090 Gene_Symbol = Pdia6 Putative | 116 | 48659 |
| | | uncharacterized protein | | |
| 131 | IPI00128857 | Tax_Id = 10090 Gene_Symbol = Me1 NADP-dependent | 114 | 63958 |
| | | malic enzyme | | |
| 132 | IPI00129215 | Tax_Id = 10090 Gene_Symbol = Vnn1 Pantetheinase | 112 | 57024 |
| 133 | IPI00223757 | Tax_Id = 10090 Gene_Symbol = Akr1b3 Aldose reductase | 107 | 35709 |
| 134 | IPI00127942 | Tax_Id = 10090 Gene_Symbol = Dstn Destrin | 107 | 18509 |
| 135 | IPI00118875 | Tax_Id = 10090 Gene_Symbol = Eef1d Isoform 1 of | 106 | 31274 |
| | | Elongation factor 1-delta | | |
| 136 | IPI00123744 | Tax_Id = 10090 Gene_Symbol = Cst3 Cystatin-C | 106 | 15521 |
| 137 | IPI00109482 | Tax_Id = 10090 Gene_Symbol = Ddah1 N(G), N(G)- | 102 | 31361 |
| | | dimethylarginine dimethylaminohydrolase 1 | | |
| 138 | IPI00138691 | Tax_Id = 10090 Gene_Symbol = Arpc4 Actin-related | 97 | 19654 |
| | | protein 2/3 complex subunit 4 | | |
| 139 | IPI00318841 | Tax_Id = 10090 Gene_Symbol = Eef1g Elongation factor 1- | 96 | 50029 |
| | | gamma | | |
| 140 | IPI00111265 | Tax_Id = 10090 Gene_Symbol = Capza2 F-actin-capping | 96 | 32947 |
| | | protein subunit alpha-2 | | |
| 141 | IPI00117288 | Tax_Id = 10090 Gene_Symbol = Hnrnpab Heterogeneous | 92 | 30812 |
| | | nuclear ribonucleoprotein A/B | | |
| 142 | IPI00129685 | Tax_Id = 10090 Gene_Symbol = Tpt1; Gm14456 | 91 | 20236 |
| | | Translationally-controlled tumor protein | | |
| 143 | IPI00380460 | Tax_Id = 10090 Gene_Symbol = Dsg1b Desmoglein-1-beta | 91 | 114382 |
| 144 | IPI00123709 | Tax_Id = 10090 Gene_Symbol = Akap12 Isoform 1 of A- | 90 | 180586 |
| | | kinase anchor protein 12 | | |
| 145 | IPI00117083 | Tax_Id = 10090 Gene_Symbol = Grpel1 GrpE protein | 89 | 24292 |
| | | homolog 1, mitochondrial | | |
| 146 | IPI00108271 | Tax_Id = 10090 Gene_Symbol = Elavl1 ELAV-like protein 1 | 87 | 36046 |
| 147 | IPI00125931 | Tax_Id = 10090 Gene_Symbol = Cstb Cystatin-B | 84 | 11039 |
| 148 | IPI00331564 | Tax_Id = 10090 Gene_Symbol = Dld Dihydrolipoyl | 84 | 54564 |
| | | dehydrogenase | | |
| 149 | IPI00323571 | Tax_Id = 10090 Gene_Symbol = Apoe Apolipoprotein E | 83 | 35844 |
| 150 | IPI00454052 | Tax_Id = 10090 Gene_Symbol = A2m Alpha-2- | 83 | 164222 |
| | | macroglobulin-P | | |
| 151 | IPI00120886 | Tax_Id = 10090 Gene_Symbol = Ybx1 Nuclease-sensitive | 82 | 35709 |
| | | element-binding protein 1 | | |
| 152 | IPI00115627 | Tax_Id = 10090 Gene_Symbol = Actr3 Actin-related | 80 | 47327 |
| | | protein 3 | | |
| 153 | IPI00153400 | Tax_Id = 10090 Gene_Symbol = H2afj Histone H2A.J | 78 | 14037 |
| 154 | IPI00130589 | Tax_Id = 10090 Gene_Symbol = Sod1 Superoxide | 76 | 15933 |
| | | dismutase [Cu—Zn] | | |
| 155 | IPI00108762 | Tax_Id = 10090 Gene_Symbol = - similar to SMT3B | 75 | 10737 |
| | | protein isoform 3 | | |
| 156 | IPI00318017 | Tax_Id = 10090 Gene_Symbol = Prss23 Serine protease 23 | 75 | 43044 |
| 157 | IPI00117063 | Tax_Id = 10090 Gene_Symbol = Fus RNA-binding protein | 74 | 52642 |
| | | FUS | | |
| 158 | IPI00406419 | Tax_Id = 10090 Gene_Symbol = Pepd Xaa-Pro dipeptidase | 72 | 54993 |
| 159 | IPI00457898 | Tax_Id = 10090 Gene_Symbol = Pgam1 Phosphoglycerate | 71 | 28814 |
| | | mutase 1 | | |
| 160 | IPI00664670 | Tax_Id = 10090 Gene_Symbol = Flnc Putative | 71 | 292163 |
| | | uncharacterized protein Flnc | | |
| 161 | IPI00129519 | Tax_Id = 10090 Gene_Symbol = Basp1 Brain acid soluble | 70 | 22074 |
| | | protein 1 | | |
| 162 | IPI00462291 | Tax_Id = 10090 Gene_Symbol = Hmgb2 High mobility | 69 | 24147 |
| | | group protein B2 | | |
| 163 | IPI00135996 | Tax_Id = 10090 Gene_Symbol = Pof1b Protein POF1B | 69 | 68459 |
| 164 | IPI00331436 | Tax_Id = 10090 Gene_Symbol = Lap3 Isoform 1 of Cytosol | 68 | 56106 |
| | | aminopeptidase | | |
| 165 | IPI00405058 | Tax_Id = 10090 Gene_Symbol = Hnrnpa2b1 Isoform 3 of | 68 | 32440 |
| | | Heterogeneous nuclear ribonucleoproteins A2/B1 | | |
| 166 | IPI00230427 | Tax_Id = 10090 Gene_Symbol = Mif Macrophage | 68 | 12496 |
| | | migration inhibitory factor | | |
| 167 | IPI00108774 | Tax_Id = 10090 Gene_Symbol = Rad23b UV excision | 66 | 43490 |
| | | repair protein RAD23 homolog B | | |
| 168 | IPI00109044 | Tax_Id = 10090 Gene_Symbol = 2900073G15Rik myosin | 65 | 19883 |
| | | light chain, regulatory B-like | | |
| 169 | IPI00321190 | Tax_Id = 10090 Gene_Symbol = Psap Sulfated glycoprotein 1 | 64 | 61381 |
| 170 | IPI00608020 | Tax_Id = 10090 Gene_Symbol = Ftl1 ferritin light chain 1 | 64 | 20759 |
| 171 | IPI00321648 | Tax_Id = 10090 Gene_Symbol = Mmp12 Macrophage | 63 | 54936 |
| | | metalloelastase | | |
| 172 | IPI00317740 | Tax_Id = 10090 Gene_Symbol = Gnb2l1 Guanine | 62 | 35055 |
| | | nucleotide-binding protein subunit beta-2-like 1 | | |
| 173 | IPI00228583 | Tax_Id = 10090 Gene_Symbol = Mtpn Myotrophin | 62 | 12853 |
| 174 | IPI00165854 | Tax_Id = 10090 Gene_Symbol = Ube2n Ubiquitin- | 61 | 17127 |
| | | conjugating enzyme E2 N | | |
| 175 | IPI00330231 | Tax_Id = 10090 Gene_Symbol = Rapgef1 Rap guanine | 60 | 121847 |
| | | nucleotide exchange factor (GEF) 1 isoform 3 | | |
| 176 | IPI00123996 | Tax_Id = 10090 Gene_Symbol = Nrp1 Neuropilin-1 | 59 | 102955 |
| 177 | IPI00673290 | Tax_Id = 10090 Gene_Symbol = LOC634088 similar to | 58 | 17787 |
| | | Glyceraldehyde-3-phosphate dehydrogenase | | |
| 178 | IPI00125138 | Tax_Id = 10090 Gene_Symbol = Csf1 Isoform 1 of | 58 | 60611 |
| | | Macrophage colony-stimulating factor 1 | | |
| 179 | IPI00223231 | Tax_Id = 10090 Gene_Symbol = Qsox1 Isoform 1 of | 57 | 82733 |
| | | Sulfhydryl oxidase 1 | | |
| 180 | IPI00127691 | Tax_Id = 10090 Gene_Symbol = Gss Glutathione | 57 | 52214 |
| | | synthetase | | |
| 181 | IPI00316491 | Tax_Id = 10090 Gene_Symbol = Hbb-b2 Hemoglobin | 57 | 15868 |
| | | subunit beta-2 | | |
| 182 | IPI00114052 | Tax_Id = 10090 Gene_Symbol = Snrpb Small nuclear | 56 | 23640 |
| | | ribonucleoprotein-associated protein B | | |
| 183 | IPI00129430 | Tax_Id = 10090 Gene_Symbol = Sfpq Splicing factor, | 55 | 75394 |
| | | proline- and glutamine-rich | | |
| 184 | IPI00108125 | Tax_Id = 10090 Gene_Symbol = Eif5a Eukaryotic | 55 | 16821 |
| | | translation initiation factor 5A-1 | | |
| 185 | IPI00230065 | Tax_Id = 10090 Gene_Symbol = Col11a1 collagen alpha- | 54 | 180921 |
| | | 1(XI) chain precursor | | |
| 186 | IPI00310056 | Tax_Id = 10090 Gene_Symbol = Lox Protein-lysine 6- | 51 | 46671 |
| | | oxidase | | |
| 187 | IPI00266899 | Tax_Id = 10090 Gene_Symbol = Fkbp1a Peptidyl-prolyl | 51 | 11915 |
| | | cis-trans isomerase FKBP1A | | |
| 188 | IPI00110658 | Tax_Id = 10090 Gene_Symbol = Hba-a1; Hba-a2 Putative | 50 | 15193 |
| | | uncharacterized protein | | |
| 189 | IPI00124829 | Tax_Id = 10090 Gene_Symbol = Arpc3 Actin-related | 50 | 20511 |
| | | protein 2/3 complex subunit 3 | | |
| 190 | IPI00665965 | Tax_Id = 10090 Gene_Symbol = Gm6314 similar to | 50 | 14403 |
| | | 3110003A17Rik protein | | |
| 191 | IPI00129517 | Tax_Id = 10090 Gene_Symbol = Prdx5 Isoform | 49 | 21884 |
| | | Mitochondrial of Peroxiredoxin-5, mitochondrial | | |
| 192 | IPI00227522 | Tax_Id = 10090 Gene_Symbol = 9530053A07Rik Fc | 49 | 280044 |
| | | fragment of IgG binding protein-like | | |
| 193 | IPI00269481 | Tax_Id = 10090 Gene_Symbol = Capzb Isoform 2 of F- | 48 | 30609 |
| | | actin-capping protein subunit beta | | |
| 194 | IPI00464223 | Tax_Id = 10090 Gene_Symbol = Myof Isoform 1 of | 48 | 233177 |
| | | Myoferlin | | |
| 195 | IPI00121209 | Tax_Id = 10090 Gene_Symbol = Apoa1 Apolipoprotein A-I | 48 | 30569 |
| 196 | IPI00313900 | Tax_Id = 10090 Gene_Symbol = Lum Lumican | 47 | 38241 |
| 197 | IPI00115620 | Tax_Id = 10090 Gene_Symbol = Psat1 Phosphoserine | 47 | 40447 |
| | | aminotransferase | | |
| 198 | IPI00127596 | Tax_Id = 10090 Gene_Symbol = Ckm Creatine kinase M- | 47 | 43018 |
| | | type | | |
| 199 | IPI00127679 | Tax_Id = 10090 Gene_Symbol = Dhx32 Isoform 2 of | 47 | 84719 |
| | | Putative pre-mRNA-splicing factor ATP-dependent RNA | | |
| | | helicase DHX32 | | |
| 200 | IPI00308885 | Tax_Id = 10090 Gene_Symbol = Hspd1 Isoform 1 of 60 | 46 | 60917 |
| | | kDa heat shock protein, mitochondrial | | |
| 201 | IPI00331692 | Tax_Id = 10090 Gene_Symbol = Dci 3,2-trans-enoyl-CoA | 45 | 32230 |
| | | isomerase, mitochondrial precursor | | |
| 202 | IPI00114260 | Tax_Id = 10090 Gene_Symbol = Fmn1 Isoform 1 of | 45 | 163479 |
| | | Formin-1 | | |
| 203 | IPI00111957 | Tax_Id = 10090 Gene_Symbol = Hist1h2ba Histone H2B | 45 | 14228 |
| | | type 1-A | | |
| 204 | IPI00136984 | Tax_Id = 10090 Gene_Symbol = Rps7 40S ribosomal | 44 | 22113 |
| | | protein S7 | | |
| 205 | IPI00128904 | Tax_Id = 10090 Gene_Symbol = Pcbp1 Poly(rC)-binding | 44 | 37474 |
| | | protein 1 | | |
| 206 | IPI00114958 | Tax_Id = 10090 Gene_Symbol = Kng1 Isoform HMW of | 44 | 73056 |
| | | Kininogen-1 | | |
| 207 | IPI00466919 | Tax_Id = 10090 Gene_Symbol = Pgd 6-phosphogluconate | 44 | 53213 |
| | | dehydrogenase, decarboxylating | | |
| 208 | IPI00468688 | Tax_Id = 10090 Gene_Symbol = Tars Threonyl-tRNA | 44 | 83303 |
| | | synthetase, cytoplasmic | | |
| 209 | IPI00136556 | Tax_Id = 10090 Gene_Symbol = Tcn2 Transcobalamin-2 | 43 | 47555 |
| 210 | IPI00127408 | Tax_Id = 10090 Gene_Symbol = Rac1 RAS-related C3 | 43 | 23417 |
| | | botulinum substrate 1, isoform CRA_a | | |
| 211 | IPI00125143 | Tax_Id = 10090 Gene_Symbol = Arpc1b Arpc1b protein | 43 | 41472 |
| 212 | IPI00134784 | Tax_Id = 10090 Gene_Symbol = Lsm4 U6 snRNA- | 42 | 15067 |
| | | associated Sm-like protein LSm4 | | |
| 213 | IPI00226397 | Tax_Id = 10090 Gene_Symbol = Gemin5 gem-associated | 42 | 166487 |
| | | protein 5 isoform 2 | | |
| 214 | IPI00757435 | Tax_Id = 10090 Gene_Symbol = Kcnt2 Putative | 42 | 131047 |
| | | uncharacterized protein Kcnt2 | | |
| 215 | IPI00321734 | Tax_Id = 10090 Gene_Symbol = Glo1 Lactoylglutathione | 42 | 20796 |
| | | lyase | | |
| 216 | IPI00128108 | Tax_Id = 10090 Gene_Symbol = Try4 trypsin 4 | 41 | 26257 |
| 217 | IPI00229534 | Tax_Id = 10090 Gene_Symbol = Marcks Myristoylated | 41 | 29644 |
| | | alanine-rich C-kinase substrate | | |
| 218 | IPI00322936 | Tax_Id = 10090 Gene_Symbol = Plg Plasminogen | 41 | 90723 |
| 219 | IPI00122815 | Tax_Id = 10090 Gene_Symbol = P4hb Putative | 41 | 57023 |
| | | uncharacterized protein | | |
| 220 | IPI00339474 | Tax_Id = 10090 Gene_Symbol = Rpl12-ps1 Putative | 40 | 17901 |
| | | uncharacterized protein Gm4957 | | |
| 221 | IPI00130343 | Tax_Id = 10090 Gene_Symbol = Hnrnpc Putative | 40 | 36883 |
| | | uncharacterized protein | | |
| 222 | IPI00387416 | Tax_Id = 10090 Gene_Symbol = Ubqln2 Ubiquilin-2 | 39 | 67336 |
| 223 | IPI00113863 | Tax_Id = 10090 Gene_Symbol = Timp2 Metalloproteinase | 39 | 24312 |
| | | inhibitor 2 | | |
| 224 | IPI00223769 | Tax_Id = 10090 Gene_Symbol = Cd44 CD44 antigen | 38 | 40238 |
| | | isoform c | | |
| 225 | IPI00224152 | Tax_Id = 10090 Gene_Symbol = Apex1 DNA-(apurinic or | 37 | 35468 |
| | | apyrimidinic site) lyase | | |
| 226 | IPI00126338 | Tax_Id = 10090 Gene_Symbol = Tmpo Isoform Alpha of | 37 | 75285 |
| | | Lamina-associated polypeptide 2, isoforms alpha/zeta | | |
| 227 | IPI00114488 | Tax_Id = 10090 Gene_Symbol = Ect2 Protein ECT2 | 36 | 83632 |
| 228 | IPI00757477 | Tax_Id = 10090 Gene_Symbol = Kdm3b Isoform 1 of | 36 | 170768 |
| | | Lysine-specific demethylase 3B | | |
| 229 | IPI00467447 | Tax_Id = 10090 Gene_Symbol = Iqgap1 Ras GTPase- | 36 | 188638 |
| | | activating-like protein IQGAP1 | | |
| 230 | IPI00404019 | Tax_Id = 10090 Gene_Symbol = Manf Armet protein | 36 | 19000 |
| 231 | IPI00187510 | Tax_Id = 10090 Gene_Symbol = Tnik Isoform 1 of Traf2 | 36 | 150274 |
| | | and NCK-interacting protein kinase | | |
| 232 | IPI00379009 | Tax_Id = 10090 Gene_Symbol = Vsig10l Putative | 35 | 91463 |
| | | uncharacterized protein Vsig10l | | |
| 233 | IPI00356708 | Tax_Id = 10090 Gene_Symbol = Ceacam16 CEA-related | 34 | 46322 |
| | | cell adhesion molecule 16 | | |
| 234 | IPI00307991 | Tax_Id = 10090 Gene_Symbol = Crk Isoform Crk-II of | 34 | 33794 |
| | | Adapter molecule crk | | |
| 235 | IPI00108189 | Tax_Id = 10090 Gene_Symbol = Hint1 Histidine triad | 34 | 13768 |
| | | nucleotide-binding protein 1 | | |
| 236 | IPI00125939 | Tax_Id = 10090 Gene_Symbol = Acot7 cytosolic acyl | 33 | 42799 |
| | | coenzyme A thioester hydrolase isoform 1 | | |
| 237 | IPI00226872 | Tax_Id = 10090 Gene_Symbol = Efhd2 Putative | 32 | 26784 |
| | | uncharacterized protein | | |
| 238 | IPI00115862 | Tax_Id = 10090 Gene_Symbol = Eif6 Eukaryotic | 32 | 26494 |
| | | translation initiation factor 6 | | |
| 239 | IPI00226993 | Tax_Id = 10090 Gene_Symbol = Txn1 Thioredoxin | 32 | 11668 |
| 240 | IPI00138397 | Tax_Id = 10090 Gene_Symbol = Igf2 Insulin-like growth | 32 | 20017 |
| | | factor II | | |
| 241 | IPI00262319 | Tax_Id = 10090 Gene_Symbol = Madd MAP-kinase | 32 | 176325 |
| | | activating death domain | | |
| 242 | IPI00229589 | Tax_Id = 10090 Gene_Symbol = Sox30 Transcription factor | 32 | 83886 |
| | | SOX-30 | | |
| 243 | IPI00330146 | Tax_Id = 10090 Gene_Symbol = Nufip2 Isoform 1 of | 31 | 75611 |
| | | Nuclear fragile X mental retardation-interacting protein 2 | | |
| 244 | IPI00331402 | Tax_Id = 10090 Gene_Symbol = 4930511I11Rik | 31 | 38505 |
| | | Uncharacterized protein C6orf81 homolog | | |
| 245 | IPI00849090 | Tax_Id = 10090 Gene_Symbol = 6720432D03Rik | 31 | 11595 |
| | | hypothetical protein LOC77740 | | |
| 246 | IPI00830803 | Tax_Id = 10090 Gene_Symbol = Fbln2 fibulin-2 isoform b | 31 | 126414 |
| 247 | IPI00330560 | Tax_Id = 10090 Gene_Symbol = Mogat2 2-acylglycerol O- | 31 | 38566 |
| | | acyltransferase 2 | | |
| 248 | IPI00605894 | Tax_Id = 10090 Gene_Symbol = Ahnak Desmoyokin | 31 | 132029 |
| | | (Fragment) | | |
| 249 | IPI00466642 | Tax_Id = 10090 Gene_Symbol = Zfp566 zinc finger | 30 | 45264 |
| | | protein 566 | | |
| 250 | IPI00322748 | Tax_Id = 10090 Gene_Symbol = Fbln1 Isoform D of | 30 | 77981 |
| | | Fibulin-1 | | |
| 251 | IPI00856350 | Tax_Id = 10090 Gene_Symbol = Clip1 Protein | 30 | 135913 |
| 252 | IPI00395222 | Tax_Id = 10090 Gene_Symbol = Adar Isoform 1 of Double- | 30 | 130365 |
| | | stranded RNA-specific adenosine deaminase | | |
| 253 | IPI00224605 | Tax_Id = 10090 Gene_Symbol = A430108E01Rik Putative | 30 | 20030 |
| | | uncharacterized protein | | |
| 254 | IPI00626350 | Tax_Id = 10090 Gene_Symbol = Col5a3 Procollagen, type | 30 | 171864 |
| | | V, alpha 3, isoform CRA_b | | |
| 255 | IPI00125890 | Tax_Id = 10090 Gene_Symbol = Pdcd1 Programmed cell | 30 | 31822 |
| | | death protein 1 | | |
| 256 | IPI00320420 | Tax_Id = 10090 Gene_Symbol = Clu Clusterin | 30 | 51623 |
| 257 | IPI00323059 | Tax_Id = 10090 Gene_Symbol = Ap4s1 AP-4 complex | 30 | 16807 |
| | | subunit sigma-1 | | |
| 258 | IPI00229072 | Tax_Id = 10090 Gene_Symbol = Pml Isoform 1 of Probable | 30 | 98180 |
| | | transcription factor PML | | |
| 259 | IPI00355808 | Tax_Id = 10090 Gene_Symbol = Asap2 Isoform 1 of Arf- | 30 | 106738 |
| | | GAP with SH3 domain, ANK repeat and PH domain- | | |
| | | containing protein 2 | | |
| 260 | IPI00117264 | Tax_Id = 10090 Gene_Symbol = Park7 Protein DJ-1 | 30 | 20008 |
| 261 | IPI00331612 | Tax_Id = 10090 Gene_Symbol = Hmga2 High mobility | 29 | 11812 |
| | | group protein HMGI-C | | |
| 262 | IPI00122438 | Tax_Id = 10090 Gene_Symbol = Fbn1 Fibrillin-1 | 29 | 312051 |
| 263 | IPI00403502 | Tax_Id = 10090 Gene_Symbol = Arntl Isoform 3 of Aryl | 29 | 67157 |
| | | hydrocarbon receptor nuclear translocator-like protein 1 | | |
| 264 | IPI00757449 | Tax_Id = 10090 Gene_Symbol = Cdhr3 Cadherin-related | 29 | 92027 |
| | | family member 3 | | |
| 265 | IPI00380474 | Tax_Id = 10090 Gene_Symbol = Lpar1 Isoform 1 of | 29 | 41092 |
| | | Lysophosphatidic acid receptor 1 | | |
| 266 | IPI00466824 | Tax_Id = 10090 Gene_Symbol = Fastk Putative | 29 | 61312 |
| | | uncharacterized protein | | |
| 267 | IPI00122594 | Tax_Id = 10090 Gene_Symbol = Ahctf1 Protein ELYS | 29 | 247493 |
| 268 | IPI00136936 | Tax_Id = 10090 Gene_Symbol = Vps29 Isoform 1 of | 28 | 20483 |
| | | Vacuolar protein sorting-associated protein 29 | | |
| 269 | IPI00320208 | Tax_Id = 10090 Gene_Symbol = Eef1b2 Elongation factor | 28 | 24678 |
| | | 1-beta | | |
| 270 | IPI00224014 | Tax_Id = 10090 Gene_Symbol = Adcy10 Isoform 1 of | 28 | 186285 |
| | | Adenylate cyclase type 10 | | |
| 271 | IPI00128689 | Tax_Id = 10090 Gene_Symbol = Col5a1 Collagen alpha- | 28 | 183564 |
| | | 1(V) chain | | |
| 272 | IPI00319400 | Tax_Id = 10090 Gene_Symbol = Magt1 Isoform 1 of | 28 | 37944 |
| | | Magnesium transporter protein 1 | | |
| 273 | IPI00468648 | Tax_Id = 10090 Gene_Symbol = Slfn10 Schlafen 10 | 28 | 103474 |
| 274 | IPI00849947 | Tax_Id = 10090 Gene_Symbol = LOC100047588 similar to | 28 | 89401 |
| | | MAP/microtubule affinity-regulating kinase 3 | | |
| 275 | IPI00622001 | Tax_Id = 10090 Gene_Symbol = Ccpg1 Isoform 2 of Cell | 27 | 92051 |
| | | cycle progression protein 1 | | |
| 276 | IPI00129796 | Tax_Id = 10090 Gene_Symbol = Pou3f1 POU domain, | 27 | 47011 |
| | | class 3, transcription factor 1 | | |
| 277 | IPI00664640 | Tax_Id = 10090 Gene_Symbol = Urb1 Isoform 2 of | 27 | 25015 |
| | | Nucleolar pre-ribosomal-associated protein 1 | | |
| 278 | IPI00668902 | Tax_Id = 10090 Gene_Symbol = Scaper S phase cyclin A- | 27 | 157675 |
| | | associated protein in the ER | | |
| 279 | IPI00222466 | Tax_Id = 10090 Gene_Symbol = Fbxo44 Isoform 1 of F- | 27 | 29704 |
| | | box only protein 44 | | |
| 280 | IPI00473912 | Tax_Id = 10090 Gene_Symbol = Gigyf2 Isoform 1 of | 27 | 149102 |
| | | PERQ amino acid-rich with GYF domain-containing | | |
| | | protein 2 | | |
| 281 | IPI00125108 | Tax_Id = 10090 Gene_Symbol = Chpf Chondroitin sulfate | 27 | 85481 |
| | | synthase 2 | | |
| 282 | IPI00130654 | Tax_Id = 10090 Gene_Symbol = Afm Isoform 3 of Afamin | 27 | 69621 |
| 283 | IPI00469996 | Tax_Id = 10090 Gene_Symbol = Rexo1 Isoform 1 of RNA | 27 | 130709 |
| | | exonuclease 1 homolog | | |
| 284 | IPI00134344 | Tax_Id = 10090 Gene_Symbol = Spnb3 Putative | 26 | 270768 |
| | | uncharacterized protein | | |
| 285 | IPI00120475 | Tax_Id = 10090 Gene_Symbol = Gm10063 Putative | 26 | 14092 |
| | | uncharacterized protein Gm10063 | | |
| 286 | IPI00461407 | Tax_Id = 10090 Gene_Symbol = Gm5436 Putative | 26 | 23317 |
| | | uncharacterized protein Gm5436 | | |
| 287 | IPI00356143 | Tax_Id = 10090 Gene_Symbol = Gm595 Novel protein | 26 | 76340 |
| 288 | IPI00153899 | Tax_Id = 10090 Gene_Symbol = Irak4 Interleukin-1 | 26 | 50839 |
| | | receptor-associated kinase 4 | | |
| 289 | IPI00134058 | Tax_Id = 10090 Gene_Symbol = Erp44 Endoplasmic | 26 | 46823 |
| | | reticulum resident protein 44 | | |
| 290 | IPI00124287 | Tax_Id = 10090 Gene_Symbol = Pabpc1 Polyadenylate- | 25 | 70598 |
| | | binding protein 1 | | |
| 291 | IPI00761611 | Tax_Id = 10090 Gene_Symbol = - Ring finger protein 213 | 25 | 559213 |
| 292 | IPI00128577 | Tax_Id = 10090 Gene_Symbol = Rab40c Ras-related | 25 | 31328 |
| | | protein Rab-40C | | |
| 293 | IPI00749774 | Tax_Id = 10090 Gene_Symbol = Tdrd5 Tudor domain- | 25 | 116003 |
| | | containing protein 5 | | |
| 294 | IPI00474465 | Tax_Id = 10090 Gene_Symbol = Ppp1r2 Putative | 25 | 11652 |
| | | uncharacterized protein | | |
| 295 | IPI00399464 | Tax_Id = 10090 Gene_Symbol = Col8a1 Collagen alpha- | 25 | 73559 |
| | | 1(VIII) chain | | |
| 296 | IPI00351052 | Tax_Id = 10090 Gene_Symbol = - Putative uncharacterized | 24 | 48528 |
| | | protein ENSMUSP00000031097 | | |
| 297 | IPI00227331 | Tax_Id = 10090 Gene_Symbol = A930011G23Rik Putative | 24 | 16234 |
| | | uncharacterized protein | | |
| 298 | IPI00309059 | Tax_Id = 10090 Gene_Symbol = Patl1 Protein PAT1 | 24 | 86715 |
| | | homolog 1 | | |
| 299 | IPI00134549 | Tax_Id = 10090 Gene_Symbol = Lamp2 Isoform LAMP- | 24 | 45618 |
| | | 2A of Lysosome-associated membrane glycoprotein 2 | | |
| 300 | IPI00129479 | Tax_Id = 10090 Gene_Symbol = Pip4k2c | 23 | 47306 |
| | | Phosphatidylinositol-5-phosphate 4-kinase type-2 gamma | | |
| 301 | IPI00118534 | Tax_Id = 10090 Gene_Symbol = Klf16 Krueppel-like | 23 | 25636 |
| | | factor 16 | | |
| 302 | IPI00674433 | Tax_Id = 10090 Gene_Symbol = Ptprq Phosphotidylinositol | 23 | 256624 |
| | | phosphatase PTPRQ | | |
| 303 | IPI00225140 | Tax_Id = 10090 Gene_Symbol = Pclo protein piccolo | 23 | 550496 |
| | | isoform 1 | | |
| 304 | IPI00222760 | Tax_Id = 10090 Gene_Symbol = Prpf19 Isoform 2 of Pre- | 23 | 57266 |
| | | mRNA-processing factor 19 | | |
| 305 | IPI00654288 | Tax_Id = 10090 Gene_Symbol = Lhfp Putative | 23 | 11704 |
| | | uncharacterized protein | | |
| 306 | IPI00226209 | Tax_Id = 10090 Gene_Symbol = Klhl20 Kelch-like protein 20 | 22 | 67369 |
| 307 | IPI00121292 | Tax_Id = 10090 Gene_Symbol = Cd37 Putative | 22 | 34236 |
| | | uncharacterized protein | | |
| 308 | IPI00330578 | Tax_Id = 10090 Gene_Symbol = Ubn2 Isoform 1 of | 22 | 141653 |
| | | Ubinuclein-2 | | |
| 309 | IPI00315939 | Tax_Id = 10090 Gene_Symbol = Cthrc1 Collagen triple | 22 | 26443 |
| | | helix repeat-containing protein 1 | | |
| 310 | IPI00137647 | Tax_Id = 10090 Gene_Symbol = Vamp7 Vesicle-associated | 21 | 24951 |
| | | membrane protein 7 | | |
| 311 | IPI00127811 | Tax_Id = 10090 Gene_Symbol = Cx3cl1 Fractalkine | 21 | 42014 |
| 312 | IPI00136883 | Tax_Id = 10090 Gene_Symbol = Ptbp1 Putative | 21 | 56927 |
| | | uncharacterized protein | | |
| 313 | IPI00848586 | Tax_Id = 10090 Gene_Symbol = Gm4549 similar to Pro- | 21 | 27841 |
| | | Pol-dUTPase polyprotein; RNaseH; dUTPase; integrase; | | |
| | | protease; reverse transcriptase | | |
| 314 | IPI00405095 | Tax_Id = 10090 Gene_Symbol = Ubr2 Isoform 3 of E3 | 20 | 176932 |
| | | ubiquitin-protein ligase UBR2 | | |
| 315 | IPI00458190 | Tax_Id = 10090 Gene_Symbol = Pde4d Cyclic AMP | 20 | 85498 |
| | | specific phosphodiesterase PDE4D5A | | |
| 316 | IPI00227548 | Tax_Id = 10090 Gene_Symbol = Svs1 seminal vesicle- | 19 | 93470 |
| | | secreted protein I precursor | | |
| 317 | IPI00469945 | Tax_Id = 10090 Gene_Symbol = Lrrcc1 Isoform 2 of | 16 | 117203 |
| | | Leucine-rich repeat and coiled-coil domain-containing | | |
| | | protein 1 | | |
| 318 | IPI00407863 | Tax_Id = 10090 Gene_Symbol = Clasp2 CLIP-associating | 16 | 165840 |
| | | protein 2 c | | |
| 319 | IPI00108378 | Tax_Id = 10090 Gene_Symbol = Lrguk Leucine-rich repeat | 15 | 93131 |
| | | and guanylate kinase domain-containing protein | | |
| 320 | IPI00464194 | Tax_Id = 10090 Gene_Symbol = Fgd6 Isoform 1 of FYVE, | 14 | 155072 |
| | | RhoGEF and PH domain-containing protein 6 | | |
| 321 | IPI00321096 | Tax_Id = 10090 Gene_Symbol = 1700011L22Rik | 13 | 23353 |
| | | Uncharacterized protein C4orf51 homolog | | |
| 322 | IPI00469307 | Tax_Id = 10090 Gene_Symbol = Lrpap1 Alpha-2- | 13 | 42189 |
| | | macroglobulin receptor-associated protein | | |

While the two different approaches at defining the components present within the Pan-CM gave different results there were many common elements also. It is important to note that while the secretome analysis mainly produced a large number of structural molecules this is a direct result of the two methodologies. The strength of the array analysis is the ability to focus in on the proteins of interests mainly being the different morphogens and signaling molecules present. This deliberate attempt at focusing in on molecules with a capacity to signal also had the effect of discarding the structural molecules which are commonly expressed. While the array analysis of the two different cultures both detected a large presence structural molecules, these messages were not analyzed in the same manner as protein families with known signaling capacity. It is also important to note that signaling molecules by their very nature do not have to be present at high concentrations to function adequately while structural molecules have to be present in higher concentrations since they are responsible for building the scaffolds normally utilized in forming the ECM. The secretome analysis by its very nature favors the evaluation of the highest expressed proteins present within the complex medium, but this may also limit the detection of molecules with lower expression.

Finally, it is interesting to note that several of the factors commonly used within directed differentiation protocols designed to generate insulin-producing cells were found to be expressed in the pancreas-specific MEF cultures. One of the highest expressed morphogens found in the Pan-MEF cells, Inhba, has been found to be crucial in the in vitro generation of definitive endoderm and has also been shown to greatly increase the occurrence of pancreatic progenitors in later stages. While the data shown in this example does not show any significant expression of any FGFs, FGF10 was observed within other Pan-MEF preparations indicating that Pan-MEF preparations do display a certain level of variability between preparations. This variance may be attributed to minor differences between the timing of the preparations or differential effects of the in vitro culturing of this cellular population. Also it is important to note that Hb-EGF was detected within the array analysis and since this molecule is capable of signaling through the same pathway as FGF10 it is possible that Hb-EGF could substitute the effects of the FGF10. This is not surprising considering that many directed differentiation protocols designed to generate pancreatic subtypes rely on FGF7 as a substitute ligand for the same receptor. Detection of the metabolic enzyme ALDH2, which is capable of reducing retinaldehyde, implies that the Pan-MEF culture are capable of producing retinoic acid, a compound used in regionalizing definitive endoderm towards pancreatic epithelial in both directed differentiation protocols as well as in vivo during normal pancreatic development. The presence of Nbl1 implies that the Pan-CM may be partially capable of inhibiting ‘stray’ endodermal fates from occurring in pluripotent cultures since Nbl1 has been shown to function as a Bmp inhibitor. All together the analysis of the proteins being produced by Pan-MEF cultures suggests that the Pan-CM may contain proteinous components capable of imitating directed differentiation protocols. The main difference between directed differentiation protocols and our current protocol thus becomes the capability of pluripotent cultures to respond appropriately to components that are continuously present during forward differentiation as compared to the stage-wise administration of similar components.

### Example 13

### Functional Mimicry of Pan-CM Secretome Through Defined Media Additives Reproduces Direct Induction of Pancreatic Progenitors from Pluripotent Cells

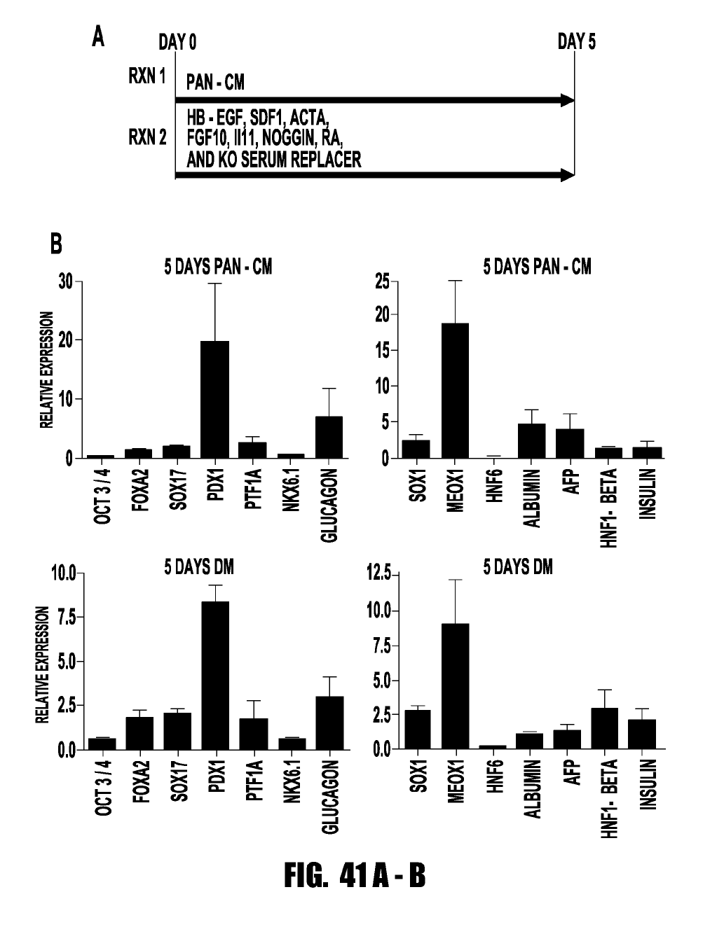

As a means of directly testing the validity of the array analysis performed within the last example to the functionality of the Pan-CM, a defined medium composition designed to replicate the Pan-CM was tested for its ability to drive the forward differentiation of the H1-hES cell line and compared to the effects observed with the Pan-CM. The composition of the defined medium consisted of the proteins informed from the array analysis of the Pan-MEF cells and are listed in panel A of FIG. 41. Pluripotent cells were incubated in either the defined medium or Pan-CM for 5 days at which time triplicate RNA samples were extracted from the two set of reactions conditions and subjected to transcript analysis for several pancreas specific and stray fates. Both reaction conditions generated a similar response in the forward differentiation of pluripotent cells as is evident in FIG. 41 panel B. The endodermal genes FoxA2 and Sox17 responded similarly within both set of conditions with relative expression levels increasing ˜1.5-˜2.0 fold indicating a similar induction of endoderm within both sets of conditions. There was also a modest up regulation of both ectodermal and mesodermal fates as indicated by the marker genes Sox1 and Meox1 respectively. While both reactions conditions displayed a similarly relative response in the levels of Sox1, up regulated ˜2.5 fold, the pluripotent cells supplemented with Pan-CM displayed a higher relative fold increase for Meox1 reaching a fold increase of ˜18.5 as compared to the fold increase of ˜9 reached in the pluripotent cultures incubated in the presence of the defined medium. Interestingly the pancreatic progenitor marker Pdx1 was among the highest up regulated genes assayed reaching a relative fold increase of ˜20 in the cultures incubated with Pan-CM and an ˜10 fold increase in the cultures incubated in the presence of the defined medium. Overall the genes assayed throughout this experiment all responded similarly in both set of conditions.

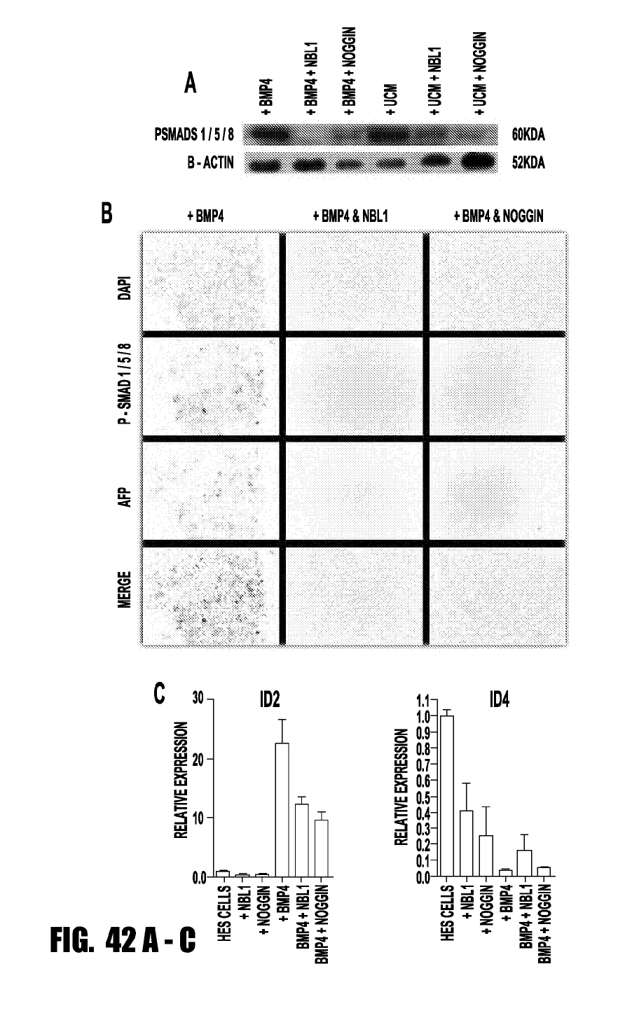

Our initial experiment testing the ability of a Pan-CM informed defined medium ability to replicate the direct induction of pancreatic fates relied on the use of Noggin as a BMP inhibitor. This was due to the fact that Nbl1 was not available at the time so a member of the same family of proteins was used. We obtained the NBL1 protein (Sino Biologicals, cat number 10169-H02H) and determined if Nbl1 operates as a Bmp signaling inhibitor. Pluripotent cultures were subjected to a Bmp4 stimulus in the presence and absence of either Noggin or Nbl1. FIG. 42 panel A shows the level of phosphorylated smad 1/5/8 (the immediate downstream consequence of the BMP signaling) in protein extracts from the various culture conditions. The initial hES culture (treated with or not treated with Bmp4) shows the presence of phosphorylated smad 1/5/8 while cultures treated with either Noggin or Nbl1 failed to produce this BMP specific phosphorylation event. This inhibitory effect was also observed by immunohistochemical analysis of the treated cultures and is shown in FIG. 42 panel B. In addition the direct gene targets of Bmp4 signaling, ID2 and ID4, were assayed and shown to be inhibited in the presence of Noggin or Nbl1 (FIG. 42 panel C) as was the downstream target of Afp (panel B). All together this clearly demonstrates that Nbl1 functions as a Bmp4 inhibitor.

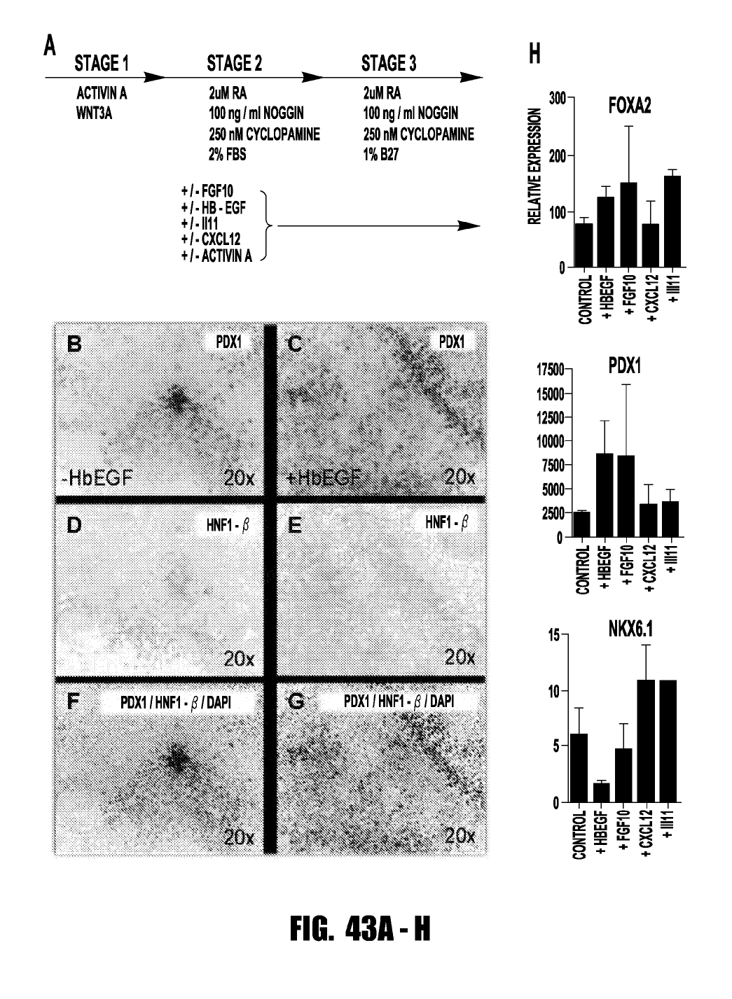

We next explored the ability of our defined medium informed from the transcriptome and sectretome analysis of the Pan-MEF cultures to induce the differentiation of pluripotent cells towards pancreatic fates. This was performed in short term cultures with incubation periods only lasting 5 days (FIG. 43). Notably, the cell conditioned medium greatly improved over the Pan-CM in inducing the endodermal genes FOXA2 and SOX17 with relative expression levels reaching nearly 30× (ranging between 200-400 fold increase over pluripotent cultures) over that observed using Pan-CM as the inducing agent (FIG. 43 panel B). Genes associated with pancreatic endoderm were similarly up-regulated over the Pan-CM induced cultures with PDX1 levels reaching a relative up-regulation of approximately of between 700-1150 fold over hES levels. Notably, the inclusion of Wnt3a on the first day of the induction, regardless of the serum concentrations used, improved TrPC marker NKX6.1 expression within the 5 day induction period.

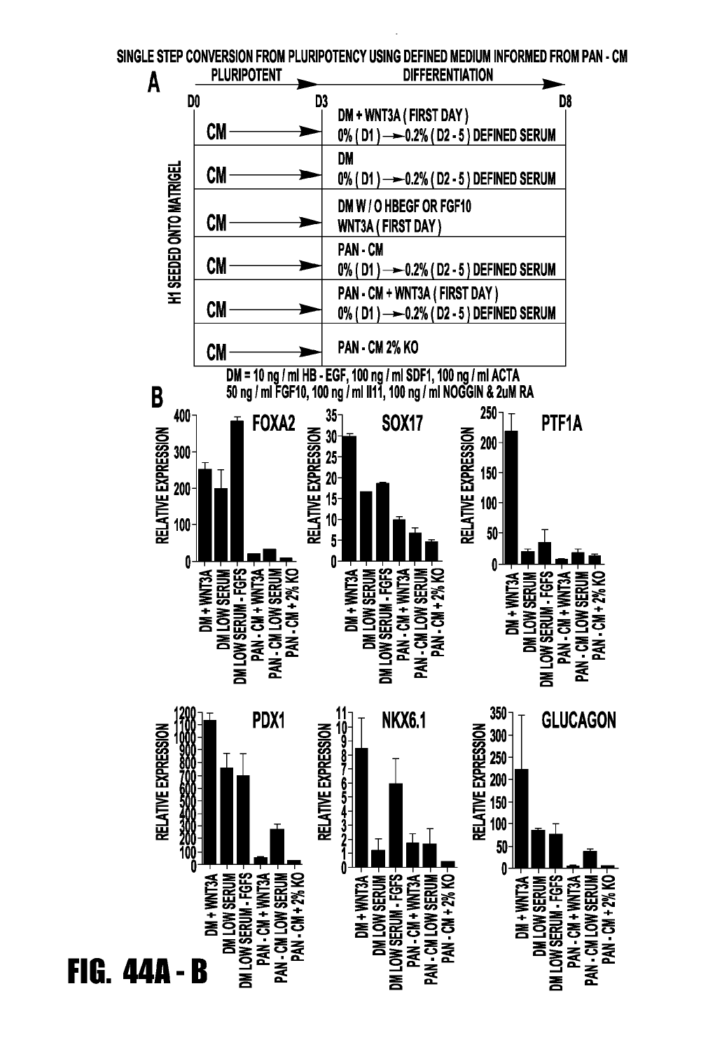

We next assayed the ability of our cell conditioned medium to induce pancreatic fate following long-term culture. Pluripotent cultures were maintained in the presence of cell conditioned medium for 3 weeks at which time these culture were subjected to immunohistochemical analysis for the endodermal protein FOXA2 and the pancreas-specific protein Pdx1. Widespread expression of FOXA2 was observed in the cultures maintained in the presence of the cell conditioned medium (FIG. 44 panels B and C). Cultures that were maintained in basal medium missing the components of the cell conditioned medium failed to achieve any significant levels of FOXA2 expression though small patches of FoOXA2+ cells were observed. While not homogenously expressed, large patches of PDX1+ regions were observed in the cultures maintained in the presence of the cell conditioned medium (FIG. 44 panel B). Altogether this demonstrates that a single-stage provision of a defined combination of factors, as described herein, is capable of inducing a pancreatic phenotype on a culture of pluripotent cells.

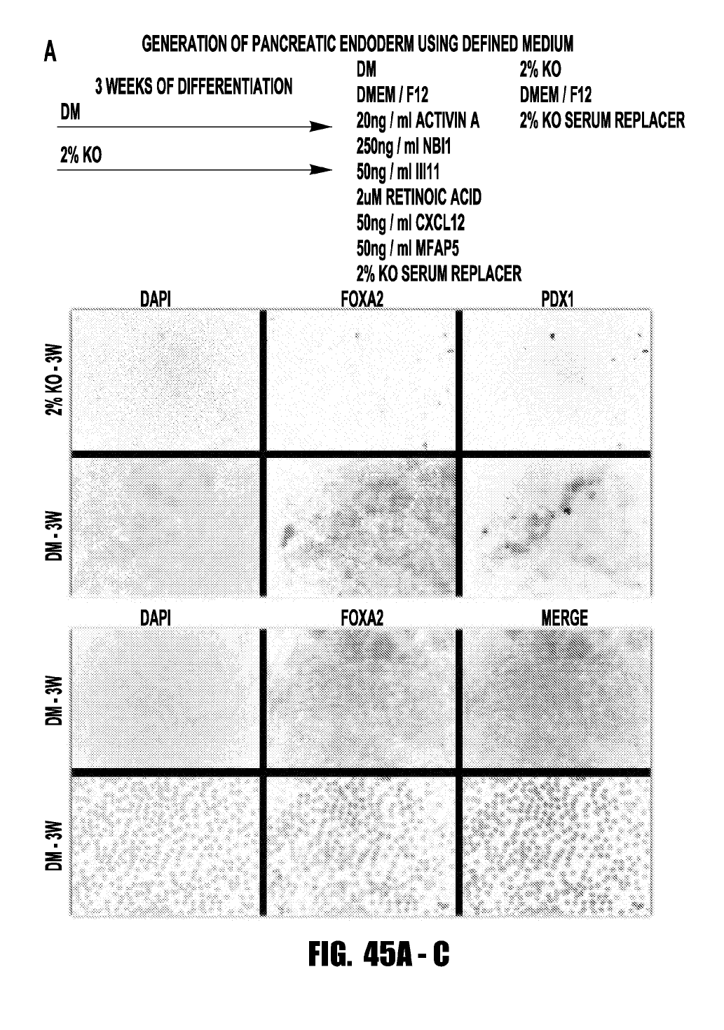

Since we have seen that the Pan-CM induced differentiation of the pluripotent culture results in an increased presence of pancreatic progenitors within forward differentiating cultures we next assayed the individual components of the cell conditioned medium as single additives throughout stage 2 and stage 3 of a directed differentiation protocol. Stage 2 and stage 3 cultures were assayed since these are the stages which normally establish pancreatic endoderm within directed differentiation protocols. An outlined schematic of the components used throughout the directed differentiation is provided in FIG. 45 panel A. Three genes were assayed throughout this series of experiments: the endodermal gene FoxA2, the early pancreatic progenitor gene Pdx1 and the endocrine progenitor gene Nkx6.1. The inclusion of Hb-EGF or Il11 did result in an increased FoxA2 activation with relative transcript levels reaching ˜125 and ˜160 fold respectively as compared to the control reaction which shows only a ˜75 fold increase over hES levels. This implies that the inclusion of Hb-EGF or Il11 may have either directly increased FoxA2 levels or preferentially allowed for the expansion of this population of cells. The most dramatic effect noted within the genes assayed throughout this series of experiments was observed within the Pdx1 transcript levels obtained within cultures that had either Hb-EGF or FGF10 included throughout stage 2 and stage 3. Control cultures had a relative transcript level that had increased ˜2500 fold over hES levels, but when either Hb-EGF or FGF10 was included throughout stage 2 and stage 3 this relative Pdx1 transcript level was increased to ˜8500 fold suggesting a necessity for FGF stimulation in the regionalization of the definitive endoderm. A representative Pdx1/Hnf1-β staining of reactions performed with Hb-EGF included throughout stage 2 through stage 3 is compared to the control reaction in FIG. 45 panels B-G. As seen when comparing panels B through G in FIG. 45 the Pdx1/Hnf1-β pancreatic endoderm generated with the administration of Hb-EGF throughout stage 2-3 has a wider spread and higher percentage of Pdx1+/Hnf1-β+ cells.

While this series of experiments have successfully established a beneficial effect for the inclusion of a FGF ligand within throughout stages 2 and 3 of a directed differentiation protocol, it should be noted many directed differentiation protocols already currently include a either FGF10 or FGF7 within either stage 2, stage 3 or both. However our array analysis of the Pan-MEF cultures did not detect any significant levels of FGF10 but rather had significant levels of Hb-EGF a novel factor not previously used in directed differentiation protocols which can stimulate the same FGF receptor as FGF10. Also it is important to note that Activin A was also used as an additive throughout stage 2 and 3 of the directed differentiation protocol, however the continued incubation of the forward differentiating cells within the presence of Activin A had a detrimental effect on the cultures and the RNA obtained from these experiments were of low quality and excluded from further analysis. This effect may have been due to the high concentration of Activin A used within these reactions 100 ng/ml. This high concentration was chosen because it is the typical concentration used in generating endoderm during stage 1 of directed differentiation protocols, however the actual concentration present within the Pan-CM was undetermined and may have been quite lower. Finally the inclusion of Cxcl12 or Il11 throughout stage 2 through 3 of the directed differentiation protocol did show a slight increase in the relative transcript level of Pdx1, though this increase was not significant it may be more important to note that there was no detrimental effect noted due to their presence within the directed differentiation protocols. This implies that the benefit due to their presence within the directed differentiation protocol may only be detectable by assaying other pancreatic markers or assaying the directed differentiation cultures at later stages, or as in them being a supportive component in a multi-factorial additive provision, yet unable to operate to induce pancreatic fates when provided alone.

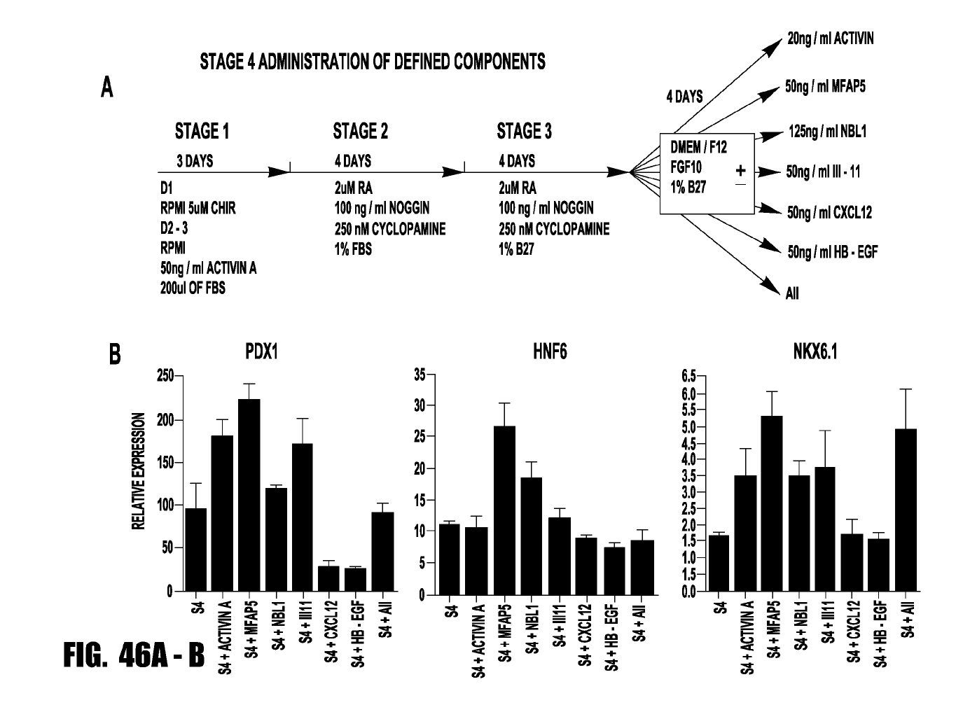

We next explored if there were any beneficial effects from inclusion of the individual components of the cell conditioned medium in stage 4 cultures. The individual components were assayed in a directed differentiation protocol for their ability to up regulate markers of the pancreatic truck progenitor state. The relative expression of three key truck progenitor markers were assayed and results are shown in FIG. 46 panel B. PDX1 expression was significantly up-regulated in the presence of Activin A, Mfap5 and Ill11 while the administration of Cxcl12 and Hb-egf down-regulated PDX1 expression at this stage suggesting that while these two components are useful in the formation of the pancreatic progenitor their continued administration through stage 4 may be prohibitive for trunk progenitor formation. It was noted that both Mfap5 and Nbl1 up regulated the expression of HNF6. Finally expression of the TrPC marker NKX6.1 was positively effected by the addition of Activin A, MFAP5, NBL1 and ILL11.

### Example 14

### Studies Providing Definitions and Molecular Control of Formation of the Trunk-Patterned Pancreatic Progenitor Cell (TrPC)

Because our previous studies, described in example 10 illustrated the possible involvement of Notch signaling in creating a competency for insulin cell development, we sought to clarify the exact role of Notch in pancreatic progenitor patterning. The relevance of this concept is well illustrated in FIG. 36K, which resulted in highly efficient insulin cell formation, after exposure to Ngn3 exogenous protein followed by Notch inhibition.

To more carefully address the role of Notch in pancreatic development in order to improve outcome of directed differentiation of human pluripotent stem cells, we created another mouse model that specifically addressed the role of Notch.

### Example 14 (i)

### Notch Signaling is Required for Pancreatic Endocrine Cell Differentiation in the Developing Embryo

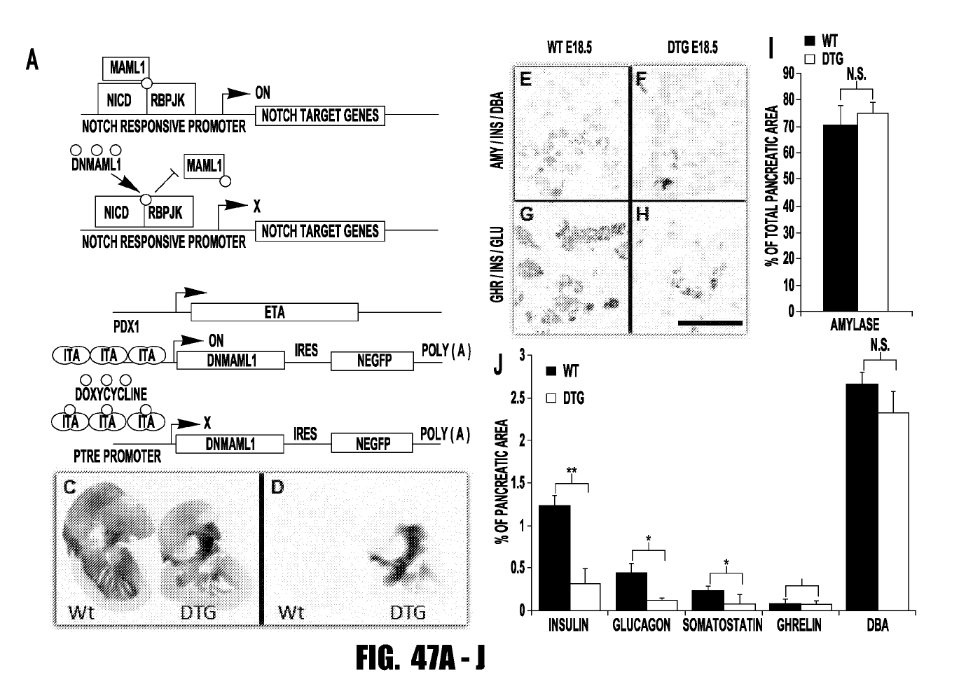

Prior to our studies, the role of Notch signaling in pancreas development was deemed ambiguous. While previous studies of mouse mutants of Notch signaling component genes such as RBP-jκ have suggested that Notch signaling is inhibitory to endocrine cell differentiation (Apelqvist et al., 1999; Jensen et al., 2000), other observations indicate that Notch signaling may positively influence the endocrine fate. Given that this difference could be explained by sequential, but temporally distinct, roles of Notch in organogenesis, we generated a conditional transgenic model that would allow temporal control of Notch signaling. Seeking to achieve specificity for the Notch pathway, this strategy was aimed at targeting the NICD DNA binding complex. NICD functions as a transcriptional co-activator, and becomes tethered to target promoter regions through the formation of a trimeric complex consisting of RBP-jκ and a bridging factor of the mastermind-like (MAML) family (FIG. 47A). MAML is required for complex formation, and a truncated version of the transcriptional co-activator, mastermind-like 1 (MAML1), consisting of the N-terminal domain (amino acids 13 to 74) that interacts with NICD and RBP-jκ, acts effectively as an antagonist of Notch signaling by sequestering NICD and RBP-jκ into an inactive transcriptional complex (FIG. 47A).

We generated transgenic mice with a truncated mastermind like-1 fragment, referred to as dominant negative mastermind like-1 (dnMAML1), cloned downstream of the tetracycline-responsive promoter pTRE, followed by an IRES-nEGFP (FIG. 47B) reporter cassette. The dominant negative mastermind like1 fragment (dnMAML1) consisting of amino acids 13 to 74 of the mouse MAML1 was generated by PCR amplification using cDNA from mouse E14.5 pancreas. Primers were designed based on the mouse mastermind-like1 (MAML1) to cover amino acids 13 to 74. The forward primer was initiated with ATG and a FLAG tag sequence was incorporated at the end of the reverse primer to generate a FLAG tagged dnMAML1. The resulting fragment was ligated into a modified version of the pTRE2 vector which contains an IRES-nGFP downstream of the multiple-cloning-site (FIG. 47B). To validate doxycycline inducibility, this construct was co-transfected with pCMV-rtTA into HEK293 cells. Nuclear GFP was detected in the presence of doxycycline and the functional ability of the construct to inhibit Notch-driven activation was assessed. The pTRE-dnMAML1-IRES-nGFP fragment was linearized and injected into fertilized one-cell embryos at the Case Western Reserve University transgenic and gene targeting facility. Four founders (F0) out of a total of 22 were identified to carry the transgene by PCR based genotyping using DNA from ear notches and primers specific to the transgene. Transmittance and pancreas specific expression of the transgene was tested by mating founders to Pdx1-tTA mice. Double transgenic (DTG) embryos (positive for dnMAML1 and Pdx1-tTA) showed pancreas-specific expression of the transgene through the expression of EGFP in the pancreas and duodenum, in accordance with the expression domain of Pdx1.

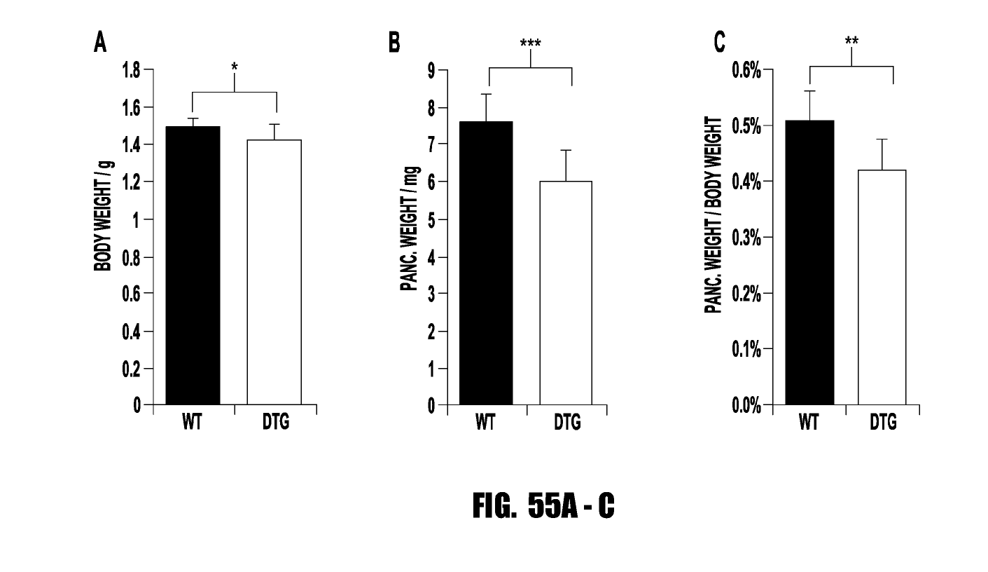

We assessed conditional expression of transgenic founders by crossing pTRE-dnMAML1-IRES-nEGFP mice to Pdx1-tTA mice, which express the pTRE transactivating protein tTA under the Pdx1 promoter. These two independent transgenic founder lines were characterized in more detail, and the phenotype was consistent between both lines. In absence of doxycycline, we observed a pancreas/distal foregut-specific activation of the transgene in double-transgenic embryos (DTG) through the expression of nEGFP (FIG. 47D). The lines did not reveal expression leakage of the TG cassette, as embryos carrying only the transgene for pTRE-dnMAML1 did not show EGFP expression or any pancreatic phenotype at any point in our study. Furthermore, the expression of the TG cassette was extinguished in DTG embryos by administration of doxycycline to pregnant dams throughout gestation. Measurement of pancreatic mass relative to body weight at E18.5 revealed that on average the pancreas of DTG embryos was 79% that of wild type litter mates (FIG. 55). Focusing here on the pancreas, we performed a quantitative analysis on the five endocrine cell types (α, β, δ, PP, ε), acinar and duct cells of pancreatic tissue of E18.5 dnMAML1 DTG embryos in comparison to wild type litter mates (FIG. 47E-J). The relative area of pancreatic acinar cells (app. 70% of total) was not changed (FIG. 47I). While pancreatic duct cell density was slightly reduced in DTG embryos, this was statistically not significant (FIG. 47J). However, immunofluorescence staining revealed a relative paucity of 4 of the 5 endocrine cell types (α, β, δ, ε) in DTG embryos as observed through histological assessment for insulin, glucagon, somatostatin and ghrelin (FIG. 47E-H). Histological analysis was performed, based on n≧3 samples at all time points. Dissected tissue was fixed in 4% paraformaldehyde (PFA) at 4° C. for 4 hours or overnight. All histological analysis was performed on 6 μm frozen sections. Antigen retrieval was achieved by microwave treatment (2×5′ in 0.01M citrate buffer, pH 6). Microwave treatment was avoided for stainings that involved visualization of nEGFP, since this lead to quenching of nEGFP. Primary antibodies were applied overnight. Secondary antibodies 1:100 dilution (pre-absorbed secondary antibodies coupled to Cy2-, Texas Red- or AMCA, Jackson Immunoresearch, West Grove, Pa.) were applied for 1 hour at room temperature. Following washing 3×5′ with PBS, slides were mounted in glycerol mount (20% glycerol in PBS). Controls without added primary antibody were included in all setups. Imaging was performed with Olympus BX51, equipped with digital image acquisition using IMAGE pro 4.1-7.0.

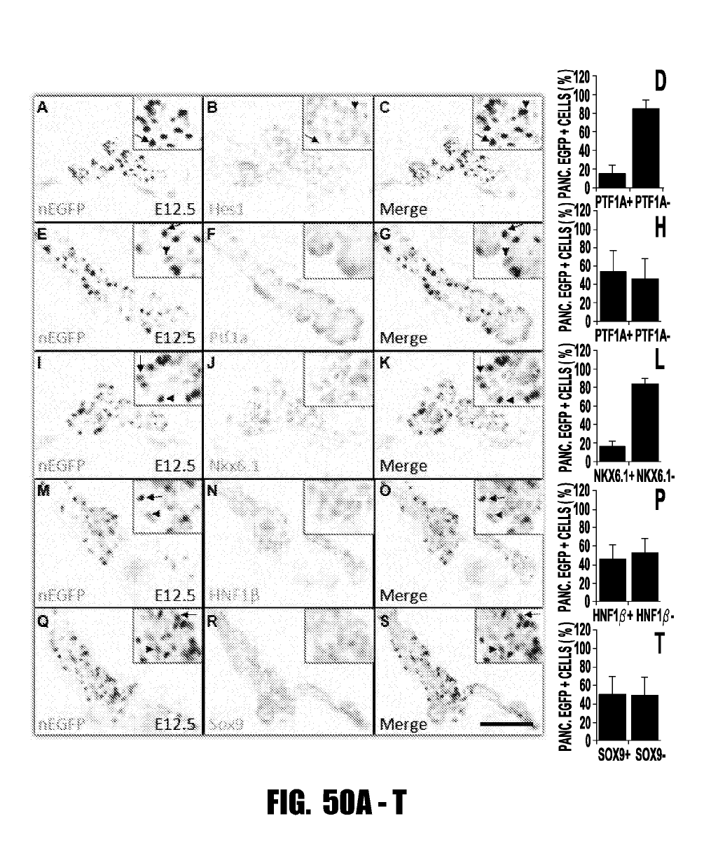

Morphometric quantification based on these stainings demonstrated a reduction in pancreatic endocrine cells in dnMAML1 DTG embryos, down to one-third of wild type litter mates (FIG. 42J). Intraperitoneal glucose tolerance testing performed on one-month old animals revealed that DTG mice expressing dnMAML1 protein exhibited impaired blood glucose clearance compared to wild type controls (FIG. 50).

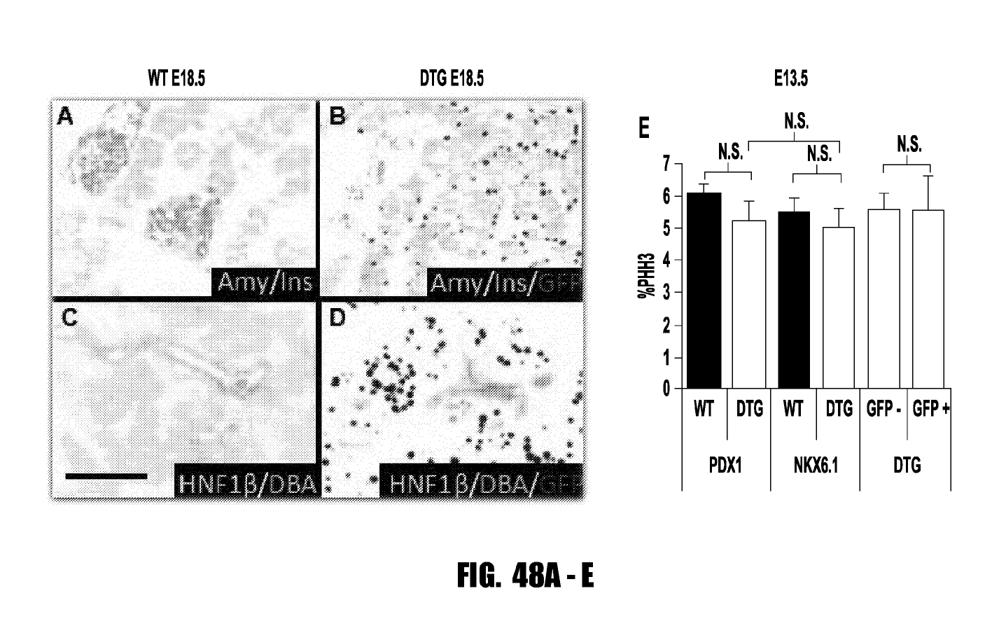

While the pancreas exhibited apparent visual homogeneity of EGFP expression (FIG. 47D), histological analysis of tissue sections revealed a mosaic expression of the transgene. Given that the dnMAML1 operates as cell-intrinsic regulator, this observation prompted us to conduct subsequent analysis of this model with emphasis on the transgene expressing cells relative to the non-transgenic cells. In an effort to characterize the effect of the transgene upon the cells that expressed it, we carried out histological analysis of pancreatic tissue from DTG embryos with that of wild type litter mates as control using terminal differentiation markers for the major cell types of the pancreas namely, endocrine, acinar, and duct cells. Interestingly, at E18.5, we observed that the transgene was expressed predominantly in acinar cells as marked by the expression of EGFP in amylase positive cells (FIG. 48B). Quantitative analysis indicated 69±10% of acinar cells expressed the transgene, while only 4.8±3.1% of insulin expressing cells was EGFP+. A similar analysis of the distribution of the transgene relative to pancreatic duct cells was performed by staining pancreatic tissue for the duct markers DBA lectin and HNF1β (encoded by Tcf2) in relation to the transgene derived EGFP cells (FIG. 48D). We did not detect any pancreatic duct cells expressing the transgene after thorough examinations.

Morphometrical assessment was done as in the following description. For quantitative morphometry, pancreata of age-matched wild-type and various genotyped embryos were sectioned through and every fifth section was picked up for immunostaining using antibodies staining described in above. The total area of stained cells and pancreas from five equally spaced sections were quantified using ImagePro Software (Media Cybernetics, Bethesda, Md.). To assess the effects of Notch inhibition in transgene expressing cells, the gene of interest was immunostained in the presence of the transgene-derived EGFP and the total number of nuclear EGFP+ and EGFP+/gene of interest+ manually counted.

### Example 14 (ii)

### Notch Signaling is Required for Pancreatic Pro-Endocrine/Duct TrPC Patterning

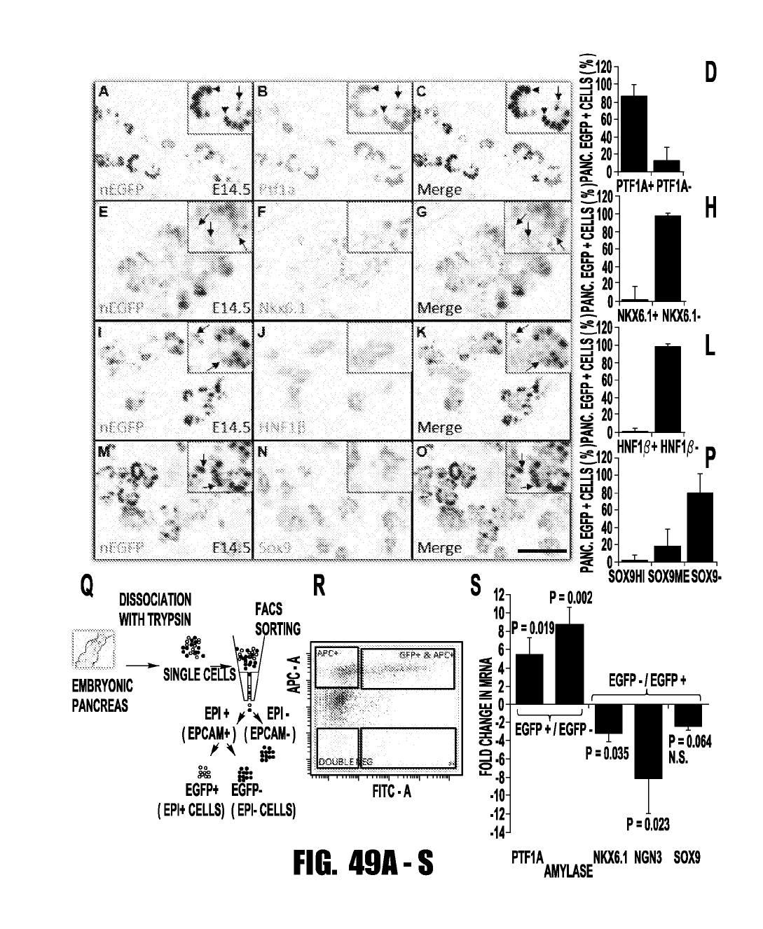

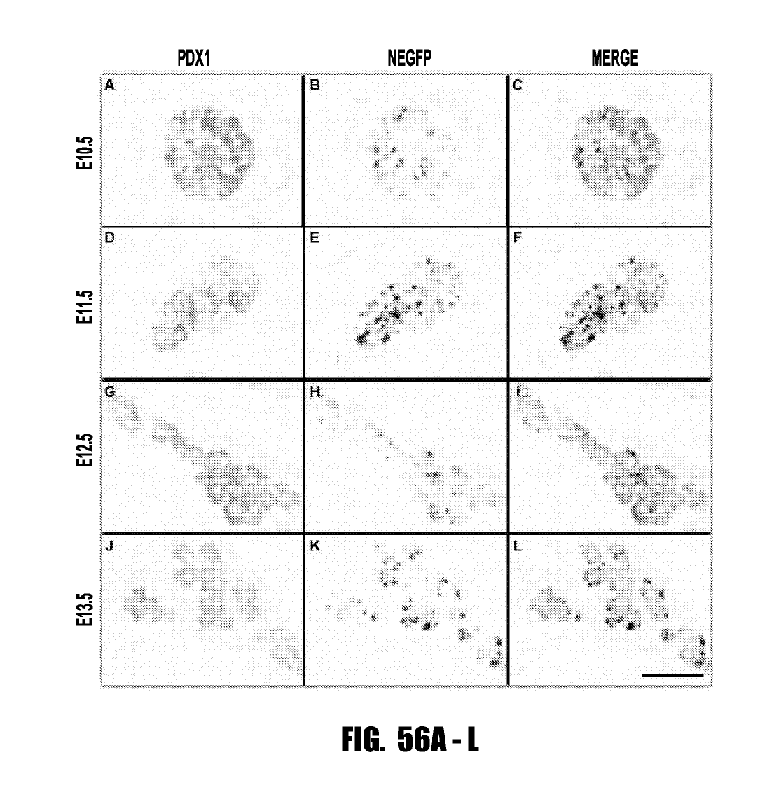

Since the prevailing late-effect of the transgene was on the endocrine pancreas, we were surprised to detect an acinar-biased distribution of the transgene expressing cells. Also, given that the transgene is indirectly governed via the Pdx1 promoter (Pdx1-tTA^{KI}) which is first active in all pancreatic progenitor cells, and leads to a random distribution of transgene positive cells throughout the early pancreatic epithelium (FIG. 56), the observed segregation of the transgene expressing population at later stages was at odds with the expected pattern, considering a hypothetical inert transgene. We tested several hypotheses that could possibly explain the observed effects. First, if the dnMAML1 protein would negatively affect cell viability in a cell-specific manner, the results could be explained by a particular loss of endocrine/ductal cells experiencing the TG protein, which possibly could undergo apoptosis at some point prior to the time of analysis at E18.5. In such a case, cells refractory to the effects of dnMAML1 protein would not be affected, and remain detectable. However, studies determining apoptotic cells at earlier stages (E12.5-E14.5) did not detect such (data not shown). Second, we hypothesized that dnMAML1 protein, and consequently loss of Notch signaling, could have positively affected cell division, but only so within the acinar population, leading to a relative increase in such cells over the others over time. Experimental evidence provided no support for this hypothesis, and cells expressing dnMAML1 protein had identical replicative rates as wt/non-transgenic cells neighboring the EGFP+ pool in the dnMAML1 model (FIG. 48E). Finally, we speculated that Notch signaling could have affected the generation of the two progenitor compartments, in a manner where endocrine/ductal fates was negatively controlled by dnMAML1 presence, and acinar fates positively so. Such an event would have occurred prior to E18.5, and likely at, or just prior to the “secondary transition”. Emerging evidence points to a process of pre-patterning of the pancreatic progenitor field prior to the onset of the secondary transition, and this leads to future bias in terminal fate commitment. During early embryogenesis, the branched pancreatic epithelium becomes regionalized into branched “tip” and “trunk” domains. Although initially progenitor cells at the tip position contribute to all lineages of the developing pancreas, by E14.5 cells at the tip position contribute exclusively to acinar cells whereas cells within the trunk differentiate into endocrine and duct lineages. For this reason, we speculated that the experimental outcomes could be explained if dnMAML1 served to enhance the expression of acinar lineage-specific genes (tip domain formation) while suppressing endocrine/duct progenitor cell specific genes (trunk domain formation). To test this hypothesis, we focused on early embryonic stages with the aid of transgenic EGFP expression and pancreatic progenitor markers for tip and trunk cells. In so doing, we took advantage of the mosaic model because we could scrutinize a possible fate bias within a cell experiencing Notch-inhibition and compare such to neighbors not under such influence. By E14.5, Ptf1a positive progenitor cells are lineage-restricted to pancreatic acinar fate, and we therefore analyzed expression of Notch depleted dnMAML1 cells relative to Ptf1a by immunofluorescence staining. At this time point, nEGFP/dnMAML1+ cells predominantly (over 80%) express Ptf1a and notably, these transgenic cells are mostly localized at the tip position (FIG. 49A-C) leaving a trunk-region devoid of nEGFP/dnMAML1+ cells. Also, at E14.5, dnMAML1-expressing cells fail to express trunk-specific makers (Nkx6.1, HNF1β and Sox9 (FIG. 49E-P)). A few Notch-suppressed cells showed a weak expression of Sox9, suggesting that these cells are in an intermediate state of losing Sox9 expression (FIG. 49O, P). To further validate the observation that inhibition of Notch signaling via dnMAML1 results in loss of trunk-specific gene expression, we isolated epithelial dnMAML1+ expressing cells at E13.5 by fluorescence-activated cell sorting (FACS) based on the transgene-derived EGFP and co-labeling for the epithelial marker Epcam (FIG. 49Q,R). The FACS-based cell isolation was done as described in the following: Embryonic pancreas was isolated in cold HBSS medium, DTG and wild type tissues were sorted based on EGFP and pooled into batches of 6-10 pancreata. Pancreatic tissue was chopped into pieces in 500 μl of collagenase P (1 mg/ml in HBSS) and incubated for 5′ at 37° C. with shaking. Collagenase activity was stopped by adding 500 μl of 5% FBS in HBSS and centrifuged at 1800 rpm for 4′ at 4° C. Tissue samples were then treated with 0.05% Trypsin/EDTA for 5′ at 37° C. followed by a rinse in 5% FBS. Dissociated cells were pelleted at 1800 rpm for 4′ at 4° C. and stained for EpCam by incubating with APC-EpCam antibodies for 40′ on ice. Stained cells were spun down and resuspended in HBSS with 5% FBS and filtered with 30 μm cell strainer prior to FACS sorting.

RNA was extracted from the isolated fractions and subjected to quantitative RT-PCR analysis (FIG. 49S). The quantitative RT-PCR analysis was done as described in the following: DTG pancreatic epithelial cells were sorted into transgene expressing (EGFP+) and non-transgene expressing (EGFP−). Total RNA was isolated, treated with DNase and reverse-transcribed using Superscript II (Invitrogen). Quantitative real time PCR was performed using an ABI 7500 Real Time PCR System (Applied Biosystems, Foster City, Calif.). PCR Reactions (25 μl) were performed using RT² Real-Time PCR SYBR Green/ROX Master Mix (SA Biosciences). The relative concentration of RNA for each gene to GAPDH mRNA was determined using the expression of 2^{ΔCT}, where ΔCT=(CTmRNA−CT_{GAPDH} mRNA).

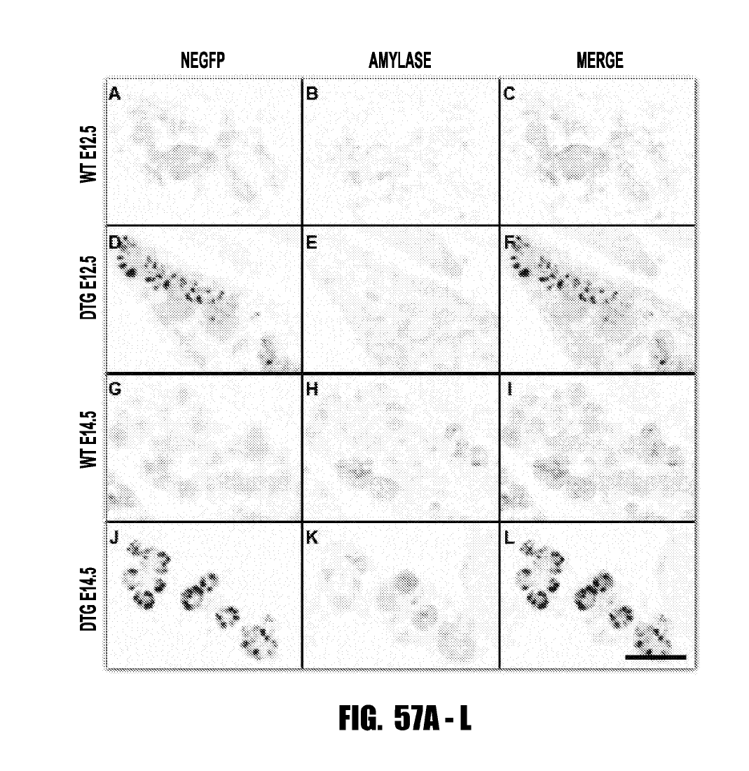

The qRT-PCR analysis revealed that transgenic cells predominantly express the acinar-specific transcription factor Ptf1a and the acinar product amylase, which confirms that these cells have adopted an acinar fate. Correspondingly, this effect was accompanied by a decreased expression of trunk-specific genes such as Nkx6.1, Sox9 as well as the endocrine progenitor maker Ngn3 (FIG. 49S). These sets of observations strongly suggest that suppression of Notch signaling leads to loss of “trunk” progenitor fate and a corresponding gain of “tip” fate with conceivably a concomitant morphogenetic effect resulting in the placement of the Notch-suppressed cells at the tip position, as these complete the acinar program. While immunofluorescence staining confirms that dnMAML1 expressing cells initiate acinar specific gene expression at the onset of terminal differentiation of acinar cells at E14.5, we did not detect induction of premature acinar cell differentiation in the DTG pancreas (FIG. 57). We conclude that suppression of Notch signaling results in the patterning of MPC towards the acinar fate, followed by a normal differentiation time course to terminal cell fate.

### Example 14 (iii)

### TrPC Formation Via Notch Signaling is Mediated by Direct Activation of the TrPC Determinant Nkx6.1

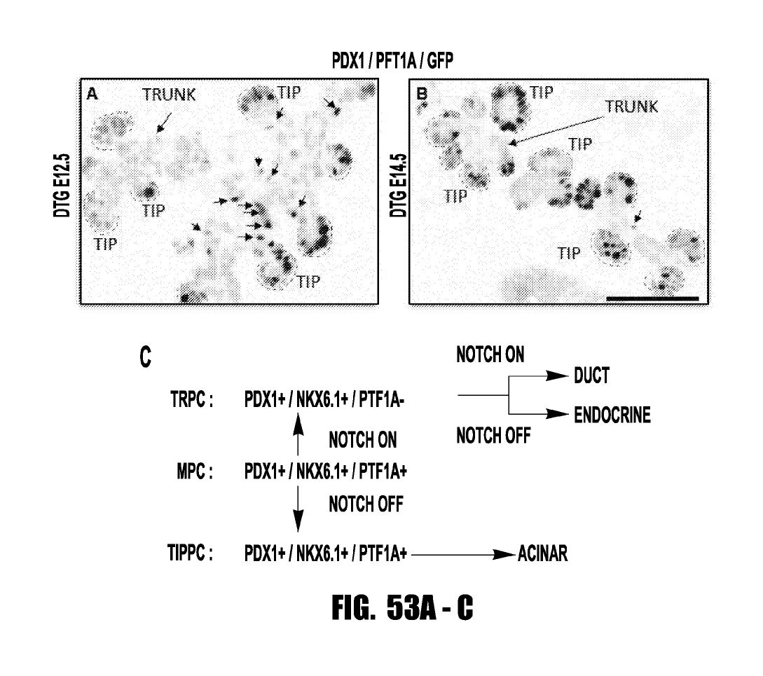

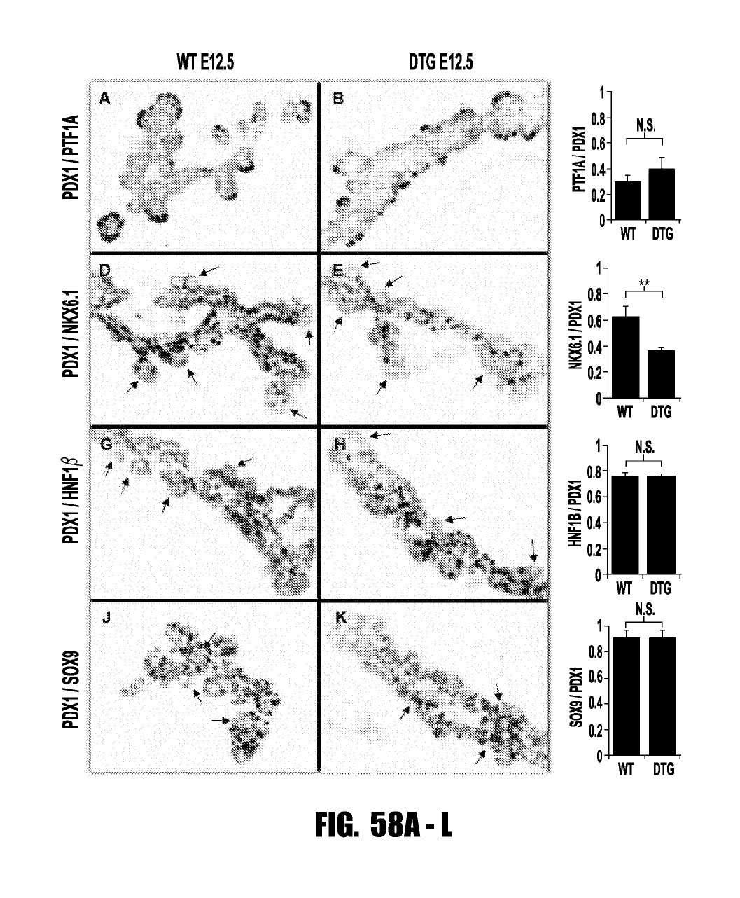

While the above analysis suggests that Notch inhibition does not compromise tip fate allocation, but instead impairs trunk-derived fates, E14.5 marks a stage where the “tip” and “trunk” cells are completely segregated and terminal differentiation has initiated. We therefore analyzed pancreatic tissue from earlier-stage transgenic mice to uncover possible Notch-mediated events preceding the complete segregation of transgene expressing cells from wild type cells. In the E12.5 pancreas, which is far less lobulated/branched than the organ at E14.5, there was a tendency of clustering of most transgene expressing cells towards the epithelial/mesenchymal border, but we also found a considerable number of such interspersed within wild type cells in the trunk domain. Immunofluorescence analysis revealed that unlike the E14.5 transgenic pancreas, only about 50% of the transgenic cell population were strongly Ptf1a+ expressing (FIG. 50E-H), suggesting that the gradual increase in Ptf1a/EGFP double positive cells is an ongoing process in which the acinar compartment is increasingly enriched by Notch-suppressed cells. The presence of transgenic cells within the trunk at this stage further supported the notion that the subsequent sorting of these cells to the tip position is as a result of loss of trunk identity, and that cells within the trunk domain became recruited towards the acinar fate (FIG. 53A-B). This would consequently lead to a depletion of transgenic cells able to enter downstream endocrine/ductal fates. To validate this hypothesis, we analyzed the effect of dnMAML1 on other trunk-specific marker genes like Nkx6.1, HNF1β and Sox9 (FIG. 50I-T). Indeed, unlike E14.5 where almost all transgene expressing cells had lost these marker genes, at E12.5 we observed that almost 50% of dnMAML1 positive cells still expressed HNF1β and Sox9. Yet, only 20% of transgene positive cells expressed Nkx6.1 (FIG. 50I-L). Indeed quantification of absolute levels of these markers in DTG pancreas reveals that unlike the other markers, there is a significant reduction in the level of Nkx6.1+ cells. Ptf1a levels increase slightly, yet not statistically significant (FIG. 58). Thus, at this stage, suppression of Notch signaling is most dramatically reflected by a loss of Nkx6.1 expression. Analysis of dnMAML1 expressing cells at E12.5 for Hes1 expression reveals an identical percentage (about 80% of dnMAML1) fail to express Hes1 (FIG. 50A-D) as for Nkx6.1 (FIG. 50I-L). This suggests Notch signaling is required for Nkx6.1 expression, and when ablated results in the loss of Nkx6.1 and the concomitant loss of trunk lineage determination.

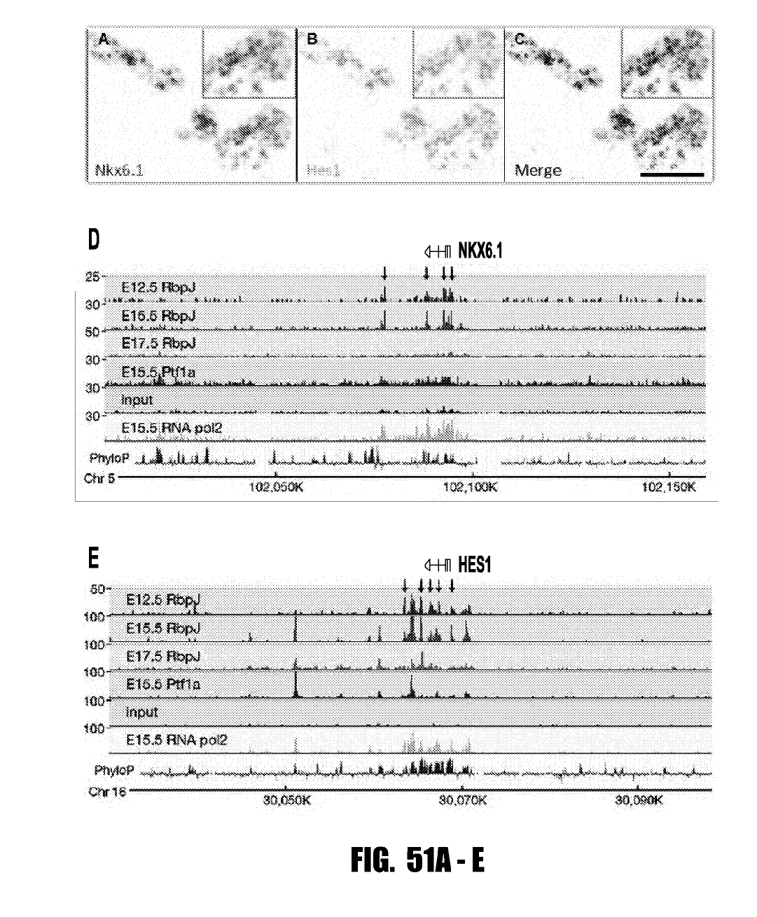

The largely overlapping expression of Hes1 and Nkx6.1 within E12.5 trunk progenitor cells (FIG. 51A-C) together with the induction of Nkx6.1 by NICD overexpression in the pancreas provides further evidence to suggest that Nkx6.1 expression might be under the control of Notch signaling. To test the hypothesis that Nkx6.1 is a direct target of Notch, we performed chromatin immuno-precipitation sequencing (ChIP-Seq) using anti-RBP-jk antibody on chromatin from E12.5, E15.5 and E17.5 pancreas. Analysis of the ChIP-Seq data revealed a binding peak for RBP-jk upstream of the Nkx6.1 gene transcriptional start site (TSS) at both E12.5 and E15.5. We found additional binding at specific locations in exon 1, proximal to the exon1/intron1 junction and also immediately following the terminal exon-3, consequently following the transcriptional stop. Finally, a distinct peak was identified approximately 10 kb downstream of the Nkx6.1 gene. We identified putative RBP-jκ binding sites within these RBP-jk bound regions. Unlike E12.5 and E15.5, we did not detect RBP-jk binding to these regions in E17.5 pancreatic chromatin, suggesting that Notch mediated control of the Nkx6.1 gene is limited to early developmental stages (FIG. 51D). Consistent with Hes1 being a Notch target gene, we found RBP-jk to bind regions proximal to the TSS of the Hes1 gene (FIG. 51E). Similar to the Nkx6.1 gene we also identified RBP-jk occupancy within the coding region of Hes1 (FIG. 51D, E).

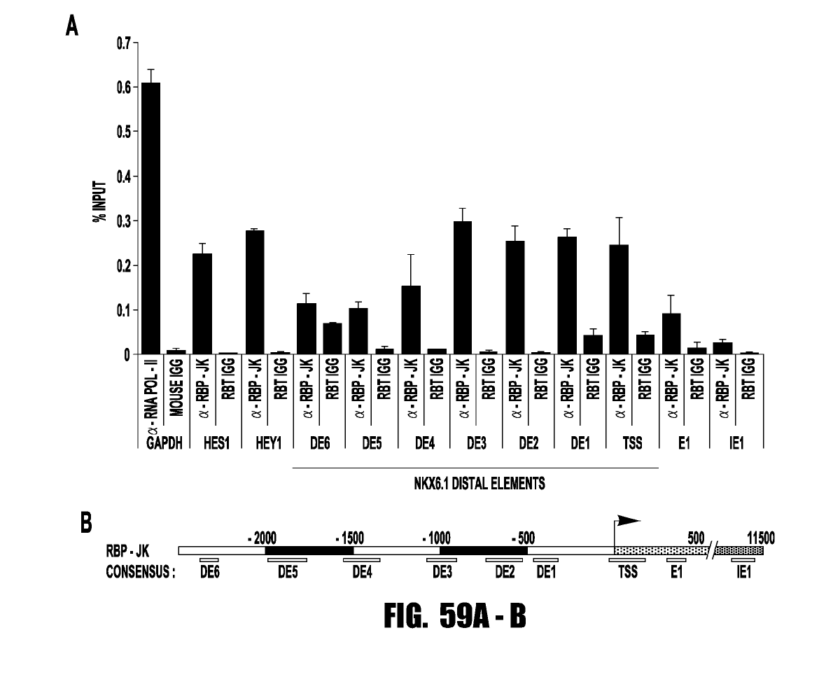

RBP-jκ can also partner with Ptf1a to trans-activate Ptf1a specific target genes, however the Ptf1a-RBP-jk sites are distinct from RBP-jκ sites involved in Notch signaling. In a parallel ChIP-Seq with anti-Ptf1a antibody, we found significant Ptf1a occupancy of regions corresponding to some of the RBPjk binding sites on the Hes1 gene, but not the Nkx6.1 gene, suggesting that the RBP-jk occupancy in the Nkx6.1 gene is Notch-mediated. RBP-jk occupancy of the proximal region of the Nkx6.1 gene was confirmed in independent experiments in which chromatin from E13.5 pancreas was immunoprecipitated with a second anti-RBP-jk antibody followed by qPCR with several primers covering app. 1 kb region of the Nkx6.1 TSS and flank (FIG. 59).

### Example 14 (iv)

### Lack of Premature Endocrine Differentiation in dnMAML1-Mediated Notch-Suppressed Cells Support a Role of Notch Signaling in TrPC Formation Rather that Pancreatic Progenitor Maintenance

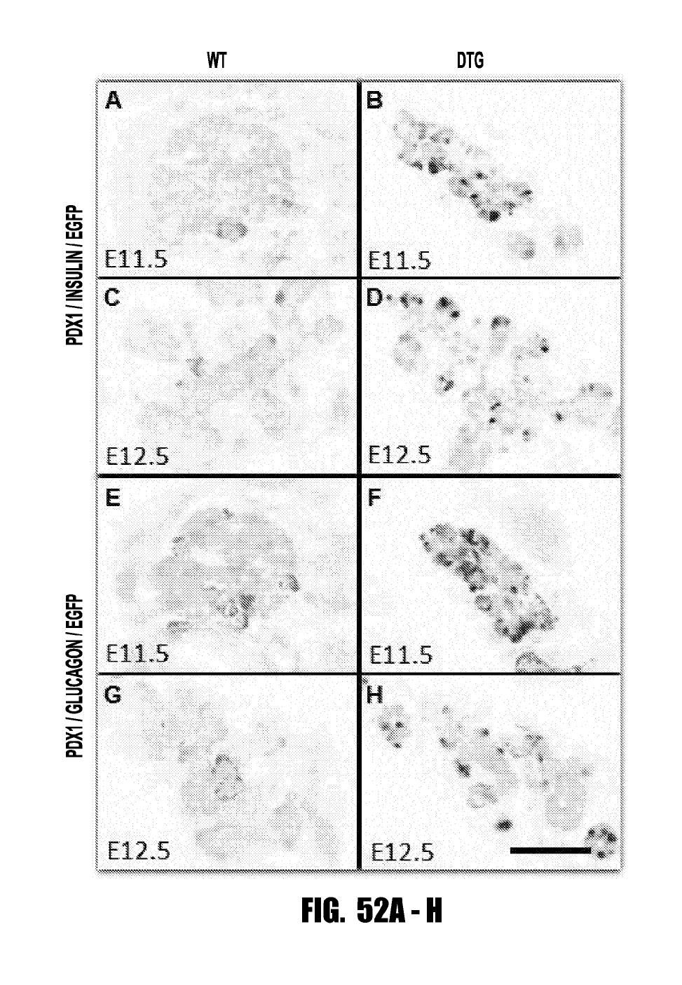

Our analyses thus far were in stark contrast to previous reports by ourselves and others on Notch signaling in pancreas development which suggests that Notch signaling is critical for the maintenance of the pancreatic progenitor cell state and when abrogated, cells differentiate prematurely resulting in a hypoplastic tissue that consist predominantly of endocrine cell fate. We therefore carried out a detailed analysis of pancreatic tissue in early embryos for evidence of dnMAML1-induced premature differentiation at the primary transition stage, which is when precocious differentiation of progenitor cells have been reported to commence in other models where Notch signaling was abrogated in the pancreas (Hes1−/−, Dll1−/−, as examples). We examined dnMAML1 expressing cells based on the presence of nuclear EGFP for the preferential expression of early glucagon- or insulin-expressing cells (FIG. 52). Endocrine cell clusters at the primary transition stage of pancreas development are often double-hormone positive. With the exception of rare instances where few clusters of glucagon-positive cells were located near dnMAML1 expressing nuclei at E11.5 (FIG. 52F), the majority of endocrine clusters (insulin or glucagon positive) were negative for the dnMAML1 transgene, indicating the lack of premature differentiation in transgene expressing cells. Furthermore, the mosaic suppression of Notch through dnMAML1 did not affect the architecture of the early pancreatic epithelium. The stratified nature of the E11.5 epithelium (FIG. 52A, E) was retained in the transgenic pancreas (FIG. 52B, F) and by E12.5 the transgenic epithelium branched normally (FIG. 52D, H) as in wild type pancreas (FIG. 52C, G). Taken together, our data did not support the induction of premature endocrine (or acinar) cell differentiation in individual cells experiencing a loss of Notch signaling.

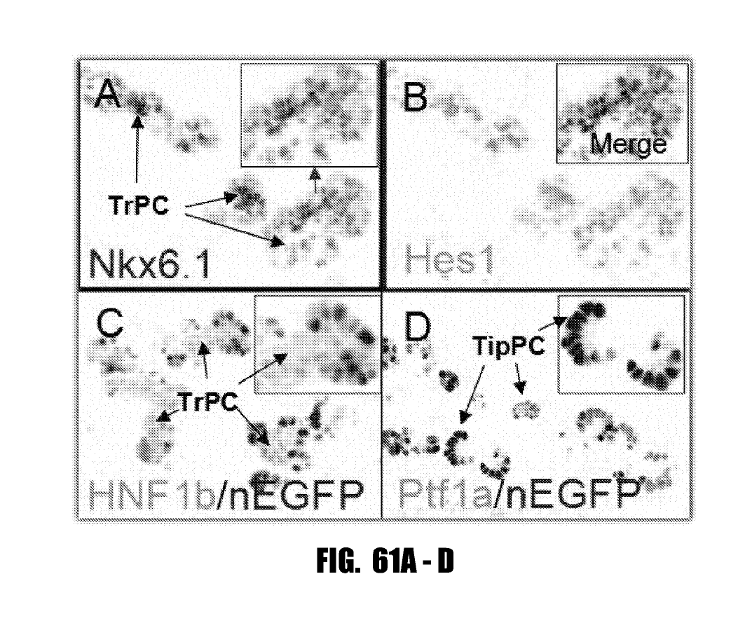

We conclude that notch signaling is critical for TrPC formation from MPCs in normal pancreatic development. The outcome of the appropriate TrPC formation is illustrated by segregation of two fields in from which acinar, and beta cell differentiation commence at E13.5 (FIG. 60A). The role of Notch signaling is depicted schematically in FIG. 60B, which also serves to illustrate the segregation of specific marker genes that become selectively expressed by the TipPC and TrPC populations. FIG. 61 illustrates by direct staining the segregations of some of the most critical determinants in the TrPC/TipPC fate assignment at the time when this normally occurs in the developing murine pancreas. The markers shown are Ptf1A, nkx6.1, Hnf1b, and the Notch target gene Hes1.

The inventors have theorized on the role of Notch and described such roles in the following review: Notch signaling in the pancreas: patterning and cell fate specification, Solomon Afelik, Jan Jensen, Published Online: December 06 201, DOI:10.1002/wdev.99, the disclosure of which is incorporated herein by reference. The considerations to notch signaling in relation to TipPC and TrPC formation is hereby described to a further detail.

It is the conclusion of the inventors that 1) TrPC state formation is a requirement for insulin cell formation in the normal pancreas, and can not be bypassed. 2) Notch signaling is a requirement for TrPC formation, and can not be bypassed, 3) the forward differentiation of insulin cells from a TrPC state requires loss of active Notch signaling. 4) The inventors posit that similar restrictions and requirements listed under point 1-4 above, also define forward differentiation of human embryonic stem cells to insulin producing cells. To the understanding of the inventors, the definition and prerequisite role of TrPC formation for human embryonic stem cells is novel, and is not covered by patent literature. To the understanding of the inventors, the proposition that Notch signaling activation prior to differentiation of insulin producing cells, is novel.

Considering the combinational codes of signaling during vertebrate regulative development, the inventors then hypothesized that Notch signaling would not independently define trPC state formation during pancreatic organ domain patterning, and therefore sought to identify other pathways that may help promote TrPC formation in the developing murine organ. Example 15 illustrates by principle the identification of canonical Wnt signaling as one such pathway.

### Example 14 (v)

### Generation of Pluripotent Derived Pancreatic Trunk Progenitor Cells

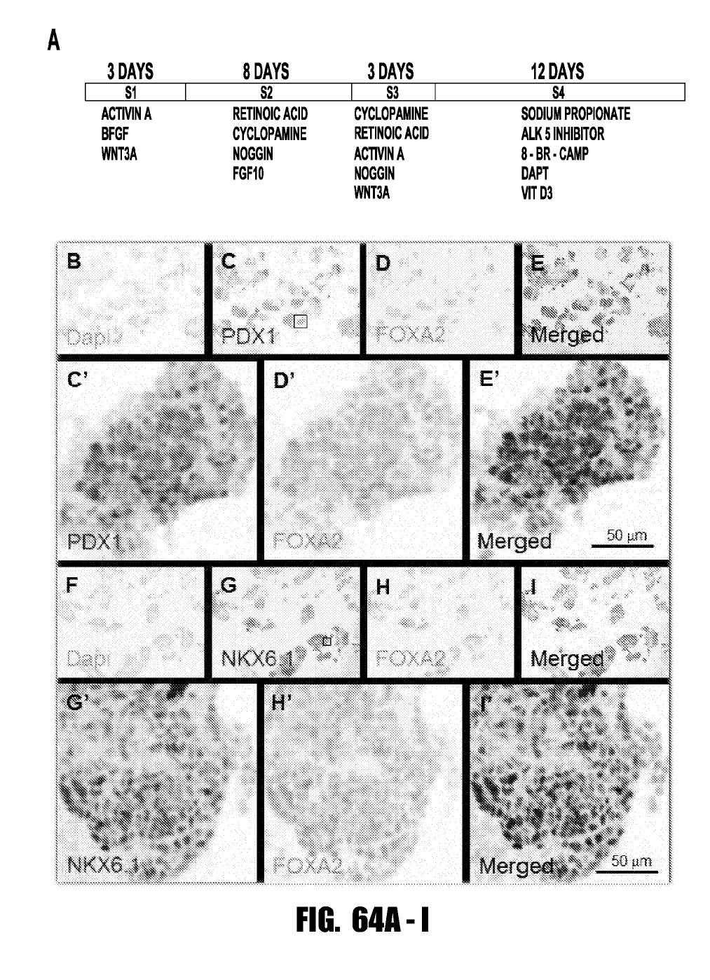

We next sought to evaluate a directed differention protocol for its ability to generate tunk progenitor cells. Pluripotent cultures were subjected to a 4 stage protocol as outlined in FIG. 64 panel A. Immunohistochemical analysis of these cultures was then performed to assess expression of the pancreatic progenitor marker of PDX1 and the Trunk progenitor marker NKX6.1. The widespread expression of the general endodermal marker FOXA2 demonstrated that the majority of the culture had taken an endodermal characteristic (FIG. 64 panels D and H). Further the majority of FOXA2+ cells found throughout the culture are both PDX1+ and NKX6.1+ as shown through in FIG. 64 panels C and panel G respectively. All together this indicates that the protocol used was efficiently generating pancreatic trunk progenitor cells.

### Example 15

### Identification and Utilization of Canonical Wnt Signaling During TrPC Induction

### Example 15 (i)

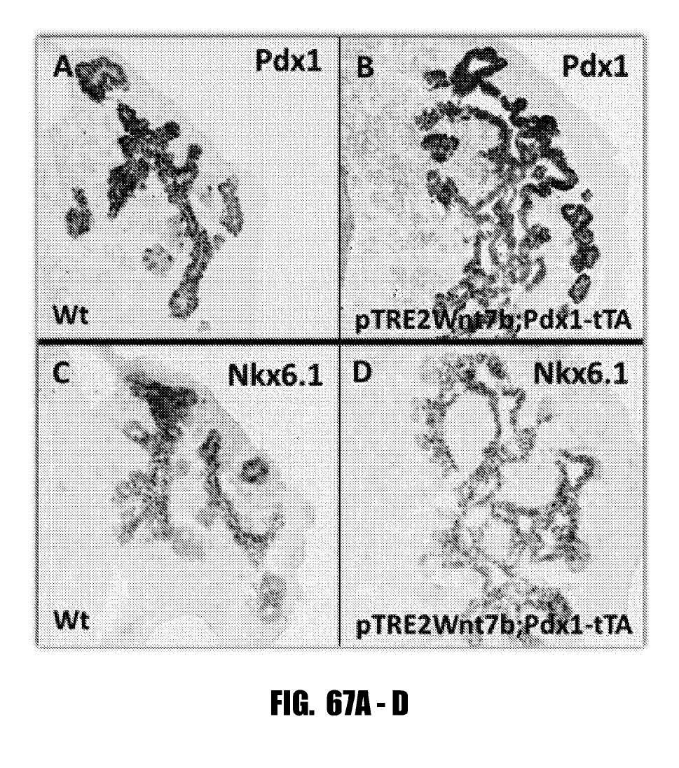

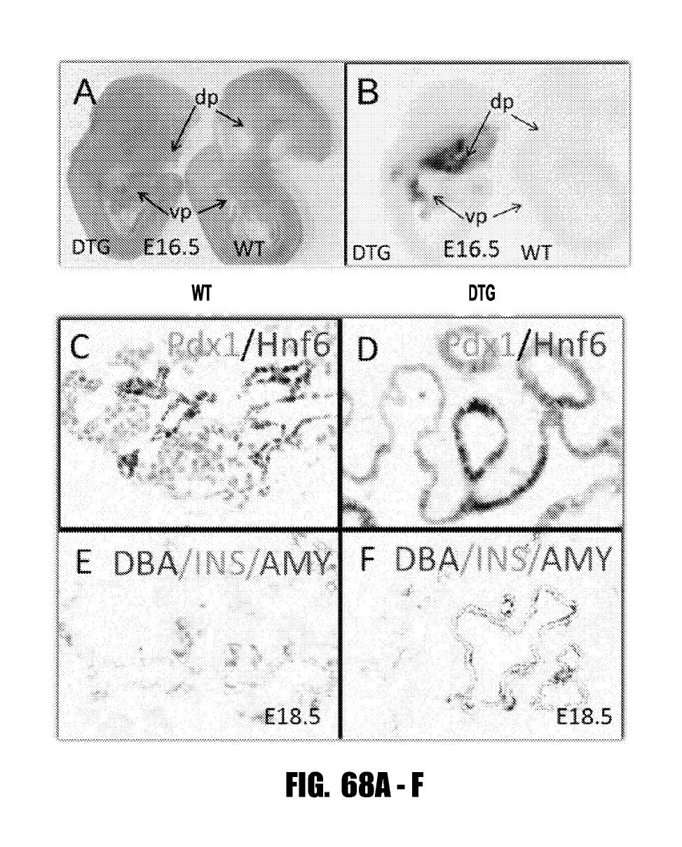

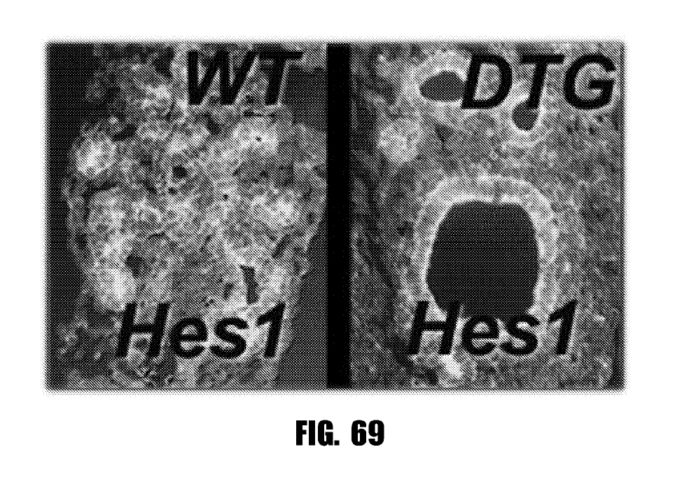

We sought to identify candidate signaling pathways operating in parallel with Notch during TrPC fate determination, and to that end created several murine gain-of-function models, similar to those described above for DN-MAML1 and Ngn3. Out of more than 10 models so screened, Wnt7b was observed to positively influence TrPC formation in pancreas. Wnt7b is expressed in pancreatic MPCs and not pancreas MEF-cells, arguing that TrPC formation involves at least one epithelial determinant factor, which secondarily helps explain why pancreas MEF cells, although useful for induction of pancreatic MPCs, is less useful for induction of TrPCs. Analysing the phenotype of pancreas overexpressing Wnt7b, we found that the canonical Wnt7b factor strongly induced a ductal program in pancreas, at expense of acinar and endocrine cells. This occurred through activation of Axin2 expression (not shown), which demonstrates further that the signaling downstream of Wnt7b is canonical type, as Axin2 induction is a B-catenin-induced canonical Wnt pathway target gene. We hypothesized that early TrPC induction had occurred, and by addressing TrPC markers (Nkx6.1/Hnf1beta/Hnf6) in the Wnt7b overexpressing organ at an earlier stage (E13.5), this was confirmed (FIGS. 67-69). The ongoing Wnt-signal negatively impacted the endocrine fate choice, so that only ducts descended from the TrPCs generated. However, when interrogating Notch activity, we found that Hes1 mRNA was widely expressed in the epithelial cells at E13.5 (FIGS. 67-69), showing that Notch signaling was propagated during canonical Wnt-signaling. These data strongly indicate a functional relationship between Notch, and Wnt, during TrPC formation in pancreas where elevation of either pathway results in increased ductal formation. Wnt/Notch pathway synergism is observed in other stem cell populations, such as intestine, as well as skin, eye and blood. Interestingly, Mam1-proteins are involved in direct activation of the Wnt-target beta-catenin (Alves-Guerra et al., Cancer Res. 67, 8690 (2007)) and the pathway interaction may involve Jagged (V. Rodilla et al., Proc. Natl. Acad. Sci. U.S.A 106, 6315 (2009)).

### Example 15 (ii)

### The Effect of Canonical Wnt Signaling Addition at the TrPC Induction Step During Directed Differentiation of Pluripotent Cells, and its Ability to Induce Single-Positive Insulin-Producing Cells

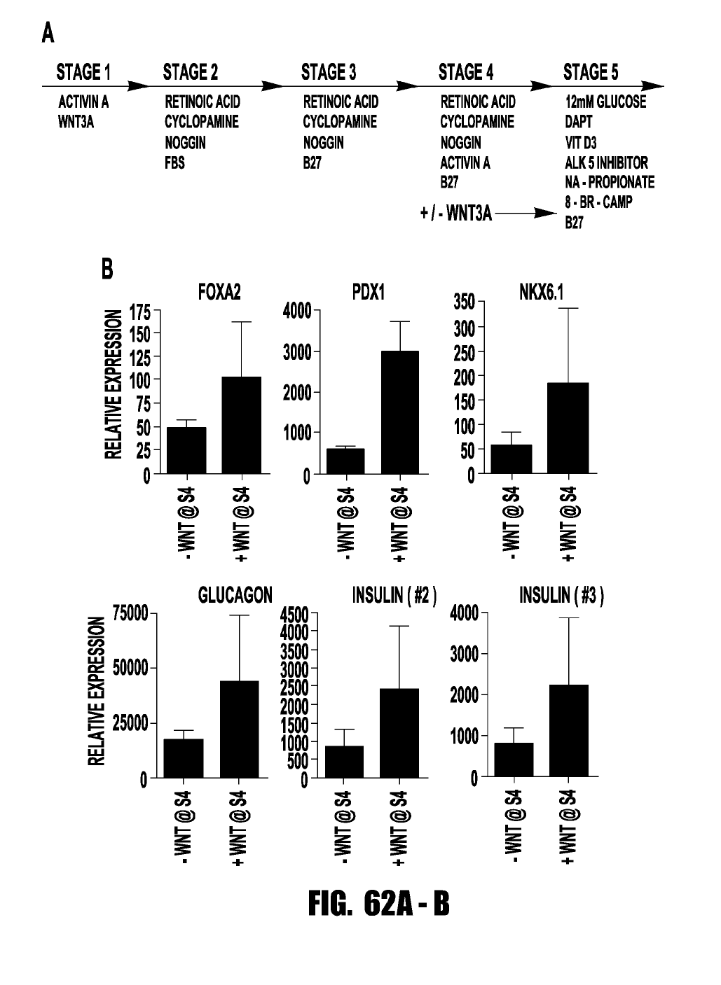

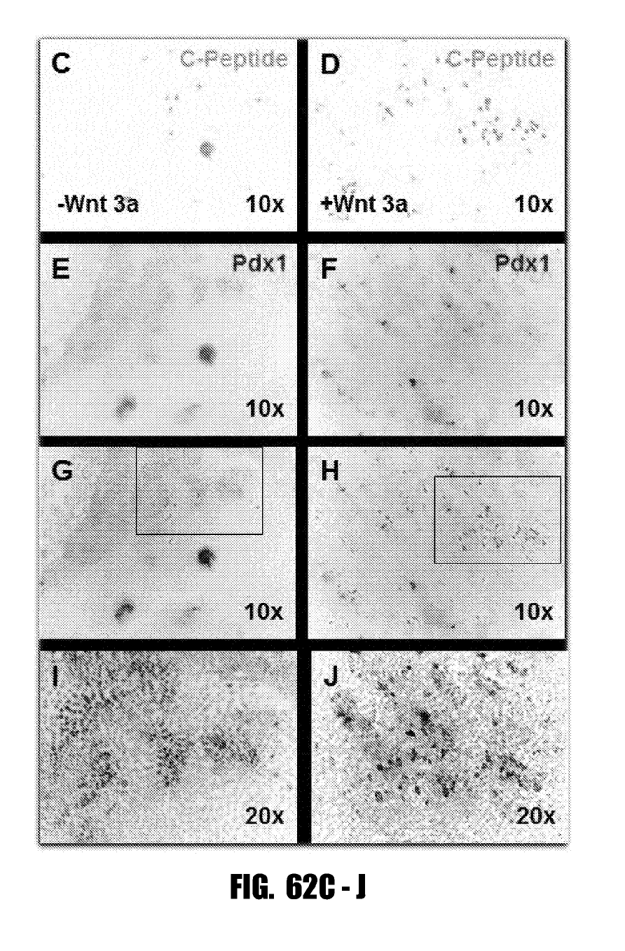

To investigate the role of a canonical Wnt input on a PDX1+ progenitor population, Wnt3a was administered to a stage 4 forward directed differentiating hES culture. Cultures in which Wnt3a was included at stage 4 displayed a significant increase in key TrPC factors including Pdx1 and Nkx6.1 (FIG. 62 panel B). The relative expression of Pdx1 was increased over ˜5 fold over stage 4 cultures lacking the canonical input and relative transcript levels reached ˜3000 fold over hES controls. While Nkx6.1 levels were modest in comparison, only reaching a ˜3.3 fold increase over the stage 4 cultures lacking the canonical input and a relative transcript fold increase of ˜200 over hES controls. Together this demonstrates a strong increase in a TrPC patterning event which is a prerequisite for the generation of endocrine lineages. Further, the forward differentiating cultures treated with a stage 4 canonical input displayed a complimentary increase in genes representative of both the alpha and beta cell fates as would be expected in a culture with an increased TrPC state. Both Glucagon and Insulin relative transcript levels were ˜2 fold greater in stage 4 cultures treated with Wnt3a as compared to stage 4 cultures lacking the canonical Wnt drive (FIG. 62 panel B). While there was a ˜5000 fold increase in Glucagon transcript levels and a ˜2500 fold increase in Insulin transcript levels when compared to hES levels. As a means to confirm the accuracy of the relative Insulin transcript levels two different set of primers were used and both primer sets produced similar results. In addition immunohistochemical analysis of these two different reactions was used to confirm the transcript analysis. Cultures which were treated with Wnt3a at stage 4 displayed a greater percentage of Pdx1⁺ cells throughout the culture (compare panels E and F of FIG. 62) as well as the expected increase in C-peptide⁺ cells (compare panels C and D of FIG. 62). All together this demonstrates that the inclusion of a canonical Wnt agonist in stage 4 directed differentiating cultures increases the percentage of pancreatic progenitors which take on a TrPC characteristic translating into an enhanced capacity to form endocrine subtypes, which in turn results in an increased presence of both Glucagon⁺ and Insulin⁺ cells within a stage 5 culture.

### Example 16

### Efficiency Comparison Between Directed Differentiation (Prior Art) and Direct Induction Protocols and the Effect of a Maturation Media Additive Set on the Generation of Pancreatic Endocrine Fates from Directed Differentiation of Pluripotent Cells

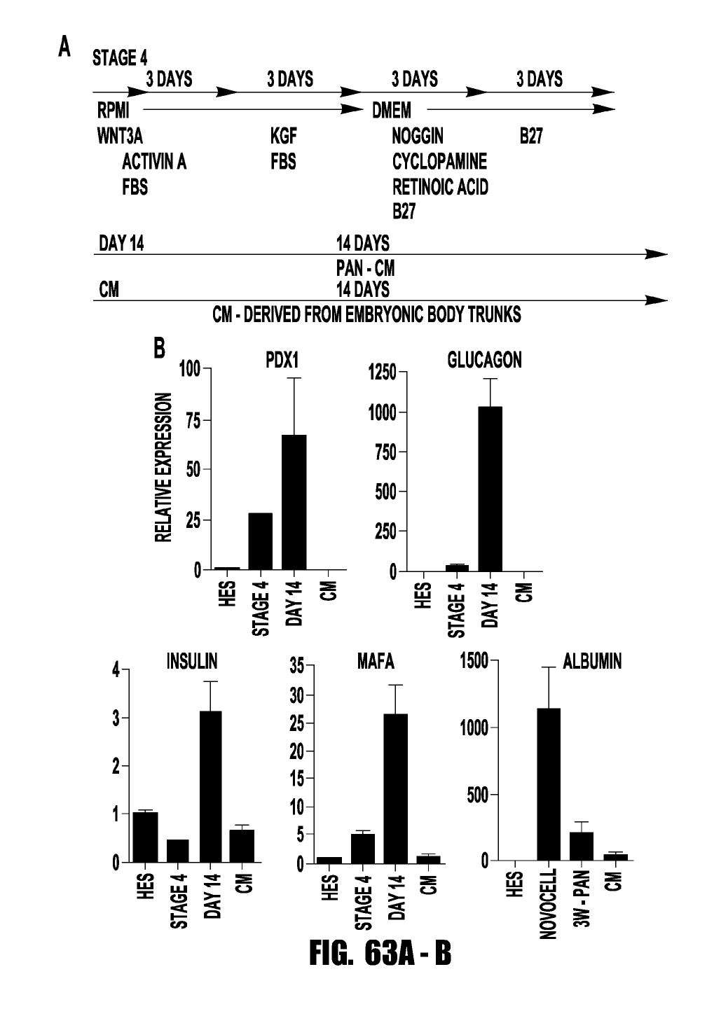

To ascertain the efficiency with which Pan-CM induces the formation of a pancreatic state within a forward differentiating culture as compared to that of a directed differentiation protocol pluripotent cultures maintained in Pan-CM were compared to pluripotent cultures taken through an established published protocol. The directed differentiation protocol used is outlined in Kroon et al. and the direct comparison between pluripotent cultures taken through the Kroon et al. protocol and pluripotent cultures which were incubated in the presence of Pan-CM for an equivalent length of time demonstrated that the latter differentiation protocol increased the resulting pancreatic phenotype. The relative Pdx1 levels obtained within cultures maintained in the presence of Pan-CM reached an approximate ˜75 increase over hES levels while cultures taken through the Kroon et al. protocol only showed an ˜25 fold increase (FIG. 63 panel B). The expression of Glucagon was largely increased over that observed in the directed differentiation protocol with relative levels reaching ˜1000 fold increased as compared to the ˜40 fold increase observed through the directed differentiation protocol. While neither of the beta-cell specific markers (Insulin and Mafa) assayed were substantial increased in either protocol, they both were slightly higher in cultures which were maintained in the Pan-CM with relative levels reaching ˜3 fold and ˜25 fold respectively over controls. It is important to note that while the pluripotent cultures maintained in the presence of Pan-CM displayed a stronger pancreatic phenotype induction over this directed differentiation protocol, that the effects observed were specific to the Pan-MEF culture and that pluripotent cultures maintained in conditioned medium obtained from MEF cells derived from embryonic body trunks did not display any significant increase in any of the markers assayed. All together this shows that pluripotent cultures maintained in the presence of Pan-CM were more capable of generating a pancreatic phenotype as compared to the directed differentiation protocol. However, it is important to note that the Kroon et al. protocol was performed on the H1 cell line in this series of experiments and that this protocol has been shown to have a reduced ability to function on the H1 cell line. This implies that the comparison may have been performed on a non-optimal cell line for the directed differentiation.

### Example 17

### Maturation Medium Improves the De Novo Insulin Production as Well as the Percentage of Single Positive Endocrine Cells

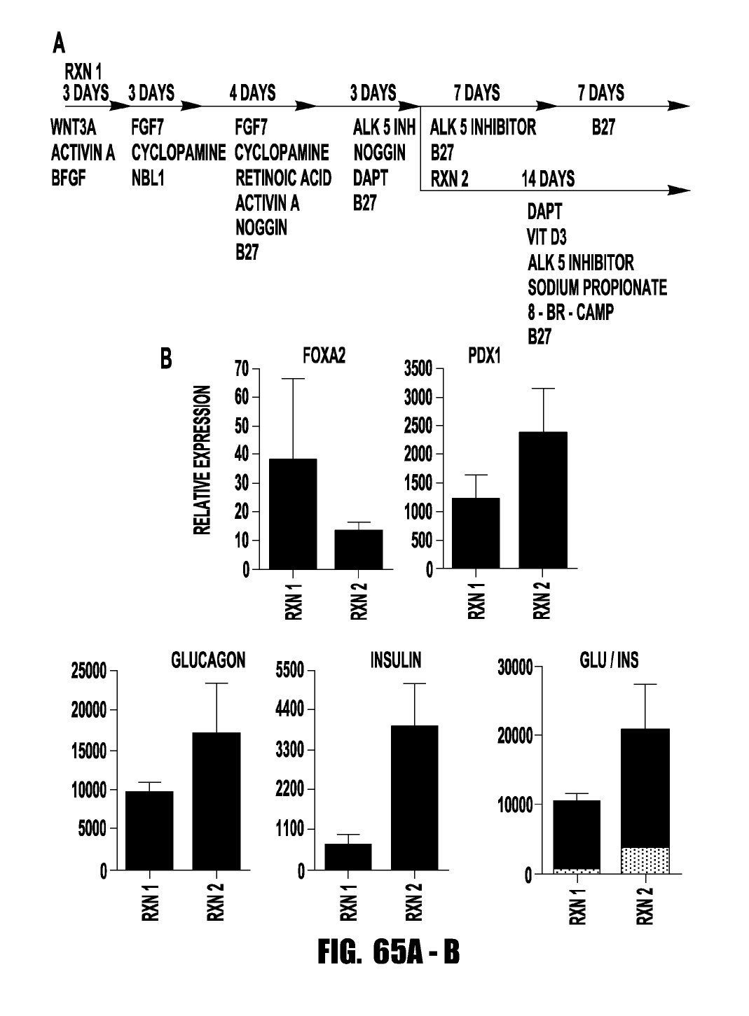

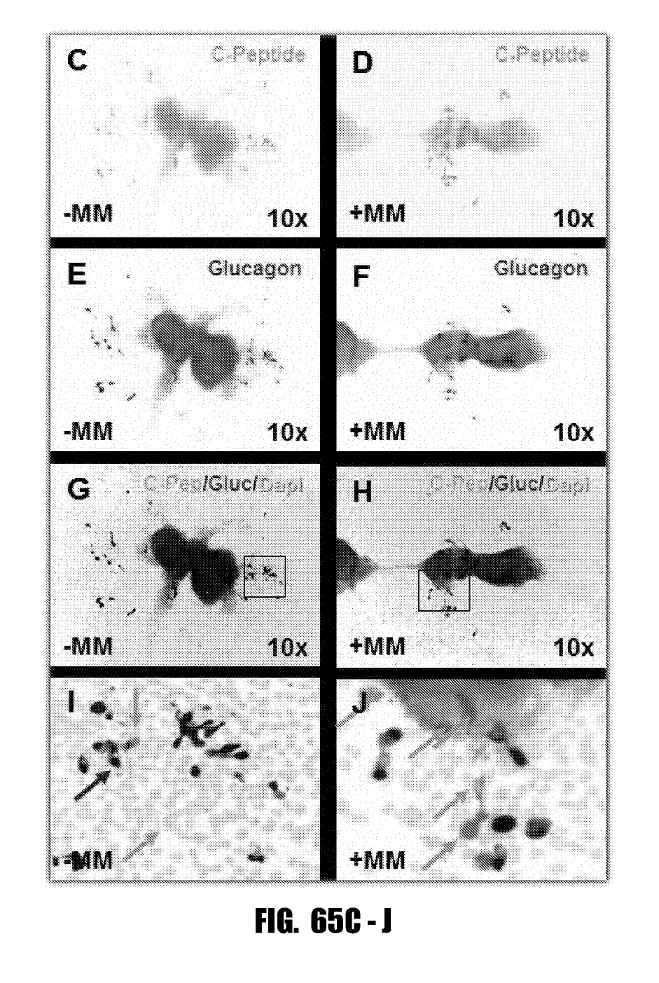

To establish the effects the maturation medium has on the generation of Insulin⁺ cells parallel directed differentiation protocols were performed on forward differentiating pluripotent cultures with stage 4 cultures either being incubated in the presence of our maturation medium or in the medium composition described in said directed differentiation protocol. The directed differentiation protocol used was slightly modified as shown in FIG. 67 panel A. RNA was extracted after stage 6 and analyzed for the endodermal marker FoxA2, the pancreatic progenitor marker Pdx1 and the two endocrine markers Insulin (a marker for the beta cell) and Glucagon (a marker for the alpha cell). A notably increase in relative Pdx1 levels is observed when the maturation medium is used throughout stages 5 and 6 with relative transcript levels reaching an ˜2500 fold increase as compared to the ˜1000 fold increase observed in the control reaction. An increase in the transcript levels of the endocrine genes was also observed with glucagon relative levels reaching ˜17000 fold increase and insulin reaching an ˜4000 fold increase as compared to the ˜10000 and ˜700 respective fold increases observed in the control reactions. When comparing the relative ratio of the two endocrine genes together this represents a dramatic increase in favor of the insulin producing cell. The relative expression of Glucagon was increased ˜1.5 fold as compared to the relative ˜6 fold increase in Insulin levels and the ratio between Glucagon and Insulin changed from 14.3 to 4.3 when the maturation medium was used throughout stages 5 and 6. While this still makes the relative expression of the Glucagon transcript 4.3 times more common than the relative level of the Insulin transcript it does represent a significant shift in favor of Insulin production and the Immunohistochemical analysis of these cultures demonstrates a great increase in the Ins+/Glu− cell (Compare panels I and J in FIG. 65). FIG. 65 shows representative cultures from both the control culture (denoted −MM) and the cultures grown in the presence of the maturation medium throughout stages 5 and 6 (denoted +MM). Panels G-H show the co-expression of the endocrine genes Glucagon and C-peptide well panels I-J show close up regions of these images focusing on the increased number of Ins+/Glu− (denoted by arrows) cells found in the cultures which were maintained in the maturation medium throughout stages 5 and 6. Occasional Ins+/Glu− cells are found within the control cultures (as indicated in FIG. 65 panel I) however the majority of the endocrine cells present within this culture is polyhormonal Gluc+/Ins+ cells.

### Example 18

### Molecular Design and Utilization of Stabilized Ngn3 to Bypass Ubiquitin-Mediated Degradation to Increase Endocrine Induction

We showed that Ngn3 is destabilized through the proteasomal system, and this is mediated by Notch, as well as Hes1. Proteasomal degradation relies on ubiquilnation of key lysine residues in proteins targeted for destruction. The addition of ubiquilin occurs due to the activity of an appropriate E3 ubiquitin ligase that is able to specifically bind the target protein, and perform the Ub-conjugation. Several bHLH factors are known to be regulated by ubiquilin-mediated turn-over, including close homologues to Ngn3, such as Ngn1 and MASH1. Importantly, it has been observed that it is possible to stabilize the bHLH target upon specific mutation (conversion of lysine into Arginine, preserving the net positive charge in the chain). We will here seek to take advantage of this knowledge and create Ngn3^{ST} Stabilized Ngn3) which is supposed to be refractory to Notch/Hes1 mediated destabilization.

### Methods

Ngn3 is a conserved protein of 214 amino acids in man, rat and mouse. The extremely conserved bHLH domain is composed of aa62•73 (basic domain), aa74•107 (long Helix 1), aa108•120 (Loop 1), and aa121-141 (Short Helix 2). There are a total of 7 lysines in Ngn3. One (K80) is located in the N-terminal, none are located in the C-terminal. These are of interest, and are mainly located in Helix 1 (K79, K88, K87), and the loop region (K118, K121).

The K-residues in Helix 1 are known from the ayslal structure of bHLH factors to contact DNA, where the Helix 1 helps in positioning on the DNA target of the heterodimer. On the other hand, the two lysine residues in the loop region (K118, K121) are accessible, even following complex formation with another bHLH partner. We did a threading analysis of Ngn3, obtaining the bHLH fold in 3D, and visualized the location of the lysines within the bHLH domain. We also compared this fold structure to 10 other (b)HLH DNA binding domains derived from other proteins (E2A, Hes1, Id1, Mist1/bhlhb8, Ptf1a, Olig2, NeuraD, MyoD, Myf5, Myogenin) and evaluated the presence of individual lysines in relation to expected/known stability of the target (e.g., E2A, Hes1, MyoD unstable, Mist1, NeuroD, Myogenin stable, others: unknown stability). We also compared the N-terminal residues in their ability to be acetylated; a protective modification occurring to cellular proteins, rendering them stable. To do this, we used the TerminNator neural network prediction server provided by the CNRS. For most proteins, acetylation of N-terminal Methionine occurs, in others, this modification does not happen. It is also possible that the N-terminal Methionine is removed, and acetylation occurs at the following residue (unless this is a Proline). The prediction for Ngn3 is that acetylation may occur at Alanine2, which predicts an intermediate stability of the factor. Absence of N-terminal acetylation leads to proteasomal decay, and prediction results for Ngn1 and Ngn2 indicated complete lack of N-terminal acetylation=unstable proteins. Hes1, a known unstable protein in all conditions analyzed, is not acetylated, and contains a Proline in Pos. 2.

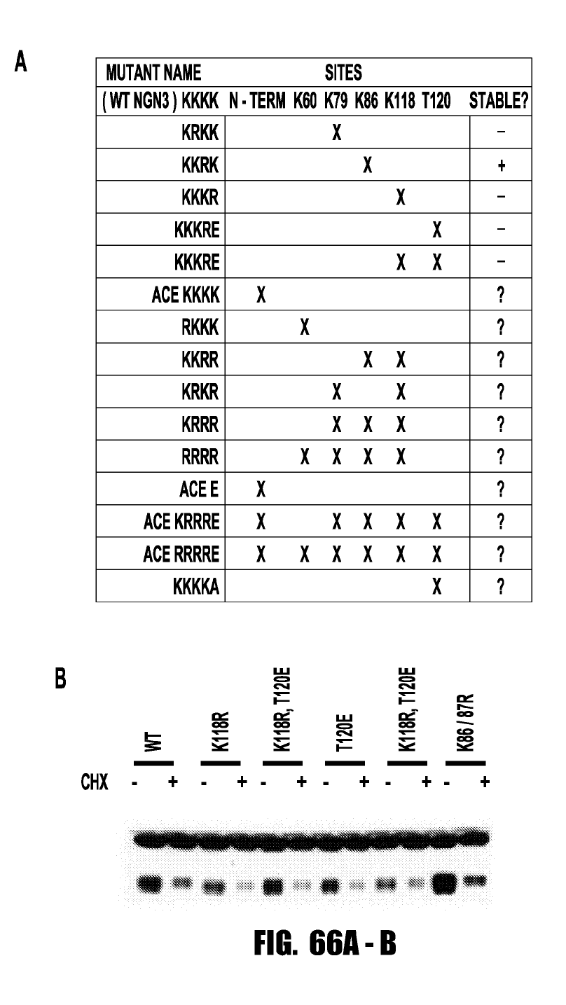

Combined, these analyses served to focus us on the presence of lysines in the Helix 1/loop region and highlighted the importance of N-terminal acetylation protection as well. Of note, The K¹¹⁸LTK¹²¹ motif in the loop is completely conserved in Ngn3 in vertebrates, including teleosts and amphibians, and also between Ngn family members. Our approach to stabilizing Ngn3 is therefore to create an exhaustive point-substitution series of K>R to cover the entire protein (FIG. 66A), and also create deletion mutants of the N-, and C-termini flanking the bHLH domain. Because K121 is completely conserved in the bHLH family group, and positioned so that it aligns with the Helix 1 axis, this residue is likely critical for the folding/binding dynamics of the HLH domain, and mutation of this may likely impact DNA binding/function. Such constraints are not evident for K¹¹⁸. We will also substitute the T120 residue in the KLTK loop to a Glutamic acid (E) to mimic a possible stabilizing phosphorylation event, which, based on phylogenetic comparisons to MASH1 may be of importance. We believe the reduction in net positive charge at the KLTK motif through phosphorylation T120 may impact the ubiquitination event, possibly interfering with E3 ligase binding. Finally, we will replace the N-terminal 4 residues with those of a stable bHLH factor (Myf5, sequence: M¹DMT⁴) which is predicted to convey Met1 acetylation and a much prolonged half-life. To generate a functional Ngn3ST version, we will derive a mutational series of Ngn3^{ST} versions, all of which will be compared to WT Ngn3 in the HepG2 stability assay. Data on the successful stabilization of Ngn3 is shown (FIG. 66B). This analysis indicates that as a lead, substituting K86/87 to Arginines lead to stabilization.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. A method of inducing pancreatic fates from human multipotent or pluripotent cells, the method comprising:
obtaining a cell population comprising human multipotent or pluripotent cells; and
providing the cell population with at least three of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of pancreatic precursor cells from the human multipotent or pluripotent cells.

2. The method of claim 1, wherein at least four of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor are provided to the cell population for a time effective to allow the differentiation of pancreatic precursor cells from the human multipotent or pluripotent cells.

3. The method of claim 1, wherein at least five of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor are provided to the cell population for a time effective to allow the differentiation of pancreatic precursor cells from the human multipotent or pluripotent cells.

4. The method of claim 1, wherein at least six of (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor are provided to the cell population for a time effective to allow the differentiation of pancreatic precursor cells from the human multipotent or pluripotent cells.

5-32. (canceled)

33. The method of claim 1, the pancreatic precursor cells expressing Hnf6, Nkx6.1, and Hnf1b.

34. The method of claim 33, the pancreatic precursor cells further expressing Sox9, Pdx1, and FoxA2.

35. The method of claim 1, the pancreatic precursor cell comprising an enriched population of trunk progenitor cells.

36-49. (canceled)

50. A method of producing an enriched population of insulin producing cells, the method comprising:
obtaining a cell population comprising human multipotent or pluripotent cells differentiated into the pancreatic lineage;
providing the cell population with (i) an CXCR4 agonist, (ii) an EGFR agonist, (iii) an FGFR agonist, (iv) an Activin receptor agonist or an agent that stimulates SMAD3, (v) an IL11R agonist or IL6R agonist, (vi) a notch agonist, (vii) an RXR agonist or RAR agonist, or (viii) a BMP inhibitor for a time effective to allow the differentiation of pancreatic precursor cells from the human multipotent or pluripotent cells; and
providing the pancreatic precursor cells with a maturation medium that promotes differentiation of the pancreatic precursor cells to insulin producing cells.

51. The method of claim 50, the maturation medium comprising an agent that increases the generation or stabilization of Ngn3 in the pancreas precursor cells.

52. The method of claim 50, the maturation medium comprising an agent that inhibits notch signaling of the pancreas precursor cells.

53. The method of claim 52, wherein the agent comprises at least one of MG132 or a γ-secretase inhibitor.

54. The method of claim 53, the maturation medium further comprising an agent that promotes the generation of intracellular cAMP.

55. The method of 53, the agent that promotes the generation of intracellular cAMP comprising at least one of 8-Br-cAMP, Forskolin, an Adra2a agonist, epinephrine, adrenaline, Galanin, Galr1 activators, Glp1R agonists, Glp1, or exendin.

56. The method of claim 50, the maturation medium further comprising a Ffar2 agonist.

57. The method of claim 55, the Ffar2 agonist comprising at least one short-chain fatty acid including propionate or butyrate.

58. The method of claim 50, the maturation medium further comprising a VDR agonist.

59. The method of claim 57, the VDR agonist comprising Vitamin D3 or metabolites thereof.

60. The method of claim 50, the maturation medium further comprising glucose.

61. The method of claim 50, the maturation medium further comprising at least one of propionate, 8Br-cAMP, vitamin D3, or glucose.

62. The method of claim 50, further comprising providing the pancreatic precursor cells differentiated from the human multipotent or pluripotent cells with a cell growth medium comprising a Wnt signaling pathway activation agent prior to providing the pancreatic precursor cells with a maturation medium that promotes differentiation of the pancreatic precursor cells to insulin producing cells.

63-161. (canceled)

162. A method of treating a subject, the method comprising of administering a pancreatic cell produced by the method of claim 1 and administering the pancreatic cells to the subject.

163. A method of treating a subject, the method comprising administering a pancreatic insulin producing cell produced by the method of claim 50 and administering the pancreatic insulin producing cells to the subject.

164-165. (canceled)

166. The method of claim 163, in which the alginate encapsulation of the cells has occurred via an alginate-microbead formation process.

167. The method of claim 163, the immunoprotective barrier comprising a macroencapsulation device.

168-171. (canceled)
